(19) United States
(12) Patent Application Publication
(10) Pub. No.: US 2020/0088748 A1
(43) Pub. Date: Mar. 19, 2020

(54) METABOLIC PROFILING OF FIXED SAMPLES

(71) Applicant: Dana-Farber Cancer Institute, Inc., Boston, MA (US)

(72) Inventors: Massimo Loda, Belmont, MA (US); Stefano Cacciatore, Woodstock (ZA)

(73) Assignee: Dana-Farber Cancer Institute, Inc., Boston, MA (US)

(21) Appl. No.: 16/467,897

(22) PCT Filed: Dec. 8, 2017
(86) PCT No.: PCT/US2017/065294

§ 371 Date: Jun. 7, 2019

Related U.S. Application Data
(60) Provisional application No. 62/431,627, filed on Dec. 8, 2016.

(51) Int. Cl. G01N 33/92; G01N 1/30; G01N 33/50

(52) U.S. Cl. CPC G01N 33/92; G01N 1/30; G01N 2800/60; G01N 2001/305; G01N 2570/00; G01N 33/5005

(57) ABSTRACT

The invention provides methods and compositions to evaluate biological samples. The methods comprise obtaining a formalin fixed paraffin embedded (FFPE) preparation of the biological sample, and detecting the presence of one or more metabolites in the FFPE preparation.

### RELATED APPLICATIONS

This application claims the benefit of U.S. provisional application No. 62/431,627, filed on Dec. 8, 2016, the entire disclosure of which are incorporated by reference herein.

### FEDERALLY SPONSORED RESEARCH

This invention was made with government support under grant number 11498838 awarded by the Department of Defense and grant numbers 2R01CA131945 and P50 CA90381 awarded by National Institutes of Health. The government has certain rights in the invention.

### BACKGROUND

Metabolic profiling has significantly contributed to a deeper understanding of the biochemical metabolic networks and pathways in cells. A metabolite profile provides a snapshot of the complex interactions between genetic alterations, enzymatic activity, and biochemical reactions—revealing unique patterns of information that may be driven by specific genetic events. Metabolic profiling represents an extraordinary tool to profile cellular abnormalities and advance personalized medicine.

### SUMMARY

Aspects of the technology disclosed herein relate to methods of evaluating a biological sample, e.g., a formalin-fixed paraffin-embedded (FFPE) preparation of a biological sample. In some aspects, the method comprises obtaining an FFPE preparation of the biological sample and detecting the presence of one or more metabolites in the FFPE preparation, wherein the one or more metabolites are members of a class selected from the classes listed in Table 1. In some aspects, the method comprises obtaining an FFPE preparation of the biological sample and detecting the presence of one or more metabolites in the FFPE preparation, wherein the one or more metabolites are members of a subclass selected from the subclasses listed in Table 1. In some aspects, the method comprises obtaining an FFPE preparation of the biological sample and detecting the presence of one or more metabolites in the FFPE preparation, wherein the one or more metabolites comprise a substituent group selected from the substituents listed in Table 1.

In some embodiments, the one or more metabolites are lipids. In some embodiments, the one or more metabolites are unsaturated fatty acids. In some embodiments, the one or more metabolites are hydrophobic metabolites. In some embodiments, the one or more metabolites are selected from taurine, 1-palmitoylglycerophosphoinositol, pyroglutamine, oxidized glutathione, dihomo-linoleate, creatinine, 1-linoleoylglycerophosphoethanolamine, eicosenoate, and 10-nonadecenoate.

In some embodiments, the one or more metabolites do not include one or more metabolites that are members of a class listed in Table 2. In some embodiments, the one or more metabolites do not include one or more metabolites that are members of a subclass listed in Table 2. In some embodiments, the one or more metabolites are not peptides. In some embodiments, the one or more metabolites are not steroids.

In some embodiments, the presence of 2 or more metabolites are detected in the FFPE preparation. In some embodiments, the presence of 5 or more metabolites are detected in the FFPE preparation. In some embodiments, the presence of 10 or more metabolites are detected in the FFPE preparation. In some embodiments, the presence of 25 or more metabolites are detected in the FFPE preparation.

In some embodiments, methods provided herein further comprise measuring an expression level of the one or more metabolites in the FFPE preparation. In some embodiments, the methods further comprise comparing the expression level of the one or more metabolites measured in the FFPE preparation to an expression level of the one or more metabolites measured in a control sample. In some embodiments, the one or more metabolites are selected from the metabolites listed in Table 3. In some embodiments, the FFPE preparation and the control sample are biological samples of the same subject. In some embodiments, the FFPE preparation and the control sample are biological samples of different subjects.

In some embodiments, the control sample is a biological sample of non-cancerous tissue. In such embodiments, methods provided herein further comprise identifying the FFPE preparation as comprising cancerous tissue when the one or more metabolites are differentially expressed in the FFPE preparation when compared to the control sample.

In some embodiments, the control sample is a biological sample of cancerous tissue. In such embodiments, methods provided herein further comprise identifying the FFPE preparation as not comprising cancerous tissue when the one or more metabolites are differentially expressed in the FFPE preparation when compared to the control sample.

In some embodiments, the one or more differentially expressed metabolites are selected using a criteria of p-value <0.05. In some embodiments, the one or more differentially expressed metabolites are selected using a criteria of false discovery rate <0.1.

In some embodiments, methods provided herein further comprise determining tumor status of the biological sample based on the measuring of one or more metabolites in the FFPE preparation.

In some embodiments, the biological sample is a tissue sample. In some embodiments, the tissue sample is a prostate tissue sample.

In some embodiments, methods provided herein further comprise extracting the one or more metabolites from the FFPE biological sample. In some embodiments, the one or more metabolites are extracted using a methanol solution. In some embodiments, the methanol solution comprises 80% methanol.

In some embodiments, methods provided herein further comprise staining the FFPE biological sample for histological analysis. In some embodiments, the FFPE biological sample is stained using H&E stain.

In some embodiments, methods provided herein further comprise measuring the one or more metabolites in two or more portions of the FFPE preparation of the biological sample.

In some embodiments of any one of the methods described herein, the FFPE preparation is mounted on a slide. In some embodiments, FFPE preparation that is mounted on a slide is a section of tissue. In some embodiments, FFPE preparation that is mounted on a slide comprises cells (e.g., those cultured on a surface). In some embodiments, extracting one or more metabolites from an FFPE biological sample that is mounted on or attached to a slide when the slide is situated in a cassette. In some embodiments, a cassette reduces the volume of extraction solution so as to increase the yield of extracted metabolites in the solution. In some embodiments, a cassette has the design depicted in FIG. 6. Accordingly, provided herein is a cassette to minimize the extraction volume, or increase the extraction yield of metabolites, when extracting one or more metabolites from an FFPE biological sample when it is attached to a slide. In some embodiments, the volume of extraction solution that is added into a cassette with a slide to which an FFPE biological sample is attached is 0.5-20 ml (e.g., 0.5-10, 1-5, 2-12, 5-10, 5-15, 10-20, 12-20, or 16-20 ml). In some embodiments, the volume of extraction solution that is added into a cassette with a slide to which an FFPE biological sample is attached is 10 ml.

In some embodiments of any one of the methods described herein, one or more metabolites are detected in an FFPE preparation and normalized (e.g., when comparing to another FFPE preparation) by weight of the sample assessed (e.g., per ng of tissue). In some embodiments, normalization is done using a housekeeping metabolite. In some embodiments, a housekeeping metabolite is cytidine 50-diphosphocholine. In some embodiments, normalization between a test sample (e.g., diseased tissue) and a control sample (e.g., non-diseased tissue) is done using a housekeeping metabolite (e.g., cytidine 50-diphosphocholine). In some embodiments, a housekeeping metabolite is a metabolite selected from Table 27. A house keeping metabolite is one whose expression does not change between the conditions that are being compared (e.g., diseased and non-diseased tissue).

In some embodiments of any one of the methods described herein, one or more metabolites are detected in an FFPE preparation and normalized (e.g., when comparing to another FFPE preparation) by the number of a particular tissue compartment (e.g., epithelial cells), cellular compartment (e.g., nucleus), or a particular area of tissue compartment (e.g., area of epithelium or stromal compartment). In some embodiments, metabolite expression data is normalized using one or more metabolites selected from Table 31, Table 32 or Table 33. In some embodiments, metabolite expression data is normalized using fructose, glycine, guanine, or phenylalanine. Metabolites that can be used to normalize metabolite expression data may be identified using a combination of metabolite expression analysis and image analysis, and optionally correlating the metabolite expression levels to the image analysis unit (e.g., number of cells, area of cells, or number of nuclei).

### BRIEF DESCRIPTION OF THE DRAWINGS

The skilled artisan will understand that the figures, described herein, are for illustration purposes only. It is to be understood that, in some instances, various aspects of the invention may be shown exaggerated or enlarged to facilitate an understanding of the invention. In the drawings, like reference characters generally refer to like features, functionally similar and/or structurally similar elements throughout the various figures. The drawings are not necessarily to scale, emphasis instead being placed upon illustrating the principles of the teachings. The drawings are not intended to limit the scope of the present teachings in any way.

The features and advantages of the present invention will become more apparent from the detailed description set forth below when taken in conjunction with the drawings.

When describing embodiments in reference to the drawings, direction references (“above,” “below,” “top,” “bottom,” “left,” “right,” “horizontal,” “vertical,” etc.) may be used. Such references are intended merely as an aid to the reader viewing the drawings in a normal orientation. These directional references are not intended to describe a preferred or only orientation of an embodied device. A device may be embodied in other orientations.

As is apparent from the detailed description, the examples depicted in the figures and further described for the purpose of illustration throughout the application describe non-limiting embodiments, and in some cases may simplify certain processes or omit features or steps for the purpose of clearer illustration.

FIG. 1. Paraffinization and extraction protocol. A schematic overview of the steps of formalin-fixation, paraffin-embedding, and metabolite extraction

FIG. 2A. A schematic overview of the protocol used to prepare frozen and FFPE cell samples of isogenic cell lines.

FIG. 2B. Venn diagram showing the intersection between frozen and FFPE metabolomic data in the experimental settings.

FIG. 2C. Box-and-whisker plot representing the relative signal intensity of all shared metabolites found in frozen and FFPE samples.

FIG. 2D. Bar plot of the metabolite number found in frozen and FFPE samples. The metabolites are categorized according to the class membership. The percentage above each bar represents the number of detectable metabolites (of each class) found in FFPE compared to frozen samples.

FIG. 2E. Correlation plots between FFPE cell replicates and between frozen and FFPE cell samples.

FIG. 2F. Box-and-whisker plots of the correlation coefficients, categorized by class membership, between frozen replicates, FFPE replicates, and frozen and FFPE samples.

FIG. 2G. Heatmap of selected metabolites from cell line samples. Hierarchical clustering (Ward method) based on KODAMA dissimilarity matrix is used for unsupervised classification. The phenotypic labels of the samples (i.e., LNCaP and LNCaP-Abl) are indicated as a band on top of the heatmap.

FIG. 2H. Heatmap of selected metabolites from cell line samples shows forty-two metabolites that were significantly different in both frozen and FFPE samples between LNCaP and LNCaP-Abl cell lines.

FIG. 3A. A schematic diagram of the human prostate samples used.

FIG. 3B. Venn diagram showing the intersection between frozen and FFPE metabolomic data in the experimental settings.

FIG. 3C. Bar plot of the metabolite number found in frozen and FFPE samples. The metabolites are categorized according to the class membership. The percentage above each bar represents the number of detectable metabolites (of each class) found in FFPE compared to frozen samples.

FIG. 3D. Correlation plots between FFPE cell replicates and between frozen and FFPE cell samples.

FIG. 3E. Heatmap of selected metabolites from cell line samples. Hierarchical clustering (Ward method) based on KODAMA dissimilarity matrix is used for unsupervised/semi-supervised classification. The phenotypic labels of the samples (i.e., normal and tumor tissue) are indicated as a band on top of the heatmap.

FIG. 4A. The top panel shows a schematic overview of the samples analyzed in the trainings set. On the right side, OSC-PLS scores plot of the FFPE biopsy punches of the trainings set. The bottom panel shows a schematic overview of a modified Leave-One-Out cross-validation procedure.

FIG. 4B. A schematic overview of the samples analyzed in the validation set (i.e., FFPE biopsy punches and section). On the right side, OSC-PLS projection scores plot of the FFPE samples of the validation set.

FIG. 4C. Tissue images for tissue segmentation analysis.

FIG. 4D. Tissue images for tissue segmentation analysis.

FIG. 5. Modified Leave-One-Out Cross-validation. A schematic overview of the procedure for cross-validation used.

FIG. 6. A schematic of top view of one embodiment of a cassette for metabolite extraction.

FIG. 6A A cross-sectional view of the cassette of FIG. 6 taken along line 6A.

FIG. 6B A cross-sectional view of the cassette of FIG. 6B taken along line 6B.

FIG. 7 A cross-sectional view of a slide being inserted into a cassette for metabolite extraction.

FIG. 8A. A schematic overview of a protocol used to prepare frozen, FF, and FFPE cell samples and to collect the supernatant formalin solution.

FIG. 8B. Venn diagrams showing the intersection among sample sets and relative bar plot of the metabolite number categorized according to the class membership.

FIG. 9. Identification of molecular signatures. Non-negative matrix factorization (NMF) was run with profiles of 39 metabolites from 16 human prostate FFPE samples (8 biopsy punch samples and 8 tissue section from the validation set).

FIG. 10. NMF molecular signatures. Molecular signature present in FFPE tissue section and correlation with tumor percentage.

### DETAILED DESCRIPTION

Among other aspects, the present disclosure provides techniques capable of identifying metabolites in FFPE samples. The process of generating an FFPE preparation of a biological specimen generally requires the use of chemically reactive conditions, which can make obtaining reliable metabolic data from these preparations difficult. The methods provided in the disclosure relate, at least in part, to the recognition that certain metabolites are capable of being detected and/or measured in FFPE preparations of biological samples. As described herein, such methods were utilized to successfully measure levels of differentially expressed metabolites, e.g., to determine tumor status in the biological sample. Surprisingly, the mild conditions applied in the preparation and/or extraction techniques presented herein allow for secondary analyses to be conducted on the same FFPE preparation of the biological sample, permitting a comprehensive analysis of the metabolic state and tissue architecture in a single biological sample.

Metabolites are small molecule compounds, such as substrates for enzymes of metabolic pathways, intermediates of such pathways or the products obtained by a metabolic pathway. Metabolic pathways are well known in the art, and include, for example, citric acid cycle, respiratory chain, glycolysis, gluconeogenesis, hexose monophosphate pathway, oxidative pentose phosphate pathway, production and (3-oxidation of fatty acids, urea cycle, amino acid biosynthesis pathways, protein degradation pathways, amino acid degrading pathways, and biosynthesis or degradation of lipids, proteins, and nucleic acids. Accordingly, small molecule compound metabolites may be composed of, but are not limited to, the following classes of compounds: alcohols, alkanes, alkenes, alkynes, aromatic compounds, ketones, aldehydes, carboxylic acids, esters, amines, imines, amides, cyanides, amino acids, peptides, thiols, thioesters, phosphate esters, sulfate esters, thioethers, sulfoxides, ethers, or combinations or derivatives of the aforementioned compounds.

In some embodiments, a metabolite has a molecular weight of 50 Da (Dalton) to 30,000 Da, e.g., less than 30,000 Da, less than 20,000 Da, less than 15,000 Da, less than 10,000 Da, less than 8,000 Da, less than 7,000 Da, less than 6,000 Da, less than 5,000 Da, less than 4,000 Da, less than 3,000 Da, less than 2,000 Da, less than 1,000 Da, less than 500 Da, less than 300 Da, less than 200 Da, less than 100 Da. In some embodiments, a metabolite has a molecular weight of at least 50 Da. In some embodiments, a metabolite has a molecular weight of 50 Da up to 1,500 Da. In some embodiments, a metabolite contemplated in the techniques described herein is any metabolite isolated from or identified in a biological sample.

As used herein, in some embodiments, the term “biological sample” refers to a sample derived from a subject, e.g., a patient. Non-limiting examples of a biological sample include blood, serum, urine, and tissue. In some embodiments, the biological sample is tissue. Obtaining a biological sample of a subject means taking possession of a biological sample of the subject. Obtaining a biological sample from a subject, in some embodiments, means removing a biological sample from the subject. Therefore, the person obtaining a biological sample of a subject and measuring a profile of metabolites in the biological sample does not necessarily obtain the biological sample from the subject. In some embodiments, the biological sample may be removed from the subject by a medical practitioner (e.g., a doctor, nurse, or a clinical laboratory practitioner), and then provided to the person measuring a profile of metabolites. The biological sample may be provided to the person measuring a profile of metabolites by the subject or by a medical practitioner (e.g., a doctor, nurse, or a clinical laboratory practitioner). In some embodiments, the person measuring a profile of metabolites obtains a biological sample from the subject by removing the sample from the subject.

As used herein, a “subject” refers to any mammal, including humans and non-humans, such as primates. In some embodiments, the subject is a human, and has been diagnosed or is suspected of having a tumor. In some embodiments, the subject may be diagnosed or is suspected of having a prostate tumor.

It is to be understood that a biological sample may be processed in any appropriate manner to facilitate measuring expression levels of metabolic profiles. For example, in some embodiments, biochemical, mechanical and/or thermal processing methods may be appropriately used to isolate a biomolecule of interest from a biological sample. The expression levels of the metabolites may also be determined in a biological sample directly. The expression levels of the metabolites may be measured by performing an assay, such as but not limited to, mass spectroscopy, positron emission tomography, gas chromatography (GC-MS) or HPLC liquid chromatography (LC-MS). Other appropriate methods for determining levels of metabolites will be apparent to the skilled artisan.

In some aspects, techniques described herein may be used to detect the presence of one or more metabolites in a biological sample (e.g., an FFPE preparation of a biological sample). In some embodiments, the one or more metabolites may be classified according to conventional classification constructs, nomenclature known in the art, and/or structural features of the one or more metabolites. For example, in some embodiments, the one or more metabolites may include 10-nonadecenoate and 1-palmitoyl glycerophosphoinositol. In some embodiments, 10-nonadecenoate and 1-palmitoyl glycerophosphoinositol can both be classified as fatty acids (e.g., Class: “Fatty Acids”). In some embodiments, 10-nonadecenoate and 1-palmitoyl glycerophosphoinositol can be further subdivided into subclasses according to the structural properties of each molecule. In such embodiments, 10-nonadecenoate may be classified as an unsaturated fatty acid (e.g., Subclass: “Unsaturated Fatty Acids”) and 1-palmitoyl glycerophosphoinositol may be classified as a lysophosphatidylinositol (e.g., Subclass: “Lysophosphatidylinositols”). In some embodiments, 10-nonadecenoate and 1-palmitoyl glycerophosphoinositol can be further subdivided according to the substituents present in each molecule. In such embodiments 10-nonadecenoate may be classified according to its carboxylate substituent (e.g., Substituent: “Carboxylic Acid”) and 1-palmitoyl glycerophosphoinositol may be classified according to its ester substituent (e.g., Substituent: “Fatty Acid Ester”). Accordingly, in some embodiments, classifying the one or more metabolites may be used to assess the biological sample and/or the techniques used in detecting the one or more metabolites (e.g., methods of extraction, methods of measuring metabolites, etc.).

In some embodiments, the one or more metabolites are members of a class selected from the classes listed in Table 1. In some embodiments, the one or more metabolites are members of a subclass selected from the subclasses listed in Table 1. In some embodiments, the one or more metabolites comprise a substituent group selected from the substituents listed in Table 1.

TABLE 1
Classification of Metabolites Detected in FFPE Biological Samples

CLASS
Peptides
Fatty Acids and Conjugates
Glycerophospholipids
Glycerolipids
Monosaccharides
Purine Nucleotides
Pyrimidine Nucleotides
Amino Acids and Derivatives
Benzyl Alcohols and Derivatives
Hydroxy Acids and Derivatives
Lineolic Acids and Derivatives
Imidazopyrimidines
Carboxylic Acids and Derivatives
Pyrimidine Nucleosides and Analogues
Purine Nucleosides and Analogues
Pteridines and Derivatives
Pyridines and Derivatives
Alkylamines
Sphingolipids
Alcohols and Polyols
Organic Phosphoric Acids and Derivatives
Fatty Acid Esters
Sugar Alcohols
Cyclic Alcohols and Derivatives
Sugar Acids and Derivatives
Benzoic Acid and Derivatives
Fatty Amides
Keto-Acids and Derivatives
Peptidomimetics
Trisaccharides
SUBCLASS
Unsaturated Fatty Acids
Straight Chain Fatty Acids
Branched Fatty Acids
Alpha Amino Acids and Derivatives
Beta Amino Acids and Derivatives
N-acyl-alpha Amino Acids and Derivatives
GlycoAmino Acids and Derivatives
Phosphatidylinositols
Phosphatidylserines
Lysophosphatidylcholines
Lysophosphatidylethanolamines
Lysophosphatidylserines
Lysophosphatidylinositols
Hexoses
Trihexoses
Pentoses
Purine Nucleosides and Analogues
Purine Ribonucleoside Monophosphates
Purine Ribonucleoside Diphosphates
Purine 2′-deoxyribonucleosides and Analogues
Pyrimidine Nucleosides and Analogues
Pyrimidine Nucleotide Sugars
Pyrimidine Ribonucleoside Diphosphates
Pyrimidine Ribonucleoside Triphosphates
Pyrimidine 2′-deoxyribonucleosides and Analogues
Phenylpyruvic Acid Derivatives
Lineolic Acids and Derivatives
Sphingolipids
Sphingomyelins
Monoacylglycerols
Acyl Carnitines
Acyl Glycines
Polyamines
Xanthines
Sugar Alcohols
Sugar Acids and Derivatives
Alpha Hydroxy Acids and Derivatives
Beta Hydroxy Acids and Derivatives
Cyclitols and Derivatives
Gamma Keto-Acids and Derivatives
Hybrid Peptides
Dicarboxylic Acids and Derivatives
Tricarboxylic Acids and Derivatives
SUBSTITUENT
secondary carboxylic acid amide
carboxamide group
N-substituted alpha amino acid
N-acyl alpha amino acid
alpha amino acid or derivative
saccharide
fatty acid ester
pyrimidine
organic hypophosphite
phosphoric acid ester
organic phosphite
triose monosaccharide
acyclic alkene
pyrimidone
aminopyrimidine
carboxylic acid
organic pyrophosphate
bicyclohexane
oxolane
n glycosyl compound
carboxylic acid ester
decaline
acetal
Carboxylic acid salt
X1 phosphoribosyl imidazole
sesterterpene
O glycosyl compound
Amphetamine or derivative
Hemiacetal
Hydropyrimidine
Purine
Imidazopyrimidine
Polyamine
Quaternary ammonium salt
X1.3 aminoalcohol
Disaccharide phosphate
Phenol derivative
Phenol
phosphoethanolamine
Oxane
Pyrrole
Dicarboxylic acid derivative
choline
imidazole
Monosaccharide phosphate
Pentose monosaccharide
Glycosyl compound
phosphocholine
Alpha hydroxy acid
Glycerol 3 phosphocholine
cyclohexane
hypoxanthine
Cyclic alcohol
guanidine
Imidazolyl carboxylic acid derivative
phenethylamine
benzoyl
thioether
carnitine
X1.2 aminoalcohol
X1.2 diol
N acylglycine
Secondary alcohol
Primary carboxylic acid amide
ketone
Urea
Short chain hydroxy acid
allyl alcohol
primary aliphatic amine (alkylamine)

In some embodiments, methods described herein relate to the detection of at least one metabolite that is capable of being classified according to at least one of the classes, at least one of the subclasses, and at least one of the substituents listed in Table 1. In some embodiments, methods described herein relate to the detection of a plurality of metabolites, each of which are capable of being classified according to at least one of the classes, subclasses, and substituents listed in Table 1. In some embodiments, the plurality of metabolites contemplated in the methods described herein include a set of metabolites that are representative of at least 2, at least 3, at least 4, at least 5, at least 10, at least 15, at least 20, at least 25, at least 30 of the classes listed in Table 1. In some embodiments, the plurality of metabolites contemplated in the methods described herein include a set of metabolites that are representative of at least 2, at least 3, at least 4, at least 5, at least 10, at least 15, at least 20, at least 25, at least 30, at least 40, at least 50 of the subclasses listed in Table 1. In some embodiments, the plurality of metabolites contemplated in the methods described herein include a set of metabolites that are representative of at least 2, at least 3, at least 4, at least 5, at least 10, at least 15, at least 20, at least 25, at least 30, at least 40, at least 50 of the substituents listed in Table 1.

In some embodiments, the one or more metabolites do not include one or more metabolites that are members of a class selected from the classes listed in Table 2. In some embodiments, the one or more metabolites do not include one or more metabolites that are members of a subclass selected from the subclasses listed in Table 2. In some embodiments, the one or more metabolites do not comprise a substituent group selected from the substituents listed in Table 2.

TABLE 2
Classification of Metabolites Not Detected in FFPE

CLASS
Steroids and steroid derivatives
Azoles
Indoles
Diazines
Disaccharides
Prenol Lipids
Eicosanoids
Glycosyl Compounds
SUBCLASS
Medium-chain Hydroxy Acids and derivatives
Phosphatidylcholines
Lysophosphatidylglycerols
Taurited Bile Acids and derivatives
Glycited Bile Acids and derivatives
Imidazolyl Carboxylic Acids and derivatives
Pyrimidones
SUBSTITUENT
Beta hydroxy acid
Secondary aliphatic amine (dialkylamine)
Primary alcohol
Primary aliphatic amine (alkylamine)

In some aspects, techniques provided by the present disclosure may be performed in a comparative format. For example, in some embodiments, the one or more metabolites detected in the methods described herein are differentially expressed in a tumor sample versus a control sample. By “differentially expressed” it means that the average expression of a metabolite in a tumor sample has a statistically significant difference from that in a control sample. For example, a significant difference that indicates differentially expressed metabolites may be detected when the expression level of the metabolite in a tumor sample is at least 1%, at least 5%, at least 10%, at least 25%, at least 50%, at least 100%, at least 250%, at least 500%, or at least 1000% higher, or lower, than that of a control sample. Similarly, a significant difference may be detected when the expression level of a metabolite in a tumor sample is at least 2-fold, at least 3-fold, at least 4-fold, at least 5-fold, at least 6-fold, at least 7-fold, at least 8-fold, at least 9-fold, at least 10-fold, at least 20-fold, at least 30-fold, at least 40-fold, at least 50-fold, at least 100-fold, or more higher, or lower, than that of a control sample. Significant differences may be identified by using an appropriate statistical test. Tests for statistical significance are well known in the art and are exemplified in Applied Statistics for Engineers and Scientists by Petruccelli, Chen and Nandram 1999 Reprint Ed. In some embodiments, the differentially expressed metabolites are selected using a criteria of false discovery rate <0.2. In some embodiments, the differentially expressed metabolites are selected using a criteria of p-value <0.05. P-value looks at the average concentration of the metabolite in the two groups and reports the likelihood that the difference in the concentration between the two groups occurs by chance. As described in further detail in the Examples, a number of differentially expressed metabolites have already been identified using some of the methods provided herein. These metabolites, which were differentially expressed in tumor tissue (e.g., prostate cancer) versus control tissue with a p-value <0.05, are reported in Table 3.

In some embodiments, a control sample may be used in a comparative analysis in evaluating an FFPE preparation of a biological sample (e.g., a tumor sample). In some embodiments, a sample of interest (e.g., a tumor sample) and a control sample are biological samples of the same subject. In some embodiments, the sample of interest and the control sample are biological samples of different subjects. In some embodiments, the control sample is a biological sample of non-cancerous tissue. In some embodiments, the control sample is a biological sample of cancerous tissue. In some embodiments, the sample of interest is a biological sample having or suspected of having tumorous tissue. In some embodiments, the sample of interest is a prostate tissue sample. In some embodiments, the control sample is a prostate tissue sample.

TABLE 3
Metabolites Differentially Expressed in tumor versus
control FFPE samples
METABOLITE

Taurine
1-palmitoylglycerophosphoinositol
pyroglutamine
glutathione, oxidized
dihomo-linoleate
creatinine
1-linoleoylglycerophosphoethanolamine
eicosenoate
10-nonadecenoate
1-oleoylglycerophosphoinositol
myristate
threonine
docosapentaenoate (n3)
stearate
docosahexaenoate
13-methylmyristic acid
2-arachidonoylglycerophosphoethanolamine
1-stearoylglycerophosphoethanolamine
1-stearoylglycerophosphoinositol
dihomo-linolenate
ophthalmate
arachidonate
glucose
valine
Isobar: UDP-acetylglucosamine, UDP-acetylgalactosamine
2-stearoylglycerophosphoinositol
1-palmitoylglycerol
Serine
glycerophosphorylcholine
2-methylbutyrylcarnitine
1-arachidonoylglycerophosphoethanolamine
creatine
adenine
2-palmitoylglycerophosphoethanolamine
adenosine 5′-monophosphate
phosphoethanolamine
choline phosphate
phosphate
alanine
glutamate
5-oxoproline
guanine
citrate
cytidine
nicotinamide
spermidine
uridine 5′-monophosphate
fumarate
glycerol 3-phosphate
ethanolamine
6-sialyl-N-acetyllactosamine
sorbitol
glycine
linolenate (alpha or gamma)
asparagine
2-palmitoylglycerol
lysine
isoleucine
5-methylthioadenosine
glycerol
aspartate
fructose
adenosine
arginine
2-hydroxyglutarate
acetylcarnitine
beta-alanine
phenylalanine
succinate
malate
1-stearoylglycerol
uridine
leucine
tyrosine
guanosine
putrescine
carnitine

In some embodiments, the one or more metabolites detected in the methods described herein are selected from Table 3. In some embodiments, any subset of at least 2, at least 3, at least 4, at least 5, at least 10, at least 15, at least 20, at least 25, at least 30, at least 35, at least 40, at least 45, at least 50 of the metabolites of Table 3 are detected in the methods described herein. Examples of a subset of metabolites used in the methods described herein include, but are not limited to, the first 5, 10, 15, 20, 25, 30, 35, 40, 45, or 50 metabolites or the last 5, 10, 15, 20, 25, 30, 35, 40, 45, or 50 metabolites or any combination of 5, 10, 15, 20, 25, 30, 35, 40, 45, or 50 metabolites of Table 3. A non-limiting example of a subset of at least 10 metabolites used in the methods described herein is Taurine, 1-palmitoylglycerophosphoinositol, pyroglutamine, glutathione, oxidized, dihomo-linoleate, creatinine, 1-linoleoylglycerophosphoethanolamine, eicosenoate, 10-nonadecenoate, and 1-oleoylglycerophosphoinositol.

FFPE cell or tissue samples may be prepared according to protocols commonly used in the art (e.g., see Canene-Adams, K. Methods Enzymol. 2013; 533:225-33; and Hewitt, S. M., et al. Arch Pathol Lab Med. 2008; 132:1929-35). Typically, sections of paraffin-embedded cells or tissues are obtained by: (a) preserving a tissue in fixative, (b) dehydrating the fixed tissue, (c) infiltrating the tissue with fixative, (d) orienting the tissue such that the cut surface accurately represents the tissue, (e) embedding the tissue in paraffin (e.g., making a paraffin block), and (f) cutting tissue paraffin block with microtome into sections. In some embodiments, an FFPE preparation of a biological sample is analyzed by punching a core from the tissue paraffin block.

In some embodiments, methods described herein relate to the evaluation of an FFPE preparation of a biological sample. In some embodiments, multiple portions of a single FFPE preparation can be evaluated. For example, in some embodiments, two or more portions (e.g., punches, slices, etc.) of an FFPE preparation of a biological sample are obtained, and each portion is subjected to a separate analysis (e.g., evaluating the presence or absence of one or more metabolites). Such an approach can advantageously allow the practitioner to delineate normal tissue (e.g., healthy) and abnormal tissue (e.g., tumorous) within the three-dimensional architecture of the FFPE preparation. In some embodiments, the FFPE preparation is subjected to a metabolite extraction.

Metabolite extractions may be conducted according to any suitable methods known in the art. For example, in some embodiments, the conditions of an extraction method may be dependent upon the chemical and/or physical properties of the molecules (e.g., metabolites) that are targeted for a particular analysis. For example, in some embodiments, it may be desirable to extract polar metabolites. In such instances, a methanol solution may be used to extract polar metabolites in an FFPE preparation. Alternatively, in some embodiments, it may be desirable to favor extraction of non-polar metabolites. In such instances, a chloroform solution may be used to extract non-polar metabolites.

In some embodiments, methods described herein involve a metabolite extraction step. In some embodiments, metabolites are extracted from an FFPE preparation using a methanol solution (e.g., methanol in water). In some embodiments, the methanol solution is approximately 80% methanol. In some embodiments, the methanol solution is between about 50% methanol and about 60% methanol, between about 60% methanol and about 65% methanol, between about 65% methanol and about 70% methanol, between about 70% methanol and about 75% methanol, between about 75% methanol and about 80% methanol, between about 80% methanol and about 85% methanol, between about 85% methanol and about 90% methanol, between about 90% methanol and about 95% methanol, or between about 95% methanol and about 99% methanol. The methods disclosed herein typically comprise determining the presence of one or more metabolites in an FFPE preparation of a biological sample. In some embodiments, at least 5, at least 10, at least 25, at least 50, at least 75, at least 100, at least 125, at least 150, at least 175, at least 200, at least 225, at least 250, at least 500, at least 750, at least 1000 or at least 1500 metabolites are measured. In some embodiments, provided methods include measuring a level of expression of differentially expressed metabolites in a tumor sample versus a control sample. In some embodiments, at least 5, at least 10, at least 25, at least 50, at least 75, at least 100, at least 125, at least 150, at least 175, at least 200, at least 225, at least 250, at least 500, at least 750, at least 1000 or at least 1500 differentially expressed metabolites are measured.

In some embodiments, techniques described herein may be used to evaluate tumor status of a biological sample. As used herein, “tumor status” refers to the biological state of a sample with respect to any tumorous tissue. For example, in some embodiments, the tumor status of a tissue refers to the overall presence or absence of a tumor in the tissue sample. In some embodiments, methods of the disclosure may be used to provide additional information related to the tumor status of a tissue sample, such as whether the sample has benign, pre-malignant, or malignant tumorous tissue. In some embodiments, methods of the disclosure can further indicate the severity of a tumor in a tissue sample (e.g., indolent versus aggressive cancer). In some embodiments, tumor status is assessed based on a comparative analysis that involves evaluating differential expression of metabolites in tumor versus control samples.

Methods of the disclosure relate, in some embodiments, to the evaluation of an FFPE biological sample. As described herein, such samples may be evaluated using minimally invasive methods, e.g., chemical extraction of metabolites. In some embodiments, these techniques preserve the architectural landscape of the FFPE sample such that it may be subjected to additional evaluative procedures. For example, in some embodiments, the FFPE biological sample is subjected to metabolite extraction and subsequently stained for histological analysis (e.g., using any suitable histological stain such as alcian blue, Fuchsin, haematoxylin and eosin (H&E), Masson trichrome, toluidine blue, Wright's/Giemsa stain, and combinations thereof). Accordingly, in some embodiments, the methods described herein provide a comprehensive analysis at both the biochemical level and cellular level.

A report summarizing the results of the analysis, e.g., tumor status of the sample and any other information pertaining to the analysis could optionally be generated as part of the analysis (which may be interchangeably referred to herein as “providing” a report, “producing” a report, or “generating” a report). Examples of reports may include, but are not limited to, reports in paper (such as computer-generated printouts of test results) or equivalent formats and reports stored on computer readable medium (such as a CD, computer hard drive, or computer network server, etc.). Reports, particularly those stored on computer readable medium, can be part of a database (such as a database of patient records, which may be a “secure database” that has security features that limit access to the report, such as to allow only the patient and the patient's medical practitioners to view the report, for example). In addition to, or as an alternative to, generating a tangible report, reports can also be displayed on a computer screen (or the display of another electronic device or instrument).

A report can further be transmitted, communicated or reported (these terms may be used herein interchangeably), such as to the individual who was tested, a medical practitioner (e.g., a doctor, nurse, clinical laboratory practitioner, genetic counselor, etc.), a healthcare organization, a clinical laboratory, and/or any other party intended to view or possess the report. The act of ‘transmitting’ or ‘communicating’ a report can be by any means known in the art, based on the form of the report, and includes both oral and non-oral transmission. Furthermore, “transmitting” or “communicating” a report can include delivering a report (“pushing”) and/or retrieving (“pulling”) a report. For example, non-oral reports can be transmitted/communicated by such means as being physically transferred between parties (such as for reports in paper format), such as by being physically delivered from one party to another, or by being transmitted electronically or in signal form (e.g., via e-mail or over the internet, by facsimile, and/or by any wired or wireless communication methods known in the art), such as by being retrieved from a database stored on a computer network server, etc.

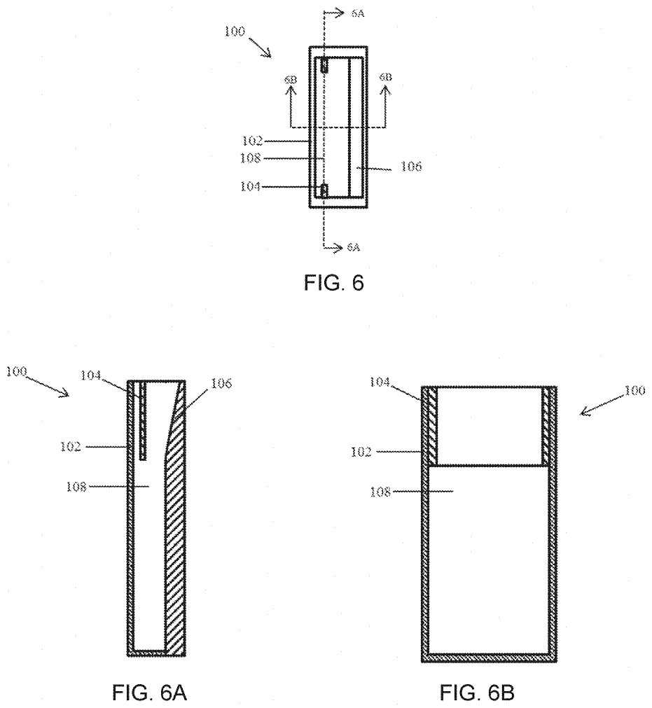

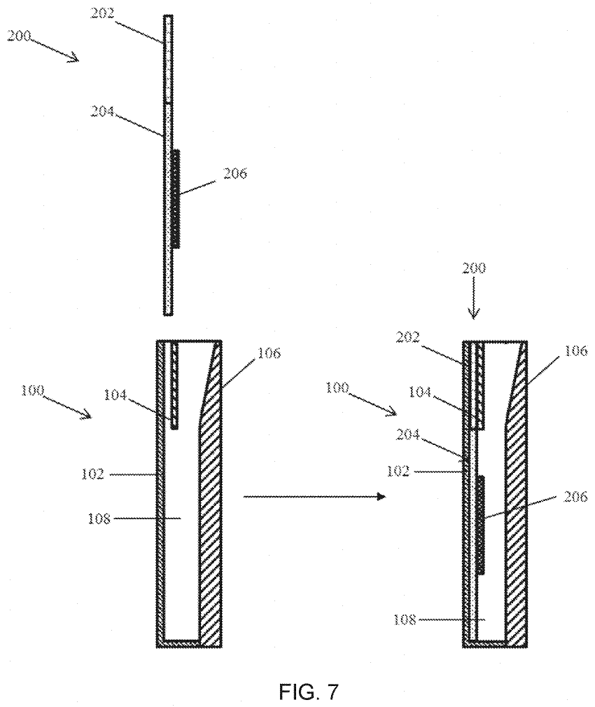

In some aspects, methods provided in the present disclosure may be conducted in an apparatus (e.g., a cassette) designed to accommodate a tissue section attached to a slide, as shown in the schematic embodiment depicted in FIGS. 6-7. Such an apparatus may be useful, for example, to minimize the loss of solution during extraction and maximize yields of extracted metabolites. In the depicted embodiment, a cassette 100 includes a housing 102. The housing includes an opening on one side that extends into a chamber 108 defined by the housing. The housing may include one or more restraints 104, that extends at least partially across a width of the cassette within the chamber. These restraints may also extend along at least a portion of the chamber between the opening and an opposing interior surface of the chamber. For example as depicted in the figure, the restraints correspond to two opposing tabs that extend inwards from opposing interior surfaces of the chamber towards one another. These tabs extend from an upper surface of the chamber adjacent the opening in a downward direction towards the opposing bottom surface of the chamber. The tabs only extend along a portion of the length of the chamber leaving a bottom portion of the chamber free from any structures that might impede access to a sample located on an associated slide, or alter the quality of the sample in any way (e.g., being rubbed or scraped). Embodiments in which the restraints extend along an entire length of the interior chamber are also contemplated. The cassette may also include a ramp 106 oriented inwards towards the chamber interior. During use, the ramp may help guide and accommodate the presence of a pipette, not depicted, inserted into an interior of the chamber for removing suspended metabolite from the cassette. To facilitate access of the pipette to an interior volume of the chamber containing the suspended metabolite, the one or more restraints may be removed from, and thus may maintain a corresponding slide, distanced from an opposing side of the chamber by a dimension sufficient for accommodating presence of the pipette.

FIG. 7 depicts the combination of a slide 200 being inserted into a corresponding cassette 100. In the depicted embodiment, the slide includes a label portion 202 that may include information regarding the sample 206 disposed on a lower sample portion of the slide 204. When desired, the slide is inserted into a first portion of the chamber 108 defined between a first side of the chamber and the one or more restraints 104. Thus, the slide may be retained in the first portion of the chamber such that a first-sample side of the slide may be disposed against the first side of the chamber and the opposing side of the slide containing the sample may be oriented towards an interior second portion of the chamber where the sample may be exposed to an appropriate solvent for extraction of the metabolite from the sample. In some embodiments, the restraints may extend over a length of the slide corresponding to the label portion of the slide, thus leaving the sample portion of the slide uncovered and fully accessible to any solvent present in the chamber. Once the metabolite has been extracted a pipette may be inserted into the chamber using ramp 106 to both guide the pipette into the chamber and/or to accommodate any structural features of the pipette. The metabolite sample may then be extracted through the pipette and the pipette may be removed.

It should be understood that the cassettes described above may have any appropriate combination of dimensions and/or volumes. For example, in one embodiment, the various structures of the cassette and may be constructed and arranged such that the cassette uses a relatively small volume of solvent for extraction of the metabolite. In such an embodiment, the volume of a portion of a chamber between a sample side of a slide or one or more restraints and an opposing side of the chamber may be between or equal to 0.5 and 3 mL, 1 and 2 mL, 1.5 and 5 mL, 2 and 10 mL and/or any other appropriate volume.

In one embodiment, a cassette may have an overall length between an opening and opposing bottom chamber surface of the chamber of about 75 mm. The distance between the one or more restraints and the bottom surface of the chamber may be about 50 mm. The distance between the one or more restraints and a side of the chamber a slide may be disposed against may be about 1.5 mm. A distance between the one or more restraints and a side of the chamber opposite the slide defining a volume the sample is exposed to may be between about 1.5 and 5 mm, 1.5 mm and 4 mm, 2 m, and 3 mm, and/or any other appropriate distance. The above described ramp may extend over a width of the chamber of about 5 mm and about 25 mm inwards from the opening into an interior of the chamber towards the opposing bottom surface of the chamber. While particular dimensions are noted above, it should be understood that any appropriate combination and/or ranges of dimensions may be used including dimensions both greater and small than those dimensions noted above as the disclosure is not so limited.

The present invention is further illustrated by the following Examples, which in no way should be construed as further limiting. The entire contents of all of the references (including literature references, issued patents, published patent applications, and co pending patent applications) cited throughout this application are hereby expressly incorporated by reference.

### EXAMPLES

### Example 1: Materials and Methods for Metabolic Profiling Prostate Cancer Tissues

### Cell Line Model

LNCaP prostate cells were grown in RPMI-1640 media supplemented with 10% fetal bovine serum and 1% penicillin-streptomycin. LNCaP-Ab1 (passage #81) cells were grown in RPMI-1640 media supplemented with 10% FBS Charcoal Dextran Stripped and 1% penicillin-streptomycin at 37° C. and 5% CO₂. Both cell lines were authenticated and tested mycoplasma free. About 5×10⁻⁶ cells were plated in a 10-cm dish. Prior to sample preparation (48 hrs after seeding), cells on the dish were washed three times with phosphate buffer solution (PBS).

To prepare frozen samples, adherent cells were directly quenched with 1 mL of 80% methanol in the dish culture to avoid trypsin use, and cells were gently detached using a cell lifter. The methanol solution containing the quenched cells was pipetted into a 2 mL centrifuge tube for extraction. In the case of FFPE samples, the adherent cells were directly quenched with 1 mL of 4% formalin. The formalin solution was kept in the culture dish for 20 minutes at room temperature. Then, the adherent cells were washed three times with PBS, detached using a cell lifter, and then embedded in paraffin following the standard procedure.

The detailed protocol to produce flash-frozen cell line samples is the following: 1) Change the medium of the cell dishes 2 hours before metabolite extraction; 2) Aspirate the medium completely; 3) Wash the dishes 2-3 times with PBS; 4) Put the dishes on dry ice and add 1 mL of 80% methanol (cooled to −80° C.); 5) Incubate the dishes at −80° C. for 20 minutes; 6) Scrape the dishes on dry ice with cell scraper; 7) Transfer the cell lysate/methanol mixture to a 15 mL conical tube on dry ice; 8) Centrifuge the tube at 14,000 g for 5 minutes to pellet the cell debris; 9) Transfer the metabolite-containing supernatant to a new tube; 10) Dry the metabolite-containing supernatant using no heat; 11) The dried metabolite samples can be stored at −80° C. for several weeks.

The detailed protocol to produce FFPE cell line samples is the following: 1) Change the medium of the cell dishes 2 hours before metabolite extraction; 2) Aspirate the medium completely; 3) Wash the dishes 2-3 times with PBS; 4) Add 1 mL of 4% formalin to each dish; 5) Incubate the dishes at room temperature for 20 minutes; 6) Aspirate the 4% formalin solution completely; 7) Wash the dishes 2-3 times with PBS; 8) Scrape the dishes with cell scraper; 9) Transfer the fixated cells into a cassette; 10) Embed the fixated cells in paraffin using the standard procedure; 11) Place FFPE cells in a 1.5 mL micro-centrifuge tube; 12) Prepare the FFPE extracts following the protocol to extract the metabolites from FFPE material; 13) The dried metabolite samples can be stored at −80° C. for several weeks.

### Human Prostate Tissue

Samples from radical prostatectomies were utilized in the study. Both Optimal Cutting Temperature (OCT)-embedded and FFPE tissue blocks were collected from each prostatectomy. Tissue blocks were sectioned at 5 μm and were stained with H&E to identify tumor and normal area in each block. Sections of 20 μm were stained with H&E to evaluate the tissue architecture. Histopathology evaluation was performed to assess the percentage of tumor and the Gleason score in each of the tissue samples. From each tissue block were collected 2-mm biopsy punch samples from both the tumor and normal tissue compartment.

### Macro-Dissection

Slide-mounted tissue sections, regions enriched for normal and tumor epithelial cells were dissected manually. An Area Of Interest (AOI) was hand annotated by a pathologist (M.L.) on an H&E stained, cover slipped, slide-mounted tissue section. This section was then manually aligned and traced onto the back of a second non-cover slipped slide containing a serially cut tissue section from the same tissue block. Manual macro-dissection was then performed on the second slide using a scalpel or razor blade to remove the tissue out of AOI. H&E slides were scanned using Vectra Intelligent Slide Analysis System 2.0.8.

### Digital Scanning

H&E slides were scanned using Vectra Intelligent Slide Analysis System 2.0.8 (Perkin Elmer) using the tissue scanning protocol at optimal setting. Bright-field images acquired at 4× and 20× were then used to develop semi-automated image analysis algorithms using inform Advanced Image Analysis Software 2.0.5 (Perkin Elmer). Full slide batches of images were processed automatically and edited for quality. Images acquired at 4× (full-resolution RGB) with resolution factored two times higher were used in trainable tissue segmentation. Developed algorithm was confident in distinguishing epithelium and stroma, but not tumor and benign tissue. Each image was reviewed by a pathologist and manually edited to distinguish tumor and benign tissue. An algorithm was developed on 20× images (full-resolution RGB) converted to optical density for trainable cell segmentation. Pre-set spectral libraries of Hematoxylin (blue hematox) and eosin from Nuance 3.0 (Perkin Elmer) were applied against a blank slide as white background. Nuclear segmentation based on blue hematox component, minimum signal 0.30 (on a value scale ranging from 0 to 1), minimum size 40 pixels and maximum size 400 pixels, and refined splitting with minimum circularity of 0.2. Total cells were counted in epithelium and stroma with the percentage area of the nuclei.

Metabolite Extraction with Methanol

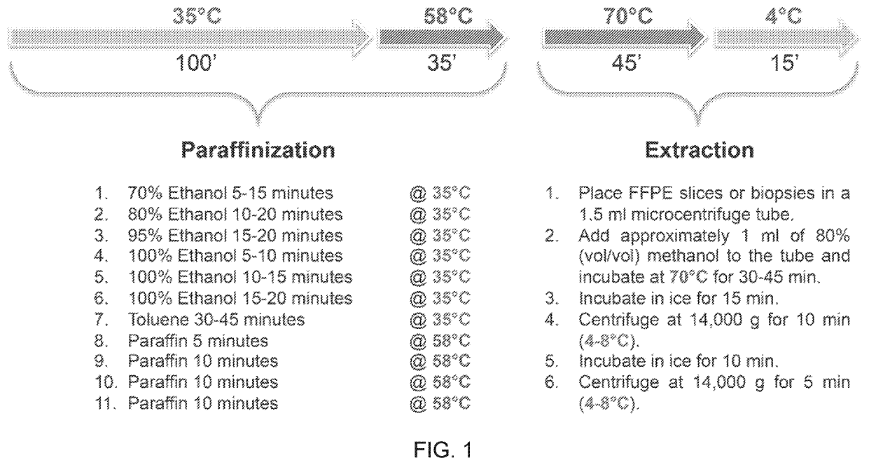

The metabolome from frozen samples was extracted incubating the tissue in 1 mL of 80% methanol at room temperature on a benchtop for 4 hrs. After centrifugation at 14,000 g (10 minutes), the supernatant was collected and stored at −80° C. Metabolite extraction from FFPE samples was performed by adding 1 mL of 80% methanol directly to the sample and incubating at 70° C. for 30-45 minutes in a 1.5-mL micro-centrifuge tube without any de-paraffinization procedure (12). The sample was then placed on ice for 15 minutes and centrifuged at 14,000 g for 10 minutes (4-8° C.). The supernatant was transferred into a new 1.5-mL micro-centrifuge tube and chilled on ice for 10 minutes, followed by centrifugation at 14,000 g for 5 minutes (4-8° C.). Finally, the supernatant was collected and stored at −80° C. A schematic overview of the procedure is shown in FIG. 1.

### Metabolite Profiling

Metabolite profiling was conducted as previously described and further detailed in the below (13).

Sample Preparation:

The sample preparation process was carried out using the automated MicroLab STAR® system. Recovery standards were added prior to the first step in the extraction process for Quality Control (QC) purposes. Sample preparation was conducted using a series of organic and aqueous extractions to remove the protein fraction while allowing maximum recovery for small molecules. The resulting extract was divided into two fractions; one for analysis by LC and one for analysis by GC. Samples were placed briefly on a TurboVap® to remove the organic solvent. Each sample was then frozen and dried under vacuum. Samples were then prepared for either LC-MS or GC-MS, accordingly.

For Quality Assurance (QA)/QC purposes, a number of additional samples were included with each day's analysis. Furthermore, a selection of QC compounds was added to every sample, including those under test. These compounds were carefully chosen so as not to interfere with the measurement of the endogenous compounds. These QC samples were primarily used to evaluate the process control for each study as well as aiding in the data curation.

Ultrahigh performance liquid chromatography/Mass Spectroscopy (UPLC-MS/MS):

The LC-MS portion of the platform was based on a Waters ACQUITY ultra-performance liquid chromatography (UPLC) and a Thermo-Finnigan linear trap quadrupole (LTQ) mass spectrometer, which consists of an electrospray ionization (ESI) source and linear ion-trap (LIT) mass analyzer. The sample extract was dried and then reconstituted in acidic or basic LC-compatible solvents, each of which contained 8 or more injection standards at fixed concentrations to ensure injection and chromatographic consistency. One aliquot was analyzed using acidic positive ion optimized conditions and the other using basic negative ion optimized conditions in two independent injections using separate dedicated columns. Extracts reconstituted in acidic conditions were gradient eluted using water and methanol containing 0.1% formic acid, while the basic extracts, which also used water/methanol, contained 6.5 mM Ammonium Bicarbonate. The MS analysis alternated between MS and data-dependent MS2 scans using dynamic exclusion.

Gas chromatography/Mass Spectroscopy (GC-MS):

The samples destined for GC-MS analysis were re-dried under vacuum desiccation for a minimum of 24 hrs, prior to being derivatized under dried nitrogen using bistrimethyl-silyl-triflouroacetamide (BSTFA). The GC column was 5% phenyl and the temperature ramp was from 40° C. to 300° C. in a 16 minute period. Samples were analyzed on a Thermo-Finnigan Trace DSQ fast-scanning single-quadrupole mass spectrometer using electron impact ionization. The instrument was tuned and calibrated for mass resolution and mass accuracy on a daily basis.

Accurate mass determination and MS/MS fragmentation (LC-MS), (LC-MS/MS):

The LC-MS portion of the platform was based on a Water ACQUITY UPLC and a Thermo-Finnigan LTQ-FT mass spectrometer, which had a linear ion-trap (LIT) front-end and a Fourier transform ion cyclotron resonance (FT-ICR) mass spectrometer backend. For ions with counts greater than 2 million, an accurate mass measurement could be performed. Accurate mass measurements could be made on the parent ion as well fragments. The typical mass error was less than 5 ppm.

Data quality: instrument and process variability:

Instrument variability was determined by calculating the median relative standard deviation (RSD) for the internal standards that were added to each sample prior to injection into the mass spectrometers. Overall process variability was determined by calculating the median RSD for all endogenous metabolites (i.e., non-instrument standards) present in 100% of the samples, which are technical replicates of pooled samples. Values for instrument and process variability meet acceptance criteria of 6% and 13% of median RSD for, respectively, for internal standards and endogenous biochemical.

Compound identification:

Compounds were identified by comparison to library entries of purified standards or recurrent unknown entities was based on comparison to metabolomic library entries of purified standards. The combination of chromatographic properties and mass spectra gave an indication of a match to the specific compound or an isobaric entity. Additional entities could be identified by virtue of their recurrent nature (both chromatographic and mass spectral). These compounds have the potential to be identified by future acquisition of a matching purified standard or by classical structural analysis.

### Statistical Analysis

In data pre-processing, contaminants present in FFPE samples (i.e., dimethyl sulfoxide, lauryl sulfate, and melanine) and OCT-embedded samples (i.e., heptaethylene glycol, hexaethylene glycol, octaethylene glycol, pentaethylene glycol, and tetraethylene glycol) were not considered in the analysis. Compounds with more than 90% of missing value were not considered to be reliable and were excluded. Probabilistic Quotient Normalization (PQN) (21) was used to normalize data due to dilution effects in the extraction procedure. For multivariate analysis, compounds with more than 25% of missing values were not used. Otherwise, missing metabolite measurements were imputed using k nearest neighbor (kNN) algorithm (22) with k=5. Data were log-transformed, mean-centered, and scaled to unit variance. The cell line data were centered to the mean of all samples and human samples were centered to the mean of each patient.

Fisher's exact test was used for testing the null hypothesis of independence of rows and columns in a contingency table. Pairwise comparisons were made using the Mann-Whitney test for independent data. Correlation was assessed using the Spearman's rho statistic. The threshold for significance was P<0.05 for all tests. To account for multiple testing, a False Discovery Rate (FDR) of <10% was applied to reduce identification of false positives. FDRs were calculated using the q conversion algorithm (14) in multiple comparison.

Furthermore, Orthogonal Signal Correction (OSC) applied to the Partial Least Square (PLS) model (15), a supervised pattern recognition approach, was used to visualize differences in metabolite composition in samples and as a predictive model in cross-validation analysis using the values of the orthogonal latent variable.

Metabolite Set Enrichment Analysis (MSEA) was carried out using the tool GSEA (Gene Pattern software, Broad Institute, http://genepattern.broadinstitute.org). The metabolite sets were built using the human pathway information available in the Human Metabolome Database (http://www.hmdb.ca). The loadings of OSC-PLS were used for the ranking in the MSEA.

Heatmaps were ordered according to hierarchical clustering (Ward linkage) based on the KODAMA dissimilarity matrix (16) implemented in R package KODAMA. For human FFPE samples of the training set, KODAMA was performed with sample replicates constrained to cluster together. Analyses were carried out using R software (17) with scripts developed in-house.

### Example 2: Metabolite Recovery in Isogenic Prostate Cancer Cell Line FFPE Samples

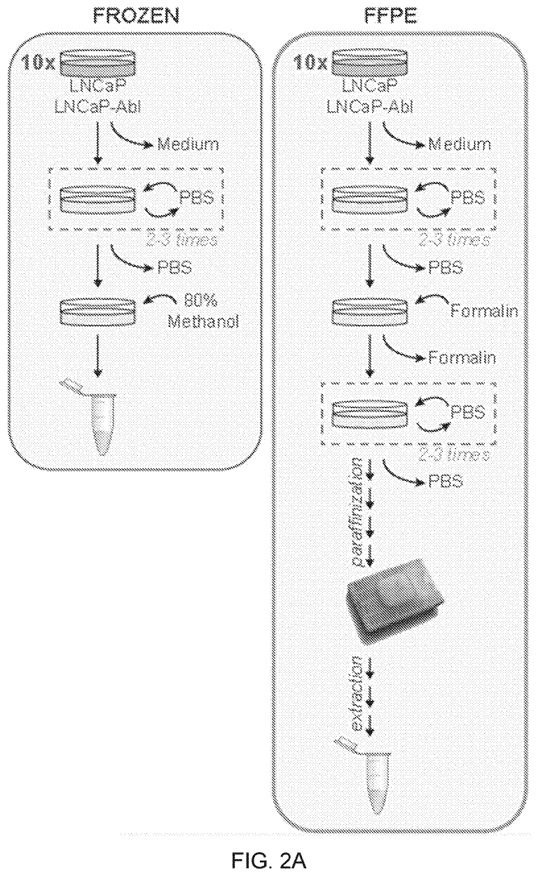

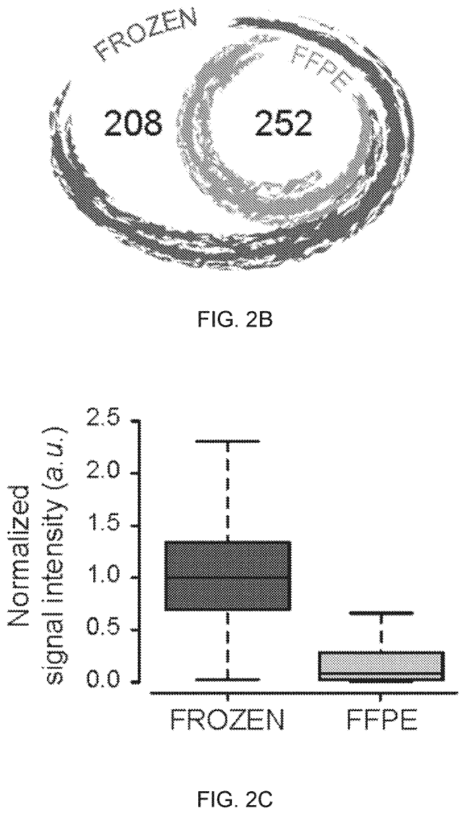

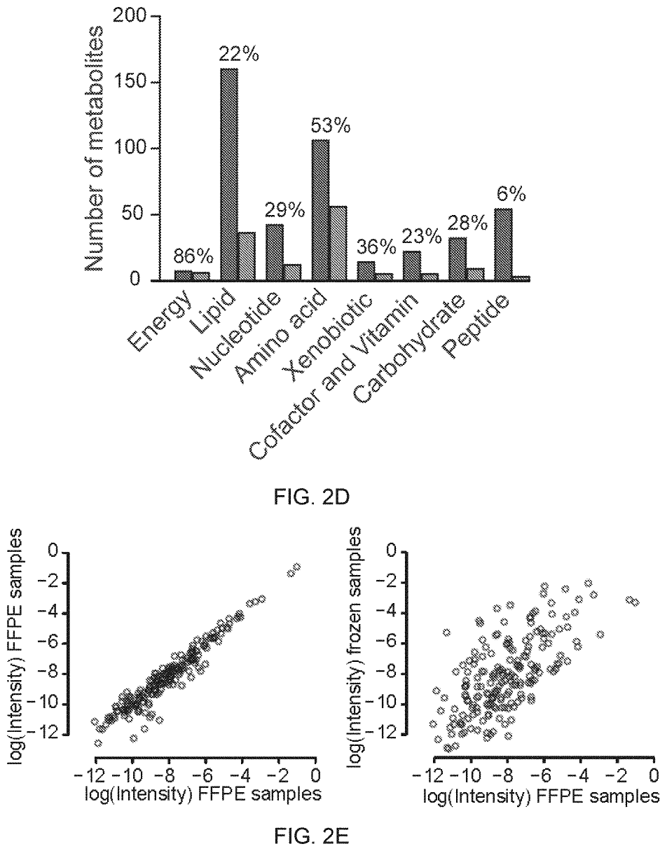

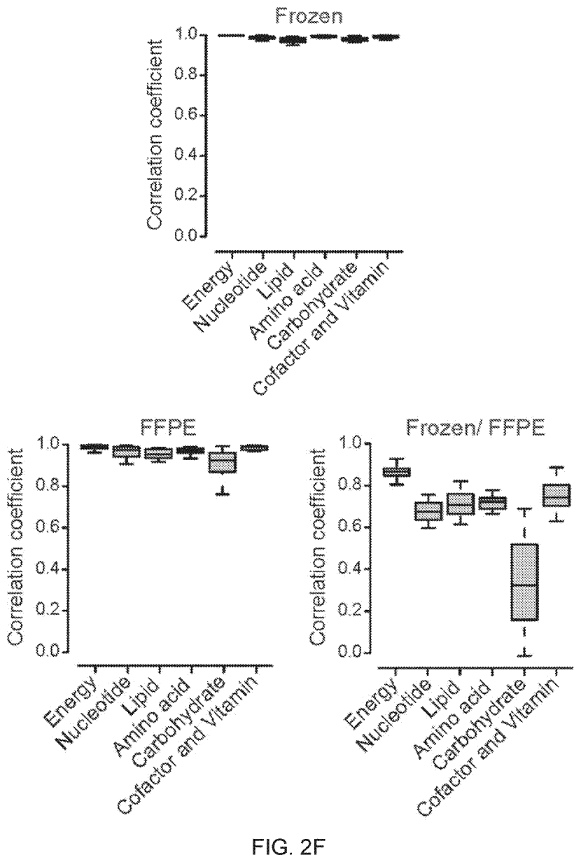

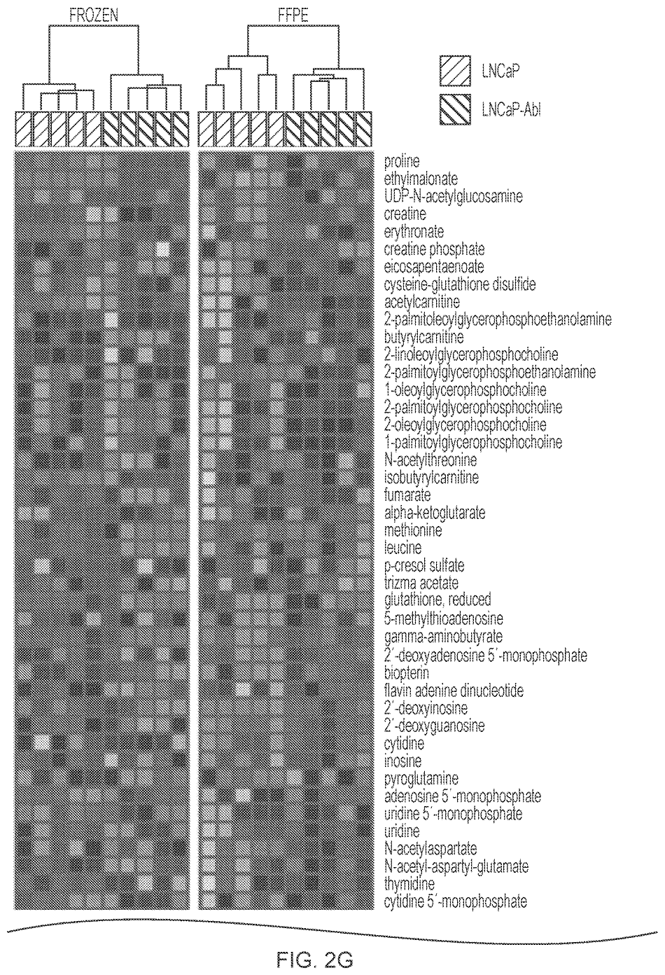

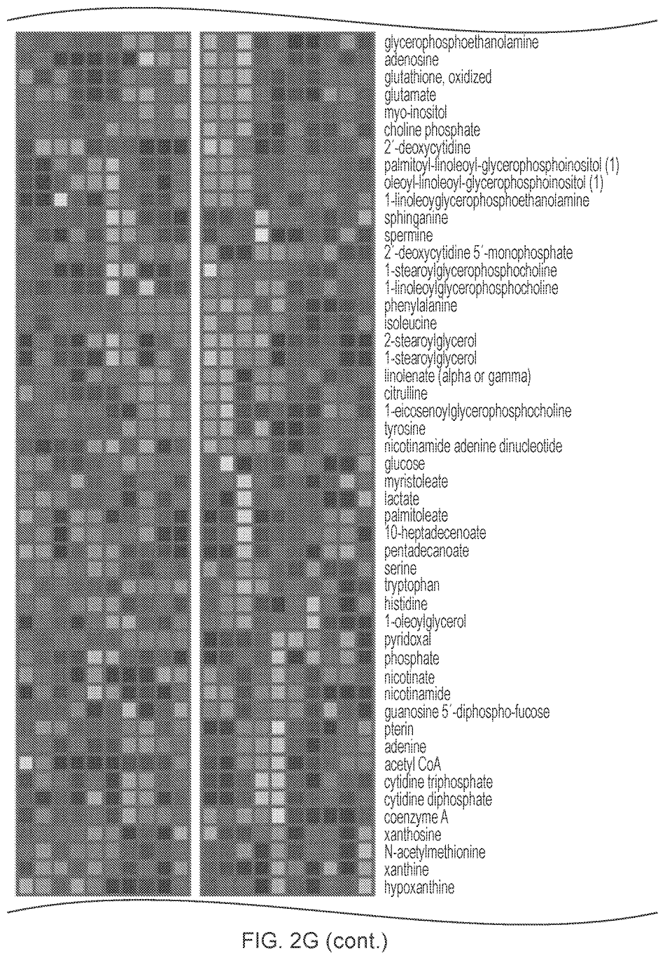

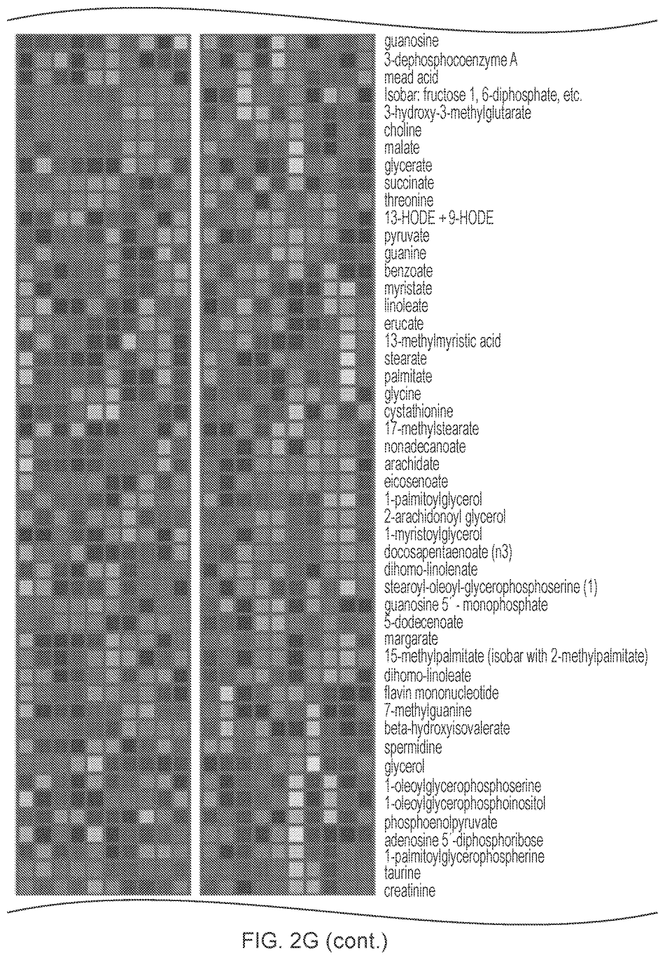

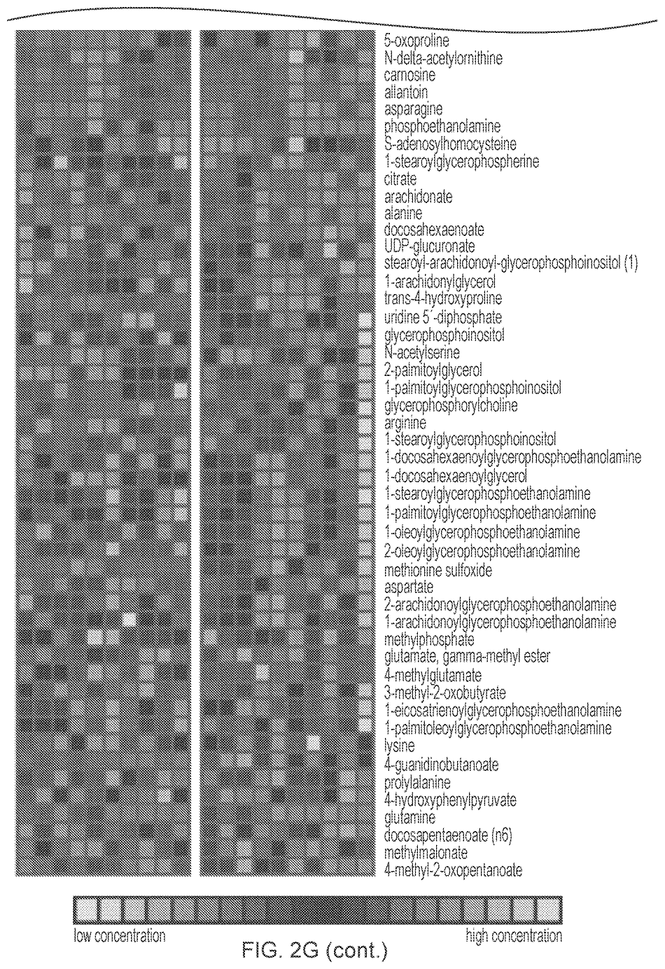

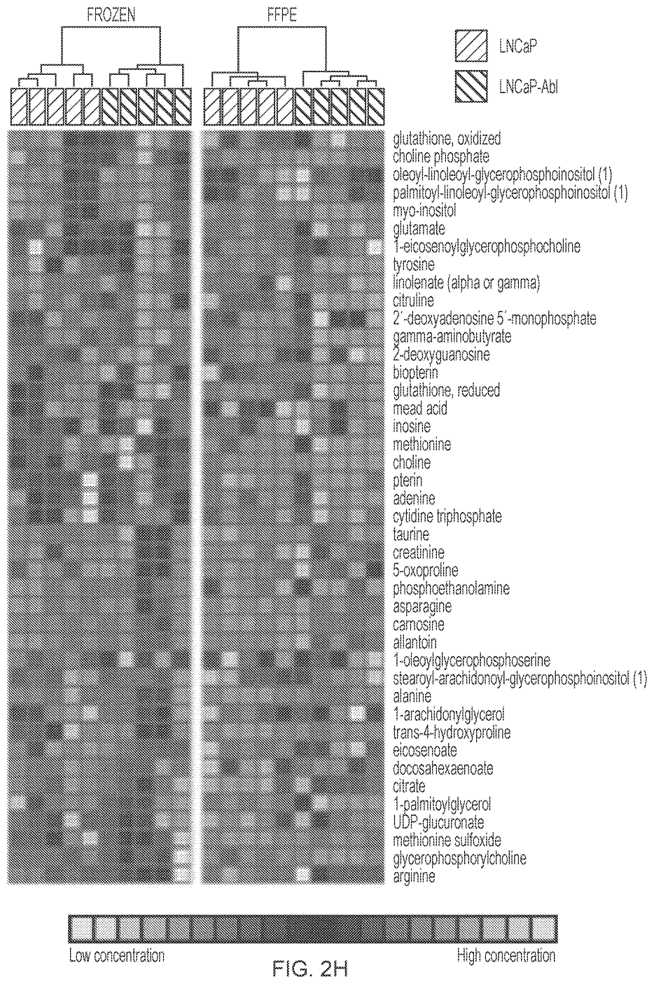

To compare metabolomic data generated from frozen and FFPE material, prostate cancer isogenic cell lines (i.e., hormone-sensitive LNCaP and castration-resistant LNCaP-Abl) were profiled using untargeted ultrahigh performance liquid chromatography (UPLC)-MS and GC-MS. Using the protocol schematized in FIG. 2A, a total of 252 metabolites were detected and quantified in both frozen and FFPE samples. An additional 208 metabolites were identified in frozen samples (FIG. 2B). Both FFPE and frozen cell line samples were generated from replicates of 10 cm culture dishes (48 hrs after seeding 5×10⁻⁶ cells). Extraction yield from FFPE samples was estimated to be 12-fold less than frozen samples as determined by comparing intensity values of recovered metabolite signals (FIG. 2C).

Next, metabolite categorization (i.e., superclass, class, subclass, and metabolic pathway), substituents (an atom or group of atoms taking the place of another atom group or occupying a specific position in a molecule), and chemical/physical properties as annotated in the Human Metabolome Database (HMDB, http://www.hmdb.ca/), Small Molecule Pathway Database (SMPDB, http://smpdb.ca), and Kyoto Encyclopedia of Genes and Genomes (KEGG, http://www.genome.jp/kegg) were used to provide a detailed analysis of the metabolites detectable in FFPE samples. As shown in FIG. 2D, the rate of detection in FFPE samples compared to the corresponding frozen material varied according to the class and the chemical/physical properties of the metabolite. Fisher's exact test was used to evaluate the differences in the number of metabolites belonging to a specific category detected or non-detected in FFPE samples. Significant differences are listed in Table 4, Table 5, Table 6, Table 7, Table 8, and Table 9, which list the metabolites found/missed in FFPE sample categorized by superclass, class, subclass, substituent, physical/chemical properties and pathway, respectively.

TABLE 4
Metabolites found and missed in FFPE categorized by superclass.

| | non-preserved in | preserved in | FFPE/ | | |
|---|---|---|---|---|---|
| Superclass | FFPE, n (%) | FFPE, n (%) | FROZEN, % | P | FDR |
| Peptide | 50 (24%) | 6 (2.4%) | 10.7% | 4.56E−13 | 3.65E−12 |
| Lipid | 57 (27.4%) | 114 (45.2%) | 66.7% | 1.01E−04 | 4.03E−04 |
| Nucleotide | 11 (5.3%) | 34 (13.5%) | 75.6% | 4.11E−03 | 1.10E−02 |
| Xenobiotics | 11 (5.3%) | 5 (2%) | 31.2% | 7.25E−02 | 1.45E−01 |
| Energy | 2 (1%) | 6 (2.4%) | 75.0% | 3.03E−01 | 4.85E−01 |
| Carbohydrate | 17 (8.2%) | 15 (6%) | 46.9% | 3.64E−01 | 4.85E−01 |
| Cofactors and Vitamins | 11 (5.3%) | 12 (4.8%) | 52.2% | 8.32E−01 | 9.51E−01 |
| Amino Acid | 49 (23.6%) | 60 (23.8%) | 55.0% | 1.00E+00 | 1.00E+00 |

TABLE 5
Metabolites found and missed in FFPE categorized by superclass.

| | non-preserved in | preserved in | FFPE/ | | |
|---|---|---|---|---|---|
| Class | FFPE, n (%) | FFPE, n (%) | FROZEN, % | P | FDR |
| Peptides | 47 (29.4%) | 7 (3.4%) | 13.0% | 4.17E−12 | 1.04E−10 |
| Fatty Acids and Conjugates | 2 (1.2%) | 28 (13.7%) | 93.3% | 4.74E−06 | 5.93E−05 |
| Glycerolipids | 0 (0%) | 10 (4.9%) | 100.0% | 2.97E−03 | 2.47E−02 |
| Pyrimidine Nucleotides | 1 (0.6%) | 12 (5.9%) | 92.3% | 8.24E−03 | 5.15E−02 |
| Monosaccharides | 10 (6.2%) | 3 (1.5%) | 23.1% | 2.11E−02 | 1.05E−01 |
| Purine Nucleotides | 2 (1.2%) | 11 (5.4%) | 84.6% | 4.50E−02 | 1.87E−01 |
| Imidazopyrimidines | 0 (0%) | 5 (2.5%) | 100.0% | 6.97E−02 | 2.42E−01 |
| Azoles | 3 (1.9%) | 0 (0%) | 0.0% | 8.48E−02 | 2.42E−01 |
| Fatty Acid Esters | 11 (6.9%) | 6 (2.9%) | 35.3% | 8.70E−02 | 2.42E−01 |
| Lineolic Acids and Derivatives | 0 (0%) | 4 (2%) | 100.0% | 1.34E−01 | 3.34E−01 |
| Hydroxy Acids and Derivatives | 6 (3.8%) | 3 (1.5%) | 33.3% | 1.91E−01 | 4.34E−01 |
| Glycerophospholipids | 18 (11.2%) | 32 (15.7%) | 64.0% | 2.28E−01 | 4.74E−01 |
| Sugar Acids and Derivatives | 5 (3.1%) | 3 (1.5%) | 37.5% | 3.09E−01 | 5.94E−01 |
| Sphingolipids | 4 (2.5%) | 2 (1%) | 33.3% | 4.12E−01 | 7.35E−01 |
| Pyrimidine Nucleosides and | 2 (1.2%) | 5 (2.5%) | 71.4% | 4.72E−01 | 7.48E−01 |
| Analogues | | | | | |
| Purine Nucleosides and | 3 (1.9%) | 7 (3.4%) | 70.0% | 5.23E−01 | 7.48E−01 |
| Analogues | | | | | |
| Sugar Alcohols | 2 (1.2%) | 1 (0.5%) | 33.3% | 5.85E−01 | 7.48E−01 |
| Benzyl Alcohols and Derivatives | 1 (0.6%) | 3 (1.5%) | 75.0% | 6.34E−01 | 7.48E−01 |
| Cyclic Alcohols and Derivatives | 1 (0.6%) | 3 (1.5%) | 75.0% | 6.34E−01 | 7.48E−01 |
| Pteridines and Derivatives | 1 (0.6%) | 3 (1.5%) | 75.0% | 6.34E−01 | 7.48E−01 |
| Pyridines and Derivatives | 1 (0.6%) | 3 (1.5%) | 75.0% | 6.34E−01 | 7.48E−01 |
| Alkylamines | 3 (1.9%) | 2 (1%) | 40.0% | 6.58E−01 | 7.48E−01 |
| Carboxylic Acids and Derivatives | 2 (1.2%) | 4 (2%) | 66.7% | 6.98E−01 | 7.59E−01 |
| Amino Acids and Derivatives | 34 (21.2%) | 45 (22.1%) | 57.0% | 8.99E−01 | 9.37E−01 |
| Organic Phosphoric Acids and | 1 (0.6%) | 2 (1%) | 66.7% | 1.00E+00 | 1.00E+00 |
| Derivatives | | | | | |

TABLE 6
Metabolites found and missed in FFPE categorized by superclass.

| | non-preserved in | preserved in | FFPE/ | | |
|---|---|---|---|---|---|
| Subclass | FFPE, n (%) | FFPE, n (%) | FROZEN, % | P | FDR |
| Peptides | 47 (34.1%) | 7 (4.3%) | 13.0% | 4.17E−12 | 1.42E−10 |
| Unsaturated Fatty Acids | 0 (0%) | 15 (9.1%) | 100.0% | 2.02E−04 | 3.44E−03 |
| Lysophosphatidylethanolamines | 1 (0.7%) | 17 (10.4%) | 94.4% | 4.18E−04 | 4.73E−03 |
| Phosphatidylcholines | 8 (5.8%) | 0 (0%) | 0.0% | 1.30E−03 | 1.11E−02 |
| Monoacylglycerols | 0 (0%) | 10 (6.1%) | 100.0% | 2.97E−03 | 2.02E−02 |
| Straight Chain Fatty Acids | 0 (0%) | 8 (4.9%) | 100.0% | 1.03E−02 | 5.86E−02 |
| Hexoses | 6 (4.3%) | 1 (0.6%) | 14.3% | 4.72E−02 | 2.29E−01 |
| Alpha Amino Acids and | 13 (9.4%) | 30 (18.3%) | 69.8% | 7.26E−02 | 2.87E−01 |
| Derivatives | | | | | |
| N-acyl-alpha Amino Acids and | 14 (10.1%) | 8 (4.9%) | 36.4% | 7.61E−02 | 2.87E−01 |
| Derivatives | | | | | |
| Imidazolyl Carboxylic Acids and | 3 (2.2%) | 0 (0%) | 0.0% | 8.48E−02 | 2.88E−01 |
| Derivatives | | | | | |
| Acyl Carnitines | 10 (7.2%) | 5 (3%) | 33.3% | 1.09E−01 | 3.25E−01 |
| Lineolic Acids and Derivatives | 0 (0%) | 4 (2.4%) | 100.0% | 1.34E−01 | 3.25E−01 |
| Purine 2′-deoxyribonucleosides | 0 (0%) | 4 (2.4%) | 100.0% | 1.34E−01 | 3.25E−01 |
| and Analogues | | | | | |
| Pyrimidine Nucleotide Sugars | 0 (0%) | 4 (2.4%) | 100.0% | 1.34E−01 | 3.25E−01 |
| Dicarboxylic Acids and | 0 (0%) | 3 (1.8%) | 100.0% | 2.59E−01 | 4.89E−01 |
| Derivatives | | | | | |
| Phosphatidylinositols | 0 (0%) | 3 (1.8%) | 100.0% | 2.59E−01 | 4.89E−01 |
| Pyrimidine 2′- | | | | | |
| deoxyribonucleosides and | 0 (0%) | 3 (1.8%) | 100.0% | 2.59E−01 | 4.89E−01 |
| Analogues | | | | | |
| Pyrimidine Ribonucleoside | 0 (0%) | 3 (1.8%) | 100.0% | 2.59E−01 | 4.89E−01 |
| Diphosphates | | | | | |
| Pentoses | 3 (2.2%) | 1 (0.6%) | 25.0% | 3.25E−01 | 5.81E−01 |
| Branched Fatty Acids | 1 (0.7%) | 4 (2.4%) | 80.0% | 3.89E−01 | 6.30E−01 |
| Purine Ribonucleoside | 1 (0.7%) | 4 (2.4%) | 80.0% | 3.89E−01 | 6.30E−01 |
| Diphosphates | | | | | |
| Sugar Acids and Derivatives | 4 (2.9%) | 2 (1.2%) | 33.3% | 4.12E−01 | 6.37E−01 |
| Lysophosphatidylcholines | 7 (5.1%) | 6 (3.7%) | 46.2% | 5.74E−01 | 7.65E−01 |
| Beta Amino Acids and | 2 (1.4%) | 1 (0.6%) | 33.3% | 5.85E−01 | 7.65E−01 |
| Derivatives | | | | | |
| Glycoamino Acids and | 2 (1.4%) | 1 (0.6%) | 33.3% | 5.85E−01 | 7.65E−01 |
| Derivatives | | | | | |
| Sugar Alcohols | 2 (1.4%) | 1 (0.6%) | 33.3% | 5.85E−01 | 7.65E−01 |
| Phenylpyruvic Acid Derivatives | 1 (0.7%) | 3 (1.8%) | 75.0% | 6.34E−01 | 7.69E−01 |
| Purine Ribonucleoside | 1 (0.7%) | 3 (1.8%) | 75.0% | 6.34E−01 | 7.69E−01 |
| Monophosphates | | | | | |
| Beta Hydroxy Acids and | 3 (2.2%) | 2 (1.2%) | 40.0% | 6.58E−01 | 7.72E−01 |
| Derivatives | | | | | |
| Acyl Glycines | 2 (1.4%) | 2 (1.2%) | 50.0% | 1.00E+00 | 1.00E+00 |
| Polyamines | 1 (0.7%) | 2 (1.2%) | 66.7% | 1.00E+00 | 1.00E+00 |
| Purine Nucleosides and | 3 (2.2%) | 3 (1.8%) | 50.0% | 1.00E+00 | 1.00E+00 |
| Analogues | | | | | |
| Pyrimidine Nucleosides and | 2 (1.4%) | 2 (1.2%) | 50.0% | 1.00E+00 | 1.00E+00 |
| Analogues | | | | | |
| Sphingolipids | 1 (0.7%) | 2 (1.2%) | 66.7% | 1.00E+00 | 1.00E+00 |

TABLE 7
Metabolites found and missed in FFPE categorized by superclass.

| | non-preserved in | | preserved in FFPE, n | | | | |
|---|---|---|---|---|---|---|---|
| Substituent | FFPE, n (%) | | (%) | | FFPE/FROZEN, % | P | FDR |
| n-substituted-alpha-amino acid | 47 | (26.9%) | 7 | (3.2%) | 13.0% | 4.17E−12 | 6.42E−10 |
| carboxamide_group | 78 | (44.6%) | 30 | (13.5%) | 27.8% | 7.43E−12 | 6.42E−10 |
| secondary carboxylic acid amide | 70 | (40%) | 24 | (10.8%) | 25.5% | 1.03E−11 | 6.42E−10 |
| alpha-amino acid or derivative | 49 | (28%) | 9 | (4.1%) | 15.5% | 1.12E−11 | 6.42E−10 |
| n-acyl-alpha-amino-acid | 44 | (25.1%) | 7 | (3.2%) | 13.7% | 4.42E−11 | 2.02E−09 |
| saccharide | 13 | (7.4%) | 47 | (21.2%) | 78.3% | 1.21E−04 | 3.96E−03 |
| pyrimidine | 13 | (7.4%) | 47 | (21.2%) | 78.3% | 1.21E−04 | 3.96E−03 |
| dicarboxylic acid derivative | 41 | (23.4%) | 22 | (9.9%) | 34.9% | 2.99E−04 | 8.55E−03 |
| pyrimidone | 6 | (3.4%) | 31 | (14%) | 83.8% | 3.72E−04 | 9.45E−03 |
| quaternary ammonium salt | 32 | (18.3%) | 16 | (7.2%) | 33.3% | 1.01E−03 | 2.30E−02 |
| carboxylic acid salt | 19 | (10.9%) | 6 | (2.7%) | 24.0% | 1.34E−03 | 2.62E−02 |
| choline | 28 | (16%) | 13 | (5.9%) | 31.7% | 1.37E−03 | 2.62E−02 |
| 1,3-aminoalcohol | 16 | (9.1%) | 5 | (2.3%) | 23.8% | 2.85E−03 | 5.02E−02 |
| oxolane | 15 | (8.6%) | 40 | (18%) | 72.7% | 8.10E−03 | 1.33E−01 |
| aminopyrimidine | 9 | (5.1%) | 29 | (13.1%) | 76.3% | 9.33E−03 | 1.43E−01 |
| n-glycosyl compound | 10 | (5.7%) | 30 | (13.5%) | 75.0% | 1.15E−02 | 1.64E−01 |
| glycero-3-phosphocholine | 15 | (8.6%) | 6 | (2.7%) | 28.6% | 1.22E−02 | 1.64E−01 |
| primary aliphatic amine | 70 | (40%) | 62 | (27.9%) | 47.0% | 1.35E−02 | 1.68E−01 |
| (alkylamine) | | | | | | | |
| hydropyrimidine | 4 | (2.3%) | 18 | (8.1%) | 81.8% | 1.39E−02 | 1.68E−01 |
| carboxylic acid | 106 | (60.6%) | 107 | (48.2%) | 50.2% | 1.52E−02 | 1.71E−01 |
| organic pyrophosphate | 3 | (1.7%) | 16 | (7.2%) | 84.2% | 1.57E−02 | 1.71E−01 |
| glycosyl compound | 11 | (6.3%) | 30 | (13.5%) | 73.2% | 2.02E−02 | 1.93E−01 |
| acyclic alkene | 20 | (11.4%) | 45 | (20.3%) | 69.2% | 2.02E−02 | 1.93E−01 |
| phosphocholine | 17 | (9.7%) | 8 | (3.6%) | 32.0% | 2.04E−02 | 1.93E−01 |
| amphetamine or derivative | 10 | (5.7%) | 3 | (1.4%) | 23.1% | 2.11E−02 | 1.93E−01 |
| fatty acid ester | 18 | (10.3%) | 42 | (18.9%) | 70.0% | 2.34E−02 | 2.06E−01 |
| imidazole | 10 | (5.7%) | 28 | (12.6%) | 73.7% | 2.48E−02 | 2.11E−01 |
| triose monosaccharide | 3 | (1.7%) | 15 | (6.8%) | 83.3% | 2.61E−02 | 2.12E−01 |
| 1-phosphoribosyl-imidazole | 2 | (1.1%) | 12 | (5.4%) | 85.7% | 2.69E−02 | 2.12E−01 |
| beta-hydroxy acid | 14 | (8%) | 7 | (3.2%) | 33.3% | 4.13E−02 | 3.16E−01 |
| imidazopyrimidine | 7 | (4%) | 21 | (9.5%) | 75.0% | 4.70E−02 | 3.36E−01 |
| purine | 7 | (4%) | 21 | (9.5%) | 75.0% | 4.70E−02 | 3.36E−01 |
| hemiacetal | 8 | (4.6%) | 3 | (1.4%) | 27.3% | 6.64E−02 | 4.56E−01 |
| hypoxanthine | 1 | (0.6%) | 7 | (3.2%) | 87.5% | 8.29E−02 | 4.98E−01 |
| disaccharide phosphate | 1 | (0.6%) | 8 | (3.6%) | 88.9% | 8.40E−02 | 4.98E−01 |
| organic hypophosphite | 38 | (21.7%) | 66 | (29.7%) | 63.5% | 8.46E−02 | 4.98E−01 |
| tertiary carboxylic acid amide | 5 | (2.9%) | 1 | (0.5%) | 16.7% | 9.14E−02 | 5.23E−01 |
| phosphoric acid ester | 37 | (21.1%) | 63 | (28.4%) | 63.0% | 1.04E−01 | 5.83E−01 |
| carnitine | 10 | (5.7%) | 5 | (2.3%) | 33.3% | 1.09E−01 | 5.96E−01 |
| polyamine | 4 | (2.3%) | 12 | (5.4%) | 75.0% | 1.31E−01 | 6.80E−01 |
| organic phosphite | 38 | (21.7%) | 64 | (28.8%) | 62.7% | 1.32E−01 | 6.80E−01 |
| purinone | 1 | (0.6%) | 6 | (2.7%) | 85.7% | 1.40E−01 | 6.98E−01 |
| 1,2-aminoalcohol | 9 | (5.1%) | 5 | (2.3%) | 35.7% | 1.70E−01 | 8.28E−01 |
| secondary aliphatic amine | 6 | (3.4%) | 3 | (1.4%) | 33.3% | 1.91E−01 | 9.05E−01 |
| (dialkylamine) | | | | | | | |
| pentose monosaccharide | 11 | (6.3%) | 23 | (10.4%) | 67.6% | 2.06E−01 | 9.41E−01 |
| 1,2-diol | 32 | (18.3%) | 53 | (23.9%) | 62.4% | 2.18E−01 | 9.78E−01 |
| monosaccharide phosphate | 8 | (4.6%) | 16 | (7.2%) | 66.7% | 2.98E−01 | 1.00E+00 |
| carboxylic acid ester | 29 | (16.6%) | 47 | (21.2%) | 61.8% | 3.04E−01 | 1.00E+00 |
| alpha-hydroxy acid | 8 | (4.6%) | 6 | (2.7%) | 42.9% | 4.13E−01 | 1.00E+00 |
| secondary alcohol | 63 | (36%) | 89 | (40.1%) | 58.6% | 4.67E−01 | 1.00E+00 |
| ketone | 2 | (1.1%) | 6 | (2.7%) | 75.0% | 4.75E−01 | 1.00E+00 |
| succinic_acid | 5 | (2.9%) | 4 | (1.8%) | 44.4% | 5.16E−01 | 1.00E+00 |
| phosphoethanolamine | 21 | (12%) | 32 | (14.4%) | 60.4% | 5.53E−01 | 1.00E+00 |
| oxane | 8 | (4.6%) | 7 | (3.2%) | 46.7% | 5.98E−01 | 1.00E+00 |
| allyl alcohol | 4 | (2.3%) | 3 | (1.4%) | 42.9% | 7.04E−01 | 1.00E+00 |
| pyrrolidine carboxylic acid | 4 | (2.3%) | 3 | (1.4%) | 42.9% | 7.04E−01 | 1.00E+00 |
| alkylthiol | 4 | (2.3%) | 4 | (1.8%) | 50.0% | 7.36E−01 | 1.00E+00 |
| thiol (sulfanyl compound) | 4 | (2.3%) | 4 | (1.8%) | 50.0% | 7.36E−01 | 1.00E+00 |
| pyrrolidine | 4 | (2.3%) | 4 | (1.8%) | 50.0% | 7.36E−01 | 1.00E+00 |
| cyclohexane | 3 | (1.7%) | 6 | (2.7%) | 66.7% | 7.37E−01 | 1.00E+00 |
| guanidine | 3 | (1.7%) | 6 | (2.7%) | 66.7% | 7.37E−01 | 1.00E+00 |
| n-acylglycine | 5 | (2.9%) | 5 | (2.3%) | 50.0% | 7.55E−01 | 1.00E+00 |
| primary carboxylic acid amide | 6 | (3.4%) | 6 | (2.7%) | 50.0% | 7.71E−01 | 1.00E+00 |
| primary alcohol | 36 | (20.6%) | 49 | (22.1%) | 57.6% | 8.05E−01 | 1.00E+00 |
| short-chain hydroxy acid | 4 | (2.3%) | 5 | (2.3%) | 55.6% | 1.00E+00 | 1.00E+00 |
| phenol | 3 | (1.7%) | 3 | (1.4%) | 50.0% | 1.00E+00 | 1.00E+00 |
| phenol derivative | 3 | (1.7%) | 3 | (1.4%) | 50.0% | 1.00E+00 | 1.00E+00 |
| thioether | 5 | (2.9%) | 6 | (2.7%) | 54.5% | 1.00E+00 | 1.00E+00 |
| urea | 3 | (1.7%) | 4 | (1.8%) | 57.1% | 1.00E+00 | 1.00E+00 |

TABLE 8
Metabolites found and missed in FFPE categorized by superclass.

| | non-preserved in | preserved in | | |
|---|---|---|---|---|
| Propriety | FFPE, n (%) | FFPE, n (%) | P | FDR |
| logp_ALOGPS | −0.75 | 0.75 | 8.09E−05 | 1.29E−03 |
| logp_ChemAxon | −1.30 | 0.05 | 1.78E−03 | 1.42E−02 |
| physiological_charge_ChemAxon | −0.51 | −0.68 | 4.34E−02 | 2.32E−01 |
| polar_surface_area_ChemAxon | 108.63 | 110.53 | 1.46E−01 | 4.76E−01 |
| solubility_ALOGPS | 65.75 | 61.01 | 1.60E−01 | 4.76E−01 |
| logs_ALOGPS | −2.22 | −2.61 | 1.79E−01 | 4.76E−01 |
| donor_count_ChemAxon | 3.13 | 3.13 | 3.12E−01 | 6.43E−01 |
| pka_strongest_basic_ChemAxon | 2.76 | 2.59 | 3.51E−01 | 6.43E−01 |
| acceptor_count_ChemAxon | 5.02 | 5.23 | 3.62E−01 | 6.43E−01 |
| refractivity_ChemAxon | 74.30 | 75.15 | 6.38E−01 | 8.83E−01 |
| polarizability_ChemAxon | 29.72 | 30.63 | 6.67E−01 | 8.83E−01 |
| average_mass_ChemAxon | 282.87 | 294.64 | 7.14E−01 | 8.83E−01 |
| mono_mass_ChemAxon | 282.70 | 294.46 | 7.18E−01 | 8.83E−01 |
| rotatable_bond_count_ChemAxon | 8.93 | 9.56 | 8.19E−01 | 9.26E−01 |
| formal_charge_ChemAxon | 0.01 | 0.00 | 8.68E−01 | 9.26E−01 |
| pka_strongest_acidic_ChemAxon | 3.94 | 4.45 | 9.51E−01 | 9.51E−01 |

TABLE 9
Metabolites found and missed in FFPE categorized by superclass.

| | non-preserved in | preserved in | FFPE/ | | |
|---|---|---|---|---|---|
| Pathway | FFPE, n (%) | FFPE, n (%) | FROZEN, % | P | FDR |
| Transcription/Translation | 2 (1.1%) | 22 (9.9%) | 91.7% | 1.73E−04 | 1.52E−02 |
| Purine Metabolism | 2 (1.1%) | 18 (8.1%) | 90.0% | 1.85E−03 | 8.13E−02 |
| Ammonia Recycling | 1 (0.6%) | 12 (5.4%) | 92.3% | 8.24E−03 | 2.27E−01 |
| Alpha Linolenic Acid and | 0 (0%) | 8 (3.6%) | 100.0% | 1.03E−02 | 2.27E−01 |
| Linoleic Acid Metabolism | | | | | |
| Urea Cycle | 1 (0.6%) | 11 (5%) | 91.7% | 1.49E−02 | 2.62E−01 |
| Pyrimidine Metabolism | 3 (1.7%) | 15 (6.8%) | 83.3% | 2.61E−02 | 3.82E−01 |
| Citric Acid Cycle | 2 (1.1%) | 11 (5%) | 84.6% | 4.50E−02 | 5.65E−01 |
| Valine, Leucine and Isoleucine | 1 (0.6%) | 7 (3.2%) | 87.5% | 8.29E−02 | 5.68E−01 |
| Degradation | | | | | |
| Aspartate Metabolism | 1 (0.6%) | 7 (3.2%) | 87.5% | 8.29E−02 | 5.68E−01 |
| Arginine and Proline Metabolism | 1 (0.6%) | 8 (3.6%) | 88.9% | 8.40E−02 | 5.68E−01 |
| Transfer of Acetyl Groups into | 1 (0.6%) | 8 (3.6%) | 88.9% | 8.40E−02 | 5.68E−01 |
| Mitochondria | | | | | |
| Pentose Phosphate Pathway | 5 (2.9%) | 1 (0.5%) | 16.7% | 9.14E−02 | 5.75E−01 |
| Glycine and Serine Metabolism | 7 (4%) | 18 (8.1%) | 72.0% | 1.01E−01 | 5.87E−01 |
| Glutamate Metabolism | 2 (1.1%) | 9 (4.1%) | 81.8% | 1.22E−01 | 5.87E−01 |
| Lactose Synthesis | 1 (0.6%) | 6 (2.7%) | 85.7% | 1.40E−01 | 5.87E−01 |
| Glucose-Alanine Cycle | 1 (0.6%) | 6 (2.7%) | 85.7% | 1.40E−01 | 5.87E−01 |
| Plasmalogen Synthesis | 1 (0.6%) | 6 (2.7%) | 85.7% | 1.40E−01 | 5.87E−01 |
| Pyruvate Metabolism | 1 (0.6%) | 5 (2.3%) | 83.3% | 2.35E−01 | 8.75E−01 |
| Beta-Alanine Metabolism | 1 (0.6%) | 5 (2.3%) | 83.3% | 2.35E−01 | 8.75E−01 |
| Glutathione Metabolism | 2 (1.1%) | 6 (2.7%) | 75.0% | 4.75E−01 | 1.00E+00 |
| Galactose Metabolism | 2 (1.1%) | 6 (2.7%) | 75.0% | 4.75E−01 | 1.00E+00 |
| Methionine Metabolism | 5 (2.9%) | 9 (4.1%) | 64.3% | 5.93E−01 | 1.00E+00 |
| Nicotinate and Nicotinamide | 2 (1.1%) | 4 (1.8%) | 66.7% | 6.98E−01 | 1.00E+00 |
| Metabolism | | | | | |
| Phospholipid Biosynthesis | 2 (1.1%) | 4 (1.8%) | 66.7% | 6.98E−01 | 1.00E+00 |
| Spermidine and Spermine | 2 (1.1%) | 4 (1.8%) | 66.7% | 6.98E−01 | 1.00E+00 |
| Biosynthesis | | | | | |
| Mitochondrial Electron Transport | 2 (1.1%) | 4 (1.8%) | 66.7% | 6.98E−01 | 1.00E+00 |
| Chain | | | | | |
| Histidine Metabolism | 3 (1.7%) | 6 (2.7%) | 66.7% | 7.37E−01 | 1.00E+00 |
| Glycerolipid Metabolism | 3 (1.7%) | 6 (2.7%) | 66.7% | 7.37E−01 | 1.00E+00 |
| Gluconeogenesis | 5 (2.9%) | 8 (3.6%) | 61.5% | 7.81E−01 | 1.00E+00 |
| Amino Sugar Metabolism | 6 (3.4%) | 9 (4.1%) | 60.0% | 7.97E−01 | 1.00E+00 |
| Glycolysis | 5 (2.9%) | 6 (2.7%) | 54.5% | 1.00E+00 | 1.00E+00 |
| Betaine Metabolism | 3 (1.7%) | 3 (1.4%) | 50.0% | 1.00E+00 | 1.00E+00 |
| Carnitine Synthesis | 5 (2.9%) | 6 (2.7%) | 54.5% | 1.00E+00 | 1.00E+00 |
| Fructose and Mannose | 3 (1.7%) | 3 (1.4%) | 50.0% | 1.00E+00 | 1.00E+00 |
| Degradation | | | | | |
| Mitochondrial Beta-Oxidation of | 3 (1.7%) | 5 (2.3%) | 62.5% | 1.00E+00 | 1.00E+00 |
| Long Chain Saturated Fatty Acids | | | | | |
| Mitochondrial Beta-Oxidation of | 3 (1.7%) | 4 (1.8%) | 57.1% | 1.00E+00 | 1.00E+00 |
| Short Chain Saturated Fatty Acids | | | | | |

At the extremes, only 6 peptides of 56 were detected (11%, P=4.56×10⁻¹³; FDR=3.65×10⁻¹²), whereas 114 lipids of 171 analyzed (67%, P=1.01×10⁴; FDR=4.03×10⁻⁴) were preserved in FFPE samples. The majority of fatty acids (93%, P=4.74×10⁻⁶; FDR=5.93×10⁻⁵), including lysophosphatidylethanolamine (94%, P=4.18×10⁴; FDR=4.73×10⁻³), glycerolipids (100%, P=2.97×10⁻³; FDR=2.47×10⁻²), pyrimidine nucleotides (92%, P=8.24×10⁻³; FDR=5.15×10⁻²), and purine nucleotides (85%, P=4.50×10⁻²; FDR=1.87×10⁻¹), were detectable in FFPE samples, whereas monosaccharides (23%, P=2.11×10⁻²; FDR=1.05×10⁻¹), phosphatidylcholines (0%, P=1.30×10⁻³; FDR=1.11×10⁻²), and lysophosphatidylcholines (46%, P=5.74×10⁻¹; FDR=7.65×10⁻¹) were poorly detectable in FFPE samples. FFPE samples showed a decrease of metabolites with characteristic functional groups, such as secondary carboxylic acid amide (28%, P=7.43×10⁻¹²; FDR=6.42×10⁻¹⁰), present in peptides and quaternary ammonium salts (33%, P=1.01×10⁻³; FDR=2.30×10⁻²) present in glycerophosphocholines and absent in glycerophosphoethanolamines. No specific depletion of metabolic pathway information was observed. Nonparametric Wilcoxon-Mann-Whitney test was used to evaluate the difference between chemical/physical properties. Lipophilic metabolites showed high detectability in FFPE samples (P=8.09×10⁻⁵; FDR=1.29×10⁻³).

### Example 3: Metabolic Data Reproducibility and Consistency Between FFPE and Frozen Isogenic Prostate Cancer Cell Line Samples

To evaluate data reproducibility in different biological replicates, correlation analyses were performed among the shared metabolites in the five cell culture sets. Pair-wise correlation coefficients were consistently high for both frozen and FFPE samples indicating a minimal variability among replicates. The correlation coefficients, calculated in FFPE cell line samples (FIG. 2E, left plot), ranged between 0.904 and 0.986 (median value of 0.956), which were slightly lower than those in frozen samples ranging between 0.968 and 0.994 (median value of 0.989).

In order to expand metabolomics analyses to retrospective studies confidently, metabolic data from FFPE samples should be consistent with those obtained from frozen material. To test this, the relative concentration of metabolites between frozen and FFPE samples were correlated. A good correlation between the metabolomic data from frozen and FFPE samples was maintained. The correlation coefficients, calculated in cell line samples, ranged between 0.550 and 0.709 (median value of 0.651) (FIG. 2E, right plot).

The reproducibility in the detection of different metabolite classes between FFPE and frozen samples were compared. The correlation coefficients were calculated for each metabolic class (i.e., energy, nucleotides, lipids, amino acids, carbohydrates, cofactors and vitamins) between cell lines replicates. The results, shown in FIG. 2F, indicate that data reproducibility is maintained in all analyzed classes in both frozen and FFPE replicates. When the correlation coefficients between frozen and FFPE samples were compared, a favorable correlation was observed for nearly all of the classes (median correlation value ranges between 0.676 and 0.867) except for carbohydrates (correlation value of 0.322).

### Example 4: Metabolic Profiling of Isogenic Prostate Cancer Cell Lines

Metabolic profiling was used to distinguish androgen dependent LNCaP cells from their isogenic, androgen-independent LNCaP-Abl using both frozen and formalin-fixed samples. To perform a comparative analysis between LNCaP and LNCaP-Abl cells, only the shared metabolites found with less than 25% missing values in both frozen and FFPE samples were considered. From among the 189 metabolites retained for analysis, hierarchical clustering based on the KODAMA dissimilarity matrix was applied to show the clear metabolic profiles of LNCaP and LNCaP-Abl cells. This unsupervised method was chosen since it has been previously shown to be very robust even when applied to noisy data (1, 16). Using the 189 shared metabolites between frozen and FFPE samples, the two cell lines were distinguished, with a high degree of accuracy, on the basis of their metabolic profiling in both fixed and frozen states (FIG. 2G).

Comparing LNCaP and LNCaP-Abl cells, significantly different (Wilcoxon test P<0.05; 10% FDR) 108 metabolites in frozen samples and 65 in FFPE samples were found, with 42 statistically significant in both frozen and FFPE analysis (FIG. 2H). Almost the totality of the statistically significant metabolites were concordant in the directionality of their expression (down-regulated or up-regulated). The levels of some amino acids, such as alanine (Pfrozen=7.94×10⁻³; PFFPE=7.94×10⁻³), asparagine (Pfrozen=7.94×10⁻³; PFFPE=7.94×10⁻³), and glutamate (Pfrozen=7.94×10⁻³; PFFPE=7.94×10⁻³), were significantly decreased in androgen-independent LNCaP-Abl cells. This could support the observation that androgen signaling regulates amino acid metabolism, as described in the art (18, 19). The complete list of metabolites is reported in Table 10 and Table 11, which list metabolite statistical analysis of the differences between LNCaP/LNCaP-Abl in frozen and FFPE samples, respectively.

TABLE 10
Metabolite statistical analysis of LNCaP and LNCaP-Abl in frozen samples

| | FROZEN | | | | | |
|---|---|---|---|---|---|---|
| Compound | p | FDR | mean A | mean B | log ratio | loadings |
| 1-arachidonylglycerol | 7.94E−03 | 1.65E−02 | 7.03E−05 | 5.38E−05 | −0.39 | −0.08 |
| 1-palmitoylglycerol | 7.94E−03 | 1.65E−02 | 1.94E−03 | 3.44E−03 | 0.83 | 0.09 |
| 5-oxoproline | 7.94E−03 | 1.65E−02 | 4.74E−03 | 2.68E−03 | −0.82 | −0.09 |
| alanine | 7.94E−03 | 1.65E−02 | 6.22E−03 | 1.00E−03 | −2.63 | −0.10 |
| allantoin | 7.94E−03 | 1.65E−02 | 3.57E−04 | 8.41E−05 | −2.09 | −0.10 |
| arginine | 7.94E−03 | 1.65E−02 | 1.89E−03 | 1.30E−03 | −0.53 | −0.09 |
| asparagine | 7.94E−03 | 1.65E−02 | 6.35E−02 | 2.15E−02 | −1.56 | −0.10 |
| biopterin | 7.94E−03 | 1.65E−02 | 1.81E−05 | 6.39E−05 | 1.82 | 0.09 |
| carnosine | 7.94E−03 | 1.65E−02 | 3.01E−04 | 3.60E−05 | −3.06 | −0.10 |
| choline | 7.94E−03 | 1.65E−02 | 1.86E−04 | 1.56E−03 | 3.08 | 0.10 |
| choline phosphate | 7.94E−03 | 1.65E−02 | 2.01E−02 | 5.79E−02 | 1.53 | 0.10 |
| citrate | 7.94E−03 | 1.65E−02 | 5.21E−02 | 2.57E−02 | −1.02 | −0.08 |
| citrulline | 7.94E−03 | 1.65E−02 | 3.87E−04 | 5.12E−04 | 0.40 | 0.09 |
| creatinine | 7.94E−03 | 1.65E−02 | 4.79E−03 | 2.96E−03 | −0.70 | −0.10 |
| docosahexaenoate | 7.94E−03 | 1.65E−02 | 1.67E−04 | 6.11E−05 | −1.45 | −0.09 |
| eicosenoate | 7.94E−03 | 1.65E−02 | 6.80E−04 | 2.02E−04 | −1.75 | −0.09 |
| gamma-aminobutyrate | 7.94E−03 | 1.65E−02 | 5.83E−04 | 2.04E−03 | 1.81 | 0.10 |
| glutamate | 7.94E−03 | 1.65E−02 | 4.43E−02 | 5.93E−02 | 0.42 | 0.09 |
| glutathione, oxidized | 7.94E−03 | 1.65E−02 | 1.47E−02 | 4.64E−02 | 1.66 | 0.09 |
| glutathione, reduced | 7.94E−03 | 1.65E−02 | 9.09E−02 | 2.04E−01 | 1.17 | 0.10 |
| glycerophosphorylcholine | 7.94E−03 | 1.65E−02 | 1.87E−03 | 7.44E−03 | 1.99 | 0.10 |
| inosine | 7.94E−03 | 1.65E−02 | 4.74E−04 | 7.34E−04 | 0.63 | 0.08 |
| linolenate (alpha or | 7.94E−03 | 1.65E−02 | 5.36E−05 | 1.66E−04 | 1.63 | 0.10 |
| gamma) | | | | | | |
| myo-inositol | 7.94E−03 | 1.65E−02 | 2.78E−03 | 1.63E−02 | 2.55 | 0.10 |
| palmitoyl-linoleoyl- | 7.94E−03 | 1.65E−02 | 8.70E−06 | 2.29E−05 | 1.40 | 0.08 |
| glycerophosphoinositol | | | | | | |
| (1) | | | | | | |
| phosphoethanolamine | 7.94E−03 | 1.65E−02 | 7.44E−05 | 3.42E−05 | −1.12 | −0.08 |
| taurine | 7.94E−03 | 1.65E−02 | 5.78E−04 | 1.54E−04 | −1.91 | −0.10 |
| adenine | 7.94E−03 | 1.65E−02 | 1.92E−04 | 4.43E−04 | 1.21 | 0.10 |
| methionine | 7.94E−03 | 1.65E−02 | 5.04E−03 | 1.09E−02 | 1.11 | 0.09 |
| methionine sulfoxide | 7.94E−03 | 1.65E−02 | 7.64E−04 | 2.48E−04 | −1.63 | −0.10 |
| trans-4-hydroxyproline | 7.94E−03 | 1.65E−02 | 3.43E−02 | 6.22E−03 | −2.46 | −0.10 |
| tyrosine | 7.94E−03 | 1.65E−02 | 1.42E−03 | 9.62E−03 | 2.76 | 0.10 |
| UDP-glucuronate | 7.94E−03 | 1.65E−02 | 2.62E−03 | 7.00E−04 | −1.90 | −0.09 |
| 1- | 7.94E−03 | 1.65E−02 | 7.17E−05 | 2.40E−05 | −1.58 | −0.08 |
| eicosenoylglycerophosphocholine | | | | | | |
| 3-hydroxy-3- | 7.94E−03 | 1.65E−02 | 1.27E−03 | 4.29E−03 | 1.76 | 0.10 |
| methylglutarate | | | | | | |
| aspartate | 7.94E−03 | 1.65E−02 | 5.00E−03 | 7.62E−03 | 0.61 | 0.09 |
| leucine | 7.94E−03 | 1.65E−02 | 1.46E−02 | 7.12E−02 | 2.29 | 0.10 |
| N-delta-acetylornithine | 7.94E−03 | 1.65E−02 | 1.27E−03 | 7.20E−04 | −0.81 | −0.09 |
| threonine | 7.94E−03 | 1.65E−02 | 3.82E−03 | 2.06E−03 | −0.89 | −0.10 |
| uridine 5′-monophosphate | 7.94E−03 | 1.65E−02 | 3.77E−04 | 1.07E−04 | −1.81 | −0.10 |
| cytidine 5′- | 7.94E−03 | 1.65E−02 | 2.84E−04 | 1.55E−04 | −0.87 | −0.09 |
| monophosphate | | | | | | |
| glycerophosphoethanolamine | 7.94E−03 | 1.65E−02 | 7.42E−04 | 2.43E−03 | 1.71 | 0.10 |
| p-cresol sulfate | 7.94E−03 | 1.65E−02 | 2.27E−05 | 1.69E−05 | −0.42 | −0.07 |
| 1- | 7.94E−03 | 1.65E−02 | 6.64E−06 | 2.60E−05 | 1.97 | 0.06 |
| linoleoylglycerophosphoethanolamine | | | | | | |
| eicosapentaenoate | 7.94E−03 | 1.65E−02 | 3.45E−05 | 7.53E−05 | 1.13 | 0.09 |
| glutamine | 7.94E−03 | 1.65E−02 | 2.66E−05 | 1.56E−03 | 5.87 | 0.10 |
| glycine | 7.94E−03 | 1.65E−02 | 2.21E−03 | 1.14E−03 | −0.95 | −0.09 |
| myristoleate | 7.94E−03 | 1.65E−02 | 2.87E−04 | 1.25E−04 | −1.20 | −0.09 |
| serine | 7.94E−03 | 1.65E−02 | 1.05E−02 | 5.48E−03 | −0.94 | −0.10 |
| trizma acetate | 7.94E−03 | 1.65E−02 | 3.52E−05 | 9.15E−05 | 1.38 | 0.09 |
| xanthosine | 7.94E−03 | 1.65E−02 | 7.42E−06 | 4.32E−06 | −0.78 | −0.08 |
| adenosine 5′- | 7.94E−03 | 1.65E−02 | 6.84E−03 | 2.20E−03 | −1.64 | −0.10 |
| monophosphate | | | | | | |
| fumarate | 7.94E−03 | 1.65E−02 | 1.04E−03 | 1.94E−03 | 0.90 | 0.09 |
| glucose | 7.94E−03 | 1.65E−02 | 5.12E−05 | 3.29E−04 | 2.68 | 0.10 |
| lactate | 7.94E−03 | 1.65E−02 | 3.05E−03 | 9.69E−04 | −1.65 | −0.10 |
| malate | 7.94E−03 | 1.65E−02 | 6.56E−03 | 1.39E−02 | 1.08 | 0.10 |
| succinate | 7.94E−03 | 1.65E−02 | 1.56E−03 | 9.30E−04 | −0.74 | −0.09 |
| tryptophan | 7.94E−03 | 1.65E−02 | 5.85E−04 | 2.19E−03 | 1.90 | 0.10 |
| 1- | 7.94E−03 | 1.65E−02 | 8.44E−06 | 2.32E−05 | 1.46 | 0.07 |
| oleoylglycerophosphoinositol | | | | | | |
| Isobar: fructose 1,6- | 7.94E−03 | 1.65E−02 | 1.07E−04 | 7.45E−04 | 2.80 | 0.10 |
| diphosphate, glucose 1,6- | | | | | | |
| diphosphate, myo-inositol | | | | | | |
| 1,4 or 1,3-diphosphate | | | | | | |
| N-acetylmethionine | 7.94E−03 | 1.65E−02 | 2.14E−04 | 1.18E−04 | −0.85 | −0.10 |
| N-acetylserine | 7.94E−03 | 1.65E−02 | 1.05E−02 | 5.42E−03 | −0.96 | −0.10 |
| xanthine | 7.94E−03 | 1.65E−02 | 1.88E−04 | 7.02E−05 | −1.42 | −0.09 |
| pyroglutamine | 7.94E−03 | 1.65E−02 | 9.46E−04 | 1.31E−03 | 0.47 | 0.08 |
| beta-hydroxyisovalerate | 7.94E−03 | 1.65E−02 | 8.09E−04 | 2.41E−03 | 1.58 | 0.10 |
| docosapentaenoate (n6) | 7.94E−03 | 1.65E−02 | 4.93E−05 | 9.24E−06 | −2.41 | −0.09 |
| erythronate | 7.94E−03 | 1.65E−02 | 3.07E−02 | 1.42E−02 | −1.11 | −0.10 |
| flavin mononucleotide | 7.94E−03 | 1.65E−02 | 1.04E−05 | 6.39E−06 | −0.70 | −0.10 |
| guanosine 5′- | 7.94E−03 | 1.65E−02 | 4.52E−04 | 3.18E−05 | −3.83 | −0.10 |
| monophosphate | | | | | | |
| guanosine 5′-diphospho- | 7.94E−03 | 1.65E−02 | 4.95E−06 | 1.10E−05 | 1.16 | 0.08 |
| fucose | | | | | | |
| methylmalonate | 7.94E−03 | 1.65E−02 | 1.29E−04 | 5.63E−05 | −1.19 | −0.09 |
| proline | 7.94E−03 | 1.65E−02 | 1.04E−01 | 3.63E−02 | −1.52 | −0.10 |
| 1- | 7.94E−03 | 1.65E−02 | 1.69E−05 | 8.15E−05 | 2.27 | 0.09 |
| eicosatrienoylglycerophosphoethanolamine | | | | | | |
| 4-guanidinobutanoate | 7.94E−03 | 1.65E−02 | 3.73E−04 | 2.45E−03 | 2.72 | 0.10 |
| phenylalanine | 7.94E−03 | 1.65E−02 | 3.20E−03 | 1.99E−02 | 2.64 | 0.10 |
| 4-methyl-2- | 7.94E−03 | 1.65E−02 | 5.24E−05 | 2.22E−04 | 2.08 | 0.10 |
| oxopentanoate | | | | | | |
| 5-dodecenoate | 7.94E−03 | 1.65E−02 | 1.30E−04 | 2.21E−05 | −2.56 | −0.09 |
| acetylcarnitine | 7.94E−03 | 1.65E−02 | 2.55E−02 | 1.19E−02 | −1.10 | −0.10 |
| benzoate | 7.94E−03 | 1.65E−02 | 1.58E−04 | 2.18E−04 | 0.46 | 0.09 |
| dihomo-linolenate | 7.94E−03 | 1.65E−02 | 7.89E−05 | 1.55E−04 | 0.97 | 0.08 |
| ethylmalonate | 7.94E−03 | 1.65E−02 | 9.34E−03 | 3.21E−03 | −1.54 | −0.10 |
| glutamate, gamma- | 7.94E−03 | 1.65E−02 | 4.78E−03 | 2.88E−02 | 2.59 | 0.09 |
| methyl ester | | | | | | |
| guanine | 7.94E−03 | 1.65E−02 | 2.43E−04 | 5.83E−05 | −2.06 | −0.08 |
| isoleucine | 7.94E−03 | 1.65E−02 | 1.84E−02 | 8.07E−02 | 2.13 | 0.10 |
| UDP-N- | 7.94E−03 | 1.65E−02 | 8.70E−03 | 2.14E−03 | −2.02 | −0.10 |
| acetylglucosamine | | | | | | |
| 3-methyl-2-oxobutyrate | 7.94E−03 | 1.65E−02 | 2.74E−05 | 5.17E−05 | 0.91 | 0.09 |
| creatine | 7.94E−03 | 1.65E−02 | 1.33E−01 | 1.08E−01 | −0.30 | −0.08 |
| erucate | 7.94E−03 | 1.65E−02 | 1.17E−04 | 5.15E−05 | −1.18 | −0.08 |
| histidine | 7.94E−03 | 1.65E−02 | 1.87E−03 | 7.81E−04 | −1.26 | −0.10 |
| isobutyrylcarnitine | 7.94E−03 | 1.65E−02 | 5.35E−04 | 1.80E−04 | −1.57 | −0.10 |
| pyridoxal | 7.94E−03 | 1.65E−02 | 8.80E−05 | 6.13E−04 | 2.80 | 0.10 |
| 2′-deoxyadenosine 5′- | 1.59E−02 | 3.00E−02 | 6.78E−06 | 9.29E−06 | 0.45 | 0.07 |
| monophosphate | | | | | | |
| stearoyl-arachidonoyl- | 1.59E−02 | 3.00E−02 | 1.93E−05 | 7.28E−06 | −1.41 | −0.08 |
| glycerophosphoinositol | | | | | | |
| (1) | | | | | | |
| cytidine triphosphate | 1.59E−02 | 3.00E−02 | 4.04E−04 | 8.77E−04 | 1.12 | 0.08 |
| mead acid | 1.59E−02 | 3.00E−02 | 3.29E−04 | 6.54E−04 | 0.99 | 0.08 |
| prolylalanine | 1.59E−02 | 3.00E−02 | 1.50E−05 | 2.91E−05 | 0.95 | 0.08 |
| 2- | 1.59E−02 | 3.00E−02 | 7.40E−05 | 2.20E−04 | 1.57 | 0.06 |
| palmitoleoylglycerophosphoethanolamine | | | | | | |
| glycerophosphoinositol | 1.59E−02 | 3.00E−02 | 1.48E−04 | 1.06E−04 | −0.48 | −0.07 |
| coenzyme A | 1.59E−02 | 3.00E−02 | 1.94E−04 | 4.33E−04 | 1.16 | 0.08 |
| nicotinamide | 1.59E−02 | 3.00E−02 | 1.98E−03 | 1.42E−03 | −0.47 | −0.08 |
| 1- | 3.17E−02 | 5.56E−02 | 2.42E−05 | 1.36E−05 | −0.83 | −0.08 |
| oleoylglycerophosphoserine | | | | | | |
| 2′-deoxyguanosine | 3.17E−02 | 5.56E−02 | 3.23E−06 | 5.55E−06 | 0.78 | 0.07 |
| oleoyl-linoleoyl- | 3.17E−02 | 5.56E−02 | 9.47E−06 | 1.85E−05 | 0.97 | 0.07 |
| glycerophosphoinositol | | | | | | |
| (1) | | | | | | |
| pterin | 3.17E−02 | 5.56E−02 | 3.69E−05 | 6.71E−05 | 0.86 | 0.08 |
| alpha-ketoglutarate | 3.17E−02 | 5.56E−02 | 1.13E−03 | 8.84E−04 | −0.36 | −0.07 |
| 4-hydroxyphenylpyruvate | 3.17E−02 | 5.56E−02 | 2.40E−05 | 3.12E−05 | 0.38 | 0.07 |
| glycerate | 3.17E−02 | 5.56E−02 | 2.50E−04 | 3.57E−04 | 0.51 | 0.07 |
| methylphosphate | 3.17E−02 | 5.56E−02 | 1.59E−04 | 1.17E−04 | −0.44 | −0.06 |
| 1- | 5.56E−02 | 9.38E−02 | 2.95E−05 | 2.07E−05 | −0.51 | −0.06 |
| docosahexaenoylglycerol | | | | | | |
| cytidine | 5.56E−02 | 9.38E−02 | 3.34E−05 | 2.91E−05 | −0.20 | −0.06 |
| 7-methylguanine | 5.56E−02 | 9.38E−02 | 2.37E−06 | 3.19E−06 | 0.43 | 0.05 |
| cysteine-glutathione | 5.56E−02 | 9.38E−02 | 7.43E−05 | 2.41E−05 | −1.62 | −0.07 |
| disulfide | | | | | | |
| 1- | 9.52E−02 | 1.55E−01 | 2.13E−04 | 1.66E−04 | −0.36 | −0.06 |
| oleoylglycerophosphoethanolamine | | | | | | |
| uridine | 9.52E−02 | 1.55E−01 | 3.90E−04 | 2.92E−04 | −0.42 | −0.06 |
| uridine 5′-diphosphate | 9.52E−02 | 1.55E−01 | 7.82E−04 | 1.12E−03 | 0.52 | 0.06 |
| 1- | 9.52E−02 | 1.55E−01 | 2.39E−05 | 4.11E−05 | 0.78 | 0.06 |
| palmitoleoylglycerophosphoethanolamine | | | | | | |
| 2′-deoxycytidine | 1.51E−01 | 2.18E−01 | 6.76E−06 | 3.08E−06 | −1.13 | −0.04 |
| flavin adenine | 1.51E−01 | 2.18E−01 | 8.19E−05 | 9.72E−05 | 0.25 | 0.05 |
| dinucleotide | | | | | | |
| 1- | 1.51E−01 | 2.18E−01 | 5.22E−05 | 3.81E−05 | −0.45 | −0.04 |
| arachidonoylglycerophosphoethanolamine | | | | | | |
| 2′-deoxycytidine 5′- | 1.51E−01 | 2.18E−01 | 2.78E−05 | 1.14E−05 | −1.29 | −0.07 |
| monophosphate | | | | | | |
| hypoxanthine | 1.51E−01 | 2.18E−01 | 9.32E−06 | 5.26E−06 | −0.82 | −0.04 |
| docosapentaenoate (n3) | 1.51E−01 | 2.18E−01 | 7.74E−05 | 5.34E−05 | −0.53 | −0.05 |
| N-acetyl-aspartyl- | 1.51E−01 | 2.18E−01 | 3.08E−03 | 3.50E−03 | 0.19 | 0.05 |
| glutamate | | | | | | |
| linoleate | 1.51E−01 | 2.18E−01 | 6.11E−04 | 8.41E−04 | 0.46 | 0.06 |
| glycerol | 1.51E−01 | 2.18E−01 | 2.16E−04 | 1.75E−04 | −0.30 | −0.02 |
| phosphoenolpyruvate | 1.51E−01 | 2.18E−01 | 1.47E−04 | 2.08E−04 | 0.50 | 0.06 |
| S-adenosylhomocysteine | 1.51E−01 | 2.18E−01 | 6.01E−05 | 1.27E−04 | 1.08 | 0.06 |
| butyrylcarnitine | 1.51E−01 | 2.18E−01 | 5.08E−03 | 5.67E−03 | 0.16 | 0.04 |
| N-acetylaspartate | 1.51E−01 | 2.18E−01 | 1.19E−02 | 1.06E−02 | −0.17 | −0.06 |
| 2- | 1.51E−01 | 2.18E−01 | 6.50E−04 | 4.45E−04 | −0.54 | −0.05 |
| oleoylglycerophosphocholine | | | | | | |
| 2- | 1.51E−01 | 2.18E−01 | 2.12E−04 | 1.56E−04 | −0.44 | −0.05 |
| palmitoylglycerophosphocholine | | | | | | |
| 17-methylstearate | 1.73E−01 | 2.48E−01 | 3.12E−05 | 2.60E−05 | −0.26 | −0.05 |
| 1- | 2.22E−01 | 3.02E−01 | 5.84E−06 | 7.68E−06 | 0.39 | 0.05 |
| palmitoylglycerophosphoinositol | | | | | | |
| cytidine diphosphate | 2.22E−01 | 3.02E−01 | 1.67E−04 | 2.69E−04 | 0.69 | 0.05 |
| 1- | 2.22E−01 | 3.02E−01 | 1.20E−05 | 8.76E−06 | −0.46 | −0.05 |
| palmitoylglycerophosphoserine | | | | | | |
| 1- | 2.22E−01 | 3.02E−01 | 5.16E−04 | 3.45E−04 | −0.58 | −0.05 |
| oleoylglycerophosphocholine | | | | | | |
| cystathionine | 2.22E−01 | 3.02E−01 | 6.63E−05 | 5.22E−05 | −0.34 | −0.05 |
| lysine | 2.22E−01 | 3.02E−01 | 1.73E−04 | 1.39E−04 | −0.32 | −0.05 |
| N-acetylthreonine | 2.22E−01 | 3.02E−01 | 5.88E−04 | 6.59E−04 | 0.16 | 0.04 |
| adenosine | 3.10E−01 | 4.03E−01 | 1.90E−04 | 3.23E−04 | 0.76 | 0.05 |
| 2-arachidonoyl glycerol | 3.10E−01 | 4.03E−01 | 3.85E−05 | 5.06E−05 | 0.40 | 0.04 |
| myristate | 3.10E−01 | 4.03E−01 | 4.84E−03 | 4.58E−03 | −0.08 | −0.04 |
| 1- | 3.10E−01 | 4.03E−01 | 9.74E−04 | 1.66E−03 | 0.77 | 0.04 |
| palmitoylglycerophosphocholine | | | | | | |
| pyruvate | 3.10E−01 | 4.03E−01 | 3.18E−04 | 4.07E−04 | 0.36 | 0.04 |
| sphinganine | 3.10E−01 | 4.03E−01 | 3.26E−05 | 3.48E−05 | 0.09 | −0.01 |
| spermine | 3.46E−01 | 4.48E−01 | 1.15E−03 | 1.45E−03 | 0.33 | 0.01 |
| 1- | 4.21E−01 | 5.13E−01 | 6.05E−05 | 5.03E−05 | −0.27 | −0.03 |
| stearoylglycerophosphoinositol | | | | | | |
| 1- | 4.21E−01 | 5.13E−01 | 5.31E−06 | 9.24E−06 | 0.80 | 0.03 |
| docosahexaenoylglycerophosphoethanolamine | | | | | | |
| dihomo-linoleate | 4.21E−01 | 5.13E−01 | 2.94E−04 | 3.34E−04 | 0.18 | 0.03 |
| nonadecanoate | 4.21E−01 | 5.13E−01 | 9.88E−05 | 8.74E−05 | −0.18 | −0.05 |
| 2- | 4.21E−01 | 5.13E−01 | 1.91E−05 | 1.59E−05 | −0.26 | −0.02 |
| palmitoylglycerophosphoethanolamine | | | | | | |
| stearate | 4.21E−01 | 5.13E−01 | 4.21E−02 | 3.66E−02 | −0.20 | −0.04 |
| arachidate | 4.21E−01 | 5.13E−01 | 5.25E−04 | 4.43E−04 | −0.24 | −0.05 |
| phosphate | 4.21E−01 | 5.13E−01 | 2.16E−02 | 1.94E−02 | −0.15 | −0.04 |
| creatine phosphate | 4.21E−01 | 5.13E−01 | 4.23E−03 | 3.97E−03 | −0.09 | −0.03 |
| 2-palmitoylglycerol | 5.48E−01 | 6.27E−01 | 4.15E−04 | 4.46E−04 | 0.10 | 0.02 |
| 2′-deoxyinosine | 5.48E−01 | 6.27E−01 | 5.24E−06 | 5.76E−06 | 0.14 | 0.02 |
| 5-methylthioadenosine | 5.48E−01 | 6.27E−01 | 2.87E−03 | 3.05E−03 | 0.09 | 0.03 |
| arachidonate | 5.48E−01 | 6.27E−01 | 3.71E−04 | 3.01E−04 | −0.30 | −0.03 |
| nicotinamide adenine | 5.48E−01 | 6.27E−01 | 2.96E−03 | 2.98E−03 | 0.01 | 0.00 |
| dinucleotide | | | | | | |
| acetyl CoA | 5.48E−01 | 6.27E−01 | 1.01E−05 | 2.89E−06 | −1.80 | −0.02 |
| 3′-dephosphocoenzyme A | 5.48E−01 | 6.27E−01 | 1.36E−05 | 1.13E−05 | −0.26 | −0.03 |
| palmitate | 5.48E−01 | 6.27E−01 | 4.15E−02 | 3.53E−02 | −0.24 | −0.04 |
| 13-HODE + 9-HODE | 5.48E−01 | 6.27E−01 | 4.87E−06 | 5.20E−06 | 0.09 | 0.01 |
| stearoyl-oleoyl- | 5.48E−01 | 6.27E−01 | 1.68E−05 | 1.33E−05 | −0.34 | −0.03 |
| glycerophosphoserine (1) | | | | | | |
| 1- | 6.90E−01 | 7.54E−01 | 8.02E−05 | 1.05E−04 | 0.39 | 0.03 |
| stearoylglycerophosphoethanolamine | | | | | | |
| 15-methylpalmitate | 6.90E−01 | 7.54E−01 | 5.92E−04 | 6.53E−04 | 0.14 | 0.02 |
| (isobar with 2- | | | | | | |
| methylpalmitate) | | | | | | |
| margarate | 6.90E−01 | 7.54E−01 | 8.50E−04 | 8.58E−04 | 0.01 | 0.00 |
| 1-oleoylglycerol | 6.90E−01 | 7.54E−01 | 7.91E−05 | 9.45E−05 | 0.26 | 0.01 |
| pentadecanoate | 6.90E−01 | 7.54E−01 | 1.00E−03 | 1.05E−03 | 0.07 | 0.02 |
| spermidine | 6.90E−01 | 7.54E−01 | 1.35E−02 | 1.44E−02 | 0.09 | 0.01 |
| 4-methylglutamate | 6.90E−01 | 7.54E−01 | 3.67E−04 | 3.47E−04 | −0.08 | −0.02 |
| nicotinate | 6.90E−01 | 7.54E−01 | 1.95E−04 | 1.90E−04 | −0.04 | −0.01 |
| 1- | 8.41E−01 | 8.69E−01 | 8.50E−05 | 9.49E−05 | 0.16 | 0.02 |
| palmitoylglycerophosphoethanolamine | | | | | | |
| 13-methylmyristic acid | 8.41E−01 | 8.69E−01 | 3.40E−04 | 3.46E−04 | 0.03 | 0.00 |
| 10-heptadecenoate | 8.41E−01 | 8.69E−01 | 1.56E−04 | 1.63E−04 | 0.06 | 0.01 |
| 1-myristoylglycerol | 8.41E−01 | 8.69E−01 | 1.59E−04 | 1.66E−04 | 0.06 | 0.02 |
| 2-stearoylglycerol | 8.41E−01 | 8.69E−01 | 4.88E−05 | 7.85E−05 | 0.69 | 0.02 |
| 2- | 8.41E−01 | 8.69E−01 | 4.23E−05 | 4.14E−05 | −0.03 | −0.01 |
| arachidonoylglycerophosphoethanolamine | | | | | | |
| 1-stearoylglycerol | 8.41E−01 | 8.69E−01 | 6.15E−04 | 7.60E−04 | 0.31 | 0.00 |
| thymidine | 8.41E−01 | 8.69E−01 | 4.08E−06 | 4.18E−06 | 0.03 | 0.00 |
| 2- | 8.41E−01 | 8.69E−01 | 4.01E−05 | 7.84E−05 | 0.97 | 0.01 |
| linoleoylglycerophosphocholine | | | | | | |
| adenosine 5′- | 8.41E−01 | 8.69E−01 | 2.27E−05 | 2.05E−05 | −0.15 | 0.00 |
| diphosphoribose | | | | | | |
| palmitoleate | 1.00E+00 | 1.00E+00 | 2.01E−03 | 1.95E−03 | −0.04 | 0.00 |
| 1- | 1.00E+00 | 1.00E+00 | 3.13E−05 | 3.59E−05 | 0.20 | 0.02 |
| stearoylglycerophosphoserine | | | | | | |
| guanosine | 1.00E+00 | 1.00E+00 | 6.04E−05 | 5.84E−05 | −0.05 | −0.02 |
| 2- | 1.00E+00 | 1.00E+00 | 9.67E−05 | 1.13E−04 | 0.23 | 0.02 |
| oleoylglycerophosphoethanolamine | | | | | | |
| 1- | 1.00E+00 | 1.00E+00 | 1.08E−04 | 1.27E−04 | 0.24 | 0.02 |
| stearoylglycerophosphocholine | | | | | | |
| 1- | 1.00E+00 | 1.00E+00 | 2.37E−05 | 3.88E−05 | 0.71 | 0.00 |
| linoleoylglycerophosphocholine | | | | | | |

TABLE 11
Metabolite statistical analysis of LNCaP and LNCaP-Abl in FFPE samples

| | FFPE | | | | | |
|---|---|---|---|---|---|---|
| Compound | p | FDR | mean A | mean B | log ratio | loadings |
| 1-arachidonylglycerol | 7.94E−03 | 3.13E−02 | 4.09E−04 | 1.14E−04 | −1.85 | −0.11 |
| 1-palmitoylglycerol | 7.94E−03 | 3.13E−02 | 2.21E−03 | 1.79E−03 | −0.30 | −0.10 |
| 5-oxoproline | 7.94E−03 | 3.13E−02 | 2.25E−03 | 1.30E−03 | −0.80 | −0.11 |
| alanine | 7.94E−03 | 3.13E−02 | 2.25E−03 | 1.02E−03 | −1.14 | −0.12 |
| allantoin | 7.94E−03 | 3.13E−02 | 2.77E−04 | 8.27E−05 | −1.75 | −0.12 |
| arginine | 7.94E−03 | 3.13E−02 | 4.98E−04 | 1.83E−04 | −1.44 | −0.09 |
| asparagine | 7.94E−03 | 3.13E−02 | 2.68E−03 | 1.12E−03 | −1.26 | −0.12 |
| biopterin | 7.94E−03 | 3.13E−02 | 2.04E−05 | 4.89E−05 | 1.26 | 0.12 |
| carnosine | 7.94E−03 | 3.13E−02 | 1.41E−04 | 1.72E−05 | −3.04 | −0.12 |
| choline | 7.94E−03 | 3.13E−02 | 6.94E−04 | 1.29E−03 | 0.90 | 0.10 |
| choline phosphate | 7.94E−03 | 3.13E−02 | 8.94E−03 | 2.14E−02 | 1.26 | 0.10 |
| citrate | 7.94E−03 | 3.13E−02 | 3.56E−02 | 2.29E−02 | −0.63 | −0.12 |
| citrulline | 7.94E−03 | 3.13E−02 | 2.38E−04 | 6.01E−04 | 1.34 | 0.11 |
| creatinine | 7.94E−03 | 3.13E−02 | 3.50E−03 | 2.17E−03 | −0.69 | −0.11 |
| docosahexaenoate | 7.94E−03 | 3.13E−02 | 2.70E−04 | 1.08E−04 | −1.32 | −0.12 |
| eicosenoate | 7.94E−03 | 3.13E−02 | 4.71E−04 | 1.89E−04 | −1.32 | −0.11 |
| gamma-aminobutyrate | 7.94E−03 | 3.13E−02 | 5.53E−04 | 2.03E−03 | 1.87 | 0.12 |
| glutamate | 7.94E−03 | 3.13E−02 | 1.35E−02 | 1.98E−02 | 0.55 | 0.10 |
| glutathione, oxidized | 7.94E−03 | 3.13E−02 | 2.93E−03 | 6.87E−03 | 1.23 | 0.11 |
| glutathione, reduced | 7.94E−03 | 3.13E−02 | 9.26E−03 | 2.13E−02 | 1.20 | 0.11 |
| glycerophosphorylcholine | 7.94E−03 | 3.13E−02 | 9.22E−04 | 5.27E−04 | −0.81 | −0.09 |
| inosine | 7.94E−03 | 3.13E−02 | 5.44E−03 | 8.74E−03 | 0.68 | 0.11 |
| linolenate (alpha or | 7.94E−03 | 3.13E−02 | 1.74E−04 | 3.81E−04 | 1.13 | 0.11 |
| gamma) | | | | | | |
| myo-inositol | 7.94E−03 | 3.13E−02 | 1.71E−04 | 1.26E−03 | 2.88 | 0.12 |
| palmitoyl-linoleoyl- | 7.94E−03 | 3.13E−02 | 5.08E−05 | 1.83E−04 | 1.85 | 0.12 |
| glycerophosphoinositol | | | | | | |
| (1) | | | | | | |
| phosphoethanolamine | 7.94E−03 | 3.13E−02 | 1.73E−04 | 3.92E−05 | −2.14 | −0.12 |
| taurine | 7.94E−03 | 3.13E−02 | 5.85E−04 | 2.86E−04 | −1.03 | −0.11 |
| adenine | 1.59E−02 | 5.26E−02 | 2.01E−03 | 3.19E−03 | 0.67 | 0.09 |
| methionine | 1.59E−02 | 5.26E−02 | 2.28E−03 | 3.00E−03 | 0.40 | 0.10 |
| methionine sulfoxide | 1.59E−02 | 5.26E−02 | 3.22E−04 | 1.46E−04 | −1.14 | −0.09 |
| trans-4-hydroxyproline | 1.59E−02 | 5.26E−02 | 3.27E−03 | 1.80E−03 | −0.86 | −0.09 |
| tyrosine | 1.59E−02 | 5.26E−02 | 5.89E−04 | 1.20E−03 | 1.03 | 0.10 |
| UDP-glucuronate | 1.59E−02 | 5.26E−02 | 3.52E−04 | 2.25E−04 | −0.65 | −0.09 |
| 1- | 3.17E−02 | 9.23E−02 | 6.69E−05 | 1.15E−04 | 0.78 | 0.08 |
| eicosenoylglycerophosphocholine | | | | | | |
| 3-hydroxy-3- | 5.56E−02 | 1.42E−01 | 1.18E−04 | 1.76E−04 | 0.58 | 0.08 |
| methylglutarate | | | | | | |
| aspartate | 5.56E−02 | 1.42E−01 | 1.34E−03 | 8.80E−04 | −0.60 | −0.09 |
| leucine | 5.56E−02 | 1.42E−01 | 7.24E−03 | 8.98E−03 | 0.31 | 0.08 |
| N-delta-acetylornithine | 5.56E−02 | 1.42E−01 | 4.32E−05 | 2.55E−05 | −0.76 | −0.08 |
| threonine | 5.56E−02 | 1.42E−01 | 1.57E−03 | 1.22E−03 | −0.36 | −0.07 |
| uridine 5′-monophosphate | 5.56E−02 | 1.42E−01 | 1.76E−05 | 3.54E−05 | 1.01 | 0.08 |
| cytidine 5′- | 9.52E−02 | 2.17E−01 | 2.75E−04 | 3.31E−04 | 0.27 | 0.08 |
| monophosphate | | | | | | |
| glycerophosphoethanolamine | 9.52E−02 | 2.17E−01 | 3.09E−04 | 3.68E−04 | 0.25 | 0.08 |
| p-cresol sulfate | 9.52E−02 | 2.17E−01 | 4.95E−05 | 6.08E−05 | 0.30 | 0.07 |
| 1- | 1.51E−01 | 3.00E−01 | 4.61E−05 | 8.81E−05 | 0.93 | 0.07 |
| linoleoylglycerophosphoethanolamine | | | | | | |
| eicosapentaenoate | 1.51E−01 | 3.00E−01 | 1.59E−04 | 2.13E−04 | 0.42 | 0.07 |
| glutamine | 1.51E−01 | 3.00E−01 | 3.43E−04 | 2.85E−04 | −0.27 | −0.04 |
| glycine | 1.51E−01 | 3.00E−01 | 3.64E−04 | 2.42E−04 | −0.59 | −0.08 |
| myristoleate | 1.51E−01 | 3.00E−01 | 8.73E−04 | 2.80E−04 | −1.64 | −0.06 |
| serine | 1.51E−01 | 3.00E−01 | 2.89E−04 | 1.93E−04 | −0.59 | −0.07 |
| trizma acetate | 1.51E−01 | 3.00E−01 | 1.23E−04 | 1.67E−04 | 0.44 | 0.06 |
| xanthosine | 1.51E−01 | 3.00E−01 | 2.24E−04 | 3.38E−04 | 0.59 | 0.06 |
| adenosine 5′- | 2.22E−01 | 3.82E−01 | 8.24E−03 | 9.47E−03 | 0.20 | 0.06 |
| monophosphate | | | | | | |
| fumarate | 2.22E−01 | 3.82E−01 | 4.54E−05 | 8.98E−05 | 0.99 | 0.06 |
| glucose | 2.22E−01 | 3.82E−01 | 3.03E−05 | 9.17E−05 | 1.60 | 0.06 |
| lactate | 2.22E−01 | 3.82E−01 | 1.00E−03 | 4.05E−04 | −1.30 | −0.05 |
| malate | 2.22E−01 | 3.82E−01 | 4.48E−03 | 6.13E−03 | 0.45 | 0.05 |
| succinate | 2.22E−01 | 3.82E−01 | 4.59E−04 | 4.95E−04 | 0.11 | 0.03 |
| tryptophan | 2.22E−01 | 3.82E−01 | 8.54E−05 | 7.51E−05 | −0.18 | 0.01 |
| 1- | 3.10E−01 | 4.72E−01 | 3.78E−05 | 2.29E−05 | −0.72 | −0.06 |
| oleoylglycerophosphoinositol | | | | | | |
| Isobar: fructose 1,6- | 3.10E−01 | 4.72E−01 | 1.93E−05 | 3.66E−05 | 0.92 | 0.05 |
| diphosphate, glucose 1,6- | | | | | | |
| diphosphate, myo-inositol | | | | | | |
| 1,4 or 1,3-diphosphate | | | | | | |
| N-acetylmethionine | 3.10E−01 | 4.72E−01 | 3.79E−05 | 4.36E−05 | 0.20 | 0.06 |
| N-acetylserine | 3.10E−01 | 4.72E−01 | 7.83E−05 | 4.62E−05 | −0.76 | −0.05 |
| xanthine | 3.10E−01 | 4.72E−01 | 8.26E−04 | 1.43E−03 | 0.79 | 0.05 |
| pyroglutamine | 4.21E−01 | 6.07E−01 | 1.14E−03 | 1.35E−03 | 0.25 | 0.04 |
| beta-hydroxyisovalerate | 5.48E−01 | 7.04E−01 | 6.26E−05 | 5.29E−05 | −0.24 | −0.04 |
| docosapentaenoate (n6) | 5.48E−01 | 7.04E−01 | 3.19E−05 | 2.62E−05 | −0.28 | −0.03 |
| erythronate | 5.48E−01 | 7.04E−01 | 3.05E−04 | 2.58E−04 | −0.24 | −0.01 |
| flavin mononucleotide | 5.48E−01 | 7.04E−01 | 1.85E−05 | 1.59E−05 | −0.22 | −0.04 |
| guanosine 5′- | 5.48E−01 | 7.04E−01 | 9.62E−04 | 8.06E−04 | −0.25 | −0.04 |
| monophosphate | | | | | | |
| guanosine 5′-diphospho- | 5.48E−01 | 7.04E−01 | 2.67E−05 | 2.22E−05 | −0.27 | −0.03 |
| fucose | | | | | | |
| methylmalonate | 5.48E−01 | 7.04E−01 | 5.53E−05 | 7.42E−05 | 0.42 | 0.03 |
| proline | 5.48E−01 | 7.04E−01 | 2.48E−03 | 1.93E−03 | −0.36 | −0.05 |
| 1- | 6.90E−01 | 8.42E−01 | 6.64E−05 | 9.96E−05 | 0.59 | 0.01 |
| eicosatrienoylglycerophosphoethanolamine | | | | | | |
| 4-guanidinobutanoate | 6.90E−01 | 8.42E−01 | 3.74E−05 | 3.65E−05 | −0.04 | 0.02 |
| phenylalanine | 6.90E−01 | 8.42E−01 | 1.12E−03 | 8.64E−04 | −0.37 | 0.00 |
| 4-methyl-2- | 8.41E−01 | 9.19E−01 | 4.59E−05 | 5.10E−05 | 0.15 | 0.02 |
| oxopentanoate | | | | | | |
| 5-dodecenoate | 8.41E−01 | 9.19E−01 | 5.73E−05 | 4.86E−05 | −0.24 | −0.02 |
| acetylcarnitine | 8.41E−01 | 9.19E−01 | 1.39E−04 | 1.49E−04 | 0.10 | 0.03 |
| benzoate | 8.41E−01 | 9.19E−01 | 5.47E−04 | 5.76E−04 | 0.08 | 0.02 |
| dihomo-linolenate | 8.41E−01 | 9.19E−01 | 1.45E−04 | 1.37E−04 | −0.08 | −0.02 |
| ethylmalonate | 8.41E−01 | 9.19E−01 | 4.66E−05 | 3.26E−05 | −0.51 | −0.03 |
| glutamate, gamma- | 8.41E−01 | 9.19E−01 | 1.29E−04 | 1.20E−04 | −0.11 | −0.01 |
| methyl ester | | | | | | |
| guanine | 8.41E−01 | 9.19E−01 | 2.04E−04 | 2.23E−04 | 0.13 | 0.03 |
| isoleucine | 8.41E−01 | 9.19E−01 | 1.01E−03 | 9.81E−04 | −0.05 | 0.01 |
| UDP-N- | 8.41E−01 | 9.19E−01 | 9.36E−04 | 8.88E−04 | −0.08 | −0.01 |
| acetylglucosamine | | | | | | |
| 3-methyl-2-oxobutyrate | 1.00E+00 | 1.00E+00 | 4.53E−05 | 4.25E−05 | −0.09 | −0.02 |
| creatine | 1.00E+00 | 1.00E+00 | 2.20E−02 | 2.19E−02 | −0.01 | 0.01 |
| erucate | 1.00E+00 | 1.00E+00 | 1.38E−04 | 1.32E−04 | −0.07 | −0.01 |
| histidine | 1.00E+00 | 1.00E+00 | 1.09E−04 | 9.64E−05 | −0.17 | −0.02 |
| isobutyrylcarnitine | 1.00E+00 | 1.00E+00 | 1.13E−05 | 9.57E−06 | −0.24 | 0.01 |
| pyridoxal | 1.00E+00 | 1.00E+00 | 4.82E−05 | 5.59E−05 | 0.21 | 0.02 |
| 2′-deoxyadenosine 5′- | 7.94E−03 | 3.13E−02 | 3.37E−05 | 1.12E−04 | 1.73 | 0.12 |
| monophosphate | | | | | | |
| stearoyl-arachidonoyl- | 7.94E−03 | 3.13E−02 | 3.43E−04 | 1.76E−04 | −0.96 | −0.11 |
| glycerophosphoinositol | | | | | | |
| (1) | | | | | | |
| cytidine triphosphate | 3.17E−02 | 9.23E−02 | 2.04E−04 | 5.12E−04 | 1.33 | 0.09 |
| mead acid | 3.17E−02 | 9.23E−02 | 3.12E−04 | 5.32E−04 | 0.77 | 0.09 |
| prolylalanine | 5.56E−02 | 1.42E−01 | 3.84E−05 | 1.28E−05 | −1.58 | −0.07 |
| 2- | 9.52E−02 | 2.17E−01 | 6.31E−04 | 1.28E−03 | 1.02 | 0.07 |
| palmitoleoylglycerophosphoethanolamine | | | | | | |
| glycerophosphoinositol | 3.10E−01 | 4.72E−01 | 4.48E−05 | 3.66E−05 | −0.29 | −0.05 |
| coenzyme A | 6.90E−01 | 8.42E−01 | 2.57E−04 | 2.88E−04 | 0.17 | 0.04 |
| nicotinamide | 1.00E+00 | 1.00E+00 | 2.35E−02 | 2.08E−02 | −0.17 | −0.02 |
| 1- | 7.94E−03 | 3.13E−02 | 5.05E−05 | 1.59E−05 | −1.67 | −0.09 |
| oleoylglycerophosphoserine | | | | | | |
| 2′-deoxyguanosine | 7.94E−03 | 3.13E−02 | 2.51E−05 | 8.69E−05 | 1.79 | 0.12 |
| oleoyl-linoleoyl- | 7.94E−03 | 3.13E−02 | 8.73E−05 | 2.46E−04 | 1.49 | 0.12 |
| glycerophosphoinositol | | | | | | |
| (1) | | | | | | |
| pterin | 7.94E−03 | 3.13E−02 | 4.00E−05 | 6.91E−05 | 0.79 | 0.09 |
| alpha-ketoglutarate | 5.56E−02 | 1.42E−01 | 7.56E−04 | 8.73E−04 | 0.21 | 0.08 |
| 4-hydroxyphenylpyruvate | 5.48E−01 | 7.04E−01 | 2.85E−05 | 2.63E−05 | −0.12 | −0.02 |
| glycerate | 8.41E−01 | 9.19E−01 | 6.25E−05 | 1.29E−04 | 1.05 | 0.03 |
| methylphosphate | 1.00E+00 | 1.00E+00 | 3.12E−05 | 3.22E−05 | 0.04 | 0.00 |
| 1- | 7.94E−03 | 3.13E−02 | 1.46E−04 | 4.06E−05 | −1.84 | −0.10 |
| docosahexaenoylglycerol | | | | | | |
| cytidine | 7.94E−03 | 3.13E−02 | 4.23E−04 | 9.32E−04 | 1.14 | 0.11 |
| 7-methylguanine | 1.51E−01 | 3.00E−01 | 1.75E−05 | 1.33E−05 | −0.39 | −0.06 |
| cysteine-glutathione | 2.22E−01 | 3.82E−01 | 5.20E−05 | 7.26E−05 | 0.48 | 0.07 |
| disulfide | | | | | | |
| 1- | 1.59E−02 | 5.26E−02 | 8.01E−04 | 3.57E−04 | −1.17 | −0.09 |
| oleoylglycerophosphoethanolamine | | | | | | |
| uridine | 2.22E−01 | 3.82E−01 | 9.29E−04 | 1.35E−03 | 0.54 | 0.06 |
| uridine 5′-diphosphate | 5.48E−01 | 7.04E−01 | 7.68E−05 | 8.30E−05 | 0.11 | −0.02 |
| 1- | 6.90E−01 | 8.42E−01 | 1.08E−04 | 1.16E−04 | 0.10 | 0.00 |
| palmitoleoylglycerophosphoethanolamine | | | | | | |
| 2′-deoxycytidine | 7.94E−03 | 3.13E−02 | 1.99E−05 | 5.47E−05 | 1.46 | 0.10 |
| flavin adenine | 7.94E−03 | 3.13E−02 | 1.27E−04 | 1.61E−04 | 0.34 | 0.10 |
| dinucleotide | | | | | | |
| 1- | 9.52E−02 | 2.17E−01 | 2.95E−04 | 1.35E−04 | −1.13 | −0.08 |
| arachidonoylglycerophosphoethanolamine | | | | | | |
| 2′-deoxycytidine 5′- | 9.52E−02 | 2.17E−01 | 1.09E−05 | 1.89E−05 | 0.80 | 0.08 |
| monophosphate | | | | | | |
| hypoxanthine | 1.51E−01 | 3.00E−01 | 4.76E−05 | 1.88E−04 | 1.98 | 0.07 |
| docosapentaenoate (n3) | 2.22E−01 | 3.82E−01 | 1.46E−04 | 9.02E−05 | −0.70 | −0.06 |
| N-acetyl-aspartyl- | 2.22E−01 | 3.82E−01 | 5.40E−05 | 8.95E−05 | 0.73 | 0.06 |
| glutamate | | | | | | |
| linoleate | 3.10E−01 | 4.72E−01 | 1.56E−03 | 1.36E−03 | −0.20 | −0.05 |
| glycerol | 4.21E−01 | 6.07E−01 | 3.33E−04 | 2.85E−04 | −0.23 | −0.05 |
| phosphoenolpyruvate | 4.21E−01 | 6.07E−01 | 7.40E−06 | 5.66E−06 | −0.39 | −0.04 |
| S-adenosylhomocysteine | 4.21E−01 | 6.07E−01 | 3.91E−05 | 3.31E−05 | −0.24 | −0.05 |
| butyrylcarnitine | 5.48E−01 | 7.04E−01 | 1.00E−05 | 1.32E−05 | 0.40 | 0.03 |
| N-acetylaspartate | 6.90E−01 | 8.42E−01 | 5.97E−04 | 6.32E−04 | 0.08 | 0.03 |
| 2- | 8.41E−01 | 9.19E−01 | 4.88E−04 | 1.77E−04 | −1.46 | 0.02 |
| oleoylglycerophosphocholine | | | | | | |
| 2- | 8.41E−01 | 9.19E−01 | 1.94E−04 | 1.20E−04 | −0.69 | 0.02 |
| palmitoylglycerophosphocholine | | | | | | |
| 17-methylstearate | 1.00E+00 | 1.00E+00 | 2.91E−04 | 2.93E−04 | 0.01 | 0.00 |
| 1- | 7.94E−03 | 3.13E−02 | 3.70E−05 | 1.67E−05 | −1.15 | −0.10 |
| palmitoylglycerophosphoinositol | | | | | | |
| cytidine diphosphate | 7.94E−03 | 3.13E−02 | 5.08E−04 | 9.57E−04 | 0.91 | 0.10 |
| 1- | 4.21E−01 | 6.07E−01 | 1.05E−05 | 8.02E−06 | −0.38 | −0.05 |
| palmitoylglycerophosphoserine | | | | | | |
| 1- | 5.48E−01 | 7.04E−01 | 3.34E−04 | 1.14E−04 | −1.55 | −0.03 |
| oleoylglycerophosphocholine | | | | | | |
| cystathionine | 5.48E−01 | 7.04E−01 | 1.18E−05 | 1.10E−05 | −0.10 | −0.04 |
| lysine | 1.00E+00 | 1.00E+00 | 5.66E−05 | 5.24E−05 | −0.11 | −0.02 |
| N-acetylthreonine | 1.00E+00 | 1.00E+00 | 2.22E−05 | 2.12E−05 | −0.06 | −0.01 |
| adenosine | 9.52E−02 | 2.17E−01 | 7.64E−04 | 1.22E−03 | 0.67 | 0.09 |
| 2-arachidonoyl glycerol | 2.22E−01 | 3.82E−01 | 5.91E−05 | 1.65E−05 | −1.84 | −0.08 |
| myristate | 3.10E−01 | 4.72E−01 | 5.84E−02 | 5.08E−02 | −0.20 | −0.05 |
| 1- | 8.41E−01 | 9.19E−01 | 6.21E−04 | 4.39E−04 | −0.50 | 0.00 |
| palmitoylglycerophosphocholine | | | | | | |
| pyruvate | 8.41E−01 | 9.19E−01 | 7.05E−04 | 7.20E−04 | 0.03 | 0.00 |
| sphinganine | 1.00E+00 | 1.00E+00 | 2.83E−05 | 2.98E−05 | 0.07 | 0.02 |
| spermine | 6.90E−01 | 8.42E−01 | 1.51E−04 | 1.98E−04 | 0.39 | 0.04 |
| 1- | 7.94E−03 | 3.13E−02 | 2.87E−04 | 1.32E−04 | −1.12 | −0.10 |
| stearoylglycerophosphoinositol | | | | | | |
| 1- | 3.17E−02 | 9.23E−02 | 7.67E−05 | 2.19E−05 | −1.81 | −0.09 |
| docosahexaenoylglycerophosphoethanolamine | | | | | | |
| dihomo-linoleate | 3.17E−02 | 9.23E−02 | 2.58E−04 | 1.51E−04 | −0.78 | −0.09 |
| nonadecanoate | 3.10E−01 | 4.72E−01 | 9.73E−04 | 1.24E−03 | 0.35 | 0.05 |
| 2- | 4.21E−01 | 6.07E−01 | 1.85E−04 | 8.79E−05 | −1.07 | −0.04 |
| palmitoylglycerophosphoethanolamine | | | | | | |
| stearate | 4.21E−01 | 6.07E−01 | 4.20E−01 | 3.78E−01 | −0.15 | −0.05 |
| arachidate | 5.48E−01 | 7.04E−01 | 3.99E−03 | 4.24E−03 | 0.09 | 0.02 |
| phosphate | 8.41E−01 | 9.19E−01 | 2.67E−03 | 2.65E−03 | −0.01 | 0.00 |
| creatine phosphate | 1.00E+00 | 1.00E+00 | 1.35E−04 | 1.51E−04 | 0.16 | 0.01 |
| 2-palmitoylglycerol | 7.94E−03 | 3.13E−02 | 3.78E−04 | 1.38E−04 | −1.45 | −0.10 |
| 2′-deoxyinosine | 7.94E−03 | 3.13E−02 | 2.00E−05 | 9.62E−05 | 2.26 | 0.12 |
| 5-methylthioadenosine | 7.94E−03 | 3.13E−02 | 1.58E−02 | 2.22E−02 | 0.49 | 0.11 |
| arachidonate | 7.94E−03 | 3.13E−02 | 1.25E−03 | 5.67E−04 | −1.14 | −0.12 |
| nicotinamide adenine | 7.94E−03 | 3.13E−02 | 7.08E−04 | 1.32E−03 | 0.90 | 0.11 |
| dinucleotide | | | | | | |
| acetyl CoA | 1.59E−02 | 5.26E−02 | 2.22E−05 | 3.68E−05 | 0.73 | 0.09 |
| 3′-dephosphocoenzyme A | 3.17E−02 | 9.23E−02 | 4.47E−05 | 7.18E−05 | 0.68 | 0.08 |
| palmitate | 1.51E−01 | 3.00E−01 | 2.76E−01 | 2.42E−01 | −0.18 | −0.06 |
| 13-HODE + 9-HODE | 8.41E−01 | 9.19E−01 | 7.79E−05 | 6.49E−05 | −0.26 | −0.02 |
| stearoyl-oleoyl- | 1.00E+00 | 1.00E+00 | 2.96E−04 | 3.00E−04 | 0.02 | 0.00 |
| glycerophosphoserine (1) | | | | | | |
| 1- | 7.94E−03 | 3.13E−02 | 1.62E−04 | 6.47E−05 | −1.32 | −0.09 |
| stearoylglycerophosphoethanolamine | | | | | | |
| 15-methylpalmitate | 2.22E−01 | 3.82E−01 | 7.63E−03 | 6.23E−03 | −0.29 | −0.06 |
| (isobar with 2- | | | | | | |
| methylpalmitate) | | | | | | |
| margarate | 2.22E−01 | 3.82E−01 | 1.14E−02 | 9.69E−03 | −0.24 | −0.06 |
| 1-oleoylglycerol | 3.10E−01 | 4.72E−01 | 2.86E−04 | 1.70E−04 | −0.75 | −0.05 |
| pentadecanoate | 3.10E−01 | 4.72E−01 | 7.34E−03 | 6.31E−03 | −0.22 | −0.05 |
| spermidine | 3.10E−01 | 4.72E−01 | 5.33E−03 | 1.51E−03 | −1.82 | −0.06 |
| 4-methylglutamate | 8.41E−01 | 9.19E−01 | 8.97E−05 | 1.03E−04 | 0.19 | 0.04 |
| nicotinate | 1.00E+00 | 1.00E+00 | 1.56E−03 | 1.54E−03 | −0.02 | 0.00 |
| 1- | 7.94E−03 | 3.13E−02 | 1.76E−04 | 5.74E−05 | −1.61 | −0.10 |
| palmitoylglycerophosphoethanolamine | | | | | | |
| 13-methylmyristic acid | 1.59E−02 | 5.26E−02 | 4.72E−03 | 3.48E−03 | −0.44 | −0.09 |
| 10-heptadecenoate | 5.56E−02 | 1.42E−01 | 7.72E−04 | 3.74E−04 | −1.05 | −0.06 |
| 1-myristoylglycerol | 9.52E−02 | 2.17E−01 | 1.65E−04 | 1.28E−04 | −0.36 | −0.08 |
| 2-stearoylglycerol | 1.51E−01 | 3.00E−01 | 3.60E−04 | 4.85E−04 | 0.43 | 0.06 |
| 2- | 2.22E−01 | 3.82E−01 | 7.68E−05 | 3.15E−05 | −1.29 | −0.07 |
| arachidonoylglycerophosphoethanolamine | | | | | | |
| 1-stearoylglycerol | 3.10E−01 | 4.72E−01 | 3.85E−03 | 5.57E−03 | 0.53 | 0.07 |
| thymidine | 3.10E−01 | 4.72E−01 | 7.21E−06 | 1.08E−05 | 0.58 | 0.06 |
| 2- | 5.48E−01 | 7.04E−01 | 4.41E−05 | 5.38E−05 | 0.29 | 0.03 |
| linoleoylglycerophosphocholine | | | | | | |
| adenosine 5′- | 5.48E−01 | 7.04E−01 | 2.88E−04 | 1.79E−04 | −0.68 | −0.05 |
| diphosphoribose | | | | | | |
| palmitoleate | 7.94E−03 | 3.13E−02 | 6.11E−03 | 2.63E−03 | −1.22 | −0.06 |
| 1- | 3.17E−02 | 9.23E−02 | 4.92E−05 | 2.65E−05 | −0.89 | −0.10 |
| stearoylglycerophosphoserine | | | | | | |
| guanosine | 3.17E−02 | 9.23E−02 | 1.76E−03 | 2.55E−03 | 0.54 | 0.08 |
| 2- | 9.52E−02 | 2.17E−01 | 2.04E−04 | 1.09E−04 | −0.91 | −0.07 |
| oleoylglycerophosphoethanolamine | | | | | | |
| 1- | 6.90E−01 | 8.42E−01 | 1.09E−04 | 1.31E−04 | 0.27 | 0.04 |
| stearoylglycerophosphocholine | | | | | | |
| 1- | 1.00E+00 | 1.00E+00 | 5.07E−05 | 4.50E−05 | −0.17 | 0.01 |
| linoleoylglycerophosphocholine | | | | | | |

### Example 5: Metabolite Recovery in Human Prostate Cancer FFPE Samples

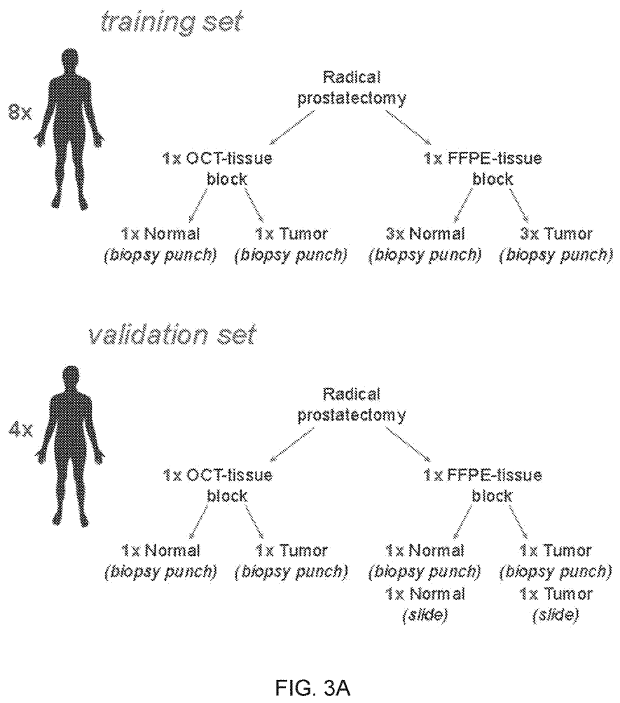

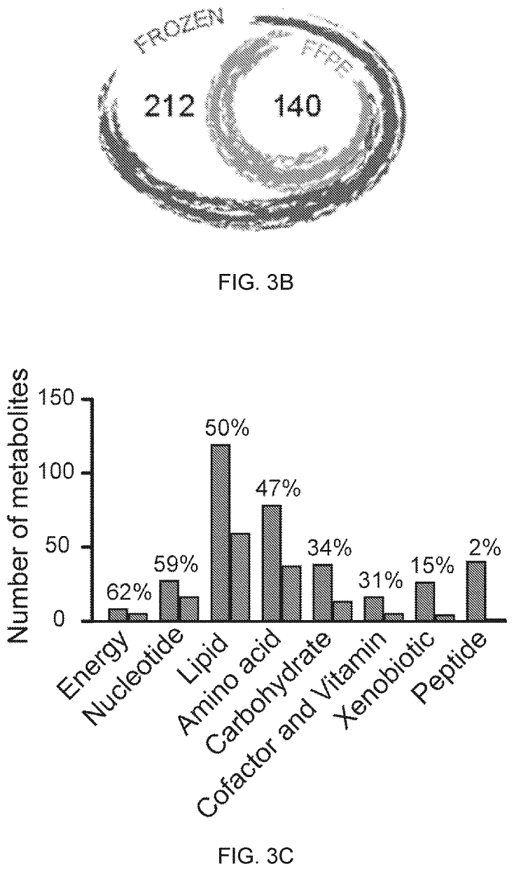

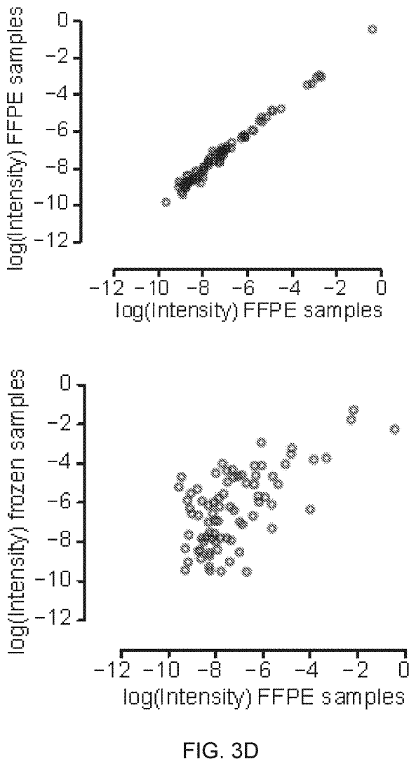

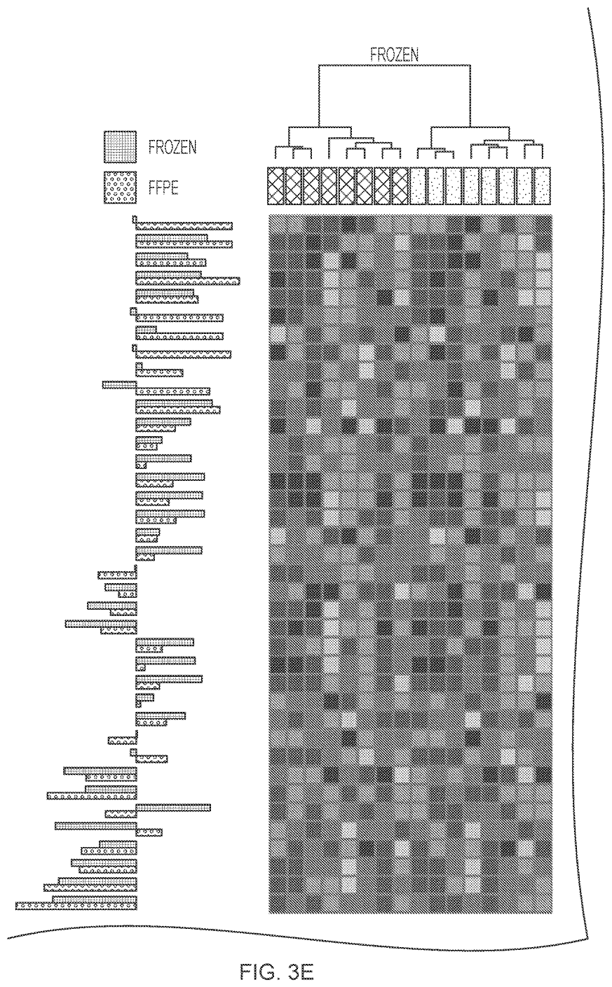

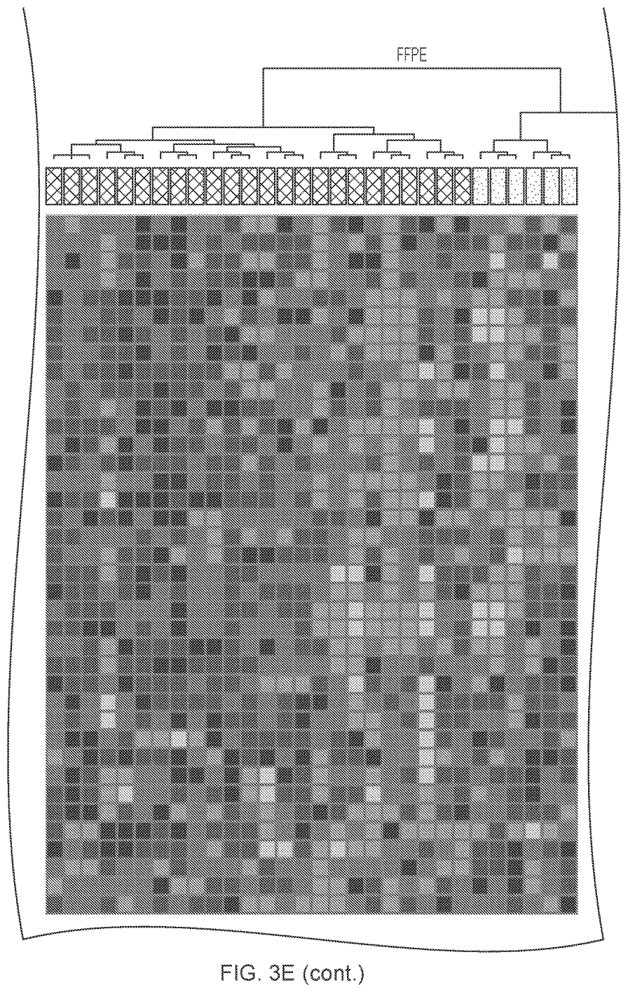

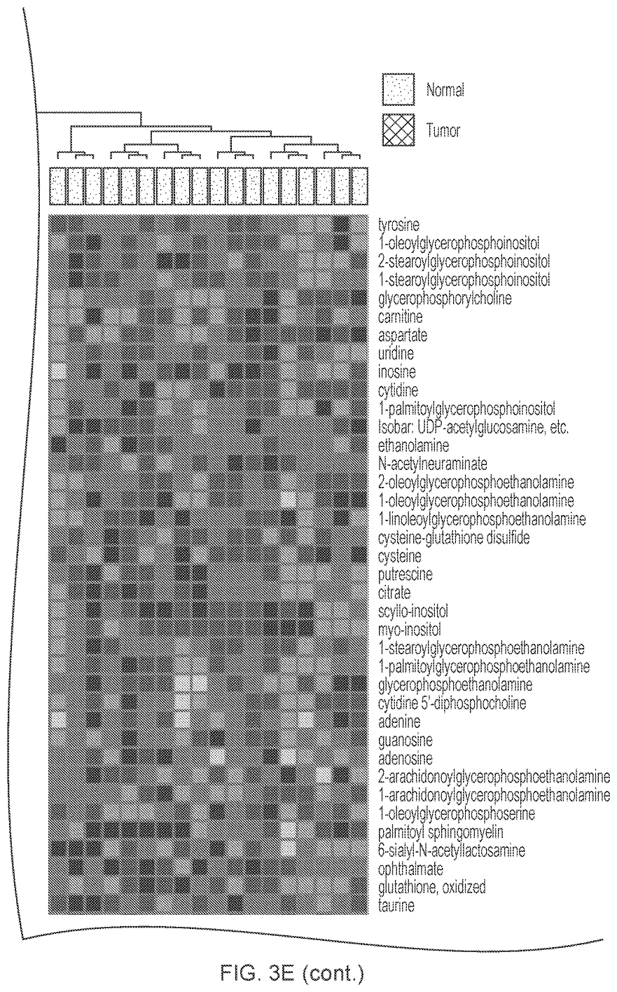

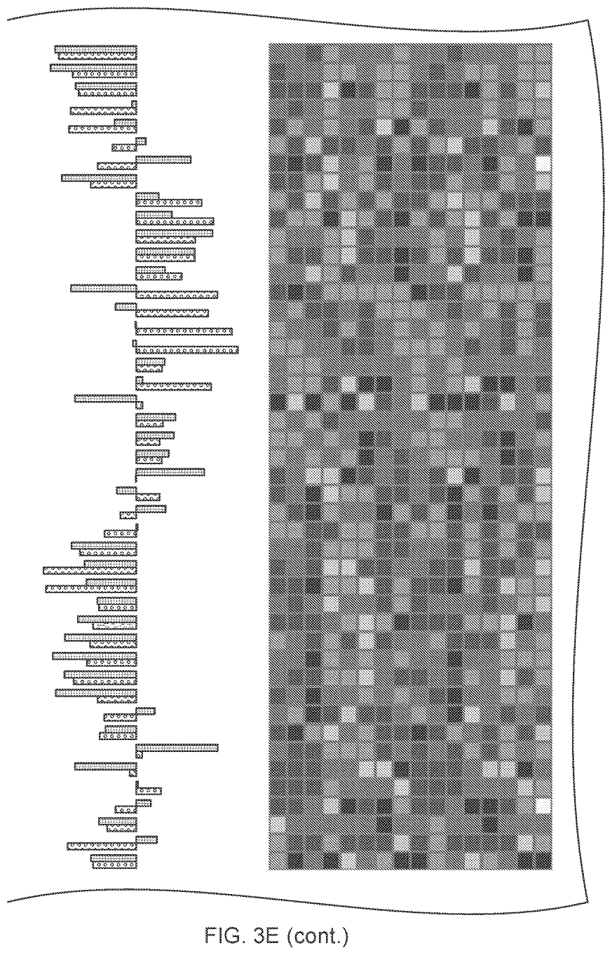

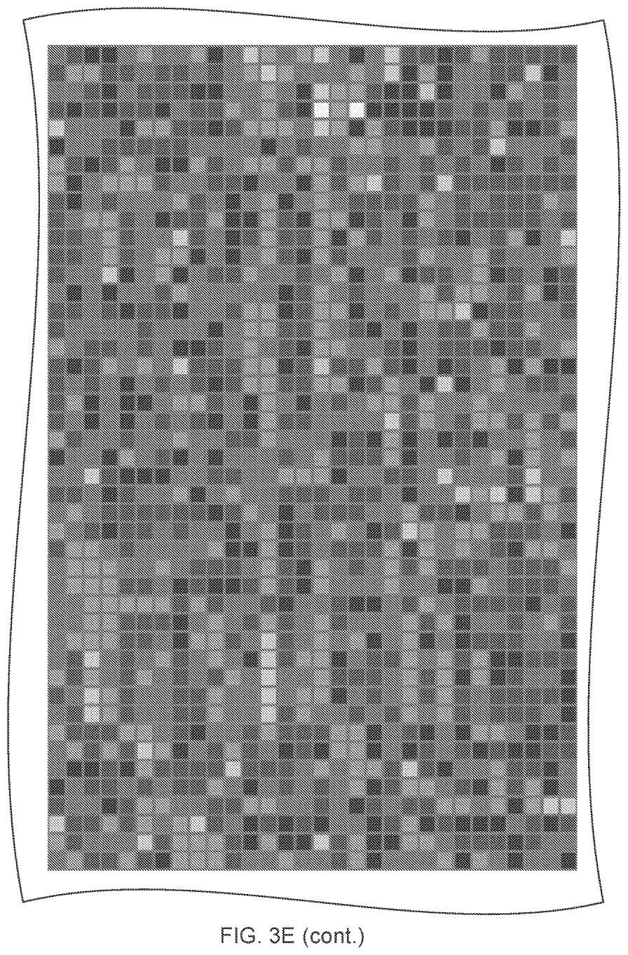

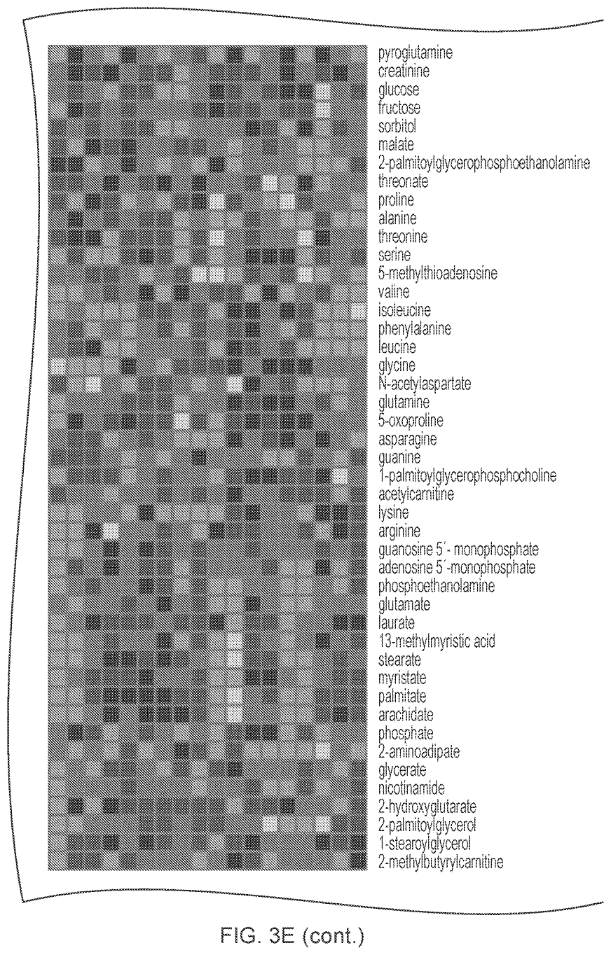

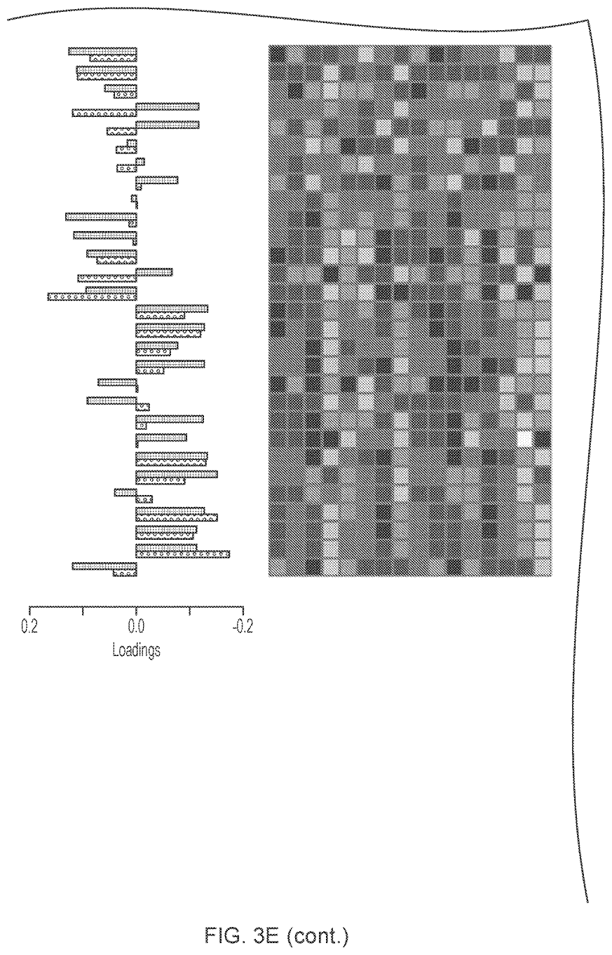

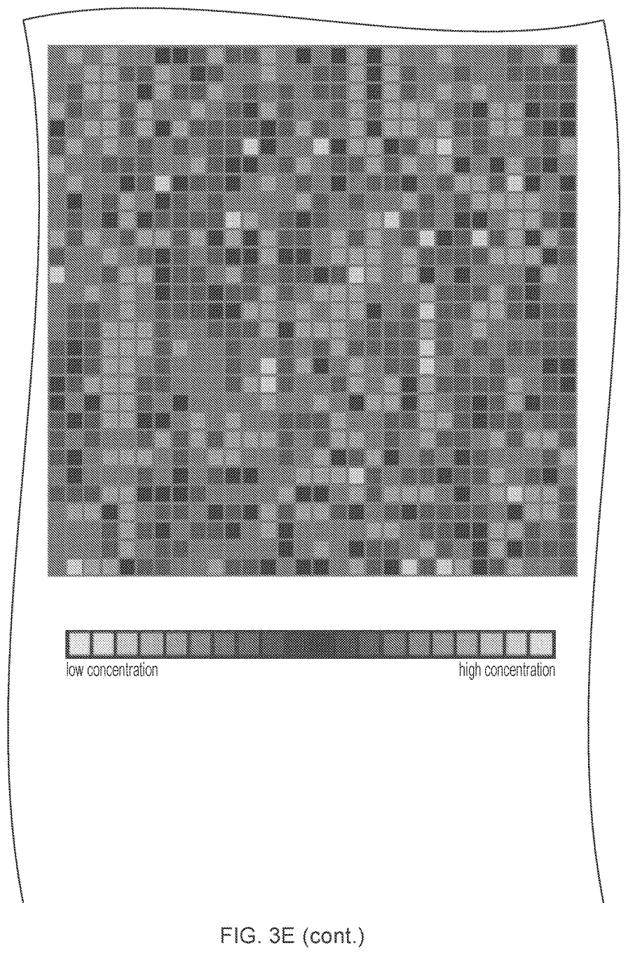

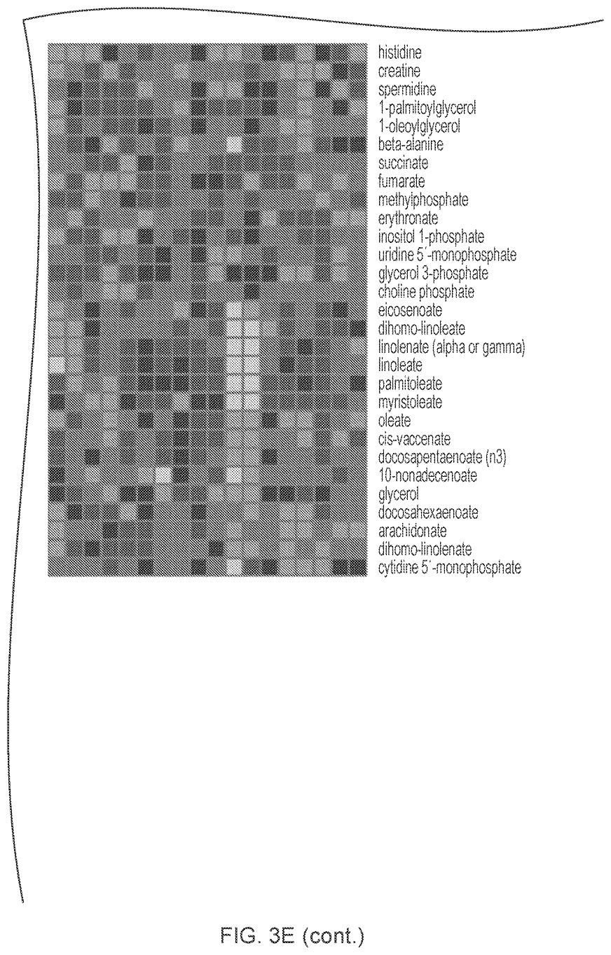

OCT-embedded and FFPE tissue blocks were collected from prostatectomy on 12 patients with prostate cancer. Metabolic profiling obtained from matched frozen and FFPE normal and tumor human prostate tissue samples were compared. Samples from 8 patients (training set) were used to define the fingerprint of prostate cancer in FFPE human tissues. Details on tissue and patient features are summarized in Table 12. Samples from the remaining 4 patients were used as an independent set (validation set). A schematic diagram on the sample collection is shown in FIG. 3A. For the training set, we collected 3 samples for each FFPE tissue type and 1 sample for each OCT-embedded tissue type. For the validation set, we collected 1 biopsy punch sample for both FFPE and OCT-embedded tissue

TABLE 12
Patient clinical characteristics and FFPE sample features

| | | Age | Gleason | Gleason | | | | | | |
|---|---|---|---|---|---|---|---|---|---|---|
| | Age | sample | Score | Score | Benign | Stroma | Tumor | T/(T + B) | | |
| ID | (year) | (month) | Primary | Independent | (%) | (%) | (%) | (%) | Stage | Recurrence |
| SC1 | 56 | 45 | 4 + 4 | 4 + 4 | 16.6 | 60.4 | 23 | 58.2 | T2a | No |
| SC2 | 52 | 72 | 3 + 4 | 3 + 3 | 24.1 | 71.1 | 4.8 | 16.6 | T2b | No |
| SC3 | 52 | 70 | 3 + 4/3 + 3 | 3 + 4 | 16 | 63 | 21 | 56.8 | T2a | No |
| SC4 | 68 | 50 | 4 + 5 | 4 + 4 | 20.7 | 66 | 13.3 | 39 | T2b | No |
| SC5 | 52 | 44 | 3 + 4 | 4 + 3 | 11.9 | 73.3 | 14.9 | 55.7 | T3a | Yes |
| SC6 | 69 | 80 | 4 + 4 | 4 + 4 | 28.5 | 70.1 | 1.4 | 4.7 | T2b | No |
| SC7 | 49 | 49 | 4 + 3 | 4 + 4 | 32.8 | 64.7 | 2.5 | 7 | T2b | No |
| SC8 | 53 | 67 | 3 + 4 | 3 + 4 | 27.4 | 60.9 | 11.8 | 30.1 | T3a | No |

A total of 352 and 140 metabolites were detected in frozen and FFPE 2 mm biopsy punch samples, respectively (FIG. 3B). Although FFPE tissue blocks were aged between 3 and 7 years, no statistically significant association between the metabolite concentrations and the age of the FFPE blocks was observed. As shown in FIG. 3C, only some classes of metabolites were preserved in FFPE material even in human tissue. Fisher's exact test was used to evaluate differences between metabolite categories detected or non-detected in FFPE samples. Significant differences are listed in Table 13, Table 14, Table 15, Table 16, Table 17 and Table 18, which list the metabolites found and missed in FFPE sample categorized by superclass, class, subclass, substituent, physical/chemical properties and pathway, respectively.

TABLE 13
Metabolites found and missed in FFPE human prostate sample categorized by
superclass.

| | non-preserved in | preserved in | FFPE/ | | |
|---|---|---|---|---|---|
| Superclass | FFPE, n (%) | FFPE, n (%) | FROZEN, % | P | FDR |
| Peptide | 39 (18.4%) | 1 (0.7%) | 2.5% | 1.57E−08 | 1.25E−07 |
| Lipid | 60 (28.3%) | 59 (42.1%) | 49.6% | 8.19E−03 | 2.96E−02 |
| Xenobiotics | 22 (10.4%) | 4 (2.9%) | 15.4% | 1.11E−02 | 2.96E−02 |
| Nucleotide | 11 (5.2%) | 16 (11.4%) | 59.3% | 4.01E−02 | 8.02E−02 |
| Amino Acid | 41 (19.3%) | 37 (26.4%) | 47.4% | 1.49E−01 | 2.38E−01 |
| Energy | 3 (1.4%) | 5 (3.6%) | 62.5% | 2.74E−01 | 3.65E−01 |
| Carbohydrate | 25 (11.8%) | 13 (9.3%) | 34.2% | 4.89E−01 | 5.58E−01 |
| Cofactors | 11 (5.2%) | 5 (3.6%) | 31.2% | 6.05E−01 | 6.05E−01 |
| and Vitamins | | | | | |

TABLE 14
Metabolites found and missed in FFPE human prostate sample categorized by class.

| | non-preserved in | preserved in | FFPE/ | | |
|---|---|---|---|---|---|
| Class | FFPE, n (%) | FFPE, n (%) | FROZEN, % | P | FDR |
| Peptides | 30 (18.5%) | 4 (3.4%) | 11.8% | 1.60E−04 | 4.17E−03 |
| Fatty Acids and Conjugates | 9 (5.6%) | 19 (16.4%) | 67.9% | 4.07E−03 | 5.29E−02 |
| Glycerolipids | 0 (0%) | 4 (3.4%) | 100.0% | 2.72E−02 | 1.77E−01 |
| Pyrimidine Nucleotides | 0 (0%) | 4 (3.4%) | 100.0% | 2.72E−02 | 1.77E−01 |
| Organic Phosphoric Acids and | 0 (0%) | 3 (2.6%) | 100.0% | 6.74E−02 | 2.36E−01 |
| Derivatives | | | | | |
| Fatty Acid Esters | 13 (8%) | 3 (2.6%) | 18.8% | 7.23E−02 | 2.36E−01 |
| Glycerophospholipids | 9 (5.6%) | 13 (11.2%) | 59.1% | 7.71E−02 | 2.36E−01 |
| Prenol Lipids | 5 (3.1%) | 0 (0%) | 0.0% | 8.15E−02 | 2.36E−01 |
| Steroids and Steroid Derivatives | 5 (3.1%) | 0 (0%) | 0.0% | 8.15E−02 | 2.36E−01 |
| Alkylamines | 2 (1.2%) | 5 (4.3%) | 71.4% | 1.27E−01 | 3.29E−01 |
| Cyclic Alcohols and Derivatives | 1 (0.6%) | 4 (3.4%) | 80.0% | 1.63E−01 | 3.85E−01 |
| Glycosyl Compounds | 3 (1.9%) | 0 (0%) | 0.0% | 2.73E−01 | 5.91E−01 |
| Hydroxy Acids and Derivatives | 5 (3.1%) | 1 (0.9%) | 16.7% | 4.07E−01 | 8.14E−01 |
| Purine Nucleosides and | 4 (2.5%) | 5 (4.3%) | 55.6% | 4.95E−01 | 9.19E−01 |
| Analogues | | | | | |
| Amino Acids and Derivatives | 32 (19.8%) | 26 (22.4%) | 44.8% | 5.55E−01 | 9.63E−01 |
| Alcohols and Polyols | 3 (1.9%) | 1 (0.9%) | 25.0% | 6.47E−01 | 9.97E−01 |
| Sugar Alcohols | 4 (2.5%) | 1 (0.9%) | 20.0% | 6.52E−01 | 9.97E−01 |
| Imidazopyrimidines | 4 (2.5%) | 4 (3.4%) | 50.0% | 7.20E−01 | 1.00E+00 |
| Purine Nucleotides | 6 (3.7%) | 3 (2.6%) | 33.3% | 7.42E−01 | 1.00E+00 |
| Benzoic Acid and Derivatives | 2 (1.2%) | 1 (0.9%) | 33.3% | 1.00E+00 | 1.00E+00 |
| Carboxylic Acids and Derivatives | 5 (3.1%) | 3 (2.6%) | 37.5% | 1.00E+00 | 1.00E+00 |
| Keto-Acids and Derivatives | 2 (1.2%) | 1 (0.9%) | 33.3% | 1.00E+00 | 1.00E+00 |
| Monosaccharides | 11 (6.8%) | 7 (6%) | 38.9% | 1.00E+00 | 1.00E+00 |
| Peptidomimetics | 2 (1.2%) | 1 (0.9%) | 33.3% | 1.00E+00 | 1.00E+00 |
| Sugar Acids and Derivatives | 3 (1.9%) | 2 (1.7%) | 40.0% | 1.00E+00 | 1.00E+00 |
| Trisaccharides | 2 (1.2%) | 1 (0.9%) | 33.3% | 1.00E+00 | 1.00E+00 |

TABLE 15
Metabolites found and missed in FFPE human prostate sample categorized by
subclass.

| | non-preserved in | preserved in | FFPE/ | | |
|---|---|---|---|---|---|
| Subclass | FFPE, n (%) | FFPE, n (%) | FROZEN, % | P | FDR |
| Unsaturated Fatty Acids | 2 (1.6%) | 14 (16.5%) | 87.5% | 1.17E−04 | 1.84E−03 |
| Peptides | 30 (24%) | 4 (4.7%) | 11.8% | 1.60E−04 | 1.84E−03 |
| Lysophosphatidylethanolamines | 1 (0.8%) | 8 (9.4%) | 88.9% | 4.01E−03 | 3.07E−02 |
| Alpha Amino Acids and | 13 (10.4%) | 20 (23.5%) | 60.6% | 2.35E−02 | 1.21E−01 |
| Derivatives | | | | | |
| Monoacylglycerols | 0 (0%) | 4 (4.7%) | 100.0% | 2.72E−02 | 1.21E−01 |
| N-acyl-alpha Amino Acids and | 11 (8.8%) | 1 (1.2%) | 8.3% | 3.16E−02 | 1.21E−01 |
| Derivatives | | | | | |
| Acyl Carnitines | 12 (9.6%) | 3 (3.5%) | 20.0% | 1.11E−01 | 3.64E−01 |
| Acyl Glycines | 5 (4%) | 1 (1.2%) | 16.7% | 4.07E−01 | 9.35E−01 |
| Pentoses | 5 (4%) | 1 (1.2%) | 16.7% | 4.07E−01 | 9.35E−01 |
| Straight Chain Fatty Acids | 4 (3.2%) | 5 (5.9%) | 55.6% | 4.95E−01 | 9.35E−01 |
| Cyclitols and Derivatives | 1 (0.8%) | 2 (2.4%) | 66.7% | 5.70E−01 | 9.35E−01 |
| Purine 2′-deoxyribonucleosides | 1 (0.8%) | 2 (2.4%) | 66.7% | 5.70E−01 | 9.35E−01 |
| and Analogues | | | | | |
| Purine Ribonucleoside | 3 (2.4%) | 1 (1.2%) | 25.0% | 6.47E−01 | 9.35E−01 |
| Diphosphates | | | | | |
| Sugar Acids and Derivatives | 4 (3.2%) | 1 (1.2%) | 20.0% | 6.52E−01 | 9.35E−01 |
| Sugar Alcohols | 4 (3.2%) | 1 (1.2%) | 20.0% | 6.52E−01 | 9.35E−01 |
| Xanthines | 4 (3.2%) | 1 (1.2%) | 20.0% | 6.52E−01 | 9.35E−01 |
| Purine Nucleosides and | 3 (2.4%) | 3 (3.5%) | 50.0% | 6.91E−01 | 9.35E−01 |
| Analogues | | | | | |
| Hexoses | 6 (4.8%) | 3 (3.5%) | 33.3% | 7.42E−01 | 9.48E−01 |
| Dicarboxylic Acids and | 3 (2.4%) | 2 (2.4%) | 40.0% | 1.00E+00 | 1.00E+00 |
| Derivatives | | | | | |
| Hybrid Peptides | 2 (1.6%) | 1 (1.2%) | 33.3% | 1.00E+00 | 1.00E+00 |
| Lysophosphatidylcholines | 7 (5.6%) | 4 (4.7%) | 36.4% | 1.00E+00 | 1.00E+00 |
| Polyamines | 2 (1.6%) | 2 (2.4%) | 50.0% | 1.00E+00 | 1.00E+00 |
| Trihexoses | 2 (1.6%) | 1 (1.2%) | 33.3% | 1.00E+00 | 1.00E+00 |

TABLE 16
Metabolites found and missed in FFPE human prostate sample categorized by
substituent.

| | non-preserved in | preserved in FFPE, n | | | |
|---|---|---|---|---|---|
| Substituent | FFPE, n (%) | (%) | FFPE/FROZEN, % | P | FDR |
| acyclic alkene | 9 (4.8%) | 24 (18.5%) | 72.7% | 1.25E−04 | 1.24E−02 |
| n-substituted-alpha-amino acid | 33 (17.6%) | 5 (3.8%) | 13.2% | 1.58E−04 | 1.24E−02 |
| saccharide | 10 (5.3%) | 25 (19.2%) | 71.4% | 1.80E−04 | 1.24E−02 |
| secondary carboxylic acid amide | 52 (27.7%) | 14 (10.8%) | 21.2% | 2.31E−04 | 1.24E−02 |
| n-acyl-alpha-amino-acid | 32 (17%) | 5 (3.8%) | 13.5% | 2.71E−04 | 1.24E−02 |
| alpha-amino acid or derivative | 33 (17.6%) | 6 (4.6%) | 15.4% | 4.20E−04 | 1.38E−02 |
| carboxamide_group | 56 (29.8%) | 17 (13.1%) | 23.3% | 4.23E−04 | 1.38E−02 |
| triose monosaccharide | 0 (0%) | 7 (5.4%) | 100.0% | 1.73E−03 | 4.95E−02 |
| fatty acid ester | 8 (4.3%) | 16 (12.3%) | 66.7% | 9.39E−03 | 2.39E−01 |
| phenol | 14 (7.4%) | 2 (1.5%) | 12.5% | 1.85E−02 | 3.43E−01 |
| phenol derivative | 14 (7.4%) | 2 (1.5%) | 12.5% | 1.85E−02 | 3.43E−01 |
| n-glycosyl compound | 9 (4.8%) | 16 (12.3%) | 64.0% | 1.90E−02 | 3.43E−01 |
| organic hypophosphite | 22 (11.7%) | 28 (21.5%) | 56.0% | 1.95E−02 | 3.43E−01 |
| phosphoethanolamine | 9 (4.8%) | 15 (11.5%) | 62.5% | 3.08E−02 | 4.82E−01 |
| ketone | 11 (5.9%) | 1 (0.8%) | 8.3% | 3.16E−02 | 4.82E−01 |
| organic phosphite | 22 (11.7%) | 27 (20.8%) | 55.1% | 3.92E−02 | 5.61E−01 |
| 1,2-diol | 36 (19.1%) | 38 (29.2%) | 51.4% | 4.30E−02 | 5.71E−01 |
| pyrimidine | 15 (8%) | 20 (15.4%) | 57.1% | 4.53E−02 | 5.71E−01 |
| dicarboxylic acid derivative | 27 (14.4%) | 9 (6.9%) | 25.0% | 4.74E−02 | 5.71E−01 |
| pentose monosaccharide | 8 (4.3%) | 13 (10%) | 61.9% | 6.39E−02 | 7.32E−01 |
| hydropyrimidine | 2 (1.1%) | 6 (4.6%) | 75.0% | 6.72E−02 | 7.33E−01 |
| phosphoric acid ester | 22 (11.7%) | 25 (19.2%) | 53.2% | 7.69E−02 | 7.68E−01 |
| aminopyrimidine | 9 (4.8%) | 13 (10%) | 59.1% | 7.71E−02 | 7.68E−01 |
| bicyclohexane | 6 (3.2%) | 0 (0%) | 0.0% | 8.49E−02 | 8.11E−01 |
| pyrimidone | 8 (4.3%) | 12 (9.2%) | 60.0% | 9.87E−02 | 9.05E−01 |
| carnitine | 12 (6.4%) | 3 (2.3%) | 20.0% | 1.11E−01 | 9.77E−01 |
| glycosyl compound | 14 (7.4%) | 17 (13.1%) | 54.8% | 1.23E−01 | 1.00E+00 |
| amphetamine or derivative | 10 (5.3%) | 2 (1.5%) | 16.7% | 1.32E−01 | 1.00E+00 |
| pyrrole | 7 (3.7%) | 1 (0.8%) | 12.5% | 1.48E−01 | 1.00E+00 |
| oxolane | 15 (8%) | 17 (13.1%) | 53.1% | 1.84E−01 | 1.00E+00 |
| carboxylic acid salt | 17 (9%) | 6 (4.6%) | 26.1% | 1.86E−01 | 1.00E+00 |
| acetal | 6 (3.2%) | 1 (0.8%) | 14.3% | 2.47E−01 | 1.00E+00 |
| secondary alcohol | 64 (34%) | 52 (40%) | 44.8% | 2.88E−01 | 1.00E+00 |
| quaternary ammonium salt | 26 (13.8%) | 12 (9.2%) | 31.6% | 2.91E−01 | 1.00E+00 |
| 1,2-aminoalcohol | 7 (3.7%) | 2 (1.5%) | 22.2% | 3.18E−01 | 1.00E+00 |
| polyamine | 4 (2.1%) | 6 (4.6%) | 60.0% | 3.27E−01 | 1.00E+00 |
| organic sulfuric acid monoester | 5 (2.7%) | 1 (0.8%) | 16.7% | 4.07E−01 | 1.00E+00 |
| sulfate-ester | 5 (2.7%) | 1 (0.8%) | 16.7% | 4.07E−01 | 1.00E+00 |
| o-glycosyl compound | 5 (2.7%) | 1 (0.8%) | 16.7% | 4.07E−01 | 1.00E+00 |
| primary alcohol | 38 (20.2%) | 32 (24.6%) | 45.7% | 4.09E−01 | 1.00E+00 |
| imidazopyrimidine | 10 (5.3%) | 10 (7.7%) | 50.0% | 4.82E−01 | 1.00E+00 |
| purine | 10 (5.3%) | 10 (7.7%) | 50.0% | 4.82E−01 | 1.00E+00 |
| primary carboxylic acid amide | 8 (4.3%) | 3 (2.3%) | 27.3% | 5.35E−01 | 1.00E+00 |
| choline | 19 (10.1%) | 10 (7.7%) | 34.5% | 5.54E−01 | 1.00E+00 |
| monosaccharide phosphate | 6 (3.2%) | 6 (4.6%) | 50.0% | 5.58E−01 | 1.00E+00 |
| imidazole | 19 (10.1%) | 16 (12.3%) | 45.7% | 5.86E−01 | 1.00E+00 |
| 1,3-aminoalcohol | 10 (5.3%) | 5 (3.8%) | 33.3% | 6.02E−01 | 1.00E+00 |
| oxane | 12 (6.4%) | 6 (4.6%) | 33.3% | 6.25E−01 | 1.00E+00 |
| carboxylic acid | 100 (53.2%) | 65 (50%) | 39.4% | 6.48E−01 | 1.00E+00 |
| purinone | 3 (1.6%) | 3 (2.3%) | 50.0% | 6.91E−01 | 1.00E+00 |
| sugar acid | 3 (1.6%) | 3 (2.3%) | 50.0% | 6.91E−01 | 1.00E+00 |
| disaccharide phosphate | 3 (1.6%) | 3 (2.3%) | 50.0% | 6.91E−01 | 1.00E+00 |
| imidazolyl carboxylic acid | 5 (2.7%) | 2 (1.5%) | 28.6% | 7.05E−01 | 1.00E+00 |
| derivative | | | | | |
| benzoyl | 5 (2.7%) | 2 (1.5%) | 28.6% | 7.05E−01 | 1.00E+00 |
| thioether | 5 (2.7%) | 2 (1.5%) | 28.6% | 7.05E−01 | 1.00E+00 |
| primary aliphatic amine | 57 (30.3%) | 36 (27.7%) | 38.7% | 7.07E−01 | 1.00E+00 |
| (alkylamine) | | | | | |
| hypoxanthine | 4 (2.1%) | 4 (3.1%) | 50.0% | 7.20E−01 | 1.00E+00 |
| carboxylic acid ester | 24 (12.8%) | 19 (14.6%) | 44.2% | 7.39E−01 | 1.00E+00 |
| short-chain hydroxy acid | 6 (3.2%) | 3 (2.3%) | 33.3% | 7.42E−01 | 1.00E+00 |
| cyclohexane | 8 (4.3%) | 4 (3.1%) | 33.3% | 7.67E−01 | 1.00E+00 |
| phosphocholine | 7 (3.7%) | 6 (4.6%) | 46.2% | 7.76E−01 | 1.00E+00 |
| alpha-hydroxy acid | 7 (3.7%) | 6 (4.6%) | 46.2% | 7.76E−01 | 1.00E+00 |
| beta-hydroxy acid | 7 (3.7%) | 6 (4.6%) | 46.2% | 7.76E−01 | 1.00E+00 |
| hemiacetal | 9 (4.8%) | 7 (5.4%) | 43.8% | 8.00E−01 | 1.00E+00 |
| glycero-3-phosphocholine | 7 (3.7%) | 4 (3.1%) | 36.4% | 1.00E+00 | 1.00E+00 |
| 1-phosphoribosyl-imidazole | 6 (3.2%) | 4 (3.1%) | 40.0% | 1.00E+00 | 1.00E+00 |
| organic pyrophosphate | 6 (3.2%) | 4 (3.1%) | 40.0% | 1.00E+00 | 1.00E+00 |
| phenethylamine | 4 (2.1%) | 2 (1.5%) | 33.3% | 1.00E+00 | 1.00E+00 |
| n-acylglycine | 5 (2.7%) | 4 (3.1%) | 44.4% | 1.00E+00 | 1.00E+00 |

TABLE 17
Metabolites found and missed in FFPE human prostate sample categorized by
property.

| | non-preserved in | preserved in | | |
|---|---|---|---|---|
| Propriety | FFPE, n (%) | FFPE, (%) | P | FDR |
| pka_strongest_basic_ChemAxon | 1.30 | 2.87 | 5.94E−03 | 9.50E−02 |
| physiological_charge_ChemAxon | −0.46 | −0.57 | 1.93E−01 | 9.58E−01 |
| pka_strongest_acidic_ChemAxon | 4.97 | 4.68 | 2.86E−01 | 9.58E−01 |
| formal_charge_ChemAxon | 0.03 | −0.02 | 2.88E−01 | 9.58E−01 |
| polarizability_ChemAxon | 26.28 | 26.24 | 5.71E−01 | 9.58E−01 |
| refractivity_ChemAxon | 65.76 | 65.08 | 6.50E−01 | 9.58E−01 |
| logp_ChemAxon | −1.22 | −0.55 | 6.52E−01 | 9.58E−01 |
| polar_surface_area_ChemAxon | 103.09 | 101.24 | 6.94E−01 | 9.58E−01 |
| average_mass_ChemAxon | 256.21 | 252.92 | 7.23E−01 | 9.58E−01 |
| mono_mass_ChemAxon | 256.06 | 252.77 | 7.24E−01 | 9.58E−01 |
| rotatable_bond_count_ChemAxon | 6.38 | 7.78 | 7.34E−01 | 9.58E−01 |
| logp_ALOGPS | −0.61 | 0.23 | 8.14E−01 | 9.58E−01 |
| donor_count_ChemAxon | 3.12 | 3.06 | 9.20E−01 | 9.58E−01 |
| acceptor_count_ChemAxon | 4.87 | 4.87 | 9.30E−01 | 9.58E−01 |
| logs_ALOGPS | −2.00 | −2.23 | 9.46E−01 | 9.58E−01 |
| solubility_ALOGPS | 104.34 | 113.84 | 9.58E−01 | 9.58E−01 |

TABLE 18
Metabolites found and missed in FFPE human prostate sample categorized by pathway.

| | non-preserved in | preserved in | FFPE/ | | |
|---|---|---|---|---|---|
| Pathway | FFPE, n (%) | FFPE, n (%) | FROZEN, % | P | FDR |
| Transcription/Translation | 3 (1.6%) | 20 (15.4%) | 87.0% | 3.23E−06 | 2.84E−04 |
| Alpha Linolenic Acid and Linoleic | 1 (0.5%) | 8 (6.2%) | 88.9% | 4.01E−03 | 1.76E−01 |
| Acid Metabolism | | | | | |
| Purine Metabolism | 4 (2.1%) | 12 (9.2%) | 75.0% | 7.20E−03 | 2.11E−01 |
| Glutathione Metabolism | 1 (0.5%) | 6 (4.6%) | 85.7% | 2.01E−02 | 4.42E−01 |
| Glutamate Metabolism | 2 (1.1%) | 7 (5.4%) | 77.8% | 3.49E−02 | 5.93E−01 |
| Aspartate Metabolism | 1 (0.5%) | 5 (3.8%) | 83.3% | 4.36E−02 | 5.93E−01 |
| Phospholipid Biosynthesis | 2 (1.1%) | 6 (4.6%) | 75.0% | 6.72E−02 | 5.93E−01 |
| Gluconeogenesis | 11 (5.9%) | 2 (1.5%) | 15.4% | 8.17E−02 | 6.54E−01 |
| Methionine Metabolism | 4 (2.1%) | 7 (5.4%) | 63.6% | 1.31E−01 | 7.36E−01 |
| Ammonia Recycling | 5 (2.7%) | 8 (6.2%) | 61.5% | 1.52E−01 | 7.36E−01 |
| Pyrimidine Metabolism | 3 (1.6%) | 6 (4.6%) | 66.7% | 1.67E−01 | 7.36E−01 |
| Arginine and Proline Metabolism | 3 (1.6%) | 6 (4.6%) | 66.7% | 1.67E−01 | 7.36E−01 |
| Galactose Metabolism | 3 (1.6%) | 6 (4.6%) | 66.7% | 1.67E−01 | 7.36E−01 |
| Glycolysis | 9 (4.8%) | 2 (1.5%) | 18.2% | 2.10E−01 | 8.80E−01 |
| Glycine and Serine Metabolism | 9 (4.8%) | 11 (8.5%) | 55.0% | 2.40E−01 | 9.20E−01 |
| Urea Cycle | 5 (2.7%) | 7 (5.4%) | 58.3% | 2.40E−01 | 9.20E−01 |
| Carnitine Synthesis | 3 (1.6%) | 5 (3.8%) | 62.5% | 2.79E−01 | 9.45E−01 |
| Amino Sugar Metabolism | 4 (2.1%) | 6 (4.6%) | 60.0% | 3.27E−01 | 9.99E−01 |
| Threonine and 2-Oxobutanoate | 5 (2.7%) | 1 (0.8%) | 16.7% | 4.07E−01 | 9.99E−01 |
| Degradation | | | | | |
| Mitochondrial Beta-Oxidation of | 6 (3.2%) | 2 (1.5%) | 25.0% | 4.79E−01 | 1.00E+00 |
| Short Chain Saturated Fatty Acids | | | | | |
| Fructose and Mannose Degradation | 6 (3.2%) | 2 (1.5%) | 25.0% | 4.79E−01 | 1.00E+00 |
| Spermidine and Spermine | 3 (1.6%) | 3 (2.3%) | 50.0% | 6.91E−01 | 1.00E+00 |
| Biosynthesis | | | | | |
| Glucose-Alanine Cycle | 3 (1.6%) | 3 (2.3%) | 50.0% | 6.91E−01 | 1.00E+00 |
| Plasmalogen Synthesis | 3 (1.6%) | 3 (2.3%) | 50.0% | 6.91E−01 | 1.00E+00 |
| Glycerolipid Metabolism | 5 (2.7%) | 5 (3.8%) | 50.0% | 7.46E−01 | 1.00E+00 |
| Mitochondrial Beta-Oxidation of | 5 (2.7%) | 3 (2.3%) | 37.5% | 1.00E+00 | 1.00E+00 |
| Long Chain Saturated Fatty Acids | | | | | |
| Transfer of Acetyl Groups into | 5 (2.7%) | 3 (2.3%) | 37.5% | 1.00E+00 | 1.00E+00 |
| Mitochondria | | | | | |
| Histidine Metabolism | 5 (2.7%) | 4 (3.1%) | 44.4% | 1.00E+00 | 1.00E+00 |
| Beta-Alanine Metabolism | 5 (2.7%) | 4 (3.1%) | 44.4% | 1.00E+00 | 1.00E+00 |
| Fatty Acid Biosynthesis | 4 (2.1%) | 3 (2.3%) | 42.9% | 1.00E+00 | 1.00E+00 |
| Beta Oxidation of Very Long | 4 (2.1%) | 3 (2.3%) | 42.9% | 1.00E+00 | 1.00E+00 |
| Chain Fatty Acids | | | | | |
| Citric Acid Cycle | 7 (3.7%) | 5 (3.8%) | 41.7% | 1.00E+00 | 1.00E+00 |
| Mitochondrial Beta-Oxidation of | 4 (2.1%) | 2 (1.5%) | 33.3% | 1.00E+00 | 1.00E+00 |
| Medium Chain Saturated Fatty | | | | | |
| Acids | | | | | |
| Mitochondrial Electron Transport | 4 (2.1%) | 3 (2.3%) | 42.9% | 1.00E+00 | 1.00E+00 |
| Chain | | | | | |

Almost all of peptides were not detectable in FFPE samples (3%, P=1.57×10⁻⁸; FDR=1.25×10⁻⁷). A heterogeneous behavior for the lipid class was observed, with metabolites with good detectability such as fatty acids (68%, P=4.07×10⁻³; FDR=5.29×10⁻²) and others like fatty acid esters (19%, P=7.23×10⁻²; FDR=2.36×10⁻¹) and steroids (0%, P=8.15×10⁻²; FDR=2.36×10⁻¹) that were poorly detectable. The presence of specific chemical substituents seems to have a clear importance with regards to the ability to detect of metabolites in FFPE samples as suggested by the inferior levels of lysophosphatidylcholines (36%, P=1.00; FDR=1.00) when compared with lysophosphatidylethanolamines (89%, P=4.01×10⁻³; FDR=3.07×10⁻²). Significant differences between frozen and FFPE samples, using both cell and human samples, are listed in Table 19, Table 20, Table 21, Table 22, Table 23, and Table 24.

TABLE 19
Metabolites found and missing is FFPE cell and human prostate sample categorized by
superclass.

| | non-preserved in | preserved in | FFPE/ | | |
|---|---|---|---|---|---|
| Superclass | FFPE, n (%) | FFPE, n (%) | FROZEN, % | p | FDR |
| Peptide | 67 (22.3%) | 6 (2.1%) | 8.2% | 5.24E−15 | 4.19E−14 |
| Lipid | 80 (26.7%) | 125 (44.3%) | 61.0% | 9.17E−06 | 3.67E−05 |
| Xenobiotics | 30 (10%) | 8 (2.8%) | 21.1% | 6.02E−04 | 1.61E−03 |
| Nucleotide | 16 (5.3%) | 35 (12.4%) | 68.6% | 3.10E−03 | 6.19E−03 |
| Amino Acid | 57 (19%) | 65 (23%) | 53.3% | 2.62E−01 | 4.20E−01 |
| Energy | 3 (1%) | 6 (2.1%) | 66.7% | 3.27E−01 | 4.36E−01 |
| Carbohydrate | 30 (10%) | 23 (8.2%) | 43.4% | 4.73E−01 | 5.41E−01 |
| Cofactors and Vitamins | 17 (5.7%) | 14 (5%) | 45.2% | 7.17E−01 | 7.17E−01 |

TABLE 20
Metabolites found and missing is FFPE cell and human prostate sample categorized by class.

| | non-preserved in | | preserved in | | FFPE/ | | |
|---|---|---|---|---|---|---|---|
| Class | FFPE, n (%) | | FFPE, n (%) | | FROZEN, % | p | FDR |
| Peptides | 57 | (23.9%) | 8 | (3.4%) | 12.3% | 2.88E−11 | 1.06E−09 |
| Fatty Acids and Conjugates | 9 | (3.8%) | 31 | (13%) | 77.5% | 2.17E−04 | 4.02E−03 |
| Pyrimidine Nucleotides | 1 | (0.4%) | 14 | (5.9%) | 93.3% | 3.76E−04 | 4.64E−03 |
| Glycerolipids | 0 | (0%) | 10 | (4.2%) | 100.0% | 7.75E−04 | 7.17E−03 |
| Steroids and Steroid Derivatives | 6 | (2.5%) | 0 | (0%) | 0.0% | 3.04E−02 | 1.90E−01 |
| Purine Nucleotides | 3 | (1.3%) | 11 | (4.6%) | 78.6% | 3.07E−02 | 1.90E−01 |
| Glycerophospholipids | 19 | (8%) | 32 | (13.4%) | 62.7% | 5.43E−02 | 2.41E−01 |
| Lineolic Acids and Derivatives | 0 | (0%) | 4 | (1.7%) | 100.0% | 5.84E−02 | 2.41E−01 |
| Prenol Lipids | 5 | (2.1%) | 0 | (0%) | 0.0% | 6.14E−02 | 2.41E−01 |
| Alkylamines | 1 | (0.4%) | 6 | (2.5%) | 85.7% | 6.51E−02 | 2.41E−01 |
| Cyclic Alcohols and Derivatives | 1 | (0.4%) | 5 | (2.1%) | 83.3% | 1.18E−01 | 3.81E−01 |
| Azoles | 4 | (1.7%) | 0 | (0%) | 0.0% | 1.24E−01 | 3.81E−01 |
| Monosaccharides | 15 | (6.3%) | 8 | (3.4%) | 34.8% | 2.01E−01 | 5.23E−01 |
| Diazines | 3 | (1.3%) | 0 | (0%) | 0.0% | 2.49E−01 | 5.23E−01 |
| Disaccharides | 3 | (1.3%) | 0 | (0%) | 0.0% | 2.49E−01 | 5.23E−01 |
| Eicosanoids | 3 | (1.3%) | 0 | (0%) | 0.0% | 2.49E−01 | 5.23E−01 |
| Glycosyl Compounds | 3 | (1.3%) | 0 | (0%) | 0.0% | 2.49E−01 | 5.23E−01 |
| Fatty Acid Esters | 13 | (5.5%) | 7 | (2.9%) | 35.0% | 2.55E−01 | 5.23E−01 |
| Pyrimidine Nucleosides and | 2 | (0.8%) | 5 | (2.1%) | 71.4% | 2.80E−01 | 5.36E−01 |
| Analogues | | | | | | | |
| Imidazopyrimidines | 3 | (1.3%) | 6 | (2.5%) | 66.7% | 3.33E−01 | 5.36E−01 |
| Benzyl Alcohols and Derivatives | 1 | (0.4%) | 3 | (1.3%) | 75.0% | 3.67E−01 | 5.36E−01 |
| Keto-Acids and Derivatives | 1 | (0.4%) | 3 | (1.3%) | 75.0% | 3.67E−01 | 5.36E−01 |
| Organic Phosphoric Acids and | 1 | (0.4%) | 3 | (1.3%) | 75.0% | 3.67E−01 | 5.36E−01 |
| Derivatives | | | | | | | |
| Pteridines and Derivatives | 1 | (0.4%) | 3 | (1.3%) | 75.0% | 3.67E−01 | 5.36E−01 |
| Sugar Alcohols | 4 | (1.7%) | 1 | (0.4%) | 20.0% | 3.73E−01 | 5.36E−01 |
| Purine Nucleosides and | 4 | (1.7%) | 7 | (2.9%) | 63.6% | 3.77E−01 | 5.36E−01 |
| Analogues | | | | | | | |
| Benzoic Acid and Derivatives | 1 | (0.4%) | 2 | (0.8%) | 66.7% | 6.19E−01 | 8.46E−01 |
| Amino Acids and Derivatives | 44 | (18.5%) | 47 | (19.7%) | 51.6% | 6.45E−01 | 8.46E−01 |
| Pyridines and Derivatives | 2 | (0.8%) | 3 | (1.3%) | 60.0% | 6.82E−01 | 8.46E−01 |
| Sphingolipids | 4 | (1.7%) | 2 | (0.8%) | 33.3% | 6.86E−01 | 8.46E−01 |
| Carboxylic Acids and Derivatives | 6 | (2.5%) | 4 | (1.7%) | 40.0% | 7.52E−01 | 8.69E−01 |
| Hydroxy Acids and Derivatives | 6 | (2.5%) | 4 | (1.7%) | 40.0% | 7.52E−01 | 8.69E−01 |
| Alcohols and Polyols | 2 | (0.8%) | 2 | (0.8%) | 50.0% | 1.00E+00 | 1.00E+00 |
| Fatty Amides | 2 | (0.8%) | 1 | (0.4%) | 33.3% | 1.00E+00 | 1.00E+00 |
| Peptidomimetics | 2 | (0.8%) | 1 | (0.4%) | 33.3% | 1.00E+00 | 1.00E+00 |
| Sugar Acids and Derivatives | 4 | (1.7%) | 4 | (1.7%) | 50.0% | 1.00E+00 | 1.00E+00 |
| Trisaccharides | 2 | (0.8%) | 1 | (0.4%) | 33.3% | 1.00E+00 | 1.00E+00 |

TABLE 21
Metabolites found and missing is FFPE cell and human prostate sample categorized by subclass.

| | non-preserved in | | preserved in | | FFPE/ | | |
|---|---|---|---|---|---|---|---|
| Subclass | FFPE, n (%) | | FFPE, n (%) | | FROZEN, % | p | FDR |
| Peptides | 57 | (29.7%) | 8 | (4.3%) | 12.3% | 2.88E−11 | 1.15E−09 |
| Lysophosphatidylethanolamines | 1 | (0.5%) | 17 | (9.2%) | 94.4% | 4.86E−05 | 6.48E−04 |
| Unsaturated Fatty Acids | 1 | (0.5%) | 17 | (9.2%) | 94.4% | 4.86E−05 | 6.48E−04 |
| Monoacylglycerols | 0 | (0%) | 10 | (5.4%) | 100.0% | 7.75E−04 | 7.75E−03 |
| Alpha Amino Acids and | 14 | (7.3%) | 32 | (17.4%) | 69.6% | 4.99E−03 | 4.00E−02 |
| Derivatives | | | | | | | |
| Phosphatidylcholines | 8 | (4.2%) | 0 | (0%) | 0.0% | 7.43E−03 | 4.95E−02 |
| Pyrimidine Nucleotide Sugars | 0 | (0%) | 5 | (2.7%) | 100.0% | 2.86E−02 | 1.63E−01 |
| Lineolic Acids and Derivatives | 0 | (0%) | 4 | (2.2%) | 100.0% | 5.84E−02 | 2.34E−01 |
| Purine 2′-deoxyribonucleosides | 0 | (0%) | 4 | (2.2%) | 100.0% | 5.84E−02 | 2.34E−01 |
| and Analogues | | | | | | | |
| Pyrimidine Ribonucleoside | 0 | (0%) | 4 | (2.2%) | 100.0% | 5.84E−02 | 2.34E−01 |
| Diphosphates | | | | | | | |
| N-acyl-alpha Amino Acids and | 17 | (8.9%) | 8 | (4.3%) | 32.0% | 9.99E−02 | 3.63E−01 |
| Derivatives | | | | | | | |
| Phosphatidylinositols | 0 | (0%) | 3 | (1.6%) | 100.0% | 1.19E−01 | 3.67E−01 |
| Pyrimidine 2′- | 0 | (0%) | 3 | (1.6%) | 100.0% | 1.19E−01 | 3.67E−01 |
| deoxyribonucleosides and | | | | | | | |
| Analogues | | | | | | | |
| Hexoses | 9 | (4.7%) | 3 | (1.6%) | 25.0% | 1.42E−01 | 4.05E−01 |
| Straight Chain Fatty Acids | 4 | (2.1%) | 9 | (4.9%) | 69.2% | 1.69E−01 | 4.50E−01 |
| Purine Ribonucleoside | 1 | (0.5%) | 4 | (2.2%) | 80.0% | 2.11E−01 | 5.24E−01 |
| Diphosphates | | | | | | | |
| Acyl Carnitines | 12 | (6.2%) | 6 | (3.3%) | 33.3% | 2.30E−01 | 5.24E−01 |
| Imidazolyl Carboxylic Acids and | 3 | (1.6%) | 0 | (0%) | 0.0% | 2.49E−01 | 5.24E−01 |
| Derivatives | | | | | | | |
| Pyrimidones | 3 | (1.6%) | 0 | (0%) | 0.0% | 2.49E−01 | 5.24E−01 |
| Acyl Glycines | 6 | (3.1%) | 2 | (1.1%) | 25.0% | 2.86E−01 | 5.44E−01 |
| Pentoses | 6 | (3.1%) | 2 | (1.1%) | 25.0% | 2.86E−01 | 5.44E−01 |
| Phenylpyruvic Acid Derivatives | 1 | (0.5%) | 3 | (1.6%) | 75.0% | 3.67E−01 | 5.96E−01 |
| Polyamines | 1 | (0.5%) | 3 | (1.6%) | 75.0% | 3.67E−01 | 5.96E−01 |
| Purine Ribonucleoside | 1 | (0.5%) | 3 | (1.6%) | 75.0% | 3.67E−01 | 5.96E−01 |
| Monophosphates | | | | | | | |
| Sugar Alcohols | 4 | (2.1%) | 1 | (0.5%) | 20.0% | 3.73E−01 | 5.96E−01 |
| Branched Fatty Acids | 2 | (1%) | 4 | (2.2%) | 66.7% | 4.45E−01 | 6.67E−01 |
| Sugar Acids and Derivatives | 5 | (2.6%) | 2 | (1.1%) | 28.6% | 4.50E−01 | 6.67E−01 |
| Cyclitols and Derivatives | 1 | (0.5%) | 2 | (1.1%) | 66.7% | 6.19E−01 | 8.32E−01 |
| Sphingolipids | 1 | (0.5%) | 2 | (1.1%) | 66.7% | 6.19E−01 | 8.32E−01 |
| Beta Amino Acids and | 3 | (1.6%) | 1 | (0.5%) | 25.0% | 6.24E−01 | 8.32E−01 |
| Derivatives | | | | | | | |
| Lysophosphatidylcholines | 8 | (4.2%) | 6 | (3.3%) | 42.9% | 7.88E−01 | 1.00E+00 |
| Beta Hydroxy Acids and | 3 | (1.6%) | 2 | (1.1%) | 40.0% | 1.00E+00 | 1.00E+00 |
| Derivatives | | | | | | | |
| Dicarboxylic Acids and | 3 | (1.6%) | 3 | (1.6%) | 50.0% | 1.00E+00 | 1.00E+00 |
| Derivatives | | | | | | | |
| Glycoamino Acids and | 2 | (1%) | 1 | (0.5%) | 33.3% | 1.00E+00 | 1.00E+00 |
| Derivatives | | | | | | | |
| Hybrid Peptides | 2 | (1%) | 1 | (0.5%) | 33.3% | 1.00E+00 | 1.00E+00 |
| Purine Nucleosides and | 4 | (2.1%) | 3 | (1.6%) | 42.9% | 1.00E+00 | 1.00E+00 |
| Analogues | | | | | | | |
| Pyrimidine Nucleosides and | 2 | (1%) | 2 | (1.1%) | 50.0% | 1.00E+00 | 1.00E+00 |
| Analogues | | | | | | | |
| Tricarboxylic Acids and | 2 | (1%) | 1 | (0.5%) | 33.3% | 1.00E+00 | 1.00E+00 |
| Derivatives | | | | | | | |
| Trihexoses | 2 | (1%) | 1 | (0.5%) | 33.3% | 1.00E+00 | 1.00E+00 |
| Xanthines | 3 | (1.6%) | 2 | (1.1%) | 40.0% | 1.00E+00 | 1.00E+00 |

TABLE 22
Metabolites found and missing is FFPE cell and human prostate sample categorized by substituent.

| | non-preserved in | | preserved in | | FFPE/ | | |
|---|---|---|---|---|---|---|---|
| Substituent | FFPE, n (%) | | FFPE, n (%) | | FROZEN, % | p | FDR |
| n-substituted-alpha-amino acid | 60 | (23.3%) | 8 | (3.2%) | 11.8% | 3.78E−12 | 8.66E−10 |
| n-acyl-alpha-amino-acid | 57 | (22.1%) | 8 | (3.2%) | 12.3% | 2.88E−11 | 3.30E−09 |
| alpha-amino acid or derivative | 62 | (24%) | 11 | (4.4%) | 15.1% | 6.43E−11 | 4.91E−09 |
| carboxamide_group | 99 | (38.4%) | 36 | (14.3%) | 26.7% | 6.01E−10 | 3.44E−08 |
| secondary carboxylic acid amide | 90 | (34.9%) | 30 | (12%) | 25.0% | 8.02E−10 | 3.67E−08 |
| saccharide | 15 | (5.8%) | 51 | (20.3%) | 77.3% | 1.17E−06 | 4.48E−05 |
| pyrimidine | 18 | (7%) | 50 | (19.9%) | 73.5% | 2.26E−05 | 7.39E−04 |
| pyrimidone | 10 | (3.9%) | 34 | (13.5%) | 77.3% | 1.11E−04 | 3.18E−03 |
| triose monosaccharide | 2 | (0.8%) | 16 | (6.4%) | 88.9% | 5.14E−04 | 1.18E−02 |
| oxolane | 18 | (7%) | 43 | (17.1%) | 70.5% | 5.56E−04 | 1.18E−02 |
| organic hypophosphite | 40 | (15.5%) | 71 | (28.3%) | 64.0% | 5.65E−04 | 1.18E−02 |
| acyclic alkene | 22 | (8.5%) | 47 | (18.7%) | 68.1% | 1.09E−03 | 2.02E−02 |
| organic phosphite | 40 | (15.5%) | 69 | (27.5%) | 63.3% | 1.15E−03 | 2.02E−02 |
| n-glycosyl compound | 12 | (4.7%) | 32 | (12.7%) | 72.7% | 1.40E−03 | 2.19E−02 |
| phosphoric acid ester | 39 | (15.1%) | 67 | (26.7%) | 63.2% | 1.50E−03 | 2.19E−02 |
| fatty acid ester | 19 | (7.4%) | 42 | (16.7%) | 68.9% | 1.53E−03 | 2.19E−02 |
| aminopyrimidine | 11 | (4.3%) | 30 | (12%) | 73.2% | 1.72E−03 | 2.21E−02 |
| hydropyrimidine | 5 | (1.9%) | 20 | (8%) | 80.0% | 1.74E−03 | 2.21E−02 |
| organic pyrophosphate | 4 | (1.6%) | 18 | (7.2%) | 81.8% | 1.87E−03 | 2.26E−02 |
| amphetamine or derivative | 17 | (6.6%) | 3 | (1.2%) | 15.0% | 2.17E−03 | 2.49E−02 |
| dicarboxylic acid derivative | 48 | (18.6%) | 25 | (10%) | 34.2% | 5.49E−03 | 5.99E−02 |
| disaccharide phosphate | 1 | (0.4%) | 9 | (3.6%) | 90.0% | 1.01E−02 | 1.05E−01 |
| quaternary ammonium salt | 39 | (15.1%) | 20 | (8%) | 33.9% | 1.27E−02 | 1.27E−01 |
| mixed pentose/hexose | 0 | (0%) | 6 | (2.4%) | 100.0% | 1.39E−02 | 1.33E−01 |
| disaccharide | | | | | | | |
| bicyclohexane | 7 | (2.7%) | 0 | (0%) | 0.0% | 1.50E−02 | 1.38E−01 |
| phenol | 15 | (5.8%) | 4 | (1.6%) | 21.1% | 1.73E−02 | 1.47E−01 |
| phenol derivative | 15 | (5.8%) | 4 | (1.6%) | 21.1% | 1.73E−02 | 1.47E−01 |
| 1-phosphoribosyl-imidazole | 3 | (1.2%) | 12 | (4.8%) | 80.0% | 1.81E−02 | 1.48E−01 |
| imidazopyrimidine | 9 | (3.5%) | 21 | (8.4%) | 70.0% | 2.34E−02 | 1.78E−01 |
| purine | 9 | (3.5%) | 21 | (8.4%) | 70.0% | 2.34E−02 | 1.78E−01 |
| 1,3-aminoalcohol | 19 | (7.4%) | 7 | (2.8%) | 26.9% | 2.54E−02 | 1.85E−01 |
| glycosyl compound | 18 | (7%) | 33 | (13.1%) | 64.7% | 2.62E−02 | 1.85E−01 |
| carboxylic acid | 149 | (57.8%) | 120 | (47.8%) | 44.6% | 2.66E−02 | 1.85E−01 |
| carboxylic acid salt | 22 | (8.5%) | 10 | (4%) | 31.2% | 4.37E−02 | 2.91E−01 |
| choline | 30 | (11.6%) | 16 | (6.4%) | 34.8% | 4.45E−02 | 2.91E−01 |
| glycero-3-phosphocholine | 16 | (6.2%) | 6 | (2.4%) | 27.3% | 4.80E−02 | 3.05E−01 |
| primary aliphatic amine | 88 | (34.1%) | 65 | (25.9%) | 42.5% | 5.30E−02 | 3.28E−01 |
| (alkylamine) | | | | | | | |
| pentose monosaccharide | 13 | (5%) | 24 | (9.6%) | 64.9% | 6.00E−02 | 3.51E−01 |
| acetal | 7 | (2.7%) | 1 | (0.4%) | 12.5% | 6.84E−02 | 3.82E−01 |
| 1,2-diol | 49 | (19%) | 65 | (25.9%) | 57.0% | 7.07E−02 | 3.85E−01 |
| monosaccharide phosphate | 9 | (3.5%) | 18 | (7.2%) | 66.7% | 7.56E−02 | 4.03E−01 |
| secondary alcohol | 87 | (33.7%) | 104 | (41.4%) | 54.5% | 8.20E−02 | 4.27E−01 |
| polyamine | 5 | (1.9%) | 12 | (4.8%) | 70.6% | 8.70E−02 | 4.43E−01 |
| carboxylic acid ester | 35 | (13.6%) | 48 | (19.1%) | 57.8% | 9.43E−02 | 4.69E−01 |
| hypoxanthine | 2 | (0.8%) | 7 | (2.8%) | 77.8% | 1.02E−01 | 4.97E−01 |
| phosphocholine | 18 | (7%) | 9 | (3.6%) | 33.3% | 1.13E−01 | 5.34E−01 |
| phosphoethanolamine | 22 | (8.5%) | 33 | (13.1%) | 60.0% | 1.16E−01 | 5.34E−01 |
| alpha-keto acid | 1 | (0.4%) | 5 | (2%) | 83.3% | 1.18E−01 | 5.34E−01 |
| o-glycosyl compound | 6 | (2.3%) | 1 | (0.4%) | 14.3% | 1.23E−01 | 5.34E−01 |
| imidazole | 19 | (7.4%) | 29 | (11.6%) | 60.4% | 1.29E−01 | 5.48E−01 |
| 1,2-aminoalcohol | 12 | (4.7%) | 5 | (2%) | 29.4% | 1.37E−01 | 5.72E−01 |
| purinone | 2 | (0.8%) | 6 | (2.4%) | 75.0% | 1.71E−01 | 6.99E−01 |
| imidazolyl carboxylic acid | 7 | (2.7%) | 2 | (0.8%) | 22.2% | 1.76E−01 | 7.08E−01 |
| derivative | | | | | | | |
| organic sulfuric acid monoester | 5 | (1.9%) | 1 | (0.4%) | 16.7% | 2.16E−01 | 7.80E−01 |
| sulfate-ester | 5 | (1.9%) | 1 | (0.4%) | 16.7% | 2.16E−01 | 7.80E−01 |
| tertiary carboxylic acid amide | 5 | (1.9%) | 1 | (0.4%) | 16.7% | 2.16E−01 | 7.80E−01 |
| beta-hydroxy acid | 16 | (6.2%) | 9 | (3.6%) | 36.0% | 2.19E−01 | 7.80E−01 |
| carnitine | 12 | (4.7%) | 6 | (2.4%) | 33.3% | 2.30E−01 | 7.80E−01 |
| pyrrole | 6 | (2.3%) | 2 | (0.8%) | 25.0% | 2.86E−01 | 8.72E−01 |
| phenethylamine | 6 | (2.3%) | 2 | (0.8%) | 25.0% | 2.86E−01 | 8.72E−01 |
| guanidine | 3 | (1.2%) | 6 | (2.4%) | 66.7% | 3.33E−01 | 9.02E−01 |
| secondary aliphatic amine | 7 | (2.7%) | 3 | (1.2%) | 30.0% | 3.39E−01 | 9.02E−01 |
| (dialkylamine) | | | | | | | |
| hemiacetal | 13 | (5%) | 8 | (3.2%) | 38.1% | 3.74E−01 | 9.02E−01 |
| primary alcohol | 53 | (20.5%) | 60 | (23.9%) | 53.1% | 3.94E−01 | 9.02E−01 |
| alkylthiol | 3 | (1.2%) | 5 | (2%) | 62.5% | 4.99E−01 | 9.02E−01 |
| allyl alcohol | 6 | (2.3%) | 3 | (1.2%) | 33.3% | 5.04E−01 | 9.02E−01 |
| cyclic alcohol | 6 | (2.3%) | 3 | (1.2%) | 33.3% | 5.04E−01 | 9.02E−01 |
| oxane | 16 | (6.2%) | 12 | (4.8%) | 42.9% | 5.62E−01 | 9.97E−01 |
| primary carboxylic acid amide | 9 | (3.5%) | 6 | (2.4%) | 40.0% | 6.02E−01 | 1.00E+00 |
| ketone | 10 | (3.9%) | 7 | (2.8%) | 41.2% | 6.24E−01 | 1.00E+00 |
| tricarboxylic acid derivative | 3 | (1.2%) | 4 | (1.6%) | 57.1% | 7.21E−01 | 1.00E+00 |
| benzoyl | 5 | (1.9%) | 3 | (1.2%) | 37.5% | 7.25E−01 | 1.00E+00 |
| thiol (sulfanyl compound) | 4 | (1.6%) | 5 | (2%) | 55.6% | 7.49E−01 | 1.00E+00 |
| succinic_acid | 6 | (2.3%) | 4 | (1.6%) | 40.0% | 7.52E−01 | 1.00E+00 |
| cyclohexane | 10 | (3.9%) | 8 | (3.2%) | 44.4% | 8.11E−01 | 1.00E+00 |
| alpha-hydroxy acid | 10 | (3.9%) | 9 | (3.6%) | 47.4% | 1.00E+00 | 1.00E+00 |
| short-chain hydroxy acid | 7 | (2.7%) | 6 | (2.4%) | 46.2% | 1.00E+00 | 1.00E+00 |
| urea | 4 | (1.6%) | 4 | (1.6%) | 50.0% | 1.00E+00 | 1.00E+00 |
| thioether | 6 | (2.3%) | 6 | (2.4%) | 50.0% | 1.00E+00 | 1.00E+00 |
| sugar acid | 4 | (1.6%) | 3 | (1.2%) | 42.9% | 1.00E+00 | 1.00E+00 |
| n-acylglycine | 7 | (2.7%) | 6 | (2.4%) | 46.2% | 1.00E+00 | 1.00E+00 |
| pyrrolidine | 4 | (1.6%) | 4 | (1.6%) | 50.0% | 1.00E+00 | 1.00E+00 |
| pyrrolidine carboxylic acid | 4 | (1.6%) | 3 | (1.2%) | 42.9% | 1.00E+00 | 1.00E+00 |

TABLE 23
Metabolites found and missing is FFPE cell and human prostate sample categorized by
property.

| | non-preserved in | preserved in | | | |
|---|---|---|---|---|---|
| Property | FFPE, n (%) | FFPE, n (%) | | p | FDR |
| physiological_charge_ChemAxon | −0.48 | −0.68 | 0.0% | 1.35E−02 | 1.24E−01 |
| logp_ALOGPS | −0.46 | 0.55 | 0.0% | 1.55E−02 | 1.24E−01 |
| logp_ChemAxon | −0.98 | −0.19 | 0.0% | 9.42E−02 | 4.69E−01 |
| pka_strongest_acidic_ChemAxon | 4.64 | 4.50 | 0.0% | 1.17E−01 | 4.69E−01 |
| pka_strongest_basic_ChemAxon | 2.00 | 2.17 | 0.0% | 1.51E−01 | 4.84E−01 |
| logs_ALOGPS | −2.21 | −2.47 | 0.0% | 4.70E−01 | 9.74E−01 |
| solubility_ALOGPS | 85.05 | 74.48 | 0.0% | 5.08E−01 | 9.74E−01 |
| rotatable_bond_count_ChemAxon | 7.84 | 9.06 | 0.0% | 5.89E−01 | 9.74E−01 |
| polar_surface_area_ChemAxon | 105.45 | 110.61 | 0.0% | 6.70E−01 | 9.74E−01 |
| formal_charge_ChemAxon | 0.02 | 0.00 | 0.0% | 8.08E−01 | 9.74E−01 |
| average_mass_ChemAxon | 277.32 | 287.48 | 0.0% | 8.48E−01 | 9.74E−01 |
| mono_mass_ChemAxon | 277.15 | 287.31 | 0.0% | 8.52E−01 | 9.74E−01 |
| refractivity_ChemAxon | 72.35 | 73.01 | 0.0% | 8.54E−01 | 9.74E−01 |
| acceptor_count_ChemAxon | 4.95 | 5.25 | 0.0% | 8.62E−01 | 9.74E−01 |
| polarizability_ChemAxon | 29.07 | 29.68 | 0.0% | 9.72E−01 | 9.74E−01 |
| donor_count_ChemAxon | 3.14 | 3.21 | 0.0% | 9.74E−01 | 9.74E−01 |

TABLE 24
Metabolites found and missing is FFPE cell and human prostate sample categorized by
pathway.

| | non- | | | | |
|---|---|---|---|---|---|
| | preserved | preserved | | | |
| | in FFPE, | in FFPE, | FFPE/ | | |
| Pathway | n (%) | n (%) | FROZEN, % | p | FDR |
| Transcription/Translation | 2 (0.8%) | 23 (9.2%) | 92.0% | 5.25E−06 | 4.62E−04 |
| Purine Metabolism | 3 (1.2%) | 19 (7.6%) | 86.4% | 3.20E−04 | 1.41E−02 |
| Ammonia Recycling | 2 (0.8%) | 12 (4.8%) | 85.7% | 6.04E−03 | 1.77E−01 |
| Urea Cycle | 2 (0.8%) | 11 (4.4%) | 84.6% | 1.09E−02 | 2.22E−01 |
| Glycine and Serine Metabolism | 7 (2.7%) | 19 (7.6%) | 73.1% | 1.51E−02 | 2.22E−01 |
| Alpha Linolenic Acid and Linoleic | 1 (0.4%) | 8 (3.2%) | 88.9% | 1.90E−02 | 2.22E−01 |
| Acid Metabolism | | | | | |
| Glutamate Metabolism | 2 (0.8%) | 9 (3.6%) | 81.8% | 3.44E−02 | 2.22E−01 |
| Valine, Leucine and Isoleucine | 1 (0.4%) | 7 (2.8%) | 87.5% | 3.54E−02 | 2.22E−01 |
| Degradation | | | | | |
| Glutathione Metabolism | 1 (0.4%) | 7 (2.8%) | 87.5% | 3.54E−02 | 2.22E−01 |
| Aspartate Metabolism | 1 (0.4%) | 7 (2.8%) | 87.5% | 3.54E−02 | 2.22E−01 |
| Plasmalogen Synthesis | 1 (0.4%) | 7 (2.8%) | 87.5% | 3.54E−02 | 2.22E−01 |
| Pyrimidine Metabolism | 6 (2.3%) | 15 (6%) | 71.4% | 4.52E−02 | 2.63E−01 |
| Amino Sugar Metabolism | 5 (1.9%) | 13 (5.2%) | 72.2% | 5.61E−02 | 2.63E−01 |
| Arginine and Proline Metabolism | 2 (0.8%) | 8 (3.2%) | 80.0% | 5.97E−02 | 2.63E−01 |
| Transfer of Acetyl Groups into | 2 (0.8%) | 8 (3.2%) | 80.0% | 5.97E−02 | 2.63E−01 |
| Mitochondria | | | | | |
| Glucose-Alanine Cycle | 1 (0.4%) | 6 (2.4%) | 85.7% | 6.51E−02 | 2.65E−01 |
| Methionine Metabolism | 4 (1.6%) | 11 (4.4%) | 73.3% | 6.92E−02 | 2.65E−01 |
| Citric Acid Cycle | 4 (1.6%) | 11 (4.4%) | 73.3% | 6.92E−02 | 2.65E−01 |
| Phospholipid Biosynthesis | 2 (0.8%) | 7 (2.8%) | 77.8% | 1.02E−01 | 3.74E−01 |
| Galactose Metabolism | 3 (1.2%) | 8 (3.2%) | 72.7% | 1.37E−01 | 4.63E−01 |
| Glycerolipid Metabolism | 3 (1.2%) | 7 (2.8%) | 70.0% | 2.16E−01 | 5.77E−01 |
| Pyruvate Metabolism | 2 (0.8%) | 5 (2%) | 71.4% | 2.80E−01 | 6.13E−01 |
| Spermidine and Spermine | 2 (0.8%) | 5 (2%) | 71.4% | 2.80E−01 | 6.13E−01 |
| Biosynthesis | | | | | |
| Mitochondrial Beta-Oxidation of | 2 (0.8%) | 5 (2%) | 71.4% | 2.80E−01 | 6.13E−01 |
| Medium Chain Saturated Fatty | | | | | |
| Acids | | | | | |
| Mitochondrial Electron Transport | 2 (0.8%) | 5 (2%) | 71.4% | 2.80E−01 | 6.13E−01 |
| Chain | | | | | |
| Starch and Sucrose Metabolism | 2 (0.8%) | 5 (2%) | 71.4% | 2.80E−01 | 6.13E−01 |
| Pentose Phosphate Pathway | 6 (2.3%) | 2 (0.8%) | 25.0% | 2.86E−01 | 6.13E−01 |
| Mitochondrial Beta-Oxidation of | 3 (1.2%) | 6 (2.4%) | 66.7% | 3.33E−01 | 6.59E−01 |
| Long Chain Saturated Fatty Acids | | | | | |
| Lactose Synthesis | 3 (1.2%) | 6 (2.4%) | 66.7% | 3.33E−01 | 6.59E−01 |
| Carnitine Synthesis | 4 (1.6%) | 7 (2.8%) | 63.6% | 3.77E−01 | 6.63E−01 |
| Gluconeogenesis | 6 (2.3%) | 9 (3.6%) | 60.0% | 4.42E−01 | 7.24E−01 |
| Nicotinate and Nicotinamide | 2 (0.8%) | 4 (1.6%) | 66.7% | 4.45E−01 | 7.24E−01 |
| Metabolism | | | | | |
| Ethanol Degradation | 2 (0.8%) | 4 (1.6%) | 66.7% | 4.45E−01 | 7.24E−01 |
| Threonine and 2-Oxobutanoate | 2 (0.8%) | 4 (1.6%) | 66.7% | 4.45E−01 | 7.24E−01 |
| Degradation | | | | | |
| Beta Oxidation of Very Long | 3 (1.2%) | 5 (2%) | 62.5% | 4.99E−01 | 7.32E−01 |
| Chain Fatty Acids | | | | | |
| Beta-Alanine Metabolism | 4 (1.6%) | 6 (2.4%) | 60.0% | 5.40E−01 | 7.79E−01 |
| Histidine Metabolism | 5 (1.9%) | 6 (2.4%) | 54.5% | 7.69E−01 | 1.00E+00 |
| Glycolysis | 6 (2.3%) | 7 (2.8%) | 53.8% | 7.85E−01 | 1.00E+00 |
| Fatty Acid Biosynthesis | 4 (1.6%) | 4 (1.6%) | 50.0% | 1.00E+00 | 1.00E+00 |
| Betaine Metabolism | 3 (1.2%) | 3 (1.2%) | 50.0% | 1.00E+00 | 1.00E+00 |
| Folate Metabolism | 3 (1.2%) | 3 (1.2%) | 50.0% | 1.00E+00 | 1.00E+00 |
| Mitochondrial Beta-Oxidation of | 5 (1.9%) | 5 (2%) | 50.0% | 1.00E+00 | 1.00E+00 |
| Short Chain Saturated Fatty Acids | | | | | |
| Fructose and Mannose Degradation | 4 (1.6%) | 4 (1.6%) | 50.0% | 1.00E+00 | 1.00E+00 |

In human FFPE samples, similar to the cell lines, samples correlation coefficients between metabolite concentrations from replicates ranged between 0.920 and 0.994 (median value of 0.979), while from frozen and FFPE samples ranged between 0.471 and 0.698 (median value 0.609), as shown in FIG. 3D.

### Example 6: Metabolic Profiling of Human Prostate Cancer Tissue

Only the 112 metabolites shared in frozen and FFPE samples with less than 25% missing values were considered to delineate the prostate cancer fingerprint. Hierarchical clustering based on KODAMA dissimilarity matrix distinguished normal and tumor prostate tissues (FIG. 3E) both in frozen and FFPE material.

Tumor and normal frozen tissue samples were able to be separated by hierarchical clustering in both OCT-embedded and FFPE samples. A total of 48 out of 112 metabolites were significantly different between normal and tumor tissue in FFPE samples, whilst 61 out of 112 metabolites were significantly different in frozen samples. Thirty-two metabolites were statistically significant in both frozen and FFPE samples. Results are reported in Table 25 and Table 26, which list metabolite statistical analysis of the differences between normal and tumor prostate tissues in frozen and FFPE samples, respectively. Among the perturbed metabolites found in both OCT-embedded and FFPE samples, 17 were increased in tumor tissue and 13 were down-regulated. Agreement in the direction of metabolite abundance in frozen and FFPE comparisons served as an important indication of the reliability of metabolite detection in FFPE samples.

TABLE 25
Metabolite statistical analysis of normal and tumor prostate tissues in frozen samples

| | FROZEN | | | | | |
|---|---|---|---|---|---|---|
| | | | Normal, | Tumor, | log | |
| | p | FDR | mean | mean | ratio | loadings |
| taurine | 1.55E−04 | 1.34E−03 | 1.83E−03 | 7.60E−04 | 1.27 | 0.15 |
| 1-palmitoylglycerophosphoinositol | 1.55E−04 | 1.34E−03 | 1.48E−04 | 4.78E−04 | −1.69 | −0.14 |
| pyroglutamine | 1.55E−04 | 1.34E−03 | 5.69E−04 | 3.21E−04 | 0.83 | 0.15 |
| glutathione, oxidized | 1.55E−04 | 1.34E−03 | 2.22E−02 | 1.03E−02 | 1.11 | 0.14 |
| dihomo-linoleate | 1.55E−04 | 1.34E−03 | 6.39E−04 | 2.68E−03 | −2.07 | −0.12 |
| creatinine | 1.55E−04 | 1.34E−03 | 1.50E−03 | 6.19E−04 | 1.28 | 0.15 |
| 1- | 1.55E−04 | 1.34E−03 | 1.99E−04 | 9.98E−04 | −2.32 | −0.12 |
| linoleoylglycerophosphoethanolamine | | | | | | |
| eicosenoate | 1.55E−04 | 1.34E−03 | 4.50E−04 | 1.59E−03 | −1.82 | −0.13 |
| 10-nonadecenoate | 1.55E−04 | 1.34E−03 | 5.72E−05 | 2.31E−04 | −2.01 | −0.15 |
| 1-oleoylglycerol | 1.55E−04 | 1.34E−03 | 1.76E−05 | 2.71E−05 | −0.63 | −0.12 |
| palmitate | 1.55E−04 | 1.34E−03 | 1.78E−02 | 1.20E−02 | 0.56 | 0.14 |
| palmitoyl sphingomyelin | 1.55E−04 | 1.34E−03 | 5.97E−03 | 3.32E−03 | 0.84 | 0.14 |
| 2-aminoadipate | 1.55E−04 | 1.34E−03 | 4.72E−04 | 1.22E−03 | −1.36 | −0.15 |
| 1-oleoylglycerophosphoinositol | 3.11E−04 | 1.45E−03 | 1.16E−04 | 3.47E−04 | −1.59 | −0.13 |
| myristate | 3.11E−04 | 1.45E−03 | 1.53E−03 | 1.05E−03 | 0.54 | 0.13 |
| threonine | 3.11E−04 | 1.45E−03 | 3.30E−03 | 4.43E−03 | −0.43 | −0.14 |
| docosapentaenoate (n3) | 3.11E−04 | 1.45E−03 | 2.73E−04 | 1.14E−03 | −2.07 | −0.13 |
| stearate | 3.11E−04 | 1.45E−03 | 2.07E−02 | 1.37E−02 | 0.60 | 0.15 |
| threonate | 3.11E−04 | 1.45E−03 | 9.82E−05 | 3.43E−05 | 1.52 | 0.13 |
| histidine | 3.11E−04 | 1.45E−03 | 1.84E−04 | 1.37E−04 | 0.42 | 0.12 |
| 2- | 3.11E−04 | 1.45E−03 | 2.38E−04 | 7.05E−04 | −1.57 | −0.12 |
| oleoylglycerophosphoethanolamine | | | | | | |
| 1- | 3.11E−04 | 1.45E−03 | 2.70E−03 | 7.73E−03 | −1.52 | −0.12 |
| oleoylglycerophosphoethanolamine | | | | | | |
| myo-inositol | 3.11E−04 | 1.45E−03 | 1.99E−01 | 1.30E−01 | 0.62 | 0.13 |
| 1-palmitoylglycerophosphocholine | 3.11E−04 | 1.45E−03 | 1.12E−02 | 2.12E−02 | −0.92 | −0.12 |
| docosahexaenoate | 1.09E−03 | 4.20E−03 | 1.69E−03 | 5.52E−03 | −1.71 | −0.12 |
| 13-methylmyristic acid | 1.09E−03 | 4.20E−03 | 8.82E−04 | 5.53E−04 | 0.67 | 0.13 |
| 2- | 1.09E−03 | 4.20E−03 | 2.12E−04 | 1.27E−04 | 0.74 | 0.13 |
| arachidonoylglycerophosphoethanolamine | | | | | | |
| 1-oleoylglycerophosphoserine | 1.09E−03 | 4.20E−03 | 8.40E−05 | 1.76E−04 | −1.07 | −0.13 |
| glycerate | 1.09E−03 | 4.20E−03 | 9.48E−04 | 6.11E−04 | 0.63 | 0.11 |
| 1- | 1.86E−03 | 6.14E−03 | 1.38E−03 | 2.89E−03 | −1.07 | −0.10 |
| stearoylglycerophosphoethanolamine | | | | | | |
| laurate | 1.86E−03 | 6.14E−03 | 5.82E−04 | 3.65E−04 | 0.67 | 0.10 |
| linoleate | 1.86E−03 | 6.14E−03 | 2.98E−03 | 1.04E−02 | −1.80 | −0.12 |
| oleate | 1.86E−03 | 6.14E−03 | 9.13E−04 | 2.19E−03 | −1.26 | −0.12 |
| erythronate | 1.86E−03 | 6.14E−03 | 1.24E−04 | 7.78E−05 | 0.68 | 0.13 |
| 1-stearoylglycerophosphoinositol | 2.95E−03 | 8.07E−03 | 3.66E−04 | 1.33E−03 | −1.86 | −0.12 |
| dihomo-linolenate | 2.95E−03 | 8.07E−03 | 2.68E−03 | 8.02E−03 | −1.58 | −0.11 |
| ophthalmate | 2.95E−03 | 8.07E−03 | 1.11E−03 | 6.81E−04 | 0.70 | 0.12 |
| arachidonate | 2.95E−03 | 8.07E−03 | 9.65E−03 | 2.07E−02 | −1.10 | −0.11 |
| glycerophosphoethanolamine | 2.95E−03 | 8.07E−03 | 5.26E−04 | 1.02E−03 | −0.96 | −0.12 |
| cytidine 5′-monophosphate | 2.95E−03 | 8.07E−03 | 2.62E−04 | 1.89E−04 | 0.47 | 0.12 |
| cysteine | 2.95E−03 | 8.07E−03 | 1.99E−03 | 4.82E−03 | −1.27 | −0.12 |
| glucose | 4.66E−03 | 1.21E−02 | 3.68E−03 | 1.37E−03 | 1.43 | 0.11 |
| 1- | 4.66E−03 | 1.21E−02 | 2.46E−03 | 5.69E−03 | −1.21 | −0.11 |
| palmitoylglycerophosphoethanolamine | | | | | | |
| inositol 1-phosphate | 6.99E−03 | 1.78E−02 | 4.91E−04 | 2.44E−04 | 1.01 | 0.11 |
| valine | 1.04E−02 | 2.48E−02 | 1.27E−02 | 1.03E−02 | 0.29 | 0.12 |
| Isobar: UDP-acetylglucosamine, | 1.04E−02 | 2.48E−02 | 3.06E−04 | 4.56E−04 | −0.58 | −0.10 |
| UDP-acetylgalactosamine | | | | | | |
| 2-stearoylglycerophosphoinositol | 1.04E−02 | 2.48E−02 | 4.03E−05 | 8.15E−05 | −1.01 | −0.10 |
| 1-palmitoylglycerol | 1.48E−02 | 3.18E−02 | 9.07E−05 | 1.16E−04 | −0.36 | −0.11 |
| serine | 1.48E−02 | 3.18E−02 | 9.02E−03 | 1.08E−02 | −0.27 | −0.10 |
| guanosine 5′-monophosphate | 1.48E−02 | 3.18E−02 | 6.19E−04 | 2.74E−04 | 1.18 | 0.12 |
| glutamine | 1.48E−02 | 3.18E−02 | 8.26E−03 | 6.24E−03 | 0.40 | 0.11 |
| myristoleate | 1.48E−02 | 3.18E−02 | 7.22E−05 | 5.56E−05 | 0.38 | 0.09 |
| glycerophosphorylcholine | 2.07E−02 | 4.29E−02 | 3.76E−03 | 5.78E−03 | −0.62 | −0.10 |
| 2-methylbutyrylcarnitine | 2.07E−02 | 4.29E−02 | 2.54E−04 | 1.73E−04 | 0.55 | 0.08 |
| 1- | 2.81E−02 | 5.43E−02 | 5.58E−04 | 4.86E−04 | 0.20 | 0.09 |
| arachidonoylglycerophosphoethanolamine | | | | | | |
| creatine | 2.81E−02 | 5.43E−02 | 4.46E−02 | 3.02E−02 | 0.56 | 0.11 |
| adenine | 2.81E−02 | 5.43E−02 | 3.38E−04 | 5.51E−04 | −0.71 | −0.09 |
| 2- | 2.81E−02 | 5.43E−02 | 3.47E−04 | 1.20E−03 | −1.79 | −0.10 |
| palmitoylglycerophosphoethanolamine | | | | | | |
| adenosine 5′-monophosphate | 3.79E−02 | 7.20E−02 | 1.28E−02 | 7.21E−03 | 0.83 | 0.09 |
| N-acetylneuraminate | 4.99E−02 | 9.16E−02 | 2.12E−04 | 3.25E−04 | −0.62 | −0.10 |
| palmitoleate | 4.99E−02 | 9.16E−02 | 1.08E−03 | 9.55E−04 | 0.17 | 0.07 |
| phosphoethanolamine | 8.30E−02 | 1.43E−01 | 6.77E−03 | 3.91E−03 | 0.79 | 0.09 |
| choline phosphate | 8.30E−02 | 1.43E−01 | 3.64E−03 | 1.23E−03 | 1.56 | 0.09 |
| phosphate | 8.30E−02 | 1.43E−01 | 1.79E−01 | 1.61E−01 | 0.15 | 0.06 |
| cis-vaccenate | 8.30E−02 | 1.43E−01 | 1.94E−04 | 2.97E−04 | −0.62 | −0.09 |
| scyllo-inositol | 1.05E−01 | 1.78E−01 | 7.80E−03 | 6.44E−03 | 0.28 | 0.09 |
| alanine | 1.30E−01 | 2.12E−01 | 4.89E−02 | 5.37E−02 | −0.13 | −0.06 |
| glutamate | 1.30E−01 | 2.12E−01 | 1.15E−02 | 9.57E−03 | 0.27 | 0.07 |
| 5-oxoproline | 1.30E−01 | 2.12E−01 | 1.86E−04 | 2.93E−04 | −0.66 | −0.07 |
| guanine | 1.61E−01 | 2.53E−01 | 1.08E−03 | 1.30E−03 | −0.26 | −0.06 |
| citrate | 1.61E−01 | 2.53E−01 | 1.82E−01 | 1.33E−01 | 0.45 | 0.06 |
| cytidine | 1.95E−01 | 2.99E−01 | 4.78E−04 | 3.58E−04 | 0.42 | 0.06 |
| nicotinamide | 1.95E−01 | 2.99E−01 | 2.57E−03 | 2.60E−03 | −0.02 | 0.00 |
| spermidine | 2.34E−01 | 3.46E−01 | 3.22E−03 | 2.72E−03 | 0.24 | 0.06 |
| uridine 5′-monophosphate | 2.34E−01 | 3.46E−01 | 4.42E−04 | 2.79E−04 | 0.66 | 0.09 |
| fumarate | 2.34E−01 | 3.46E−01 | 4.10E−04 | 4.65E−04 | −0.18 | −0.07 |
| glycerol 3-phosphate | 2.79E−01 | 3.76E−01 | 1.72E−03 | 1.96E−03 | −0.19 | −0.06 |
| ethanolamine | 2.79E−01 | 3.76E−01 | 9.05E−03 | 1.35E−02 | −0.58 | −0.05 |
| 6-sialyl-N-acetyllactosamine | 2.79E−01 | 3.76E−01 | 1.45E−04 | 9.60E−05 | 0.60 | 0.07 |
| sorbitol | 2.79E−01 | 3.76E−01 | 7.26E−05 | 5.52E−05 | 0.39 | 0.04 |
| glycine | 2.79E−01 | 3.76E−01 | 2.72E−02 | 3.18E−02 | −0.23 | −0.05 |
| linolenate (alpha or gamma) | 2.79E−01 | 3.76E−01 | 1.01E−04 | 1.55E−04 | −0.62 | −0.08 |
| asparagine | 2.79E−01 | 3.76E−01 | 1.35E−04 | 1.78E−04 | −0.40 | −0.07 |
| 2-palmitoylglycerol | 3.28E−01 | 4.32E−01 | 1.09E−04 | 9.33E−05 | 0.22 | 0.06 |
| lysine | 3.28E−01 | 4.32E−01 | 1.59E−03 | 1.87E−03 | −0.24 | −0.05 |
| isoleucine | 3.82E−01 | 4.98E−01 | 9.74E−03 | 8.83E−03 | 0.14 | 0.04 |
| 5-methylthioadenosine | 4.42E−01 | 5.62E−01 | 1.96E−04 | 2.14E−04 | −0.13 | −0.05 |
| glycerol | 4.42E−01 | 5.62E−01 | 1.17E−02 | 1.12E−02 | 0.06 | 0.04 |
| aspartate | 5.05E−01 | 6.15E−01 | 7.06E−03 | 7.94E−03 | −0.17 | −0.04 |
| arachidate | 5.05E−01 | 6.15E−01 | 3.02E−05 | 3.39E−05 | −0.17 | −0.04 |
| cytidine 5′-diphosphocholine | 5.05E−01 | 6.15E−01 | 4.73E−04 | 4.89E−04 | −0.05 | −0.03 |
| cysteine-glutathione disulfide | 5.05E−01 | 6.15E−01 | 1.50E−03 | 2.83E−03 | −0.92 | −0.04 |
| proline | 6.45E−01 | 7.61E−01 | 8.64E−03 | 8.86E−03 | −0.04 | −0.04 |
| inosine | 6.45E−01 | 7.61E−01 | 2.31E−03 | 2.29E−03 | 0.01 | −0.01 |
| methylphosphate | 6.45E−01 | 7.61E−01 | 2.18E−02 | 2.37E−02 | −0.12 | 0.01 |
| fructose | 7.21E−01 | 7.99E−01 | 1.07E−03 | 6.03E−04 | 0.83 | 0.03 |
| adenosine | 7.21E−01 | 7.99E−01 | 1.45E−02 | 1.38E−02 | 0.07 | 0.01 |
| arginine | 7.21E−01 | 7.99E−01 | 3.19E−04 | 2.62E−04 | 0.28 | 0.00 |
| 2-hydroxyglutarate | 7.21E−01 | 7.99E−01 | 2.70E−04 | 4.74E−03 | −4.13 | −0.03 |
| acetylcarnitine | 7.21E−01 | 7.99E−01 | 8.60E−03 | 7.87E−03 | 0.13 | 0.03 |
| beta-alanine | 7.21E−01 | 7.99E−01 | 3.39E−04 | 3.37E−04 | 0.01 | 0.02 |
| phenylalanine | 7.98E−01 | 8.60E−01 | 1.06E−02 | 1.01E−02 | 0.07 | 0.00 |
| succinate | 7.98E−01 | 8.60E−01 | 7.98E−04 | 8.07E−04 | −0.02 | −0.01 |
| malate | 7.98E−01 | 8.60E−01 | 1.49E−03 | 1.45E−03 | 0.04 | −0.02 |
| 1-stearoylglycerol | 8.78E−01 | 9.28E−01 | 9.56E−05 | 1.24E−04 | −0.37 | −0.04 |
| N-acetylaspartate | 8.78E−01 | 9.28E−01 | 2.21E−04 | 2.23E−04 | −0.02 | −0.01 |
| uridine | 9.59E−01 | 9.68E−01 | 1.40E−03 | 1.33E−03 | 0.07 | 0.01 |
| leucine | 9.59E−01 | 9.68E−01 | 1.23E−02 | 1.20E−02 | 0.03 | 0.01 |
| tyrosine | 9.59E−01 | 9.68E−01 | 4.19E−03 | 4.07E−03 | 0.04 | 0.01 |
| guanosine | 9.59E−01 | 9.68E−01 | 5.05E−04 | 5.00E−04 | 0.02 | 0.00 |
| putrescine | 9.59E−01 | 9.68E−01 | 2.21E−02 | 2.11E−02 | 0.07 | 0.00 |
| carnitine | 1.00E+00 | 1.00E+00 | 3.24E−03 | 3.22E−03 | 0.01 | 0.01 |

TABLE 26
Metabolite statistical analysis of normal and tumor prostate tissues in FFPE samples

| | FFPE | | | | | |
|---|---|---|---|---|---|---|
| | | | Normal, | Tumor, | log | |
| | p | FDR | mean | mean | ratio | loadings |
| taurine | 5.39E−08 | 3.02E−06 | 4.43E−04 | 3.07E−04 | 0.53 | 0.22 |
| 1-palmitoylglycerophosphoinositol | 1.97E−05 | 3.08E−04 | 1.22E−04 | 1.89E−04 | −0.64 | −0.15 |
| pyroglutamine | 3.60E−04 | 2.88E−03 | 5.56E−04 | 4.26E−04 | 0.39 | 0.14 |
| glutathione, oxidized | 7.19E−04 | 4.73E−03 | 1.39E−04 | 1.09E−04 | 0.35 | 0.17 |
| dihomo-linoleate | 1.49E−03 | 7.58E−03 | 1.37E−04 | 1.97E−04 | −0.52 | −0.12 |
| creatinine | 3.64E−03 | 1.51E−02 | 2.88E−03 | 2.42E−03 | 0.25 | 0.12 |
| 1- | 8.78E−03 | 3.07E−02 | 1.08E−04 | 1.36E−04 | −0.33 | −0.07 |
| linoleoylglycerophosphoethanolamine | | | | | | |
| eicosenoate | 1.63E−02 | 5.06E−02 | 3.43E−04 | 4.44E−04 | −0.37 | −0.09 |
| 10-nonadecenoate | 2.71E−02 | 6.91E−02 | 5.16E−05 | 6.74E−05 | −0.39 | −0.10 |
| 1-oleoylglycerol | 7.10E−02 | 1.44E−01 | 1.44E−04 | 7.52E−05 | 0.94 | 0.06 |
| palmitate | 8.14E−02 | 1.57E−01 | 3.08E−02 | 2.78E−02 | 0.15 | 0.07 |
| palmitoyl sphingomyelin | 5.60E−01 | 6.82E−01 | 5.34E−04 | 5.47E−04 | −0.04 | −0.04 |
| 2-aminoadipate | 7.36E−01 | 8.16E−01 | 1.63E−04 | 1.41E−04 | 0.21 | 0.00 |
| 1-oleoylglycerophosphoinositol | 4.69E−04 | 3.28E−03 | 1.69E−04 | 2.51E−04 | −0.57 | −0.17 |
| myristate | 1.44E−02 | 4.62E−02 | 3.28E−02 | 2.64E−02 | 0.31 | 0.11 |
| threonine | 1.83E−02 | 5.25E−02 | 1.99E−04 | 2.57E−04 | −0.37 | −0.11 |
| docosapentaenoate (n3) | 2.17E−02 | 6.08E−02 | 8.88E−05 | 1.24E−04 | −0.48 | −0.13 |
| stearate | 2.30E−02 | 6.28E−02 | 5.61E−02 | 4.78E−02 | 0.23 | 0.09 |
| threonate | 5.87E−02 | 1.34E−01 | 5.31E−05 | 4.41E−05 | 0.27 | 0.09 |
| histidine | 6.77E−02 | 1.43E−01 | 2.33E−04 | 1.92E−04 | 0.28 | 0.08 |
| 2- | 1.06E−01 | 1.91E−01 | 9.81E−05 | 1.10E−04 | −0.16 | −0.07 |
| oleoylglycerophosphoethanolamine | | | | | | |
| 1- | 1.65E−01 | 2.57E−01 | 6.39E−04 | 6.98E−04 | −0.13 | −0.06 |
| oleoylglycerophosphoethanolamine | | | | | | |
| myo-inositol | 2.38E−01 | 3.42E−01 | 6.93E−02 | 5.74E−02 | 0.27 | 0.06 |
| 1-palmitoylglycerophosphocholine | 4.64E−01 | 5.89E−01 | 3.81E−04 | 3.87E−04 | −0.02 | −0.01 |
| docosahexaenoate | 1.17E−03 | 6.26E−03 | 1.88E−04 | 2.53E−04 | −0.43 | −0.15 |
| 13-methylmyristic acid | 2.57E−02 | 6.85E−02 | 3.31E−04 | 2.86E−04 | 0.21 | 0.08 |
| 2- | 3.55E−02 | 8.84E−02 | 2.02E−04 | 1.59E−04 | 0.35 | 0.09 |
| arachidonoylglycerophosphoethanolamine | | | | | | |
| 1-oleoylglycerophosphoserine | 1.31E−01 | 2.21E−01 | 4.72E−04 | 4.13E−04 | 0.19 | 0.05 |
| glycerate | 8.78E−01 | 9.02E−01 | 2.34E−04 | 2.35E−04 | −0.01 | 0.01 |
| 1- | 4.59E−02 | 1.07E−01 | 3.94E−04 | 4.86E−04 | −0.30 | −0.05 |
| stearoylglycerophosphoethanolamine | | | | | | |
| laurate | 6.16E−02 | 1.35E−01 | 3.96E−04 | 3.47E−04 | 0.19 | 0.08 |
| linoleate | 3.20E−01 | 4.38E−01 | 4.84E−03 | 6.00E−03 | −0.31 | −0.05 |
| oleate | 8.14E−01 | 8.52E−01 | 3.89E−03 | 4.25E−03 | −0.13 | −0.02 |
| erythronate | 9.43E−01 | 9.52E−01 | 4.03E−05 | 3.92E−05 | 0.04 | 0.01 |
| 1-stearoylglycerophosphoinositol | 4.76E−06 | 1.33E−04 | 3.98E−04 | 6.84E−04 | −0.78 | −0.18 |
| dihomo-linolenate | 1.76E−05 | 3.08E−04 | 1.33E−04 | 1.82E−04 | −0.46 | −0.17 |
| ophthalmate | 4.50E−03 | 1.80E−02 | 1.91E−04 | 1.68E−04 | 0.18 | 0.11 |
| arachidonate | 3.94E−02 | 9.59E−02 | 6.82E−04 | 7.55E−04 | −0.15 | −0.10 |
| glycerophosphoethanolamine | 2.22E−01 | 3.32E−01 | 2.95E−04 | 3.60E−04 | −0.29 | −0.04 |
| cytidine 5′-monophosphate | 3.12E−01 | 4.32E−01 | 1.37E−04 | 1.19E−04 | 0.20 | 0.04 |
| cysteine | 4.80E−01 | 5.98E−01 | 1.53E−04 | 1.59E−04 | −0.06 | −0.03 |
| glucose | 2.71E−02 | 6.91E−02 | 4.13E−03 | 7.05E−04 | 2.55 | 0.10 |
| 1- | 1.53E−01 | 2.45E−01 | 3.05E−04 | 3.41E−04 | −0.16 | −0.01 |
| palmitoylglycerophosphoethanolamine | | | | | | |
| inositol 1-phosphate | 7.83E−01 | 8.35E−01 | 1.61E−04 | 1.62E−04 | −0.01 | 0.01 |
| valine | 1.00E−03 | 6.22E−03 | 4.85E−04 | 6.41E−04 | −0.40 | −0.15 |
| Isobar: UDP-acetylglucosamine, | 1.06E−02 | 3.50E−02 | 1.92E−04 | 2.43E−04 | −0.34 | −0.07 |
| UDP-acetylgalactosamine | | | | | | |
| 2-stearoylglycerophosphoinositol | 1.68E−02 | 5.07E−02 | 7.44E−05 | 9.90E−05 | −0.41 | −0.11 |
| 1-palmitoylglycerol | 8.70E−05 | 8.12E−04 | 4.23E−04 | 2.73E−04 | 0.63 | 0.12 |
| serine | 8.23E−03 | 2.97E−02 | 1.01E−03 | 1.30E−03 | −0.37 | −0.11 |
| guanosine 5′-monophosphate | 7.43E−02 | 1.49E−01 | 3.16E−04 | 2.63E−04 | 0.27 | 0.10 |
| glutamine | 7.36E−01 | 8.16E−01 | 7.96E−04 | 7.98E−04 | 0.00 | −0.01 |
| myristoleate | 7.83E−01 | 8.35E−01 | 1.09E−04 | 1.22E−04 | −0.16 | −0.02 |
| glycerophosphorylcholine | 6.33E−03 | 2.44E−02 | 1.65E−03 | 1.89E−03 | −0.19 | −0.11 |
| 2-methylbutyrylcarnitine | 6.16E−02 | 1.35E−01 | 3.08E−04 | 2.52E−04 | 0.29 | 0.08 |
| 1- | 1.11E−05 | 2.48E−04 | 1.66E−03 | 1.32E−03 | 0.33 | 0.16 |
| arachidonoylglycerophosphoethanolamine | | | | | | |
| creatine | 1.61E−03 | 7.84E−03 | 3.39E−03 | 2.62E−03 | 0.37 | 0.11 |
| adenine | 2.47E−01 | 3.49E−01 | 7.70E−04 | 8.35E−04 | −0.12 | −0.06 |
| 2- | 7.97E−01 | 8.42E−01 | 5.53E−05 | 5.51E−05 | 0.00 | 0.07 |
| palmitoylglycerophosphoethanolamine | | | | | | |
| adenosine 5′-monophosphate | 8.70E−05 | 8.12E−04 | 5.66E−03 | 4.21E−03 | 0.43 | 0.17 |
| N-acetylneuraminate | 6.17E−01 | 7.35E−01 | 1.95E−04 | 2.06E−04 | −0.08 | −0.02 |
| palmitoleate | 7.21E−01 | 8.15E−01 | 1.89E−03 | 2.02E−03 | −0.09 | 0.00 |
| phosphoethanolamine | 4.69E−04 | 3.28E−03 | 1.02E−04 | 6.84E−05 | 0.58 | 0.16 |
| choline phosphate | 2.35E−03 | 1.05E−02 | 1.56E−03 | 1.06E−03 | 0.56 | 0.16 |
| phosphate | 3.39E−03 | 1.46E−02 | 7.39E−01 | 6.34E−01 | 0.22 | 0.07 |
| cis-vaccenate | 6.60E−01 | 7.62E−01 | 4.79E−04 | 5.24E−04 | −0.13 | 0.00 |
| scyllo-inositol | 3.62E−01 | 4.89E−01 | 2.48E−03 | 2.14E−03 | 0.21 | 0.05 |
| alanine | 2.03E−03 | 9.45E−03 | 5.34E−03 | 7.17E−03 | −0.42 | −0.14 |
| glutamate | 2.30E−01 | 3.35E−01 | 2.45E−03 | 2.27E−03 | 0.11 | 0.07 |
| 5-oxoproline | 7.67E−01 | 8.34E−01 | 2.33E−04 | 2.37E−04 | −0.03 | −0.05 |
| guanine | 2.30E−01 | 3.35E−01 | 5.87E−04 | 6.52E−04 | −0.15 | −0.05 |
| citrate | 9.43E−01 | 9.52E−01 | 8.05E−02 | 5.54E−02 | 0.54 | 0.03 |
| cytidine | 1.06E−02 | 3.50E−02 | 1.33E−04 | 1.82E−04 | −0.45 | −0.13 |
| nicotinamide | 3.73E−01 | 4.97E−01 | 1.62E−04 | 1.79E−04 | −0.15 | −0.04 |
| spermidine | 1.36E−01 | 2.27E−01 | 9.90E−05 | 8.89E−05 | 0.16 | 0.04 |
| uridine 5′-monophosphate | 1.53E−01 | 2.45E−01 | 7.14E−05 | 6.47E−05 | 0.14 | 0.08 |
| fumarate | 6.02E−01 | 7.25E−01 | 1.70E−04 | 1.61E−04 | 0.08 | −0.01 |
| glycerol 3-phosphate | 8.23E−03 | 2.97E−02 | 9.38E−04 | 6.99E−04 | 0.42 | 0.11 |
| ethanolamine | 6.46E−02 | 1.39E−01 | 2.03E−03 | 2.29E−03 | −0.18 | −0.04 |
| 6-sialyl-N-acetyllactosamine | 7.78E−02 | 1.53E−01 | 6.10E−05 | 4.91E−05 | 0.31 | 0.10 |
| sorbitol | 8.52E−02 | 1.62E−01 | 4.60E−04 | 1.95E−04 | 1.24 | 0.12 |
| glycine | 1.65E−01 | 2.57E−01 | 8.82E−03 | 8.81E−03 | 0.00 | −0.05 |
| linolenate (alpha or gamma) | 3.01E−01 | 4.21E−01 | 3.64E−04 | 5.16E−04 | −0.50 | −0.06 |
| asparagine | 3.96E−01 | 5.21E−01 | 1.47E−04 | 1.56E−04 | −0.08 | −0.04 |
| 2-palmitoylglycerol | 1.11E−01 | 1.93E−01 | 9.67E−05 | 6.99E−05 | 0.47 | 0.07 |
| lysine | 4.68E−01 | 5.89E−01 | 1.84E−04 | 1.72E−04 | 0.09 | 0.03 |
| isoleucine | 4.37E−02 | 1.04E−01 | 3.84E−04 | 4.53E−04 | −0.24 | −0.13 |
| 5-methylthioadenosine | 1.15E−01 | 1.98E−01 | 1.15E−04 | 1.32E−04 | −0.19 | −0.07 |
| glycerol | 6.46E−01 | 7.61E−01 | 7.64E−05 | 8.71E−05 | −0.19 | −0.03 |
| aspartate | 1.57E−08 | 1.76E−06 | 2.56E−03 | 3.58E−03 | −0.48 | −0.15 |
| arachidate | 1.78E−01 | 2.70E−01 | 1.02E−03 | 9.21E−04 | 0.14 | 0.06 |
| cytidine 5′-diphosphocholine | 6.60E−01 | 7.62E−01 | 1.75E−04 | 1.81E−04 | −0.05 | −0.01 |
| cysteine-glutathione disulfide | 7.67E−01 | 8.34E−01 | 1.42E−04 | 1.46E−04 | −0.04 | −0.04 |
| proline | 7.10E−02 | 1.44E−01 | 5.62E−04 | 6.94E−04 | −0.30 | −0.12 |
| inosine | 1.11E−01 | 1.93E−01 | 2.50E−04 | 2.70E−04 | −0.11 | −0.08 |
| methylphosphate | 7.21E−01 | 8.15E−01 | 7.25E−04 | 6.95E−04 | 0.06 | 0.00 |
| fructose | 1.17E−03 | 6.26E−03 | 6.59E−03 | 1.81E−04 | 5.18 | 0.12 |
| adenosine | 1.02E−01 | 1.86E−01 | 2.09E−03 | 2.22E−03 | −0.09 | −0.06 |
| arginine | 1.72E−01 | 2.64E−01 | 3.04E−04 | 2.88E−04 | 0.08 | 0.06 |
| 2-hydroxyglutarate | 4.19E−01 | 5.45E−01 | 5.19E−05 | 7.51E−05 | −0.53 | 0.03 |
| acetylcarnitine | 4.68E−01 | 5.89E−01 | 5.69E−04 | 5.46E−04 | 0.06 | −0.05 |
| beta-alanine | 1.00E+00 | 1.00E+00 | 7.80E−05 | 7.80E−05 | 0.00 | 0.03 |
| phenylalanine | 2.20E−05 | 3.08E−04 | 8.15E−04 | 1.01E−03 | −0.31 | −0.17 |
| succinate | 1.41E−01 | 2.33E−01 | 2.04E−04 | 1.91E−04 | 0.09 | 0.04 |
| malate | 8.78E−01 | 9.02E−01 | 4.41E−04 | 3.81E−04 | 0.21 | 0.04 |
| 1-stearoylglycerol | 1.08E−03 | 6.26E−03 | 1.31E−03 | 1.10E−03 | 0.25 | 0.12 |
| N-acetylaspartate | 1.82E−02 | 5.25E−02 | 5.46E−05 | 7.46E−05 | −0.45 | −0.12 |
| uridine | 2.21E−06 | 8.25E−05 | 2.77E−04 | 3.97E−04 | −0.52 | −0.17 |
| leucine | 3.41E−05 | 4.25E−04 | 7.55E−04 | 9.56E−04 | −0.34 | −0.18 |
| tyrosine | 5.79E−05 | 6.48E−04 | 4.53E−04 | 5.57E−04 | −0.30 | −0.17 |
| guanosine | 1.02E−01 | 1.86E−01 | 8.11E−04 | 7.18E−04 | 0.18 | 0.05 |
| putrescine | 4.93E−01 | 6.07E−01 | 3.08E−03 | 2.37E−03 | 0.38 | 0.07 |
| carnitine | 1.72E−04 | 1.48E−03 | 1.14E−03 | 1.42E−03 | −0.31 | −0.16 |

Next, the coefficient of probabilistic quotient normalization of each sample was correlated with the signal intensity of each metabolite before the normalization step (Table 54). Cytidine 50-diphosphocholine (r=0.905, P=2.77×10⁻¹⁸; FDR=1.55×10⁻¹⁶) was identified as a candidate housekeeping metabolite to adopt in orthogonal metabolic profiling when tissue weight cannot be available for normalization as in the case of FFPE material. An example of the ratio of 2 statistically different metabolites between normal and tumor tissue and cytidine 50-diphosphocholine is reported in FIG. 27 and Table 28.

TABLE 27
Ranking of housekeeping metabolites

| | | | | missing | |
|---|---|---|---|---|---|
| | | | | value | |
| metabolite | r | p-value | FDR | (%) | score |
| tyrosine | 0.920352 | 5.75E−20 | 6.44E−18 | 0 | 1 |
| cytidine 5′-diphosphocholine | 0.904671 | 2.77E−18 | 1.55E−16 | 0 | 0 |
| glycerophosphorylcholine | 0.902506 | 4.49E−18 | 1.68E−16 | 0 | 1 |
| inosine | 0.895425 | 2.01E−17 | 5.64E−16 | 0 | 0 |
| aspartate | 0.89363 | 2.90E−17 | 5.78E−16 | 0 | 1 |
| threonine | 0.8933 | 3.10E−17 | 5.78E−16 | 0 | 1 |
| dihomo-linolenate | 0.884859 | 1.57E−16 | 2.51E−15 | 0 | 0 |
| alanine | 0.878237 | 5.13E−16 | 7.18E−15 | 0 | 1 |
| carnitine | 0.874502 | 9.72E−16 | 1.21E−14 | 0 | 0 |
| arachidonate | 0.873003 | 1.25E−15 | 1.40E−14 | 0 | 0 |
| uridine | 0.872202 | 1.43E−15 | 1.45E−14 | 0 | 0 |
| adenine | 0.866667 | 3.48E−15 | 3.25E−14 | 0 | 0 |
| 1-palmitoylglycerophosphoinositol | 0.874347 | 4.33E−15 | 3.73E−14 | 4.2 | 0 |
| isoleucine | 0.861323 | 7.96E−15 | 6.36E−14 | 0 | 1 |
| serine | 0.856848 | 1.55E−14 | 1.16E−13 | 0 | 1 |
| glycerol | 0.855893 | 1.78E−14 | 1.24E−13 | 0 | 1 |
| asparagine | 0.854813 | 2.08E−14 | 1.37E−13 | 0 | 1 |
| palmitoyl sphingomyelin | 0.867652 | 2.51E−14 | 1.56E−13 | 6.2 | 0 |
| adenosine | 0.849564 | 4.36E−14 | 2.57E−13 | 0 | 0 |
| glutamate | 0.847963 | 5.44E−14 | 3.05E−13 | 0 | 1 |
| malate | 0.846888 | 6.30E−14 | 3.36E−13 | 0 | 1 |
| glycine | 0.843016 | 1.06E−13 | 5.39E−13 | 0 | 1 |
| 1-oleoylglycerophosphoinositol | 0.851624 | 1.22E−13 | 5.80E−13 | 4.2 | 0 |
| glycerol 3-phosphate | 0.841799 | 1.24E−13 | 5.80E−13 | 0 | 1 |
| inositol 1-phosphate | 0.836648 | 2.41E−13 | 1.08E−12 | 0 | 0 |
| docosahexaenoate | 0.834556 | 3.14E−13 | 1.35E−12 | 0 | 0 |
| eicosenoate | 0.83306 | 3.78E−13 | 1.57E−12 | 0 | 0 |
| methylphosphate | 0.831045 | 4.84E−13 | 1.94E−12 | 0 | 0 |
| leucine | 0.823597 | 1.18E−12 | 4.54E−12 | 0 | 1 |
| dihomo-linoleate | 0.820014 | 1.78E−12 | 6.63E−12 | 0 | 0 |
| 5-oxoproline | 0.818138 | 2.20E−12 | 7.80E−12 | 0 | 0 |
| myo-inositol | 0.818006 | 2.23E−12 | 7.80E−12 | 0 | 0 |
| valine | 0.813854 | 3.53E−12 | 1.20E−11 | 0 | 1 |
| oleate | 0.813299 | 3.75E−12 | 1.24E−11 | 0 | 0 |
| cysteine | 0.812674 | 4.02E−12 | 1.28E−11 | 0 | 1 |
| cis-vaccenate | 0.812254 | 4.21E−12 | 1.28E−11 | 0 | 0 |
| taurine | 0.812233 | 4.22E−12 | 1.28E−11 | 0 | 0 |
| acetylcarnitine | 0.843381 | 4.49E−12 | 1.32E−11 | 12.5 | 0 |
| Isobar: UDP-acetylglucosamine, UDP- | 0.800833 | 1.40E−11 | 4.01E−11 | 0 | 1 |
| acetylgalactosamine | | | | | |
| proline | 0.80054 | 1.44E−11 | 4.03E−11 | 0 | 1 |
| glutathione, oxidized | 0.80126 | 2.26E−11 | 6.17E−11 | 2.1 | 1 |
| succinate | 0.792162 | 3.30E−11 | 8.80E−11 | 0 | 1 |
| scyllo-inositol | 0.784743 | 6.68E−11 | 1.74E−10 | 0 | 0 |
| ethanolamine | 0.777387 | 1.31E−10 | 3.33E−10 | 0 | 0 |
| phenylalanine | 0.776986 | 1.36E−10 | 3.37E−10 | 0 | 1 |
| 1-oleoylglycerophosphoethanolamine | 0.770708 | 2.35E−10 | 5.73E−10 | 0 | 1 |
| glycerophosphoethanolamine | 0.767565 | 3.09E−10 | 7.35E−10 | 0 | 0 |
| cysteine-glutathione disulfide | 0.758215 | 6.73E−10 | 1.57E−09 | 0 | 1 |
| 10-nonadecenoate | 0.791012 | 7.54E−10 | 1.72E−09 | 12.5 | 0 |
| 2-oleoylglycerophosphoethanolamine | 0.756331 | 1.21E−09 | 2.71E−09 | 2.1 | 1 |
| 1- | 0.747351 | 1.59E−09 | 3.50E−09 | 0 | 1 |
| palmitoylglycerophosphoethanolamine | | | | | |
| creatinine | 0.752344 | 1.65E−09 | 3.56E−09 | 2.1 | 0 |
| docosapentaenoate (n3) | 0.774661 | 1.74E−09 | 3.68E−09 | 10.4 | 0 |
| 1-oleoylglycerophosphoserine | 0.739709 | 2.85E−09 | 5.91E−09 | 0 | 0 |
| 1-palmitoylglycerophosphocholine | 0.792823 | 2.97E−09 | 6.05E−09 | 18.8 | 1 |
| adenosine 5′-monophosphate | 0.737712 | 3.31E−09 | 6.62E−09 | 0 | 0 |
| N-acetylneuraminate | 0.741413 | 3.78E−09 | 7.43E−09 | 2.1 | 1 |
| uridine 5′-monophosphate | 0.73881 | 4.57E−09 | 8.83E−09 | 2.1 | 0 |
| choline phosphate | 0.730543 | 5.58E−09 | 1.06E−08 | 0 | 0 |
| cytidine 5′-monophosphate | 0.743501 | 7.40E−09 | 1.38E−08 | 6.2 | 0 |
| guanosine | 0.723685 | 9.06E−09 | 1.66E−08 | 0 | 0 |
| myristoleate | 0.72223 | 1.00E−08 | 1.81E−08 | 0 | 0 |
| linoleate | 0.717969 | 1.34E−08 | 2.39E−08 | 0 | 0 |
| linolenate (alpha or gamma) | 0.708609 | 2.51E−08 | 4.39E−08 | 0 | 0 |
| glutamine | 0.706584 | 2.86E−08 | 4.93E−08 | 0 | 1 |
| citrate | 0.705068 | 3.15E−08 | 5.35E−08 | 0 | 0 |
| cytidine | 0.724397 | 3.99E−08 | 6.67E−08 | 8.3 | 0 |
| putrescine | 0.699541 | 4.48E−08 | 7.38E−08 | 0 | 0 |
| 1-stearoylglycerophosphoinositol | 0.696074 | 5.57E−08 | 9.04E−08 | 0 | 0 |
| guanosine 5′-monophosphate | 0.692211 | 7.06E−08 | 1.13E−07 | 0 | 0 |
| 1- | 0.66968 | 2.64E−07 | 4.16E−07 | 0 | 1 |
| arachidonoylglycerophosphoethanolamine | | | | | |
| arginine | 0.663063 | 3.81E−07 | 5.92E−07 | 0 | 1 |
| pyroglutamine | 0.648827 | 8.12E−07 | 1.25E−06 | 0 | 0 |
| 1-stearoylglycerophosphoethanolamine | 0.642956 | 1.10E−06 | 1.66E−06 | 0 | 1 |
| ophthalmate | 0.652448 | 1.60E−06 | 2.38E−06 | 6.2 | 1 |
| 2- | 0.634846 | 1.65E−06 | 2.43E−06 | 0 | 1 |
| arachidonoylglycerophosphoethanolamine | | | | | |
| 5-methylthioadenosine | 0.680932 | 1.84E−06 | 2.67E−06 | 16.7 | 0 |
| 1-linoleoylglycerophosphoethanolamine | 0.631353 | 1.95E−06 | 2.80E−06 | 0 | 1 |
| creatine | 0.62235 | 3.01E−06 | 4.26E−06 | 0 | 1 |
| fumarate | 0.613651 | 4.50E−06 | 6.31E−06 | 0 | 0 |
| beta-alanine | 0.663483 | 1.03E−05 | 1.43E−05 | 22.9 | 0 |
| palmitoleate | 0.588353 | 1.37E−05 | 1.87E−05 | 0 | 0 |
| threonate | 0.623541 | 1.72E−05 | 2.32E−05 | 14.6 | 0 |
| N-acetylaspartate | 0.64277 | 1.79E−05 | 2.38E−05 | 20.8 | 0 |
| guanine | 0.555531 | 5.05E−05 | 6.65E−05 | 0 | 0 |
| histidine | 0.554561 | 5.24E−05 | 6.82E−05 | 0 | 1 |
| glycerate | 0.557923 | 5.61E−05 | 7.23E−05 | 2.1 | 1 |
| fructose | 0.574623 | 6.90E−05 | 8.78E−05 | 10.4 | 1 |
| laurate | 0.546074 | 7.18E−05 | 9.03E−05 | 0 | 0 |
| phosphate | 0.512001 | 0.000234 | 0.000289 | 0 | 0 |
| nicotinamide | 0.511987 | 0.000235 | 0.000289 | 0 | 0 |
| erythronate | 0.500423 | 0.000464 | 0.000565 | 4.2 | 1 |
| 2-stearoylglycerophosphoinositol | 0.518633 | 0.000607 | 0.000731 | 14.6 | 0 |
| lysine | 0.465319 | 0.000982 | 0.00117 | 0 | 1 |
| phosphoethanolamine | 0.456516 | 0.001258 | 0.001483 | 0 | 0 |
| 2- | 0.48713 | 0.002586 | 0.003017 | 22.9 | 1 |
| palmitoylglycerophosphoethanolamine | | | | | |
| sorbitol | 0.427503 | 0.003043 | 0.003514 | 2.1 | 1 |
| 6-sialyl-N-acetyllactosamine | 0.445246 | 0.005092 | 0.005819 | 18.8 | 1 |
| palmitate | 0.377993 | 0.008804 | 0.00996 | 0 | 0 |
| glucose | 0.275499 | 0.060886 | 0.068192 | 0 | 1 |
| myristate | −0.27059 | 0.065824 | 0.072993 | 0 | 0 |
| 1-oleoylglycerol | −0.27063 | 0.07921 | 0.086975 | 10.4 | 0 |
| arachidate | 0.195497 | 0.187866 | 0.204282 | 0 | 0 |
| stearate | −0.17948 | 0.227374 | 0.244864 | 0 | 0 |
| 2-hydroxyglutarate | 0.16811 | 0.287241 | 0.30639 | 10.4 | 0 |
| 13-methylmyristic acid | 0.134596 | 0.36705 | 0.387827 | 0 | 0 |
| 1-palmitoylglycerol | −0.1326 | 0.374282 | 0.391771 | 0 | 0 |
| 1-stearoylglycerol | 0.129743 | 0.384737 | 0.395679 | 0 | 0 |
| 2-palmitoylglycerol | −0.14922 | 0.38508 | 0.395679 | 22.9 | 0 |
| spermidine | −0.11022 | 0.476316 | 0.481602 | 6.2 | 0 |
| 2-methylbutyrylcarnitine | −0.10623 | 0.477302 | 0.481602 | 0 | 0 |
| 2-aminoadipate | −0.03708 | 0.820293 | 0.820293 | 14.6 | 1 |

TABLE 28
Mean, standard deviation, and significance of the signal intensity ratio between taurine
and cytidine 5′-diphosphocholine, and dihomo-linolenate and cytidine 5′-diphosphocholine
across frozen and FFPE samples.

| | FROZEN | | | FFPE | | |
|---|---|---|---|---|---|---|
| Metabolite ratio | Normal | Tumor | P | Normal | Tumor | P |
| taurine/cytidine | 4.5 ± 1.8 | 1.6 ± 0.4 | 0.000155 | 2.4 ± 1.0 | 2.1 ± 0.8 | 0.00109 |
| 5′- | | | | | | |
| diphosphocholine | | | | | | |
| dihomo-linolenate/ | 6.3 ± 4.3 | 19.8 ± 17.5 | 0.0104 | 0.93 ± 0.49 | 0.99 ± 0.56 | 0.0379 |
| cytidine 5′- | | | | | | |
| diphosphocholine | | | | | | |

OSC-PLS was used to model the metabolic profile of prostate cancer in frozen and FFPE samples. OSC-PLS is a supervised algorithm that aims to maximize the variance between groups in the latent variable in the output data (i.e., score) and calculates metabolites' loadings that measure importance of the variables in the discrimination between two groups. The OSC-PLS loadings for the discrimination between normal and tumor tissues are shown in FIG. 3E (on the left of the heatmaps). In this analysis, positive OSC-PLS loadings indicate the metabolites with higher concentration in tumor tissue and vice versa. Both OSC-PLS models built on frozen and FFPE sample data showed similar OSC-PLS loadings values. A high correlation between the values of OSC-PLS loadings of the models built on frozen and FFPE samples was observed (r=0.57).

Metabolite Set Enrichment Analysis (MSEA) was performed with the GSEA tool (Gene Pattern software) using the loadings of OSC-PLS to rank the metabolites. The metabolite sets were built using the human pathway information available in the HMDB. The MSEA was used to determine which metabolic pathways were significantly altered between prostate tumors and normal tissue. Alpha-linolenic acid and linoleic acid metabolism was up-regulated in both frozen and FFPE tumor tissues (P=0.012 and FDR=0.064 in frozen tissues, and P=0.050 and FDR=0.166 in FFPE tissues), whereas the up-regulation of protein synthesis was statistically significant only in FFPE samples (P=0.009 and FDR=0.048).

### Example 7: Prediction of Prostate Cancer Fingerprint

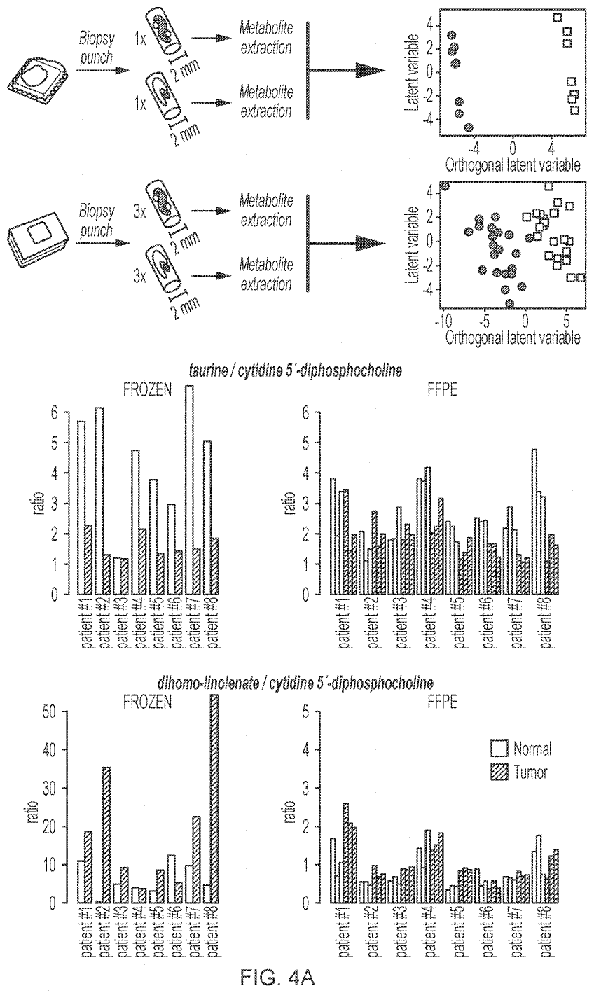

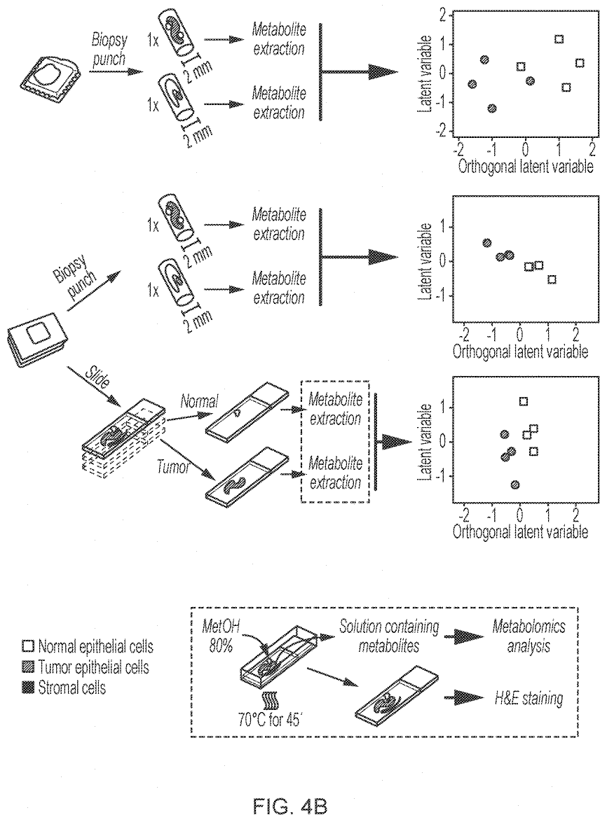

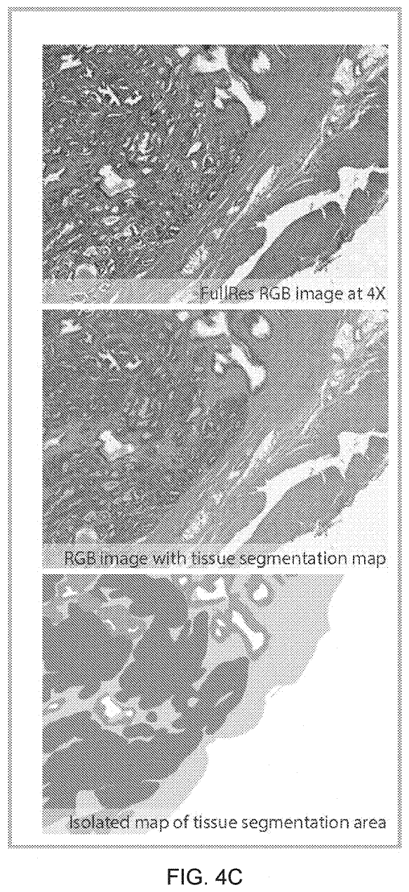

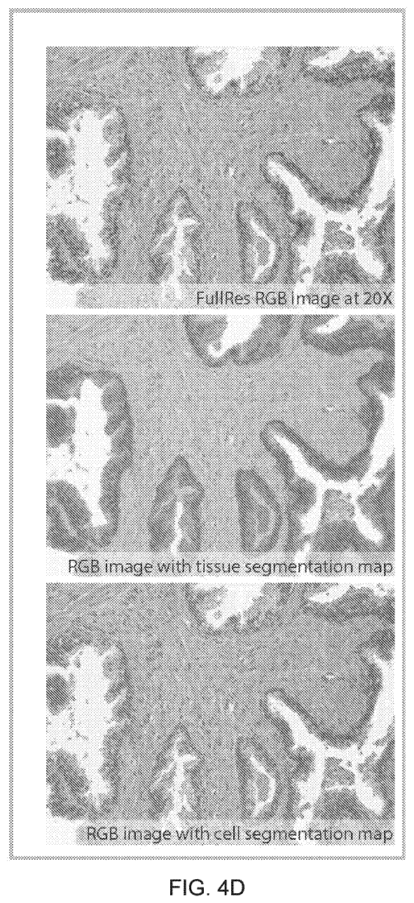

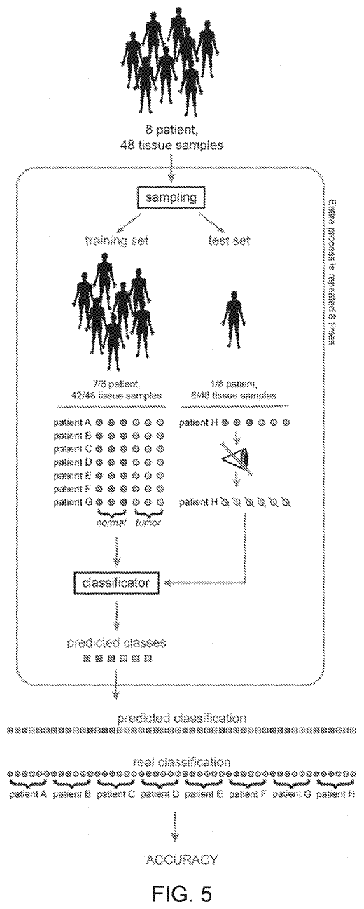

FFPE material was investigated for use in a context of multivariate analysis for diagnostic or prognostic purposes. OSC-PLS was used to model the metabolic profile of prostate cancer in FFPE samples of the training set. The relative OSC-PLS scores plot is shown in the top panel of FIG. 4A, which illustrates a distinct difference between metabolic fingerprints of normal and tumor tissues. A modified leave-one-out cross-validation was performed to evaluate the accuracy of the discrimination between tumor and normal tissue in the training set. A schematic diagram of the cross-validation procedure is provided in the bottom panel of FIG. 4A. The cross-validated accuracy was 75.0% for FFPE samples. When the average of the predicted values of each replicate was used to classify the tissue type, the accuracy increased to 87.5%. The cross-validated accuracy obtained from OCT-embedded samples was 100%.

From the validation set, biopsy punches were collected from normal and tumor tissues and manual macro-dissection on 20 μm FFPE sections was performed to generate enriched samples for normal or tumor tissue (FIG. 4B). When an OSC-PLS model previously built on the training set was applied, normal and tumor tissue samples were correctly classified in both extract from the FFPE biopsy punches and from the FFPE sections. The resulting OSC-PLS scores plots are shown in FIG. 4B.

### FFPE Tissue Sections

It was then investigated whether limited amount of material, such as FFPE sections could be utilized to obtain an accurate metabolic fingerprinting. 20-mm sections from FFPE biopsy punches of the validation set were obtained and manual macrodis section to generate samples enriched for normal or tumor tissue was performed (FIG. 4B). Akin to FFPE biopsy punches, when the OSC-PLS model previously built on the training set was applied, normal and tumor tissue samples were correctly classified. Taken together, these data suggest that a separation between normal and tumor metabolic fingerprint is still possible using a reduced amount of material, such as a tissue section.

Then, whether metabolites could be correlated with stroma and epithelia was tested as an example of mapping metabolites to a particular organelle of tissue or cells. After the metabolic extraction, FPPE tissue sections (on slides) were stained with Hematoxylin and eosin (H&E) showing that the tissue architecture was preserved. A semi-automated algorithm was used to quantify the cell number and the area of the epithelial and stroma compartments (FIG. 4C and FIG. 4D) in both normal and tumor tissues. Information obtained from image analysis is reported in Table 29 and Table 30. A distinct separation between normal and tumor metabolic fingerprint is possible even if the percentage of epithelial cells was about 20% of the total cells.

TABLE 29
Histological analysis of the FFPE sections of the validation set after methanol extraction

| | | Total cells | | | | Tissue Category (Area pixels) | | | |
|---|---|---|---|---|---|---|---|---|---|
| | | | | Stroma | Epithelial | | | Stroma | Epithelial |
| | | Stroma | Epithelial | (%) | (%) | Stroma | Epithelial | (%) | (%) |
| V1 | Normal | 109342 | 185729 | 37.1 | 62.9 | 240771695 | 121526701 | 66.5 | 33.5 |
| V1 | Tumor | 51354 | 188144 | 21.4 | 78.6 | 89569892 | 135141650 | 39.9 | 60.1 |
| V2 | Normal | 297488 | 76233 | 79.6 | 20.4 | 416972640 | 34210009 | 92.4 | 7.6 |
| V2 | Tumor | 83706 | 24877 | 77.1 | 22.9 | 42012673 | 13029436 | 76.3 | 23.7 |
| V3 | Normal | 98304 | 138182 | 41.6 | 58.4 | 228260261 | 200404762 | 53.2 | 46.8 |
| V3 | Tumor | 39451 | 27332 | 59.1 | 40.9 | 88374954 | 18966440 | 82.3 | 17.7 |
| V4 | Normal | 97622 | 30787 | 76.0 | 24.0 | 129476623 | 16001009 | 89.0 | 11.0 |
| V4 | Tumor | 177499 | 175649 | 50.3 | 49.7 | 188127685 | 104869735 | 64.2 | 35.8 |

TABLE 30
Histological analysis of the FFPE sections of the validation set after methanol extraction

| | | Cell Density | | | | Nucleolus Area (pixels) | | | |
|---|---|---|---|---|---|---|---|---|---|
| | | | | Stroma | Epithelial | | | Stroma | Epithelial |
| | | Stroma | Epithelial | (%) | (%) | Stroma | Epithelial | (%) | (%) |
| V1 | Normal | 189589.63 | 525490.9 | 26.5 | 73.5 | 10745081 | 23012449 | 31.8 | 68.2 |
| V1 | Tumor | 129153.8 | 309674.66 | 29.4 | 70.6 | 5190940 | 23253108 | 18.2 | 81.8 |
| V2 | Normal | 307820.12 | 468768.2 | 39.6 | 60.4 | 28818376 | 9190236 | 75.8 | 24.2 |
| V2 | Tumor | 95417.06 | 86077 | 52.6 | 47.4 | 8385990 | 2756673 | 75.3 | 24.7 |
| V3 | Normal | 184869.17 | 307704.79 | 37.5 | 62.5 | 9241729 | 16156190 | 36.4 | 63.6 |
| V3 | Tumor | 53901.1 | 111893.2 | 32.5 | 67.5 | 3985614 | 3117823 | 56.1 | 43.9 |
| V4 | Normal | 98335.47 | 156997.6 | 38.5 | 61.5 | 10561910 | 4179800 | 71.6 | 28.4 |
| V4 | Tumor | 315648.11 | 479271.6 | 39.7 | 60.3 | 20063353 | 21178197 | 48.6 | 51.4 |

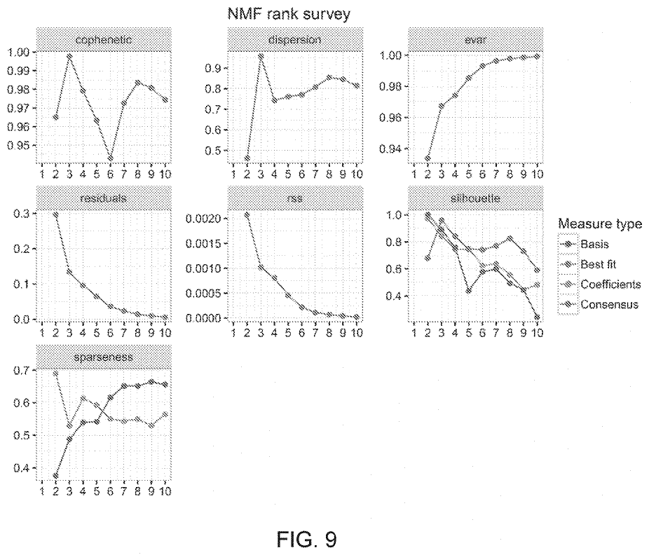

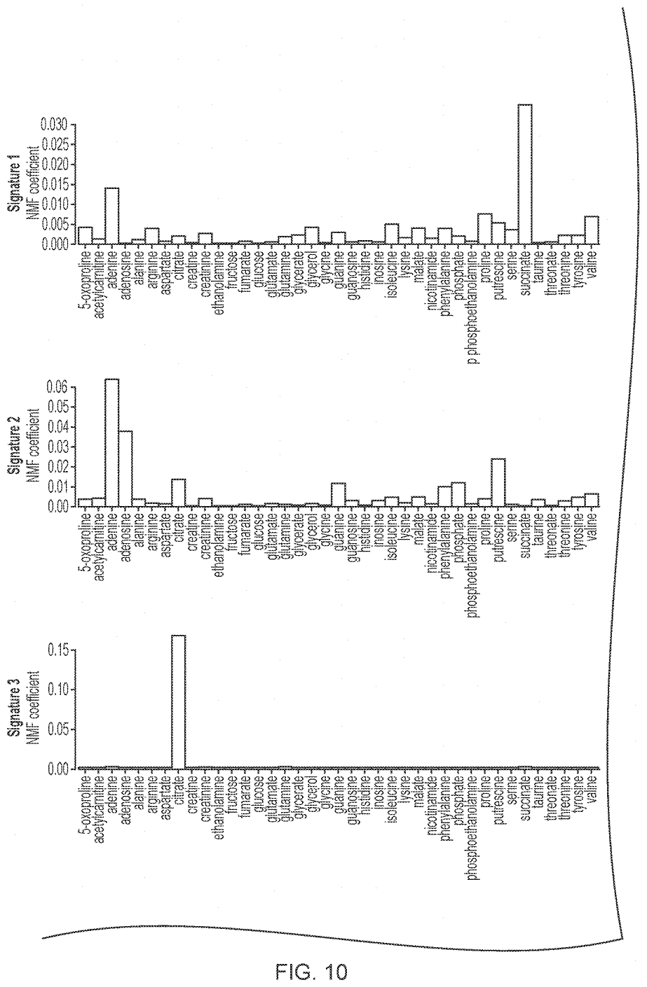

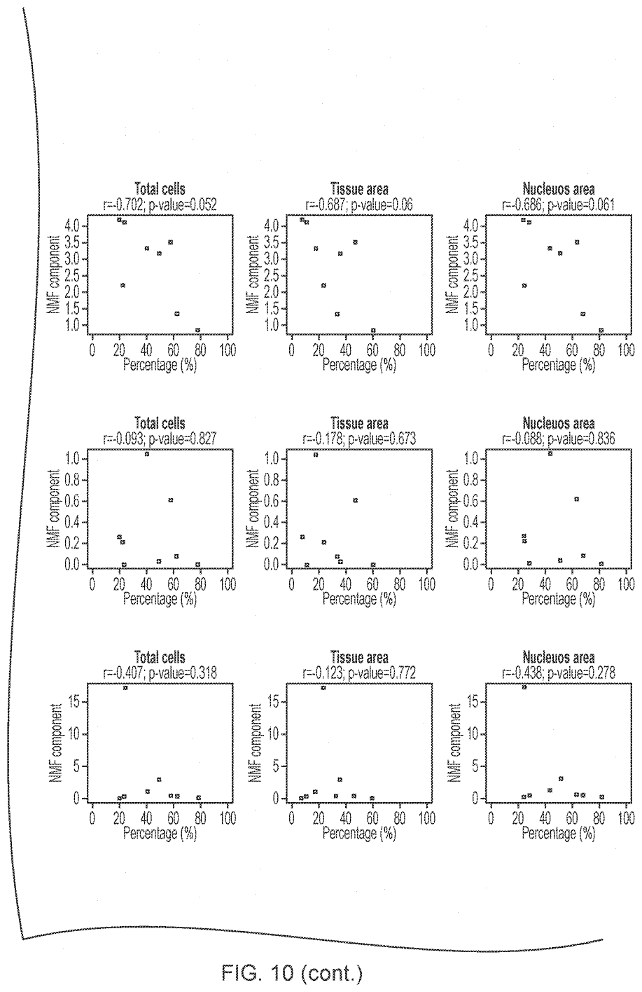

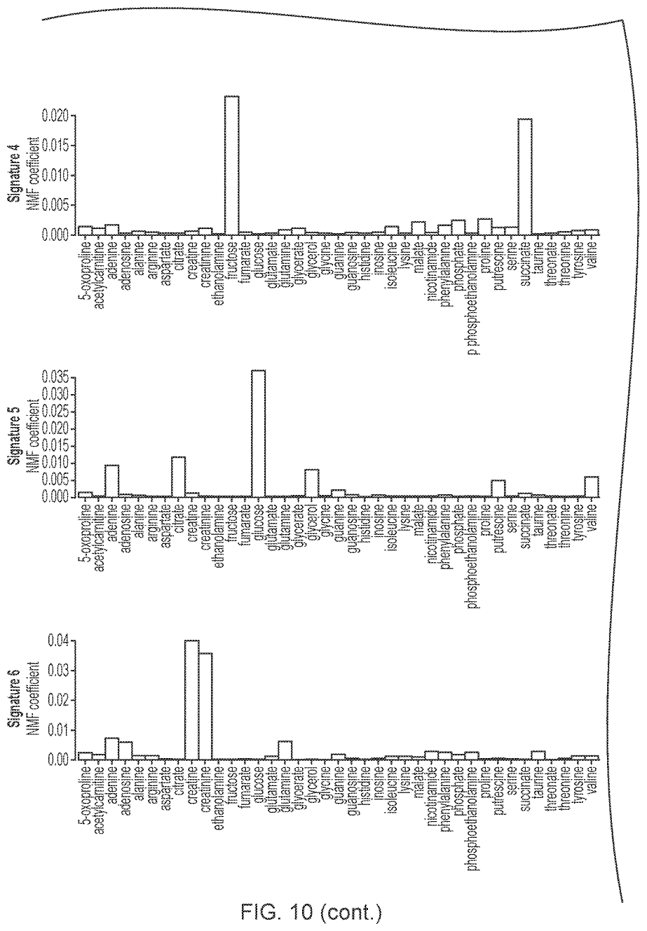

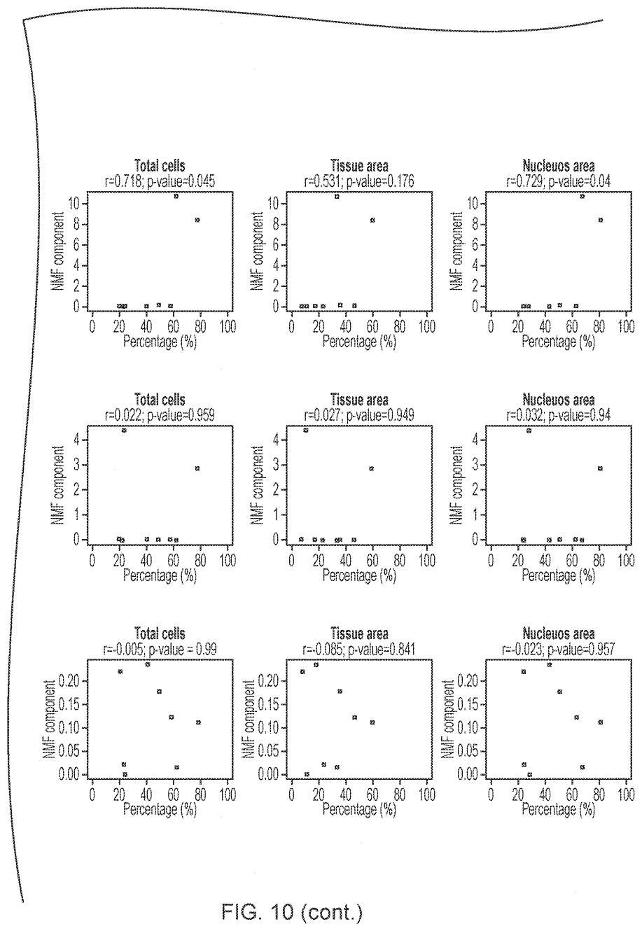

Non-negative matrix factorization (NMF) was applied to decipher metabolic signatures from stroma and epithelium. 6 metabolic signatures were identified (FIG. 9) across the 16 FFPE samples of the validation set (8 biopsy punch samples and 8 tissue section samples). Despite the limited sample size, the analysis showed a correlation between the signatures 1 and 4 with the stroma and epithelium tissue percentages, respectively (FIG. 10), suggesting that stroma and epithelium may be characterized by different metabolomic profiles. Fructose was linked to a higher presence of epithelial tissue in the sample (r=0.74, P=3.60×10⁻²; FDR=2.65×10⁻¹) as described in Table 31, Table 32 and Table 33.

TABLE 31
Correlation analysis between metabolite intensity and
percentage of epithelial tissue analyzing total cells

| | Total cells | | |
|---|---|---|---|
| metabolite | r | p-value | FDR |
| 5-oxoproline | 0.37 | 3.62E−01 | 7.06E−01 |
| acetylcarnitine | 0.56 | 1.49E−01 | 4.83E−01 |
| adenine | −0.27 | 5.14E−01 | 8.35E−01 |
| adenosine | 0.02 | 9.71E−01 | 9.71E−01 |
| alanine | 0.28 | 5.03E−01 | 8.35E−01 |
| arginine | −0.74 | 3.50E−02 | 2.65E−01 |
| aspartate | 0.42 | 2.99E−01 | 6.42E−01 |
| citrate | −0.41 | 3.13E−01 | 6.42E−01 |
| creatine | −0.28 | 5.02E−01 | 8.35E−01 |
| creatinine | −0.09 | 8.28E−01 | 8.60E−01 |
| ethanolamine | 0.12 | 7.85E−01 | 8.60E−01 |
| fructose | 0.74 | 3.60E−02 | 2.65E−01 |
| fumarate | 0.60 | 1.15E−01 | 4.46E−01 |
| glucose | 0.10 | 8.07E−01 | 8.60E−01 |
| glutamate | 0.10 | 8.13E−01 | 8.60E−01 |
| glutamine | −0.30 | 4.70E−01 | 8.35E−01 |
| glycerate | 0.45 | 2.60E−01 | 6.33E−01 |
| glycerol | −0.46 | 2.56E−01 | 6.33E−01 |
| glycine | 0.75 | 3.23E−02 | 2.65E−01 |
| guanine | −0.84 | 8.37E−03 | 2.65E−01 |
| guanosine | −0.19 | 6.60E−01 | 8.60E−01 |
| histidine | −0.16 | 7.05E−01 | 8.60E−01 |
| inosine | −0.21 | 6.17E−01 | 8.60E−01 |
| isoleucine | 0.11 | 8.04E−01 | 8.60E−01 |
| lysine | −0.55 | 1.62E−01 | 4.87E−01 |
| malate | 0.73 | 4.08E−02 | 2.65E−01 |
| nicotinamide | −0.63 | 9.38E−02 | 4.46E−01 |
| phenylalanine | 0.80 | 1.66E−02 | 2.65E−01 |
| phosphate | 0.41 | 3.07E−01 | 6.42E−01 |
| phosphoethanolamine | −0.69 | 5.62E−02 | 3.13E−01 |
| proline | −0.16 | 7.05E−01 | 8.60E−01 |
| putrescine | 0.11 | 7.98E−01 | 8.60E−01 |
| serine | 0.61 | 1.10E−01 | 4.46E−01 |
| succinate | 0.51 | 2.01E−01 | 5.60E−01 |
| taurine | −0.59 | 1.26E−01 | 4.46E−01 |
| threonate | 0.11 | 7.98E−01 | 8.60E−01 |
| threonine | −0.09 | 8.38E−01 | 8.60E−01 |
| tyrosine | 0.13 | 7.64E−01 | 8.60E−01 |
| valine | −0.21 | 6.25E−01 | 8.60E−01 |

TABLE 32
Correlation analysis between metabolite intensity and
percentage of epithelial tissue analyzing total area

| | Total area | | |
|---|---|---|---|
| metabolite | r | p-value | FDR |
| 5-oxoproline | 0.28 | 4.97E−01 | 9.14E−01 |
| acetylcarnitine | 0.32 | 4.47E−01 | 9.14E−01 |
| adenine | −0.36 | 3.84E−01 | 9.14E−01 |
| adenosine | −0.02 | 9.68E−01 | 9.68E−01 |
| alanine | 0.14 | 7.42E−01 | 9.14E−01 |
| arginine | −0.58 | 1.33E−01 | 7.77E−01 |
| aspartate | 0.51 | 2.01E−01 | 7.77E−01 |
| citrate | −0.13 | 7.67E−01 | 9.14E−01 |
| creatine | −0.09 | 8.40E−01 | 9.14E−01 |
| creatinine | −0.05 | 9.10E−01 | 9.59E−01 |
| ethanolamine | −0.09 | 8.35E−01 | 9.14E−01 |
| fructose | 0.57 | 1.43E−01 | 7.77E−01 |
| fumarate | 0.30 | 4.66E−01 | 9.14E−01 |
| glucose | 0.11 | 7.88E−01 | 9.14E−01 |
| glutamate | 0.18 | 6.61E−01 | 9.14E−01 |
| glutamine | −0.16 | 7.09E−01 | 9.14E−01 |
| glycerate | 0.18 | 6.68E−01 | 9.14E−01 |
| glycerol | −0.50 | 2.09E−01 | 7.77E−01 |
| glycine | 0.52 | 1.90E−01 | 7.77E−01 |
| guanine | −0.80 | 1.82E−02 | 3.55E−01 |
| guanosine | −0.25 | 5.44E−01 | 9.14E−01 |
| histidine | −0.48 | 2.25E−01 | 7.77E−01 |
| inosine | −0.11 | 8.01E−01 | 9.14E−01 |
| isoleucine | 0.08 | 8.44E−01 | 9.14E−01 |
| lysine | −0.19 | 6.55E−01 | 9.14E−01 |
| malate | 0.50 | 2.05E−01 | 7.77E−01 |
| nicotinamide | −0.88 | 3.56E−03 | 1.39E−01 |
| phenylalanine | 0.64 | 8.62E−02 | 7.77E−01 |
| phosphate | 0.28 | 5.09E−01 | 9.14E−01 |
| phosphoethanolamine | −0.47 | 2.39E−01 | 7.77E−01 |
| proline | −0.19 | 6.55E−01 | 9.14E−01 |
| putrescine | −0.02 | 9.65E−01 | 9.68E−01 |
| serine | 0.76 | 2.80E−02 | 3.64E−01 |
| succinate | 0.22 | 5.98E−01 | 9.14E−01 |
| taurine | −0.38 | 3.54E−01 | 9.14E−01 |
| threonate | 0.22 | 6.03E−01 | 9.14E−01 |
| threonine | −0.16 | 7.11E−01 | 9.14E−01 |
| tyrosine | 0.30 | 4.77E−01 | 9.14E−01 |
| valine | −0.11 | 7.97E−01 | 9.14E−01 |

TABLE 33
Correlation analysis between metabolite intensity and
percentage of epithelial tissue analyzing nucleous area

| | Nucleous area | | |
|---|---|---|---|
| metabolite | r | p-value | FDR |
| 5-oxoproline | 0.41 | 3.13E−01 | 6.11E−01 |
| acetylcarnitine | 0.58 | 1.35E−01 | 4.18E−01 |
| adenine | −0.27 | 5.25E−01 | 8.53E−01 |
| adenosine | 0.03 | 9.36E−01 | 9.36E−01 |
| alanine | 0.30 | 4.66E−01 | 7.89E−01 |
| arginine | −0.74 | 3.74E−02 | 2.27E−01 |
| aspartate | 0.43 | 2.88E−01 | 6.02E−01 |
| citrate | −0.44 | 2.73E−01 | 6.02E−01 |
| creatine | −0.32 | 4.35E−01 | 7.71E−01 |
| creatinine | −0.12 | 7.77E−01 | 8.92E−01 |
| ethanolamine | 0.15 | 7.21E−01 | 8.92E−01 |
| fructose | 0.75 | 3.28E−02 | 2.27E−01 |
| fumarate | 0.63 | 9.65E−02 | 3.87E−01 |
| glucose | 0.11 | 7.95E−01 | 8.92E−01 |
| glutamate | 0.04 | 9.18E−01 | 9.36E−01 |
| glutamine | −0.33 | 4.23E−01 | 7.71E−01 |
| glycerate | 0.48 | 2.28E−01 | 5.92E−01 |
| glycerol | −0.43 | 2.86E−01 | 6.02E−01 |
| glycine | 0.75 | 3.11E−02 | 2.27E−01 |
| guanine | −0.83 | 1.00E−02 | 2.27E−01 |
| guanosine | −0.19 | 6.55E−01 | 8.92E−01 |
| histidine | −0.14 | 7.45E−01 | 8.92E−01 |
| inosine | −0.23 | 5.89E−01 | 8.92E−01 |
| isoleucine | 0.14 | 7.44E−01 | 8.92E−01 |
| lysine | −0.57 | 1.39E−01 | 4.18E−01 |
| malate | 0.74 | 3.49E−02 | 2.27E−01 |
| nicotinamide | −0.61 | 1.09E−01 | 3.87E−01 |
| phenylalanine | 0.81 | 1.50E−02 | 2.27E−01 |
| phosphate | 0.43 | 2.93E−01 | 6.02E−01 |
| phosphoethanolamine | −0.73 | 4.07E−02 | 2.27E−01 |
| proline | −0.15 | 7.24E−01 | 8.92E−01 |
| putrescine | 0.13 | 7.65E−01 | 8.92E−01 |
| serine | 0.62 | 9.90E−02 | 3.87E−01 |
| succinate | 0.54 | 1.70E−01 | 4.74E−01 |
| taurine | −0.62 | 9.94E−02 | 3.87E−01 |
| threonate | 0.07 | 8.74E−01 | 9.21E−01 |
| threonine | −0.08 | 8.52E−01 | 9.21E−01 |
| tyrosine | 0.11 | 8.00E−01 | 8.92E−01 |
| valine | −0.20 | 6.35E−01 | 8.92E−01 |

### Example 8. Metabolite Extraction from Slide Samples

Although the procedure to extract metabolites from a tissue section attached to a slide is similar to extraction of the other FFPE samples, it may be complicated by the low quantity of available tissue and the desire to minimize the loss of solution during the extraction. As depicted in the schematics shown in FIGS. 6-7, a cassette has been developed to minimize the loss of solution in such instances.

To extract metabolites from a sample attached to a slide, the slide having the sample is inserted into the cassette depicted in FIG. 6. A 1 mL solution of 80% methanol is added to the cassette and incubated at 70° C. for 30-45 minutes in a 1.5 mL micro-centrifuge tube. The methanol-incubated sample is subsequently placed on ice for 15 minutes and centrifuged at 14,000 g for 10 minutes (4-8° C.). The supernatant is transferred into a new 1.5-mL micro-centrifuge tube and chilled on ice for 10 minutes, followed by centrifugation at 14,000 g for 5 minutes (4-8° C.). Finally, the supernatant is collected and stored at −80° C. Following extraction, the cellular architecture of tissue sections is intact and the tissue can be used for a histological examination.

### Example 9. Metabolites Lost During FFPE Procedure

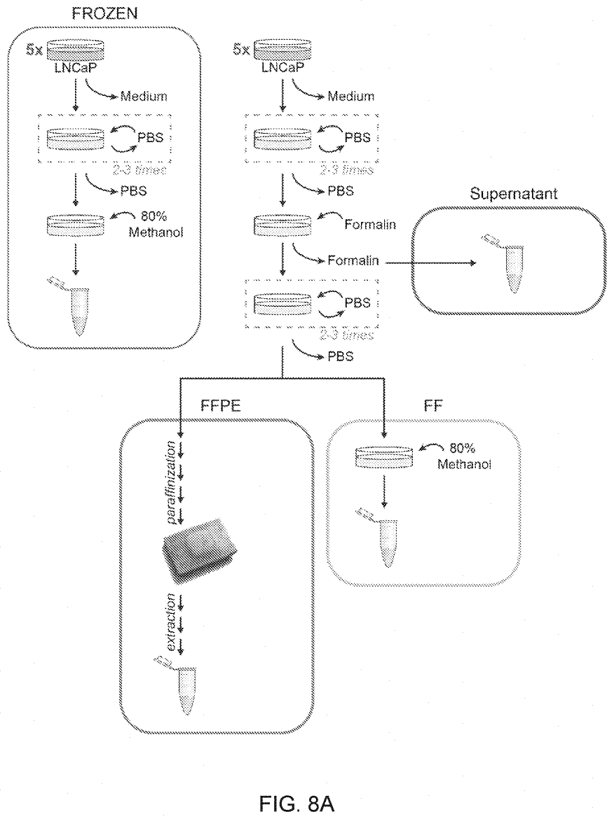

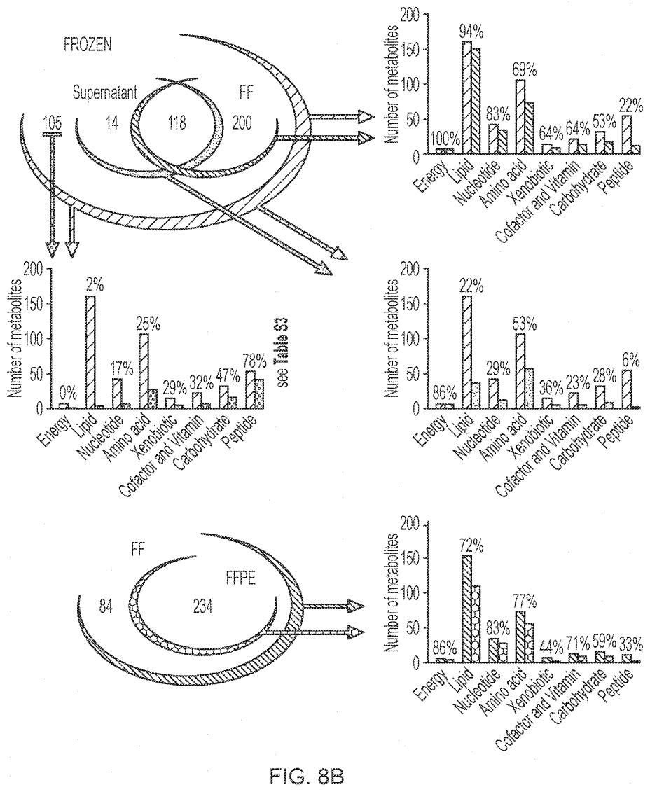

Potential chemical reasons that might affect selectively specific classes of metabolites during the formalin-fixing and paraffin-embedding process were investigated (FIG. 1). The following major factors were identified: (i) solubility in formalin solution, (ii) covalent bonding to cellular component (e.g., protein, DNA/RNA), and (iii) solubility in ethanol and xylene. Using the protocol schematized in FIG. 8A, cell samples were collected and profiled immediately after the formalin fixation before the paraffin embedding procedure and the supernatant solutions of formalin used during the fixation. In FIG. 8B, Venn diagrams show metabolomic data collected during the different steps of the procedures and their rate of detection according to the superclass to which they belong. The formalin fixation and paraffin-embedding is a multistep procedure. The first step consists of the immersion of the tissue in the formalin solution. During this step, polar metabolites may dissolve in the formalin solution whereas some metabolites may react with formaldehyde forming covalent bonds with cellular components. After fixation, the tissue is dehydrated via a series of graded ethanol solutions followed by xylenes and finally liquid paraffin. Apolar metabolites could dissolve in ethanol/xylene solvents.

First, metabolites found in the supernatant (n=132) were compared with those found in the extracts from frozen samples (n=437), as described in Table 34, Table 35, Table 36, Table 37, Table 38 and Table 39 to identify those that are soluble in formalin and could, as a result, be lost in the analysis.

TABLE 34
Metabolites found and missed in formalin solution categorized by superclass.

| | non-found in | found in | | | |
|---|---|---|---|---|---|
| | formalin | formalin | formalin solution/ | | |
| Superclass | solution, n (%) | solution, n (%) | FROZEN, % | P | FDR |
| Amino Acid | 50 (16.4%) | 56 (42.4%) | 52.8% | 2.33E−08 | 1.87E−07 |
| Peptide | 51 (16.7%) | 3 (2.3%) | 5.6% | 4.93E−06 | 1.97E−05 |
| Energy | 1 (0.3%) | 6 (4.5%) | 85.7% | 3.68E−03 | 9.80E−03 |
| Lipid | 124 (40.7%) | 36 (27.3%) | 22.5% | 9.30E−03 | 1.86E−02 |
| Cofactors and Vitamins | 17 (5.6%) | 5 (3.8%) | 22.7% | 6.34E−01 | 9.85E−01 |
| Xenobiotics | 9 (3%) | 5 (3.8%) | 35.7% | 7.68E−01 | 9.85E−01 |
| Nucleotide | 30 (9.8%) | 12 (9.1%) | 28.6% | 8.62E−01 | 9.85E−01 |
| Carbohydrate | 23 (7.5%) | 9 (6.8%) | 28.1% | 1.00E+00 | 1.00E+00 |

TABLE 35
Metabolites found and missed in formalin solution categorized by class.

| | non-found in | | | | |
|---|---|---|---|---|---|
| | formalin solution, | found in formalin | formalin solution/ | | |
| Class | n (%) | solution, n (%) | FROZEN, % | P | FDR |
| Peptides | 50 (20.5%) | 2 (1.9%) | 3.8% | 2.61E−07 | 6.53E−06 |
| Glycerophospholipids | 46 (18.9%) | 2 (1.9%) | 4.2% | 1.47E−06 | 1.83E−05 |
| Amino Acids and Derivatives | 39 (16%) | 36 (34.3%) | 48.0% | 1.31E−03 | 1.04E−02 |
| Hydroxy Acids and Derivatives | 1 (0.4%) | 7 (6.7%) | 87.5% | 1.66E−03 | 1.04E−02 |
| Fatty Acids and Conjugates | 13 (5.3%) | 16 (15.2%) | 55.2% | 6.69E−03 | 3.34E−02 |
| Pyrimidine Nucleotides | 12 (4.9%) | 0 (0%) | 0.0% | 1.18E−02 | 4.90E−02 |
| Glycerolipids | 10 (4.1% ) | 0 (0%) | 0.0% | 3.45E−02 | 1.14E−01 |
| Alkylamines | 1 (0.4%) | 4 (3.8%) | 80.0% | 3.66E−02 | 1.14E−01 |
| Carboxylic Acids and Derivatives | 2 (0.8%) | 4 (3.8%) | 66.7% | 8.29E−02 | 2.07E−01 |
| Pyrimidine Nucleosides and | 2 (0.8%) | 4 (3.8%) | 66.7% | 8.29E−02 | 2.07E−01 |
| Analogues | | | | | |
| Benzyl Alcohols and Derivatives | 1 (0.4%) | 3 (2.9%) | 75.0% | 9.58E−02 | 2.18E−01 |
| Purine Nucleotides | 11 (4.5%) | 1 (1%) | 8.3% | 1.13E−01 | 2.36E−01 |
| Sphingolipids | 5 (2%) | 0 (0%) | 0.0% | 1.84E−01 | 3.53E−01 |
| Organic Phosphoric Acids and | 1 (0.4%) | 2 (1.9%) | 66.7% | 2.37E−01 | 3.95E−01 |
| Derivatives | | | | | |
| Pyridines and Derivatives | 1 (0.4%) | 2 (1.9%) | 66.7% | 2.37E−01 | 3.95E−01 |
| Lineolic Acids and Derivatives | 4 (1.6%) | 0 (0%) | 0.0% | 3.12E−01 | 4.59E−01 |
| Pteridines and Derivatives | 4 (1.6%) | 0 (0%) | 0.0% | 3.12E−01 | 4.59E−01 |
| Fatty Acid Esters | 10 (4.1%) | 7 (6.7%) | 41.2% | 4.27E−01 | 5.93E−01 |
| Azoles | 3 (1.2%) | 0 (0%) | 0.0% | 5.55E−01 | 6.99E−01 |
| Monosaccharides | 8 (3.3%) | 5 (4.8%) | 38.5% | 5.60E−01 | 6.99E−01 |
| Imidazopyrimidines | 3 (1.2%) | 2 (1.9%) | 40.0% | 6.54E−01 | 7.78E−01 |
| Purine Nucleosides and Analogues | 6 (2.5%) | 4 (3.8%) | 40.0% | 7.32E−01 | 8.31E−01 |
| Cyclic Alcohols and Derivatives | 3 (1.2%) | 1 (1%) | 25.0% | 1.00E+00 | 1.00E+00 |
| Sugar Acids and Derivatives | 6 (2.5%) | 2 (1.9%) | 25.0% | 1.00E+00 | 1.00E+00 |
| Sugar Alcohols | 2 (0.8%) | 1 (1%) | 33.3% | 1.00E+00 | 1.00E+00 |

TABLE 36
Metabolites found and missed in formalin solution categorized by subclass.

| | non-found in | | | | |
|---|---|---|---|---|---|
| | formalin solution, | found in formalin | formalin solution/ | | |
| Subclass | n (%) | solution, n (%) | FROZEN, % | P | FDR |
| Peptides | 50 (25%) | 2 (2.3%) | 3.8% | 2.61E−07 | 8.35E−06 |
| Alpha Amino Acids and | 14 (7%) | 27 (31%) | 65.9% | 3.24E−06 | 5.19E−05 |
| Derivatives | | | | | |
| Lysophosphatidylethanolamines | 18 (9%) | 0 (0%) | 0.0% | 1.23E−03 | 1.31E−02 |
| Unsaturated Fatty Acids | 5 (2.5%) | 10 (11.5%) | 66.7% | 7.73E−03 | 6.19E−02 |
| Branched Fatty Acids | 0 (0%) | 4 (4.6%) | 100.0% | 9.73E−03 | 6.23E−02 |
| Dicarboxylic Acids and | 0 (0%) | 3 (3.4%) | 100.0% | 3.12E−02 | 1.30E−01 |
| Derivatives | | | | | |
| Pyrimidine Nucleosides and | 0 (0%) | 3 (3.4%) | 100.0% | 3.12E−02 | 1.30E−01 |
| Analogues | | | | | |
| Monoacylglycerols | 10 (5%) | 0 (0%) | 0.0% | 3.45E−02 | 1.30E−01 |
| Beta Hydroxy Acids and | (0.5%) | 4 (4.6%) | 80.0% | 3.66E−02 | 1.30E−01 |
| Derivatives | | | | | |
| Straight Chain Fatty Acids | 8 (4%) | 0 (0%) | 0.0% | 6.00E−02 | 1.92E−01 |
| Lysophosphatidylcholines | 12 (6%) | 1 (1.1%) | 7.7% | 7.04E−02 | 2.05E−01 |
| Phenylpyruvic Acid Derivatives | 1 (0.5%) | 3 (3.4%) | 75.0% | 9.58E−02 | 2.53E−01 |
| Phosphatidylcholines | 7 (3.5%) | 0 (0%) | 0.0% | 1.03E−01 | 2.53E−01 |
| Polyamines | 1 (0.5%) | 2 (2.3%) | 66.7% | 2.37E−01 | 5.41E−01 |
| Acyl Carnitines | 8 (4%) | 7 (8%) | 46.7% | 2.57E−01 | 5.48E−01 |
| Lineolic Acids and Derivatives | 4 (2%) | 0 (0%) | 0.0% | 3.12E−01 | 5.88E−01 |
| Pyrimidine Nucleotide Sugars | 4 (2%) | 0 (0%) | 0.0% | 3.12E−01 | 5.88E−01 |
| Purine Nucleosides and | 3 (1.5%) | 3 (3.4%) | 50.0% | 3.86E−01 | 6.86E−01 |
| Analogues | | | | | |
| Hexoses | 6 (3%) | 1 (1.1%) | 14.3% | 4.39E−01 | 7.39E−01 |
| Acyl Glycines | 3 (1.5%) | 0 (0%) | 0.0% | 5.55E−01 | 7.39E−01 |
| Glycoamino Acids and | 3 (1.5%) | 0 (0%) | 0.0% | 5.55E−01 | 7.39E−01 |
| Derivatives | | | | | |
| Imidazolyl Carboxylic Acids and | 3 (1.5%) | 0 (0%) | 0.0% | 5.55E−01 | 7.39E−01 |
| Derivatives | | | | | |
| Phosphatidylinositols | 3 (1.5%) | 0 (0%) | 0.0% | 5.55E−01 | 7.39E−01 |
| Purine Ribonucleoside | 3 (1.5%) | 0 (0%) | 0.0% | 5.55E−01 | 7.39E−01 |
| Monophosphates | | | | | |
| Pentoses | 2 (1%) | 2 (2.3%) | 50.0% | 5.94E−01 | 7.61E−01 |
| N-acyl-alpha Amino Acids and | 14 (7%) | 8 (9.2%) | 36.4% | 6.41E−01 | 7.89E−01 |
| Derivatives | | | | | |
| Beta Amino Acids and | 2 (1%) | 1 (1.1%) | 33.3% | 1.00E+00 | 1.00E+00 |
| Derivatives | | | | | |
| Purine 2′-deoxyribonucleosides | 3 (1.5%) | 1 (1.1%) | 25.0% | 1.00E+00 | 1.00E+00 |
| and Analogues | | | | | |
| Purine Ribonucleoside | 4 (2%) | 1 (1.1%) | 20.0% | 1.00E+00 | 1.00E+00 |
| Diphosphates | | | | | |
| Pyrimidine 2′- | 2 (1%) | 1 (1.1%) | 33.3% | 1.00E+00 | 1.00E+00 |
| deoxyribonucleosides and | | | | | |
| Analogues | | | | | |
| Sugar Acids and Derivatives | 4 (2%) | 2 (2.3%) | 33.3% | 1.00E+00 | 1.00E+00 |
| Sugar Alcohols | 2 (1%) | 1 (1.1%) | 33.3% | 1.00E+00 | 1.00E+00 |

TABLE 37
Metabolites found and missed in formalin solution categorized by substituent.

| | non-found in | | | | |
|---|---|---|---|---|---|
| | formalin solution, n | found in formalin | formalin solution/ | | |
| Substituent | (%) | solution, n (%) | FROZEN, % | P | FDR |
| phosphoric acid ester | 90 (34.5%) | 5 (4.1%) | 5.3% | 3.09E−12 | 7.07E−10 |
| organic phosphite | 91 (34.9%) | 6 (5%) | 6.2% | 1.29E−11 | 1.47E−09 |
| organic hypophosphite | 91 (34.9%) | 8 (6.6%) | 8.1% | 3.40E−10 | 2.60E−08 |
| fatty acid ester | 57 (21.8%) | 1 (0.8%) | 1.7% | 1.12E−09 | 6.41E−08 |
| alpha-amino acid or derivative | 52 (19.9%) | 3 (2.5%) | 5.5% | 1.16E−06 | 5.29E−05 |
| n-substituted-alpha-amino acid | 49 (18.8%) | 3 (2.5%) | 5.8% | 3.50E−06 | 1.34E−04 |
| n-acyl-alpha-amino-acid | 47 (18%) | 3 (2.5%) | 6.0% | 5.98E−06 | 1.80E−04 |
| carboxylic acid ester | 66 (25.3%) | 8 (6.6%) | 10.8% | 6.29E−06 | 1.80E−04 |
| phosphoethanolamine | 46 (17.6%) | 3 (2.5%) | 6.1% | 1.04E−05 | 2.65E−04 |
| primary aliphatic amine | 102 (39.1%) | 24 (19.8%) | 19.0% | 1.74E−04 | 3.91E−03 |
| (alkylamine) | | | | | |
| secondary carboxylic acid amide | 77 (29.5%) | 15 (12.4%) | 16.3% | 1.88E−04 | 3.91E−03 |
| monosaccharide phosphate | 22 (8.4%) | 0 (0%) | 0.0% | 2.27E−04 | 4.34E−03 |
| carboxamide_group | 85 (32.6%) | 19 (15.7%) | 18.3% | 5.13E−04 | 9.03E−03 |
| secondary alcohol | 114 (43.7%) | 32 (26.4%) | 21.9% | 1.48E−03 | 2.42E−02 |
| short-chain hydroxy acid | 1 (0.4%) | 7 (5.8%) | 87.5% | 1.66E−03 | 2.54E−02 |
| carboxylic acid | 125 (47.9%) | 78 (64.5%) | 38.4% | 2.90E−03 | 4.06E−02 |
| acyclic alkene | 52 (19.9%) | 10 (8.3%) | 16.1% | 4.28E−03 | 5.44E−02 |
| glycero-3-phosphocholine | 19 (7.3%) | 1 (0.8%) | 5.0% | 5.96E−03 | 7.18E−02 |
| phosphocholine | 22 (8.4%) | 2 (1.7%) | 8.3% | 1.12E−02 | 1.22E−01 |
| saccharide | 47 (18%) | 10 (8.3%) | 17.5% | 1.33E−02 | 1.38E−01 |
| ketone | 2 (0.8%) | 6 (5%) | 75.0% | 1.41E−02 | 1.41E−01 |
| organic pyrophosphate | 17 (6.5%) | 1 (0.8%) | 5.6% | 1.67E−02 | 1.59E−01 |
| secondary aliphatic amine | 3 (1.1%) | 6 (5%) | 66.7% | 3.14E−02 | 2.77E−01 |
| (dialkylamine) | | | | | |
| aminopyrimidine | 30 (11.5%) | 6 (5%) | 16.7% | 5.81E−02 | 4.93E−01 |
| pyrimidine | 45 (17.2%) | 12 (9.9%) | 21.1% | 6.54E−02 | 5.35E−01 |
| 1-phosphoribosyl-imidazole | 12 (4.6%) | 1 (0.8%) | 7.7% | 7.04E−02 | 5.56E−01 |
| oxolane | 41 (15.7%) | 11 (9.1%) | 21.2% | 1.08E−01 | 7.71E−01 |
| imidazopyrimidine | 22 (8.4%) | 5 (4.1%) | 18.5% | 1.40E−01 | 9.17E−01 |
| purine | 22 (8.4%) | 5 (4.1%) | 18.5% | 1.40E−01 | 9.17E−01 |
| 1,2-diol | 62 (23.8%) | 20 (16.5%) | 24.4% | 1.40E−01 | 9.17E−01 |
| guanidine | 4 (1.5%) | 5 (4.1%) | 55.6% | 1.49E−01 | 9.48E−01 |
| oxane | 13 (5%) | 2 (1.7%) | 13.3% | 1.60E−01 | 9.88E−01 |
| n-acylglycine | 9 (3.4%) | 1 (0.8%) | 10.0% | 1.80E−01 | 1.00E+00 |
| n-glycosyl compound | 29 (11.1%) | 8 (6.6%) | 21.6% | 1.96E−01 | 1.00E+00 |
| triose monosaccharide | 15 (5.7%) | 3 (2.5%) | 16.7% | 2.00E−01 | 1.00E+00 |
| carnitine | 8 (3.1%) | 7 (5.8%) | 46.7% | 2.57E−01 | 1.00E+00 |
| disaccharide phosphate | 8 (3.1%) | 1 (0.8%) | 11.1% | 2.83E−01 | 1.00E+00 |
| dicarboxylic acid derivative | 45 (17.2%) | 15 (12.4%) | 25.0% | 2.90E−01 | 1.00E+00 |
| pentose monosaccharide | 24 (9.2%) | 7 (5.8%) | 22.6% | 3.16E−01 | 1.00E+00 |
| pyrimidone | 27 (10.3%) | 8 (6.6%) | 22.9% | 3.40E−01 | 1.00E+00 |
| amphetamine or derivative | 10 (3.8%) | 2 (1.7%) | 16.7% | 3.53E−01 | 1.00E+00 |
| primary alcohol | 61 (23.4%) | 23 (19%) | 27.4% | 3.56E−01 | 1.00E+00 |
| glycosyl compound | 29 (11.1%) | 9 (7.4%) | 23.7% | 3.58E−01 | 1.00E+00 |
| alpha-hydroxy acid | 7 (2.7%) | 6 (5%) | 46.2% | 3.62E−01 | 1.00E+00 |
| choline | 30 (11.5%) | 10 (8.3%) | 25.0% | 3.75E−01 | 1.00E+00 |
| carboxylic acid salt | 15 (5.7%) | 10 (8.3%) | 40.0% | 3.77E−01 | 1.00E+00 |
| polyamine | 12 (4.6%) | 3 (2.5%) | 20.0% | 4.06E−01 | 1.00E+00 |
| alkylthiol | 7 (2.7%) | 1 (0.8%) | 12.5% | 4.44E−01 | 1.00E+00 |
| thiol (sulfanyl compound) | 7 (2.7%) | 1 (0.8%) | 12.5% | 4.44E−01 | 1.00E+00 |
| 1,3-aminoalcohol | 15 (5.7%) | 4 (3.3%) | 21.1% | 4.49E−01 | 1.00E+00 |
| imidazole | 27 (10.3%) | 9 (7.4%) | 25.0% | 4.53E−01 | 1.00E+00 |
| hemiacetal | 9 (3.4%) | 2 (1.7%) | 18.2% | 5.14E−01 | 1.00E+00 |
| quaternary ammonium salt | 34 (13%) | 13 (10.7%) | 27.7% | 6.17E−01 | 1.00E+00 |
| hydropyrimidine | 15 (5.7%) | 5 (4.1%) | 25.0% | 6.26E−01 | 1.00E+00 |
| beta-hydroxy acid | 13 (5%) | 8 (6.6%) | 38.1% | 6.30E−01 | 1.00E+00 |
| allyl alcohol | 5 (1.9%) | 1 (0.8%) | 16.7% | 6.69E−01 | 1.00E+00 |
| urea | 5 (1.9%) | 1 (0.8%) | 16.7% | 6.69E−01 | 1.00E+00 |
| succinic_acid | 4 (1.5%) | 3 (2.5%) | 42.9% | 6.84E−01 | 1.00E+00 |
| pyrrolidine | 4 (1.5%) | 3 (2.5%) | 42.9% | 6.84E−01 | 1.00E+00 |
| cyclohexane | 7 (2.7%) | 2 (1.7%) | 22.2% | 7.25E−01 | 1.00E+00 |
| primary carboxylic acid amide | 7 (2.7%) | 4 (3.3%) | 36.4% | 7.48E−01 | 1.00E+00 |
| thioether | 7 (2.7%) | 4 (3.3%) | 36.4% | 7.48E−01 | 1.00E+00 |
| hypoxanthine | 6 (2.3%) | 2 (1.7%) | 25.0% | 1.00E+00 | 1.00E+00 |
| purinone | 5 (1.9%) | 2 (1.7%) | 28.6% | 1.00E+00 | 1.00E+00 |
| 1,2-aminoalcohol | 9 (3.4%) | 4 (3.3%) | 30.8% | 1.00E+00 | 1.00E+00 |
| pyrrolidine carboxylic acid | 4 (1.5%) | 2 (1.7%) | 33.3% | 1.00E+00 | 1.00E+00 |

TABLE 38
Metabolites found and missed in formalin solution categorized by property.

| | non-found in | | | |
|---|---|---|---|---|
| | formalin solution, | found in formalin | | |
| Propriety | n (%) | solution, n (%) | P | FDR |
| mono_mass_ChemAxon | 337.59 | 182.31 | 2.35E−24 | 2.10E−23 |
| average_mass_ChemAxon | 337.79 | 182.41 | 2.63E−24 | 2.10E−23 |
| polarizability_ChemAxon | 35.74 | 18.05 | 6.86E−23 | 3.66E−22 |
| refractivity_ChemAxon | 87.45 | 46.87 | 5.58E−20 | 2.23E−19 |
| rotatable_bond_count_ChemAxon | 11.57 | 4.30 | 4.80E−17 | 1.54E−16 |
| logs_ALOGPS | −2.98 | −1.27 | 6.93E−13 | 1.85E−12 |
| polar_surface_area_ChemAxon | 121.48 | 83.86 | 4.34E−12 | 9.93E−12 |
| solubility_ALOGPS | 37.22 | 119.42 | 7.81E−11 | 1.56E−10 |
| acceptor_count_ChemAxon | 5.60 | 4.11 | 1.08E−07 | 1.92E−07 |
| donor_count_ChemAxon | 3.41 | 2.53 | 4.01E−05 | 6.42E−05 |
| logp_ALOGPS | 0.53 | −0.86 | 1.80E−02 | 2.47E−02 |
| pka_strongest_acidic_ChemAxon | 4.20 | 4.33 | 1.85E−02 | 2.47E−02 |
| pka_strongest_basic_ChemAxon | 3.14 | 1.57 | 2.58E−01 | 3.17E−01 |
| formal_charge_ChemAxon | 0.00 | 0.00 | 4.02E−01 | 4.59E−01 |
| logp_ChemAxon | −0.23 | −1.30 | 4.71E−01 | 5.02E−01 |
| physiological_charge_ChemAxon | −0.62 | −0.55 | 5.62E−01 | 5.62E−01 |

TABLE 39
Metabolites found and missed in formalin solution categorized by pathway.

| | non-found in | found in | | | |
|---|---|---|---|---|---|
| | formalin | formalin | formalin solution/ | | |
| Pathway | solution, n (%) | solution, n (%) | FROZEN, % | P | FDR |
| Transcription/Translation | 7 (2.7%) | 17 (14%) | 70.8% | 5.48E−05 | 4.82E−03 |
| Arginine and Proline Metabolism | 1 (0.4%) | 8 (6.6%) | 88.9% | 5.67E−04 | 2.49E−02 |
| Ammonia Recycling | 3 (1.1%) | 10 (8.3%) | 76.9% | 8.66E−04 | 2.54E−02 |
| Urea Cycle | 3 (1.1%) | 9 (7.4%) | 75.0% | 2.23E−03 | 4.42E−02 |
| Glucose-Alanine Cycle | 1 (0.4%) | 6 (5%) | 85.7% | 4.79E−03 | 6.02E−02 |
| Glycine and Serine Metabolism | 11 (4.2%) | 14 (11.6%) | 56.0% | 1.26E−02 | 1.39E−01 |
| Carnitine Synthesis | 4 (1.5%) | 7 (5.8%) | 63.6% | 4.15E−02 | 3.01E−01 |
| Methionine Metabolism | 6 (2.3%) | 8 (6.6%) | 57.1% | 4.45E−02 | 3.01E−01 |
| Spermidine and Spermine | 2 (0.8%) | 4 (3.3%) | 66.7% | 8.29E−02 | 4.56E−01 |
| Biosynthesis | | | | | |
| Mitochondrial Electron Transport | 2 (0.8%) | 4 (3.3%) | 66.7% | 8.29E−02 | 4.56E−01 |
| Chain | | | | | |
| Aspartate Metabolism | 2 (0.8%) | 4 (3.3%) | 66.7% | 8.29E−02 | 4.56E−01 |
| Valine, Leucine and Isoleucine | 3 (1.1%) | 5 (4.1%) | 62.5% | 1.16E−01 | 5.66E−01 |
| Degradation | | | | | |
| Citric Acid Cycle | 6 (2.3%) | 7 (5.8%) | 53.8% | 1.25E−01 | 5.77E−01 |
| Transfer of Acetyl Groups into | 4 (1.5%) | 5 (4.1%) | 55.6% | 1.49E−01 | 6.56E−01 |
| Mitochondria | | | | | |
| Pentose Phosphate Pathway | 6 (2.3%) | 0 (0%) | 0.0% | 1.83E−01 | 7.66E−01 |
| Histidine Metabolism | 4 (1.5%) | 4 (3.3%) | 50.0% | 2.69E−01 | 8.19E−01 |
| Glutathione Metabolism | 4 (1.5%) | 4 (3.3%) | 50.0% | 2.69E−01 | 8.19E−01 |
| Alpha Linolenic Acid and Linoleic | 4 (1.5%) | 4 (3.3%) | 50.0% | 2.69E−01 | 8.19E−01 |
| Acid Metabolism | | | | | |
| Galactose Metabolism | 4 (1.5%) | 4 (3.3%) | 50.0% | 2.69E−01 | 8.19E−01 |
| Glutamate Metabolism | 6 (2.3%) | 5 (4.1%) | 45.5% | 3.35E−01 | 8.19E−01 |
| Phospholipid Biosynthesis | 3 (1.1%) | 3 (2.5%) | 50.0% | 3.86E−01 | 8.71E−01 |
| Pyruvate Metabolism | 3 (1.1%) | 3 (2.5%) | 50.0% | 3.86E−01 | 8.71E−01 |
| Beta-Alanine Metabolism | 3 (1.1%) | 3 (2.5%) | 50.0% | 3.86E−01 | 8.71E−01 |
| Lactose Synthesis | 6 (2.3%) | 1 (0.8%) | 14.3% | 4.39E−01 | 9.67E−01 |
| Glycerolipid Metabolism | 5 (1.9%) | 4 (3.3%) | 44.4% | 4.73E−01 | 1.00E+00 |
| Gluconeogenesis | 8 (3.1%) | 5 (4.1%) | 38.5% | 5.60E−01 | 1.00E+00 |
| Plasmalogen Synthesis | 5 (1.9%) | 1 (0.8%) | 16.7% | 6.69E−01 | 1.00E+00 |
| Fructose and Mannose Degradation | 5 (1.9%) | 1 (0.8%) | 16.7% | 6.69E−01 | 1.00E+00 |
| Amino Sugar Metabolism | 11 (4.2%) | 4 (3.3%) | 26.7% | 7.83E−01 | 1.00E+00 |
| Glycolysis | 8 (3.1%) | 3 (2.5%) | 27.3% | 1.00E+00 | 1.00E+00 |
| Purine Metabolism | 13 (5%) | 6 (5%) | 31.6% | 1.00E+00 | 1.00E+00 |
| Pyrimidine Metabolism | 12 (4.6%) | 5 (4.1%) | 29.4% | 1.00E+00 | 1.00E+00 |
| Betaine Metabolism | 4 (1.5%) | 2 (1.7%) | 33.3% | 1.00E+00 | 1.00E+00 |
| Mitochondrial Beta-Oxidation of | 6 (2.3%) | 2 (1.7%) | 25.0% | 1.00E+00 | 1.00E+00 |
| Long Chain Saturated Fatty Acids | | | | | |
| Mitochondrial Beta-Oxidation of | 5 (1.9%) | 2 (1.7%) | 28.6% | 1.00E+00 | 1.00E+00 |
| Short Chain Saturated Fatty Acids | | | | | |

The majority of these metabolites were classified as amino acids (and derivatives). Specifically, in the supernatant, 53% of all amino acids present were detected in the frozen samples (P=2.33×10⁻⁸; FDR=1.87×10⁻⁷). Analyzing their chemical-physical properties, metabolites soluble in formalin are characterized by lower molecular weight (P=2.35×10⁻²⁴; FDR=2.10×10⁻²³), polarizability (P=6.86×10⁻²³; FDR=3.66×10⁻²²), refractivity (P=2.58×10⁻²⁰; FDR=2.23×10⁻¹⁹), number of rotatable bond (P=4.80×10⁻¹⁷; FDR=1.54×10⁻¹⁶), and a higher solubility (P=7.81×10⁻¹¹; FDR=1.56×10⁻¹⁰). Second, metabolites that might be lost in FPPE due to their reaction with formaldehyde when tissues are immersed in a formalin solution were identified. Metabolites interacting with formalin could form covalent bonds with cellular components (insoluble or with high molecular weight) and thus be no longer detectable by MS. Table 40, Table 41, Table 42, Table 43, Table 44, and Table 45 list the metabolites that were not detected in either formalin solution, nor in the extract from FF samples.

TABLE 40
Metabolites found in frozen cell samples and missed in supernatant and FF cell
samples categorized by superclass.

| | non-bonded, | bonded, n | bonded/ | | |
|---|---|---|---|---|---|
| Superclass | n (%) | (%) | FROZEN, % | p | FDR |
| Peptide | 12 (3.6%) | 42 (40%) | 77.8% | 2.29E−19 | 1.84E−18 |
| Lipid | 156 (47%) | 4 (3.8%) | 2.5% | 6.83E−19 | 2.73E−18 |
| Carbohydrate | 17 (5.1%) | 15 (14.3%) | 46.9% | 4.09E−03 | 1.09E−02 |
| Energy | 7 (2.1%) | 0 (0%) | 0.0% | 2.04E−01 | 4.09E−01 |
| Nucleotide | 35 (10.5%) | 7 (6.7%) | 16.7% | 3.41E−01 | 5.46E−01 |
| Cofactors and Vitamins | 15 (4.5%) | 7 (6.7%) | 31.8% | 4.41E−01 | 5.88E−01 |
| Xenobiotics | 10 (3%) | 4 (3.8%) | 28.6% | 7.51E−01 | 8.58E−01 |
| Amino Acid | 80 (24.1%) | 26 (24.8%) | 24.5% | 8.97E−01 | 8.97E−01 |

TABLE 41
Metabolites found in frozen cell samples and missed in supernatant and FF cell
samples categorized by class.

| | non-bonded, n | | bonded/ | | |
|---|---|---|---|---|---|
| Class | (%) | bonded, n (%) | FROZEN, % | p | FDR |
| Peptides | 15 (5.8%) | 37 (41.1%) | 71.2% | 2.25E−14 | 5.62E−13 |
| Glycerophospholipids | 48 (18.5%) | 0 (0%) | 0.0% | 4.75E−07 | 5.94E−06 |
| Fatty Acids and Conjugates | 29 (11.2%) | 0 (0%) | 0.0% | 2.29E−04 | 1.91E−03 |
| Monosaccharides | 6 (2.3%) | 7 (7.8%) | 53.8% | 2.16E−02 | 1.35E−01 |
| Glycerolipids | 10 (3.9%) | 0 (0%) | 0.0% | 7.29E−02 | 3.65E−01 |
| Sugar Acids and Derivatives | 4 (1.5%) | 4 (4.4%) | 50.0% | 1.10E−01 | 4.57E−01 |
| Azoles | 1 (0.4%) | 2 (2.2%) | 66.7% | 1.54E−01 | 5.50E−01 |
| Amino Acids and Derivatives | 52 (20.1%) | 23 (25.6%) | 30.7% | 2.33E−01 | 6.60E−01 |
| Pteridines and Derivatives | 2 (0.8%) | 2 (2.2%) | 50.0% | 2.59E−01 | 6.60E−01 |
| Pyrimidine Nucleotides | 11 (4.2%) | 1 (1.1%) | 8.3% | 3.08E−01 | 6.60E−01 |
| Sphingolipids | 5 (1.9%) | 0 (0%) | 0.0% | 3.38E−01 | 6.60E−01 |
| Carboxylic Acids and Derivatives | 6 (2.3%) | 0 (0%) | 0.0% | 3.43E−01 | 6.60E−01 |
| Pyrimidine Nucleosides and | 6 (2.3%) | 0 (0%) | 0.0% | 3.43E−01 | 6.60E−01 |
| Analogues | | | | | |
| Benzyl Alcohols and Derivatives | 4 (1.5%) | 0 (0%) | 0.0% | 5.76E−01 | 9.05E−01 |
| Lineolic Acids and Derivatives | 4 (1.5%) | 0 (0%) | 0.0% | 5.76E−01 | 9.05E−01 |
| Fatty Acid Esters | 14 (5.4%) | 3 (3.3%) | 17.6% | 5.79E−01 | 9.05E−01 |
| Hydroxy Acids and Derivatives | 7 (2.7%) | 1 (1.1%) | 12.5% | 6.85E−01 | 9.71E−01 |
| Purine Nucleosides and Analogues | 7 (2.7%) | 3 (3.3%) | 30.0% | 7.15E−01 | 9.71E−01 |
| Purine Nucleotides | 10 (3.9%) | 2 (2.2%) | 16.7% | 7.38E−01 | 9.71E−01 |
| Alkylamines | 4 (1.5%) | 1 (1.1%) | 20.0% | 1.00E+00 | 1.00E+00 |
| Cyclic Alcohols and Derivatives | 3 (1.2%) | 1 (1.1%) | 25.0% | 1.00E+00 | 1.00E+00 |
| Imidazopyrimidines | 4 (1.5%) | 1 (1.1%) | 20.0% | 1.00E+00 | 1.00E+00 |
| Organic Phosphoric Acids and | 2 (0.8%) | 1 (1.1%) | 33.3% | 1.00E+00 | 1.00E+00 |
| Derivatives | | | | | |
| Pyridines and Derivatives | 3 (1.2%) | 0 (0%) | 0.0% | 1.00E+00 | 1.00E+00 |
| Sugar Alcohols | 2 (0.8%) | 1 (1.1%) | 33.3% | 1.00E+00 | 1.00E+00 |

TABLE 42
Metabolites found in frozen cell samples and missed in supernatant and FF cell
samples categorized by subclass.

| | non-bonded, n | | bonded/ | | |
|---|---|---|---|---|---|
| Subclass | (%) | bonded, n (%) | FROZEN, % | p | FDR |
| Peptides | 15 (7.3%) | 37 (45.7%) | 71.2% | 2.25E−14 | 7.19E−13 |
| Hexoses | 1 (0.5%) | 6 (7.4%) | 85.7% | 1.17E−03 | 1.87E−02 |
| N-acyl-alpha Amino Acids and | 10 (4.9%) | 12 (14.8%) | 54.5% | 3.54E−03 | 3.77E−02 |
| Derivatives | | | | | |
| Lysophosphatidylethanolamines | 18 (8.7%) | 0 (0%) | 0.0% | 9.31E−03 | 7.45E−02 |
| Unsaturated Fatty Acids | 15 (7.3%) | 0 (0%) | 0.0% | 2.74E−02 | 1.75E−01 |
| Lysophosphatidylcholines | 13 (6.3%) | 0 (0%) | 0.0% | 4.43E−02 | 2.36E−01 |
| Monoacylglycerols | 10 (4.9%) | 0 (0%) | 0.0% | 7.29E−02 | 3.33E−01 |
| Imidazolyl Carboxylic Acids and | 1 (0.5%) | 2 (2.5%) | 66.7% | 1.54E−01 | 5.89E−01 |
| Derivatives | | | | | |
| Sugar Acids and Derivatives | 3 (1.5%) | 3 (3.7%) | 50.0% | 1.66E−01 | 5.89E−01 |
| Phosphatidylcholines | 7 (3.4%) | 0 (0%) | 0.0% | 2.00E−01 | 6.07E−01 |
| Straight Chain Fatty Acids | 8 (3.9%) | 0 (0%) | 0.0% | 2.09E−01 | 6.07E−01 |
| Alpha Amino Acids and | 34 (16.5%) | 7 (8.6%) | 17.1% | 2.56E−01 | 6.83E−01 |
| Derivatives | | | | | |
| Branched Fatty Acids | 4 (1.9%) | 0 (0%) | 0.0% | 5.76E−01 | 1.00E+00 |
| Lineolic Acids and Derivatives | 4 (1.9%) | 0 (0%) | 0.0% | 5.76E−01 | 1.00E+00 |
| Phenylpyruvic Acid Derivatives | 4 (1.9%) | 0 (0%) | 0.0% | 5.76E−01 | 1.00E+00 |
| Pyrimidine Nucleotide Sugars | 4 (1.9%) | 0 (0%) | 0.0% | 5.76E−01 | 1.00E+00 |
| Purine Nucleosides and Analogues | 4 (1.9%) | 2 (2.5%) | 33.3% | 6.41E−01 | 1.00E+00 |
| Acyl Carnitines | 12 (5.8%) | 3 (3.7%) | 20.0% | 1.00E+00 | 1.00E+00 |
| Acyl Glycines | 2 (1%) | 1 (1.2%) | 33.3% | 1.00E+00 | 1.00E+00 |
| Beta Amino Acids and Derivatives | 2 (1%) | 1 (1.2%) | 33.3% | 1.00E+00 | 1.00E+00 |
| Beta Hydroxy Acids and | 4 (1.9%) | 1 (1.2%) | 20.0% | 1.00E+00 | 1.00E+00 |
| Derivatives | | | | | |
| Dicarboxylic Acids and Derivatives | 3 (1.5%) | 0 (0%) | 0.0% | 1.00E+00 | 1.00E+00 |
| Glycoamino Acids and Derivatives | 2 (1%) | 1 (1.2%) | 33.3% | 1.00E+00 | 1.00E+00 |
| Pentoses | 3 (1.5%) | 1 (1.2%) | 25.0% | 1.00E+00 | 1.00E+00 |
| Phosphatidylinositols | 3 (1.5%) | 0 (0%) | 0.0% | 1.00E+00 | 1.00E+00 |
| Polyamines | 2 (1%) | 1 (1.2%) | 33.3% | 1.00E+00 | 1.00E+00 |
| Purine 2′-deoxyribonucleosides and | 3 (1.5%) | 1 (1.2%) | 25.0% | 1.00E+00 | 1.00E+00 |
| Analogues | | | | | |
| Purine Ribonucleoside | 4 (1.9%) | 1 (1.2%) | 20.0% | 1.00E+00 | 1.00E+00 |
| Diphosphates | | | | | |
| Purine Ribonucleoside | 3 (1.5%) | 0 (0%) | 0.0% | 1.00E+00 | 1.00E+00 |
| Monophosphates | | | | | |
| Pyrimidine 2′- | 3 (1.5%) | 0 (0%) | 0.0% | 1.00E+00 | 1.00E+00 |
| deoxyribonucleosides and | | | | | |
| Analogues | | | | | |
| Pyrimidine Nucleosides and | 3 (1.5%) | 0 (0%) | 0.0% | 1.00E+00 | 1.00E+00 |
| Analogues | | | | | |
| Sugar Alcohols | 2 (1%) | 1 (1.2%) | 33.3% | 1.00E+00 | 1.00E+00 |

TABLE 43
Metabolites found in frozen cell samples and missed in supernatant and FF cell
samples categorized by substituent.

| Substituent | non-bonded, n (%) | | bonded, n (%) | | bonded/FROZEN, % | p | FDR |
|---|---|---|---|---|---|---|---|
| n-substituted-alpha-amino acid | 14 | (4.9%) | 38 | (40%) | 73.1% | 1.87E−15 | 2.88E−13 |
| secondary carboxylic acid amide | 39 | (13.6%) | 53 | (55.8%) | 57.6% | 2.52E−15 | 2.88E−13 |
| carboxamide_group | 48 | (16.7%) | 56 | (58.9%) | 53.8% | 1.61E−14 | 1.23E−12 |
| n-acyl-alpha-amino-acid | 14 | (4.9%) | 36 | (37.9%) | 72.0% | 3.20E−14 | 1.83E−12 |
| alpha-amino acid or derivative | 17 | (5.9%) | 38 | (40%) | 69.1% | 4.14E−14 | 1.90E−12 |
| fatty acid ester | 58 | (20.2%) | 0 | (0%) | 0.0% | 2.33E−08 | 8.90E−07 |
| carboxylic acid ester | 71 | (24.7%) | 3 | (3.2%) | 4.1% | 3.69E−07 | 1.21E−05 |
| carboxylic acid | 132 | (46%) | 71 | (74.7%) | 35.0% | 9.37E−07 | 2.68E−05 |
| primary aliphatic amine | 75 | (26.1%) | 51 | (53.7%) | 40.5% | 1.39E−06 | 3.53E−05 |
| (alkylamine) | | | | | | | |
| acyclic alkene | 60 | (20.9%) | 2 | (2.1%) | 3.2% | 1.61E−06 | 3.69E−05 |
| phosphoethanolamine | 48 | (16.7%) | 1 | (1.1%) | 2.0% | 1.01E−05 | 2.10E−04 |
| amphetamine or derivative | 2 | (0.7%) | 10 | (10.5%) | 83.3% | 2.59E−05 | 4.93E−04 |
| quaternary ammonium salt | 44 | (15.3%) | 3 | (3.2%) | 6.4% | 9.75E−04 | 1.72E−02 |
| phosphocholine | 24 | (8.4%) | 0 | (0%) | 0.0% | 1.13E−03 | 1.84E−02 |
| glycero-3-phosphocholine | 20 | (7%) | 0 | (0%) | 0.0% | 5.71E−03 | 8.70E−02 |
| choline | 37 | (12.9%) | 3 | (3.2%) | 7.5% | 6.08E−03 | 8.70E−02 |
| hemiacetal | 4 | (1.4%) | 7 | (7.4%) | 63.6% | 6.55E−03 | 8.82E−02 |
| saccharide | 51 | (17.8%) | 6 | (6.3%) | 10.5% | 7.20E−03 | 9.16E−02 |
| triose monosaccharide | 18 | (6.3%) | 0 | (0%) | 0.0% | 9.31E−03 | 1.12E−01 |
| 1,3-aminoalcohol | 9 | (3.1%) | 10 | (10.5%) | 52.6% | 1.08E−02 | 1.24E−01 |
| oxane | 7 | (2.4%) | 8 | (8.4%) | 53.3% | 1.49E−02 | 1.56E−01 |
| beta-hydroxy acid | 11 | (3.8%) | 10 | (10.5%) | 47.6% | 1.91E−02 | 1.88E−01 |
| phosphoric acid ester | 80 | (27.9%) | 15 | (15.8%) | 15.8% | 1.97E−02 | 1.88E−01 |
| organic hypophosphite | 83 | (28.9%) | 16 | (16.8%) | 16.2% | 2.15E−02 | 1.90E−01 |
| 1,2-aminoalcohol | 6 | (2.1%) | 7 | (7.4%) | 53.8% | 2.16E−02 | 1.90E−01 |
| organic phosphite | 81 | (28.2%) | 16 | (16.8%) | 16.5% | 2.96E−02 | 2.51E−01 |
| n-glycosyl compound | 32 | (11.1%) | 5 | (5.3%) | 13.5% | 1.10E−01 | 8.81E−01 |
| glycosyl compound | 33 | (11.5%) | 5 | (5.3%) | 13.2% | 1.12E−01 | 8.81E−01 |
| pentose monosaccharide | 27 | (9.4%) | 4 | (4.2%) | 12.9% | 1.31E−01 | 9.65E−01 |
| thioether | 6 | (2.1%) | 5 | (5.3%) | 45.5% | 1.50E−01 | 9.65E−01 |
| hydropyrimidine | 18 | (6.3%) | 2 | (2.1%) | 10.0% | 1.81E−01 | 9.65E−01 |
| short-chain hydroxy acid | 8 | (2.8%) | 0 | (0%) | 0.0% | 2.09E−01 | 9.65E−01 |
| secondary alcohol | 115 | (40.1%) | 31 | (32.6%) | 21.2% | 2.24E−01 | 9.65E−01 |
| oxolane | 43 | (15%) | 9 | (9.5%) | 17.3% | 2.27E−01 | 9.65E−01 |
| n-acylglycine | 6 | (2.1%) | 4 | (4.2%) | 40.0% | 2.74E−01 | 1.00E+00 |
| dicarboxylic acid derivative | 42 | (14.6%) | 18 | (18.9%) | 30.0% | 3.31E−01 | 1.00E+00 |
| allyl alcohol | 6 | (2.1%) | 0 | (0%) | 0.0% | 3.43E−01 | 1.00E+00 |
| purinone | 4 | (1.4%) | 3 | (3.2%) | 42.9% | 3.72E−01 | 1.00E+00 |
| polyamine | 13 | (4.5%) | 2 | (2.1%) | 13.3% | 3.75E−01 | 1.00E+00 |
| hypoxanthine | 5 | (1.7%) | 3 | (3.2%) | 37.5% | 4.16E−01 | 1.00E+00 |
| alkylthiol | 5 | (1.7%) | 3 | (3.2%) | 37.5% | 4.16E−01 | 1.00E+00 |
| thiol (sulfanyl compound) | 5 | (1.7%) | 3 | (3.2%) | 37.5% | 4.16E−01 | 1.00E+00 |
| cyclohexane | 8 | (2.8%) | 1 | (1.1%) | 11.1% | 4.61E−01 | 1.00E+00 |
| primary carboxylic acid amide | 7 | (2.4%) | 4 | (4.2%) | 36.4% | 4.77E−01 | 1.00E+00 |
| pyrimidine | 45 | (15.7%) | 12 | (12.6%) | 21.1% | 5.12E−01 | 1.00E+00 |
| 1-phosphoribosyl-imidazole | 11 | (3.8%) | 2 | (2.1%) | 15.4% | 5.32E−01 | 1.00E+00 |
| 1,2-diol | 64 | (22.3%) | 18 | (18.9%) | 22.0% | 5.65E−01 | 1.00E+00 |
| organic pyrophosphate | 15 | (5.2%) | 3 | (3.2%) | 16.7% | 5.79E−01 | 1.00E+00 |
| urea | 4 | (1.4%) | 2 | (2.1%) | 33.3% | 6.41E−01 | 1.00E+00 |
| pyrrolidine carboxylic acid | 4 | (1.4%) | 2 | (2.1%) | 33.3% | 6.41E−01 | 1.00E+00 |
| primary alcohol | 65 | (22.6%) | 19 | (20%) | 22.6% | 6.69E−01 | 1.00E+00 |
| aminopyrimidine | 26 | (9.1%) | 10 | (10.5%) | 27.8% | 6.87E−01 | 1.00E+00 |
| alpha-hydroxy acid | 9 | (3.1%) | 4 | (4.2%) | 30.8% | 7.44E−01 | 1.00E+00 |
| carboxylic acid salt | 18 | (6.3%) | 7 | (7.4%) | 28.0% | 8.11E−01 | 1.00E+00 |
| carnitine | 12 | (4.2%) | 3 | (3.2%) | 20.0% | 1.00E+00 | 1.00E+00 |
| imidazole | 27 | (9.4%) | 9 | (9.5%) | 25.0% | 1.00E+00 | 1.00E+00 |
| pyrimidone | 27 | (9.4%) | 8 | (8.4%) | 22.9% | 1.00E+00 | 1.00E+00 |
| ketone | 6 | (2.1%) | 2 | (2.1%) | 25.0% | 1.00E+00 | 1.00E+00 |
| imidazopyrimidine | 21 | (7.3%) | 6 | (6.3%) | 22.2% | 1.00E+00 | 1.00E+00 |
| purine | 21 | (7.3%) | 6 | (6.3%) | 22.2% | 1.00E+00 | 1.00E+00 |
| monosaccharide phosphate | 17 | (5.9%) | 5 | (5.3%) | 22.7% | 1.00E+00 | 1.00E+00 |
| guanidine | 7 | (2.4%) | 2 | (2.1%) | 22.2% | 1.00E+00 | 1.00E+00 |
| disaccharide phosphate | 7 | (2.4%) | 2 | (2.1%) | 22.2% | 1.00E+00 | 1.00E+00 |
| succinic_acid | 6 | (2.1%) | 1 | (1.1%) | 14.3% | 1.00E+00 | 1.00E+00 |
| secondary aliphatic amine | 7 | (2.4%) | 2 | (2.1%) | 22.2% | 1.00E+00 | 1.00E+00 |
| (dialkylamine) | | | | | | | |
| pyrrolidine | 5 | (1.7%) | 2 | (2.1%) | 28.6% | 1.00E+00 | 1.00E+00 |

TABLE 44
Metabolites found in frozen cell samples and missed in supernatant and FF cell
samples categorized by physical/chemical property.

| | non-bonded, n | | | |
|---|---|---|---|---|
| Propriety | (%) | bonded, n (%) | p | FDR |
| logp_ALOGPS | 0.75 | −1.88 | 4.80E−10 | 7.68E−09 |
| logp_ChemAxon | 0.15 | −2.72 | 7.46E−09 | 5.97E−08 |
| donor_count_ChemAxon | 2.89 | 3.88 | 2.54E−08 | 1.36E−07 |
| acceptor_count_ChemAxon | 4.95 | 5.66 | 1.86E−05 | 7.44E−05 |
| polar_surface_area_ChemAxon | 105.82 | 120.88 | 4.91E−05 | 1.38E−04 |
| solubility_ALOGPS | 64.83 | 58.23 | 5.17E−05 | 1.38E−04 |
| logs_ALOGPS | −2.81 | −1.32 | 8.77E−05 | 2.01E−04 |
| refractivity_ChemAxon | 80.50 | 56.75 | 4.73E−03 | 9.47E−03 |
| pka_strongest_basic_ChemAxon | 1.82 | 5.00 | 1.03E−02 | 1.84E−02 |
| polarizability_ChemAxon | 32.55 | 22.86 | 1.26E−02 | 2.02E−02 |
| rotatable_bond_count_ChemAxon | 10.60 | 5.24 | 1.83E−02 | 2.67E−02 |
| formal_charge_ChemAxon | 0.01 | −0.03 | 2.03E−02 | 2.71E−02 |
| average_mass_ChemAxon | 304.73 | 239.78 | 7.54E−02 | 8.72E−02 |
| mono_mass_ChemAxon | 304.55 | 239.64 | 7.63E−02 | 8.72E−02 |
| physiological_charge_ChemAxon | −0.57 | −0.67 | 5.10E−01 | 5.44E−01 |
| pka_strongest_acidic_ChemAxon | 4.46 | 3.58 | 5.46E−01 | 5.46E−01 |

TABLE 45
Metabolites found in frozen cell samples and missed in
supernatant and FF cell samples categorized by pathway.

| | non-bonded, | | bonded, n | | bonded/ | | |
|---|---|---|---|---|---|---|---|
| Pathway | n (%) | | (%) | | FROZEN, % | p | FDR |
| Transcription/Translation | 24 | (8.4%) | 0 | (0%) | 0.0% | 1.13E−03 | 9.91E−02 |
| Pentose Phosphate Pathway | 1 | (0.3%) | 5 | (5.3%) | 83.3% | 4.22E−03 | 1.86E−01 |
| Glycolysis | 5 | (1.7%) | 6 | (6.3%) | 54.5% | 3.14E−02 | 9.21E−01 |
| Urea Cycle | 12 | (4.2%) | 0 | (0%) | 0.0% | 4.30E−02 | 9.46E−01 |
| Gluconeogenesis | 7 | (2.4%) | 6 | (6.3%) | 46.2% | 9.76E−02 | 1.00E+00 |
| Arginine and Proline Metabolism | 9 | (3.1%) | 0 | (0%) | 0.0% | 1.20E−01 | 1.00E+00 |
| Fructose and Mannose Degradation | 3 | (1%) | 3 | (3.2%) | 50.0% | 1.66E−01 | 1.00E+00 |
| Purine Metabolism | 17 | (5.9%) | 2 | (2.1%) | 10.5% | 1.78E−01 | 1.00E+00 |
| Citric Acid Cycle | 12 | (4.2%) | 1 | (1.1%) | 7.7% | 1.99E−01 | 1.00E+00 |
| Ammonia Recycling | 12 | (4.2%) | 1 | (1.1%) | 7.7% | 1.99E−01 | 1.00E+00 |
| Lactose Synthesis | 7 | (2.4%) | 0 | (0%) | 0.0% | 2.00E−01 | 1.00E+00 |
| Alpha Linolenic Acid and Linoleic | 8 | (2.8%) | 0 | (0%) | 0.0% | 2.09E−01 | 1.00E+00 |
| Acid Metabolism | | | | | | | |
| Pyrimidine Metabolism | 15 | (5.2%) | 2 | (2.1%) | 11.8% | 2.60E−01 | 1.00E+00 |
| Carnitine Synthesis | 10 | (3.5%) | 1 | (1.1%) | 9.1% | 3.05E−01 | 1.00E+00 |
| Phospholipid Biosynthesis | 6 | (2.1%) | 0 | (0%) | 0.0% | 3.43E−01 | 1.00E+00 |
| Beta-Alanine Metabolism | 6 | (2.1%) | 0 | (0%) | 0.0% | 3.43E−01 | 1.00E+00 |
| Aspartate Metabolism | 6 | (2.1%) | 0 | (0%) | 0.0% | 3.43E−01 | 1.00E+00 |
| Glycine and Serine Metabolism | 21 | (7.3%) | 4 | (4.2%) | 16.0% | 3.47E−01 | 1.00E+00 |
| Transfer of Acetyl Groups into | 8 | (2.8%) | 1 | (1.1%) | 11.1% | 4.61E−01 | 1.00E+00 |
| Mitochondria | | | | | | | |
| Methionine Metabolism | 12 | (4.2%) | 2 | (2.1%) | 14.3% | 5.32E−01 | 1.00E+00 |
| Amino Sugar Metabolism | 10 | (3.5%) | 5 | (5.3%) | 33.3% | 5.41E−01 | 1.00E+00 |
| Pyruvate Metabolism | 4 | (1.4%) | 2 | (2.1%) | 33.3% | 6.41E−01 | 1.00E+00 |
| Valine, Leucine and Isoleucine | 7 | (2.4%) | 1 | (1.1%) | 12.5% | 6.85E−01 | 1.00E+00 |
| Degradation | | | | | | | |
| Glutathione Metabolism | 7 | (2.4%) | 1 | (1.1%) | 12.5% | 6.85E−01 | 1.00E+00 |
| Mitochondrial Beta-Oxidation of | 7 | (2.4%) | 1 | (1.1%) | 12.5% | 6.85E−01 | 1.00E+00 |
| Long Chain Saturated Fatty Acids | | | | | | | |
| Galactose Metabolism | 7 | (2.4%) | 1 | (1.1%) | 12.5% | 6.85E−01 | 1.00E+00 |
| Histidine Metabolism | 6 | (2.1%) | 2 | (2.1%) | 25.0% | 1.00E+00 | 1.00E+00 |
| Glycerolipid Metabolism | 7 | (2.4%) | 2 | (2.1%) | 22.2% | 1.00E+00 | 1.00E+00 |
| Betaine Metabolism | 5 | (1.7%) | 1 | (1.1%) | 16.7% | 1.00E+00 | 1.00E+00 |
| Spermidine and Spermine | 5 | (1.7%) | 1 | (1.1%) | 16.7% | 1.00E+00 | 1.00E+00 |
| Biosynthesis | | | | | | | |
| Mitochondrial Beta-Oxidation of | 6 | (2.1%) | 1 | (1.1%) | 14.3% | 1.00E+00 | 1.00E+00 |
| Short Chain Saturated Fatty Acids | | | | | | | |
| Mitochondrial Electron Transport | 5 | (1.7%) | 1 | (1.1%) | 16.7% | 1.00E+00 | 1.00E+00 |
| Chain | | | | | | | |
| Glucose-Alanine Cycle | 6 | (2.1%) | 1 | (1.1%) | 14.3% | 1.00E+00 | 1.00E+00 |
| Glutamate Metabolism | 9 | (3.1%) | 2 | (2.1%) | 18.2% | 1.00E+00 | 1.00E+00 |
| Plasmalogen Synthesis | 5 | (1.7%) | 1 | (1.1%) | 16.7% | 1.00E+00 | 1.00E+00 |

Peptides (78%, P=2.29×10⁻¹⁹; FDR=1.84×10⁻¹⁸) and carbohydrates (47%, P=4.09×10⁻³; FDR=1.09×10⁻²) probably reacted with formaldehyde. Some metabolites with substituents (an atom or group of atoms taking the place of another atom or group or occupying a specific position in a molecule), such as n-substituted-alphaamino acid (73%, P=1.87×10⁻¹⁵; FDR=2.88×10⁻¹³) and carboxamide group (54%, P=1.61×10⁻¹⁴; FDR=1.23×10⁻¹²), were severely affected by the fixation procedure, whereas other classes of metabolites, such as fatty acid ester (0%, P=2.33×10⁻⁸; FDR=8.90×10⁻⁷) and phosphocholine (0%, P=1.13×10⁻³; FDR=1.84×10⁻²), remained intact.

These results confirm the analysis reported in Table 46, Table 47, Table 48, Table 49, Table 50, and Table 51 for the comparison between the metabolites found in formalin-fixated and frozen extracts where it was observed that peptides (22%, P=9.70×10⁻¹⁷; FDR=4.69×10⁻¹⁶) and carbohydrates (53%, P=1.32×10⁻²; FDR=3.51×10⁻²) were poorly detectable after the fixation procedure. Although amino acid concentration could be severely affected when tissues are immersed in an aqueous solution (i.e., formalin), they were still detectable after the fixation procedure.

TABLE 46
Metabolites found in FF cell samples categorized by superclass.

| | non-preserved | | preserved in | | | | |
|---|---|---|---|---|---|---|---|
| | in | | FF, n | | | | |
| Superclass | FF, n (%) | | (%) | | FF/FROZEN, % | p | FDR |
| Peptide | 42 | (35.3%) | 12 | (3.8%) | 22.2% | 9.70E−17 | 4.69E−16 |
| Lipid | 9 | (7.6%) | 151 | (47.5%) | 94.4% | 1.17E−16 | 4.69E−16 |
| Carbohydrate | 15 | (12.6%) | 17 | (5.3%) | 53.1% | 1.32E−02 | 3.51E−02 |
| Nucleotide | 7 | (5.9%) | 35 | (11%) | 83.3% | 1.43E−01 | 2.87E−01 |
| Energy | 0 | (0%) | 7 | (2.2%) | 100.0% | 1.97E−01 | 3.16E−01 |
| Amino Acid | 33 | (27.7%) | 73 | (23%) | 68.9% | 3.17E−01 | 3.78E−01 |
| Cofactors and | 8 | (6.7%) | 14 | (4.4%) | 63.6% | 3.31E−01 | 3.78E−01 |
| Vitamins | | | | | | | |
| Xenobiotics | 5 | (4.2%) | 9 | (2.8%) | 64.3% | 5.42E−01 | 5.42E−01 |

TABLE 47
Metabolites found in FF cell samples categorized by class.

| | non-preserved | | preserved | | | | |
|---|---|---|---|---|---|---|---|
| Class | in FF, n (%) | | in FF, n (%) | | FF/FROZEN, % | p | FDR |
| Peptides | 37 | (36.6%) | 15 | (6%) | 28.8% | 4.00E−12 | 1.00E−10 |
| Glycerophospholipids | 0 | (0%) | 48 | (19.4%) | 100.0% | 5.16E−08 | 6.45E−07 |
| Fatty Acids and Conjugates | 1 | (1%) | 28 | (11.3%) | 96.6% | 9.34E−04 | 7.78E−03 |
| Hydroxy Acids and Derivatives | 5 | (5%) | 3 | (1.2%) | 37.5% | 4.38E−02 | 2.74E−01 |
| Monosaccharides | 7 | (6.9%) | 6 | (2.4%) | 46.2% | 5.57E−02 | 2.78E−01 |
| Glycerolipids | 0 | (0%) | 10 | (4%) | 100.0% | 6.83E−02 | 2.84E−01 |
| Amino Acids and Derivatives | 27 | (26.7%) | 48 | (19.4%) | 64.0% | 1.15E−01 | 4.12E−01 |
| Carboxylic Acids and Derivatives | 0 | (0%) | 6 | (2.4%) | 100.0% | 1.90E−01 | 4.41E−01 |
| Pyrimidine Nucleosides and | 0 | (0%) | 6 | (2.4%) | 100.0% | 1.90E−01 | 4.41E−01 |
| Analogues | | | | | | | |
| Pyrimidine Nucleotides | 1 | (1%) | 11 | (4.4%) | 91.7% | 1.91E−01 | 4.41E−01 |
| Azoles | 2 | (2%) | 1 | (0.4%) | 33.3% | 1.94E−01 | 4.41E−01 |
| Sugar Acids and Derivatives | 4 | (4%) | 4 | (1.6%) | 50.0% | 2.30E−01 | 4.79E−01 |
| Pteridines and Derivatives | 2 | (2%) | 2 | (0.8%) | 50.0% | 3.18E−01 | 5.85E−01 |
| Sphingolipids | 0 | (0%) | 5 | (2%) | 100.0% | 3.27E−01 | 5.85E−01 |
| Purine Nucleotides | 2 | (2%) | 10 | (4%) | 83.3% | 5.22E−01 | 8.06E−01 |
| Pyridines and Derivatives | 0 | (0%) | 3 | (1.2%) | 100.0% | 5.62E−01 | 8.06E−01 |
| Benzyl Alcohols and Derivatives | 0 | (0%) | 4 | (1.6%) | 100.0% | 5.81E−01 | 8.06E−01 |
| Lineolic Acids and Derivatives | 0 | (0%) | 4 | (1.6%) | 100.0% | 5.81E−01 | 8.06E−01 |
| Alkylamines | 2 | (2%) | 3 | (1.2%) | 60.0% | 6.24E−01 | 8.21E−01 |
| Fatty Acid Esters | 4 | (4%) | 13 | (5.2%) | 76.5% | 7.88E−01 | 9.85E−01 |
| Cyclic Alcohols and Derivatives | 1 | (1%) | 3 | (1.2%) | 75.0% | 1.00E+00 | 1.00E+00 |
| Imidazopyrimidines | 1 | (1%) | 4 | (1.6%) | 80.0% | 1.00E+00 | 1.00E+00 |
| Organic Phosphoric Acids and | 1 | (1%) | 2 | (0.8%) | 66.7% | 1.00E+00 | 1.00E+00 |
| Derivatives | | | | | | | |
| Purine Nucleosides and | 3 | (3%) | 7 | (2.8%) | 70.0% | 1.00E+00 | 1.00E+00 |
| Analogues | | | | | | | |
| Sugar Alcohols | 1 | (1%) | 2 | (0.8%) | 66.7% | 1.00E+00 | 1.00E+00 |

TABLE 48
Metabolites found in FF cell samples categorized by subclass.

| | non-preserved | | preserved | | | | |
|---|---|---|---|---|---|---|---|
| Subclass | in FF, n (%) | | in FF, n (%) | | FF/FROZEN, % | p | FDR |
| Peptides | 37 | (41.6%) | 15 | (7.6%) | 28.8% | 4.00E−12 | 1.28E−10 |
| N-acyl-alpha Amino Acids and | 14 | (15.7%) | 8 | (4%) | 36.4% | 3.95E−04 | 6.32E−03 |
| Derivatives | | | | | | | |
| Hexoses | 6 | (6.7%) | 1 | (0.5%) | 14.3% | 2.48E−03 | 2.19E−02 |
| Lysophosphatidylethanolamines | 0 | (0%) | 18 | (9.1%) | 100.0% | 2.73E−03 | 2.19E−02 |
| Unsaturated Fatty Acids | 0 | (0%) | 15 | (7.6%) | 100.0% | 7.95E−03 | 5.09E−02 |
| Lysophosphatidylcholines | 0 | (0%) | 13 | (6.6%) | 100.0% | 2.35E−02 | 1.25E−01 |
| Monoacylglycerols | 0 | (0%) | 10 | (5.1%) | 100.0% | 6.83E−02 | 3.12E−01 |
| Straight Chain Fatty Acids | 0 | (0%) | 8 | (4%) | 100.0% | 1.12E−01 | 4.47E−01 |
| Beta Hydroxy Acids and | 3 | (3.4%) | 2 | (1%) | 40.0% | 1.40E−01 | 4.96E−01 |
| Derivatives | | | | | | | |
| Imidazolyl Carboxylic Acids and | 2 | (2.2%) | 1 | (0.5%) | 33.3% | 1.94E−01 | 5.77E−01 |
| Derivatives | | | | | | | |
| Phosphatidylcholines | 0 | (0%) | 7 | (3.5%) | 100.0% | 1.98E−01 | 5.77E−01 |
| Sugar Acids and Derivatives | 3 | (3.4%) | 3 | (1.5%) | 50.0% | 3.57E−01 | 8.85E−01 |
| Alpha Amino Acids and | 9 | (10.1%) | 32 | (16.2%) | 78.0% | 4.63E−01 | 8.85E−01 |
| Derivatives | | | | | | | |
| Dicarboxylic Acids and | 0 | (0%) | 3 | (1.5%) | 100.0% | 5.62E−01 | 8.85E−01 |
| Derivatives | | | | | | | |
| Phosphatidylinositols | 0 | (0%) | 3 | (1.5%) | 100.0% | 5.62E−01 | 8.85E−01 |
| Purine Ribonucleoside | 0 | (0%) | 3 | (1.5%) | 100.0% | 5.62E−01 | 8.85E−01 |
| Monophosphates | | | | | | | |
| Pyrimidine 2′- | 0 | (0%) | 3 | (1.5%) | 100.0% | 5.62E−01 | 8.85E−01 |
| deoxyribonucleosides and | | | | | | | |
| Analogues | | | | | | | |
| Pyrimidine Nucleosides and | 0 | (0%) | 3 | (1.5%) | 100.0% | 5.62E−01 | 8.85E−01 |
| Analogues | | | | | | | |
| Lineolic Acids and Derivatives | 0 | (0%) | 4 | (2%) | 100.0% | 5.81E−01 | 8.85E−01 |
| Phenylpyruvic Acid Derivatives | 0 | (0%) | 4 | (2%) | 100.0% | 5.81E−01 | 8.85E−01 |
| Pyrimidine Nucleotide Sugars | 0 | (0%) | 4 | (2%) | 100.0% | 5.81E−01 | 8.85E−01 |
| Purine Nucleosides and | 2 | (2.2%) | 4 | (2%) | 66.7% | 6.77E−01 | 9.85E−01 |
| Analogues | | | | | | | |
| Acyl Carnitines | 4 | (4.5%) | 11 | (5.6%) | 73.3% | 1.00E+00 | 1.00E+00 |
| Acyl Glycines | 1 | (1.1%) | 2 | (1%) | 66.7% | 1.00E+00 | 1.00E+00 |
| Beta Amino Acids and | 1 | (1.1%) | 2 | (1%) | 66.7% | 1.00E+00 | 1.00E+00 |
| Derivatives | | | | | | | |
| Branched Fatty Acids | 1 | (1.1%) | 3 | (1.5%) | 75.0% | 1.00E+00 | 1.00E+00 |
| Glycoamino Acids and | 1 | (1.1%) | 2 | (1%) | 66.7% | 1.00E+00 | 1.00E+00 |
| Derivatives | | | | | | | |
| Pentoses | 1 | (1.1%) | 3 | (1.5%) | 75.0% | 1.00E+00 | 1.00E+00 |
| Polyamines | 1 | (1.1%) | 2 | (1%) | 66.7% | 1.00E+00 | 1.00E+00 |
| Purine 2′-deoxyribonucleosides | 1 | (1.1%) | 3 | (1.5%) | 75.0% | 1.00E+00 | 1.00E+00 |
| and Analogues | | | | | | | |
| Purine Ribonucleoside | 1 | (1.1%) | 4 | (2%) | 80.0% | 1.00E+00 | 1.00E+00 |
| Diphosphates | | | | | | | |
| Sugar Alcohols | 1 | (1.1%) | 2 | (1%) | 66.7% | 1.00E+00 | 1.00E+00 |

TABLE 49
Metabolites found in FF cell samples categorized by substituent.

| | non-preserved | | preserved | | | | |
|---|---|---|---|---|---|---|---|
| Substituent | in FF, n (%) | | in FF, n (%) | | FF/FROZEN, % | p | FDR |
| secondary carboxylic acid amide | 55 | (50.9%) | 37 | (13.5%) | 40.2% | 1.53E−13 | 3.51E−11 |
| n-substituted-alpha-amino acid | 38 | (35.2%) | 14 | (5.1%) | 26.9% | 4.26E−13 | 4.88E−11 |
| carboxamide_group | 58 | (53.7%) | 46 | (16.8%) | 44.2% | 2.19E−12 | 1.67E−10 |
| n-acyl-alpha-amino-acid | 36 | (33.3%) | 14 | (5.1%) | 28.0% | 4.90E−12 | 2.81E−10 |
| alpha-amino acid or derivative | 38 | (35.2%) | 17 | (6.2%) | 30.9% | 8.22E−12 | 3.76E−10 |
| fatty acid ester | 0 | (0%) | 58 | (21.2%) | 100.0% | 7.21E−10 | 2.75E−08 |
| carboxylic acid ester | 4 | (3.7%) | 70 | (25.5%) | 94.6% | 1.08E−07 | 3.52E−06 |
| acyclic alkene | 2 | (1.9%) | 60 | (21.9%) | 96.8% | 1.23E−07 | 3.52E−06 |
| carboxylic acid | 80 | (74.1%) | 123 | (44.9%) | 60.6% | 2.33E−07 | 5.93E−06 |
| phosphoethanolamine | 1 | (0.9%) | 48 | (17.5%) | 98.0% | 7.58E−07 | 1.74E−05 |
| primary aliphatic amine | 53 | (49.1%) | 73 | (26.6%) | 57.9% | 3.81E−05 | 7.94E−04 |
| (alkylamine) | | | | | | | |
| amphetamine or derivative | 10 | (9.3%) | 2 | (0.7%) | 16.7% | 9.13E−05 | 1.74E−03 |
| phosphocholine | 0 | (0%) | 24 | (8.8%) | 100.0% | 3.15E−04 | 5.54E−03 |
| saccharide | 6 | (5.6%) | 51 | (18.6%) | 89.5% | 7.55E−04 | 1.24E−02 |
| quaternary ammonium salt | 4 | (3.7%) | 43 | (15.7%) | 91.5% | 8.36E−04 | 1.28E−02 |
| glycero-3-phosphocholine | 0 | (0%) | 20 | (7.3%) | 100.0% | 1.55E−03 | 2.11E−02 |
| phosphoric acid ester | 15 | (13.9%) | 80 | (29.2%) | 84.2% | 1.57E−03 | 2.11E−02 |
| organic hypophosphite | 16 | (14.8%) | 83 | (30.3%) | 83.8% | 1.78E−03 | 2.26E−02 |
| organic phosphite | 16 | (14.8%) | 81 | (29.6%) | 83.5% | 2.63E−03 | 3.13E−02 |
| triose monosaccharide | 0 | (0%) | 18 | (6.6%) | 100.0% | 2.73E−03 | 3.13E−02 |
| beta-hydroxy acid | 12 | (11.1%) | 9 | (3.3%) | 42.9% | 4.85E−03 | 5.29E−02 |
| 1,2-aminoalcohol | 8 | (7.4%) | 5 | (1.8%) | 38.5% | 1.12E−02 | 1.17E−01 |
| hemiacetal | 7 | (6.5%) | 4 | (1.5%) | 36.4% | 1.42E−02 | 1.41E−01 |
| oxane | 9 | (8.3%) | 6 | (2.2%) | 40.0% | 1.49E−02 | 1.42E−01 |
| choline | 5 | (4.6%) | 35 | (12.8%) | 87.5% | 2.45E−02 | 2.16E−01 |
| 1,3-aminoalcohol | 10 | (9.3%) | 9 | (3.3%) | 47.4% | 3.25E−02 | 2.76E−01 |
| n-glycosyl compound | 5 | (4.6%) | 32 | (11.7%) | 86.5% | 3.56E−02 | 2.91E−01 |
| pentose monosaccharide | 4 | (3.7%) | 27 | (9.9%) | 87.1% | 5.97E−02 | 4.71E−01 |
| hydropyrimidine | 2 | (1.9%) | 18 | (6.6%) | 90.0% | 7.45E−02 | 5.69E−01 |
| glycosyl compound | 6 | (5.6%) | 32 | (11.7%) | 84.2% | 8.73E−02 | 6.45E−01 |
| oxolane | 10 | (9.3%) | 42 | (15.3%) | 80.8% | 1.37E−01 | 9.83E−01 |
| allyl alcohol | 0 | (0%) | 6 | (2.2%) | 100.0% | 1.90E−01 | 9.96E−01 |
| pyrimidine | 12 | (11.1%) | 45 | (16.4%) | 78.9% | 2.06E−01 | 9.96E−01 |
| alkylthiol | 4 | (3.7%) | 4 | (1.5%) | 50.0% | 2.30E−01 | 9.96E−01 |
| thiol (sulfanyl compound) | 4 | (3.7%) | 4 | (1.5%) | 50.0% | 2.30E−01 | 9.96E−01 |
| secondary alcohol | 36 | (33.3%) | 110 | (40.1%) | 75.3% | 2.43E−01 | 9.96E−01 |
| 1,2-diol | 19 | (17.6%) | 63 | (23%) | 76.8% | 2.71E−01 | 9.96E−01 |
| thioether | 5 | (4.6%) | 6 | (2.2%) | 54.5% | 3.05E−01 | 1.00E+00 |
| dicarboxylic acid derivative | 20 | (18.5%) | 40 | (14.6%) | 66.7% | 3.52E−01 | 1.00E+00 |
| 1-phosphoribosyl-imidazole | 2 | (1.9%) | 11 | (4%) | 84.6% | 3.66E−01 | 1.00E+00 |
| carboxylic acid salt | 9 | (8.3%) | 16 | (5.8%) | 64.0% | 3.66E−01 | 1.00E+00 |
| purinone | 3 | (2.8%) | 4 | (1.5%) | 57.1% | 4.09E−01 | 1.00E+00 |
| organic pyrophosphate | 3 | (2.8%) | 15 | (5.5%) | 83.3% | 4.21E−01 | 1.00E+00 |
| n-acylglycine | 4 | (3.7%) | 6 | (2.2%) | 60.0% | 4.78E−01 | 1.00E+00 |
| primary alcohol | 21 | (19.4%) | 63 | (23%) | 75.0% | 4.95E−01 | 1.00E+00 |
| primary carboxylic acid amide | 4 | (3.7%) | 7 | (2.6%) | 63.6% | 5.13E−01 | 1.00E+00 |
| pyrimidone | 8 | (7.4%) | 27 | (9.9%) | 77.1% | 5.57E−01 | 1.00E+00 |
| polyamine | 3 | (2.8%) | 12 | (4.4%) | 80.0% | 5.71E−01 | 1.00E+00 |
| monosaccharide phosphate | 5 | (4.6%) | 17 | (6.2%) | 77.3% | 6.34E−01 | 1.00E+00 |
| imidazopyrimidine | 6 | (5.6%) | 21 | (7.7%) | 77.8% | 6.58E−01 | 1.00E+00 |
| purine | 6 | (5.6%) | 21 | (7.7%) | 77.8% | 6.58E−01 | 1.00E+00 |
| urea | 2 | (1.9%) | 4 | (1.5%) | 66.7% | 6.77E−01 | 1.00E+00 |
| pyrrolidine carboxylic acid | 2 | (1.9%) | 4 | (1.5%) | 66.7% | 6.77E−01 | 1.00E+00 |
| succinic_acid | 1 | (0.9%) | 6 | (2.2%) | 85.7% | 6.78E−01 | 1.00E+00 |
| hypoxanthine | 3 | (2.8%) | 5 | (1.8%) | 62.5% | 6.92E−01 | 1.00E+00 |
| imidazole | 9 | (8.3%) | 27 | (9.9%) | 75.0% | 7.03E−01 | 1.00E+00 |
| guanidine | 3 | (2.8%) | 6 | (2.2%) | 66.7% | 7.17E−01 | 1.00E+00 |
| secondary aliphatic amine | 3 | (2.8%) | 6 | (2.2%) | 66.7% | 7.17E−01 | 1.00E+00 |
| (dialkylamine) | | | | | | | |
| alpha-hydroxy acid | 4 | (3.7%) | 9 | (3.3%) | 69.2% | 7.64E−01 | 1.00E+00 |
| ketone | 2 | (1.9%) | 6 | (2.2%) | 75.0% | 1.00E+00 | 1.00E+00 |
| short-chain hydroxy acid | 2 | (1.9%) | 6 | (2.2%) | 75.0% | 1.00E+00 | 1.00E+00 |
| carnitine | 4 | (3.7%) | 11 | (4%) | 73.3% | 1.00E+00 | 1.00E+00 |
| aminopyrimidine | 10 | (9.3%) | 26 | (9.5%) | 72.2% | 1.00E+00 | 1.00E+00 |
| cyclohexane | 2 | (1.9%) | 7 | (2.6%) | 77.8% | 1.00E+00 | 1.00E+00 |
| disaccharide phosphate | 2 | (1.9%) | 7 | (2.6%) | 77.8% | 1.00E+00 | 1.00E+00 |
| pyrrolidine | 2 | (1.9%) | 5 | (1.8%) | 71.4% | 1.00E+00 | 1.00E+00 |

TABLE 50
Metabolites found in FF cell samples categorized by physical/chemical property.

| | non-preserved in | preserved in FF, | | |
|---|---|---|---|---|
| Property | FF, n (%) | n (%) | p | FDR |
| logp_ALOGPS | −1.79 | 0.84 | 5.40E−10 | 8.64E−09 |
| logp_ChemAxon | −2.60 | 0.24 | 1.74E−08 | 1.39E−07 |
| solubility_ALOGPS | 68.16 | 61.20 | 3.51E−07 | 1.87E−06 |
| logs_ALOGPS | −1.25 | −2.91 | 5.54E−07 | 2.22E−06 |
| donor_count_ChemAxon | 3.77 | 2.88 | 2.09E−06 | 6.70E−06 |
| refractivity_ChemAxon | 55.55 | 82.11 | 1.13E−04 | 3.01E−04 |
| polarizability_ChemAxon | 22.23 | 33.25 | 2.83E−04 | 6.46E−04 |
| acceptor_count_ChemAxon | 5.51 | 4.98 | 1.43E−03 | 2.86E−03 |
| rotatable_bond_count_ChemAxon | 5.10 | 10.91 | 2.00E−03 | 3.24E−03 |
| average_mass_ChemAxon | 232.18 | 310.81 | 2.19E−03 | 3.24E−03 |
| mono_mass_ChemAxon | 232.05 | 310.62 | 2.23E−03 | 3.24E−03 |
| polar_surface_area_ChemAxon | 117.02 | 106.62 | 3.94E−03 | 5.25E−03 |
| formal_charge_ChemAxon | −0.03 | 0.01 | 2.31E−02 | 2.85E−02 |
| pka_strongest_basic_ChemAxon | 4.48 | 1.89 | 3.27E−02 | 3.74E−02 |
| physiological_charge_ChemAxon | −0.64 | −0.58 | 5.28E−01 | 5.64E−01 |
| pka_strongest_acidic_ChemAxon | 3.80 | 4.41 | 6.70E−01 | 6.70E−01 |

TABLE 51
Metabolites found in FF cell samples categorized by pathway.

| | non-preserved | | preserved | | | | |
|---|---|---|---|---|---|---|---|
| Pathway | in FF, n (%) | | in FF, n (%) | | FF/FROZEN, % | p | FDR |
| Transcription/Translation | 1 | (0.9%) | 23 | (8.4%) | 95.8% | 4.29E−03 | 3.44E−01 |
| Pentose Phosphate Pathway | 5 | (4.6%) | 1 | (0.4%) | 16.7% | 7.82E−03 | 3.44E−01 |
| Glycolysis | 6 | (5.6%) | 5 | (1.8%) | 45.5% | 8.19E−02 | 1.00E+00 |
| Alpha Linolenic Acid and | 0 | (0%) | 8 | (2.9%) | 100.0% | 1.12E−01 | 1.00E+00 |
| Linoleic Acid Metabolism | | | | | | | |
| Purine Metabolism | 2 | (1.9%) | 17 | (6.2%) | 89.5% | 1.14E−01 | 1.00E+00 |
| Citric Acid Cycle | 1 | (0.9%) | 12 | (4.4%) | 92.3% | 1.21E−01 | 1.00E+00 |
| Ammonia Recycling | 1 | (0.9%) | 12 | (4.4%) | 92.3% | 1.21E−01 | 1.00E+00 |
| Pyrimidine Metabolism | 2 | (1.9%) | 15 | (5.5%) | 88.2% | 1.69E−01 | 1.00E+00 |
| Phospholipid Biosynthesis | 0 | (0%) | 6 | (2.2%) | 100.0% | 1.90E−01 | 1.00E+00 |
| Beta-Alanine Metabolism | 0 | (0%) | 6 | (2.2%) | 100.0% | 1.90E−01 | 1.00E+00 |
| Aspartate Metabolism | 0 | (0%) | 6 | (2.2%) | 100.0% | 1.90E−01 | 1.00E+00 |
| Urea Cycle | 1 | (0.9%) | 11 | (4%) | 91.7% | 1.91E−01 | 1.00E+00 |
| Lactose Synthesis | 0 | (0%) | 7 | (2.6%) | 100.0% | 1.98E−01 | 1.00E+00 |
| Gluconeogenesis | 6 | (5.6%) | 7 | (2.6%) | 53.8% | 2.06E−01 | 1.00E+00 |
| Fructose and Mannose | 3 | (2.8%) | 3 | (1.1%) | 50.0% | 3.57E−01 | 1.00E+00 |
| Degradation | | | | | | | |
| Valine, Leucine and Isoleucine | 1 | (0.9%) | 7 | (2.6%) | 87.5% | 4.50E−01 | 1.00E+00 |
| Degradation | | | | | | | |
| Galactose Metabolism | 1 | (0.9%) | 7 | (2.6%) | 87.5% | 4.50E−01 | 1.00E+00 |
| Arginine and Proline Metabolism | 1 | (0.9%) | 8 | (2.9%) | 88.9% | 4.55E−01 | 1.00E+00 |
| Transfer of Acetyl Groups into | 1 | (0.9%) | 8 | (2.9%) | 88.9% | 4.55E−01 | 1.00E+00 |
| Mitochondria | | | | | | | |
| Glycine and Serine Metabolism | 5 | (4.6%) | 20 | (7.3%) | 80.0% | 4.91E−01 | 1.00E+00 |
| Pyruvate Metabolism | 2 | (1.9%) | 4 | (1.5%) | 66.7% | 6.77E−01 | 1.00E+00 |
| Glucose-Alanine Cycle | 1 | (0.9%) | 6 | (2.2%) | 85.7% | 6.78E−01 | 1.00E+00 |
| Carnitine Synthesis | 2 | (1.9%) | 9 | (3.3%) | 81.8% | 7.35E−01 | 1.00E+00 |
| Glutamate Metabolism | 2 | (1.9%) | 9 | (3.3%) | 81.8% | 7.35E−01 | 1.00E+00 |

Finally, effects of paraffin-embedding on the metabolome were investigated. Metabolites extracted from the samples before and after the paraffin embedding were compared. A global depletion of metabolites in all classes was observed (Table 52, Table 53, Table 54, Table 55, Table 56, and Table 57).

TABLE 52
Metabolites found in FF and missed in FFPE cell samples categorized by superclass.

| | non-preserved | preserved in | | | |
|---|---|---|---|---|---|
| Superclass | in FFPE, n (%) | FFPE, n (%) | FFPE/FF, % | p | FDR |
| Peptide | 8 (8.8%) | 4 (1.8%) | 33.3% | 5.99E−03 | 4.79E−02 |
| Xenobiotics | 5 (5.5%) | 4 (1.8%) | 44.4% | 1.26E−01 | 4.37E−01 |
| Nucleotide | 6 (6.6%) | 29 (12.8%) | 82.9% | 1.64E−01 | 4.37E−01 |
| Carbohydrate | 7 (7.7%) | 10 (4.4%) | 58.8% | 2.72E−01 | 4.84E−01 |
| Amino Acid | 17 (18.7%) | 56 (24.7%) | 76.7% | 3.02E−01 | 4.84E−01 |
| Energy | 1 (1.1%) | 6 (2.6%) | 85.7% | 6.78E−01 | 9.04E−01 |
| Cofactors and Vitamins | 4 (4.4%) | 10 (4.4%) | 71.4% | 1.00E+00 | 1.00E+00 |
| Lipid | 43 (47.3%) | 108 (47.6%) | 71.5% | 1.00E+00 | 1.00E+00 |

TABLE 53
Metabolites found in FF and missed in FFPE cell samples categorized by class.

| | non-preserved in | preserved in | | | |
|---|---|---|---|---|---|
| Class | FFPE, n (%) | FFPE, n (%) | FFPE/FF, % | p | FDR |
| Fatty Acids and Conjugates | 2 (3.2%) | 26 (14.5%) | 92.9% | 1.22E−02 | 2.56E−01 |
| Peptides | 8 (12.9%) | 7 (3.9%) | 46.7% | 3.16E−02 | 3.32E−01 |
| Glycerophospholipids | 19 (30.6%) | 29 (16.2%) | 60.4% | 4.76E−02 | 3.33E−01 |
| Glycerolipids | 0 (0%) | 10 (5.6%) | 100.0% | 6.67E−02 | 3.50E−01 |
| Sphingolipids | 3 (4.8%) | 2 (1.1%) | 40.0% | 1.24E−01 | 5.20E−01 |
| Sugar Acids and Derivatives | 2 (3.2%) | 2 (1.1%) | 50.0% | 2.95E−01 | 7.80E−01 |
| Purine Nucleotides | 1 (1.6%) | 9 (5%) | 90.0% | 2.96E−01 | 7.80E−01 |
| Fatty Acid Esters | 5 (8.1%) | 8 (4.5%) | 61.5% | 3.47E−01 | 7.80E−01 |
| Monosaccharides | 3 (4.8%) | 3 (1.7%) | 50.0% | 3.49E−01 | 7.80E−01 |
| Amino Acids and Derivatives | 10 (16.1%) | 38 (21.2%) | 79.2% | 3.71E−01 | 7.80E−01 |
| Cyclic Alcohols and Derivatives | 0 (0%) | 3 (1.7%) | 100.0% | 5.66E−01 | 8.08E−01 |
| Hydroxy Acids and Derivatives | 0 (0%) | 3 (1.7%) | 100.0% | 5.66E−01 | 8.08E−01 |
| Pyridines and Derivatives | 0 (0%) | 3 (1.7%) | 100.0% | 5.66E−01 | 8.08E−01 |
| Imidazopyrimidines | 0 (0%) | 4 (2.2%) | 100.0% | 5.77E−01 | 8.08E−01 |
| Lineolic Acids and Derivatives | 0 (0%) | 4 (2.2%) | 100.0% | 5.77E−01 | 8.08E−01 |
| Carboxylic Acids and Derivatives | 2 (3.2%) | 4 (2.2%) | 66.7% | 6.63E−01 | 8.38E−01 |
| Purine Nucleosides and | 1 (1.6%) | 6 (3.4%) | 85.7% | 6.78E−01 | 8.38E−01 |
| Analogues | | | | | |
| Alkylamines | 1 (1.6%) | 2 (1.1%) | 66.7% | 1.00E+00 | 1.00E+00 |
| Benzyl Alcohols and Derivatives | 1 (1.6%) | 3 (1.7%) | 75.0% | 1.00E+00 | 1.00E+00 |
| Pyrimidine Nucleosides and | 1 (1.6%) | 5 (2.8%) | 83.3% | 1.00E+00 | 1.00E+00 |
| Analogues | | | | | |
| Pyrimidine Nucleotides | 3 (4.8%) | 8 (4.5%) | 72.7% | 1.00E+00 | 1.00E+00 |

TABLE 54
Metabolites found in FF and missed in FFPE cell samples categorized by subclass.

| | non-preserved in | preserved in | | | |
|---|---|---|---|---|---|
| Subclass | FFPE, n (%) | FFPE, n (%) | FFPE/FF, % | p | FDR |
| Phosphatidylcholines | 7 (15.6%) | 0 (0%) | 0.0% | 8.45E−05 | 2.03E−03 |
| Unsaturated Fatty Acids | 0 (0%) | 15 (10.8%) | 100.0% | 1.36E−02 | 1.63E−01 |
| Peptides | 8 (17.8%) | 7 (5%) | 46.7% | 3.16E−02 | 2.53E−01 |
| Lysophosphatidylcholines | 7 (15.6%) | 6 (4.3%) | 46.2% | 4.80E−02 | 2.67E−01 |
| Alpha Amino Acids and | 4 (8.9%) | 28 (20.1%) | 87.5% | 5.66E−02 | 2.67E−01 |
| Derivatives | | | | | |
| Monoacylglycerols | 0 (0%) | 10 (7.2%) | 100.0% | 6.67E−02 | 2.67E−01 |
| Straight Chain Fatty Acids | 0 (0%) | 8 (5.8%) | 100.0% | 1.13E−01 | 3.87E−01 |
| Pentoses | 2 (4.4%) | 1 (0.7%) | 33.3% | 1.78E−01 | 5.35E−01 |
| Pyrimidine Nucleotide Sugars | 2 (4.4%) | 2 (1.4%) | 50.0% | 2.95E−01 | 7.29E−01 |
| Lysophosphatidylethanolamines | 3 (6.7%) | 15 (10.8%) | 83.3% | 4.15E−01 | 7.29E−01 |
| N-acyl-alpha Amino Acids and | 3 (6.7%) | 5 (3.6%) | 62.5% | 4.49E−01 | 7.29E−01 |
| Derivatives | | | | | |
| Acyl Carnitines | 4 (8.9%) | 7 (5%) | 63.6% | 4.95E−01 | 7.29E−01 |
| Branched Fatty Acids | 0 (0%) | 3 (2.2%) | 100.0% | 5.66E−01 | 7.29E−01 |
| Dicarboxylic Acids and | 0 (0%) | 3 (2.2%) | 100.0% | 5.66E−01 | 7.29E−01 |
| Derivatives | | | | | |
| Phosphatidylinositols | 0 (0%) | 3 (2.2%) | 100.0% | 5.66E−01 | 7.29E−01 |
| Purine 2′-deoxyribonucleosides | 0 (0%) | 3 (2.2%) | 100.0% | 5.66E−01 | 7.29E−01 |
| and Analogues | | | | | |
| Purine Ribonucleoside | 0 (0%) | 3 (2.2%) | 100.0% | 5.66E−01 | 7.29E−01 |
| Monophosphates | | | | | |
| Pyrimidine Nucleosides and | 0 (0%) | 3 (2.2%) | 100.0% | 5.66E−01 | 7.29E−01 |
| Analogues | | | | | |
| Lineolic Acids and Derivatives | 0 (0%) | 4 (2.9%) | 100.0% | 5.77E−01 | 7.29E−01 |
| Phenylpyruvic Acid Derivatives | 1 (2.2%) | 3 (2.2%) | 75.0% | 1.00E+00 | 1.00E+00 |
| Purine Nucleosides and | 1 (2.2%) | 3 (2.2%) | 75.0% | 1.00E+00 | 1.00E+00 |
| Analogues | | | | | |
| Purine Ribonucleoside | 1 (2.2%) | 3 (2.2%) | 75.0% | 1.00E+00 | 1.00E+00 |
| Diphosphates | | | | | |
| Pyrimidine 2′- | | | | | |
| deoxyribonucleosides and | 1 (2.2%) | 2 (1.4%) | 66.7% | 1.00E+00 | 1.00E+00 |
| Analogues | | | | | |
| Sugar Acids and Derivatives | 1 (2.2%) | 2 (1.4%) | 66.7% | 1.00E+00 | 1.00E+00 |

TABLE 55
Metabolites found in FF and missed in FFPE cell samples categorized by substituent.

| | non-preserved in | preserved in FFPE, n | | | |
|---|---|---|---|---|---|
| Substituent | FFPE, n (%) | (%) | FFPE/FF, % | p | FDR |
| quaternary ammonium salt | 25 (33.8%) | 18 (9%) | 41.9% | 2.68E−06 | 6.13E−04 |
| phosphocholine | 16 (21.6%) | 8 (4%) | 33.3% | 2.35E−05 | 2.69E−03 |
| glycero-3-phosphocholine | 14 (18.9%) | 6 (3%) | 30.0% | 3.71E−05 | 2.84E−03 |
| choline | 20 (27%) | 15 (7.5%) | 42.9% | 5.81E−05 | 3.33E−03 |
| dicarboxylic acid derivative | 20 (27%) | 20 (10%) | 50.0% | 8.45E−04 | 3.50E−02 |
| carboxamide_group | 22 (29.7%) | 24 (12%) | 52.2% | 9.18E−04 | 3.50E−02 |
| secondary carboxylic acid amide | 18 (24.3%) | 19 (9.5%) | 51.4% | 2.53E−03 | 8.27E−02 |
| phosphoethanolamine | 20 (27%) | 28 (14%) | 58.3% | 1.90E−02 | 5.43E−01 |
| carboxylic acid salt | 8 (10.8%) | 8 (4%) | 50.0% | 4.29E−02 | 9.56E−01 |
| n-acyl-alpha-amino-acid | 7 (9.5%) | 7 (3.5%) | 50.0% | 6.24E−02 | 9.56E−01 |
| n-substituted-alpha-amino acid | 7 (9.5%) | 7 (3.5%) | 50.0% | 6.24E−02 | 9.56E−01 |
| 1,3-aminoalcohol | 5 (6.8%) | 4 (2%) | 44.4% | 6.30E−02 | 9.56E−01 |
| imidazole | 3 (4.1%) | 24 (12%) | 88.9% | 6.59E−02 | 9.56E−01 |
| aminopyrimidine | 3 (4.1%) | 23 (11.5%) | 88.5% | 6.63E−02 | 9.56E−01 |
| pyrimidine | 7 (9.5%) | 38 (19%) | 84.4% | 6.68E−02 | 9.56E−01 |
| alpha-amino acid or derivative | 8 (10.8%) | 9 (4.5%) | 52.9% | 8.58E−02 | 1.00E+00 |
| carboxylic acid | 27 (36.5%) | 96 (48%) | 78.0% | 1.01E−01 | 1.00E+00 |
| saccharide | 9 (12.2%) | 42 (21%) | 82.4% | 1.16E−01 | 1.00E+00 |
| carboxylic acid ester | 24 (32.4%) | 46 (23%) | 65.7% | 1.21E−01 | 1.00E+00 |
| phosphoric acid ester | 27 (36.5%) | 53 (26.5%) | 66.2% | 1.34E−01 | 1.00E+00 |
| organic phosphite | 27 (36.5%) | 54 (27%) | 66.7% | 1.38E−01 | 1.00E+00 |
| pyrimidone | 4 (5.4%) | 23 (11.5%) | 85.2% | 1.72E−01 | 1.00E+00 |
| organic hypophosphite | 27 (36.5%) | 56 (28%) | 67.5% | 1.85E−01 | 1.00E+00 |
| ketone | 0 (0%) | 6 (3%) | 100.0% | 1.95E−01 | 1.00E+00 |
| guanidine | 0 (0%) | 6 (3%) | 100.0% | 1.95E−01 | 1.00E+00 |
| thioether | 0 (0%) | 6 (3%) | 100.0% | 1.95E−01 | 1.00E+00 |
| imidazopyrimidine | 3 (4.1%) | 18 (9%) | 85.7% | 2.09E−01 | 1.00E+00 |
| purine | 3 (4.1%) | 18 (9%) | 85.7% | 2.09E−01 | 1.00E+00 |
| alpha-hydroxy acid | 4 (5.4%) | 5 (2.5%) | 55.6% | 2.58E−01 | 1.00E+00 |
| 1-phosphoribosyl-imidazole | 1 (1.4%) | 10 (5%) | 90.9% | 2.98E−01 | 1.00E+00 |
| fatty acid ester | 19 (25.7%) | 39 (19.5%) | 67.2% | 3.17E−01 | 1.00E+00 |
| oxane | 3 (4.1%) | 3 (1.5%) | 50.0% | 3.49E−01 | 1.00E+00 |
| allyl alcohol | 3 (4.1%) | 3 (1.5%) | 50.0% | 3.49E−01 | 1.00E+00 |
| succinic_acid | 3 (4.1%) | 3 (1.5%) | 50.0% | 3.49E−01 | 1.00E+00 |
| secondary aliphatic amine | 3 (4.1%) | 3 (1.5%) | 50.0% | 3.49E−01 | 1.00E+00 |
| (dialkylamine) | | | | | |
| primary carboxylic acid amide | 3 (4.1%) | 4 (2%) | 57.1% | 3.92E−01 | 1.00E+00 |
| acyclic alkene | 19 (25.7%) | 41 (20.5%) | 68.3% | 4.11E−01 | 1.00E+00 |
| triose monosaccharide | 3 (4.1%) | 15 (7.5%) | 83.3% | 4.15E−01 | 1.00E+00 |
| carnitine | 4 (5.4%) | 7 (3.5%) | 63.6% | 4.95E−01 | 1.00E+00 |
| polyamine | 2 (2.7%) | 10 (5%) | 83.3% | 5.23E−01 | 1.00E+00 |
| secondary alcohol | 32 (43.2%) | 78 (39%) | 70.9% | 5.79E−01 | 1.00E+00 |
| primary aliphatic amine | 18 (24.3%) | 55 (27.5%) | 75.3% | 6.47E−01 | 1.00E+00 |
| (alkylamine) | | | | | |
| pentose monosaccharide | 6 (8.1%) | 21 (10.5%) | 77.8% | 6.53E−01 | 1.00E+00 |
| short-chain hydroxy acid | 2 (2.7%) | 4 (2%) | 66.7% | 6.63E−01 | 1.00E+00 |
| glycosyl compound | 7 (9.5%) | 25 (12.5%) | 78.1% | 6.72E−01 | 1.00E+00 |
| n-glycosyl compound | 7 (9.5%) | 25 (12.5%) | 78.1% | 6.72E−01 | 1.00E+00 |
| cyclohexane | 1 (1.4%) | 6 (3%) | 85.7% | 6.78E−01 | 1.00E+00 |
| beta-hydroxy acid | 3 (4.1%) | 6 (3%) | 66.7% | 7.06E−01 | 1.00E+00 |
| oxolane | 10 (13.5%) | 32 (16%) | 76.2% | 7.08E−01 | 1.00E+00 |
| primary alcohol | 18 (24.3%) | 45 (22.5%) | 71.4% | 7.49E−01 | 1.00E+00 |
| hydropyrimidine | 4 (5.4%) | 14 (7%) | 77.8% | 7.87E−01 | 1.00E+00 |
| 1,2-diol | 16 (21.6%) | 47 (23.5%) | 74.6% | 8.72E−01 | 1.00E+00 |
| organic pyrophosphate | 4 (5.4%) | 11 (5.5%) | 73.3% | 1.00E+00 | 1.00E+00 |
| monosaccharide phosphate | 4 (5.4%) | 13 (6.5%) | 76.5% | 1.00E+00 | 1.00E+00 |
| disaccharide phosphate | 2 (2.7%) | 5 (2.5%) | 71.4% | 1.00E+00 | 1.00E+00 |
| n-acylglycine | 1 (1.4%) | 5 (2.5%) | 83.3% | 1.00E+00 | 1.00E+00 |

TABLE 56
Metabolites found in FF and missed in FFPE cell samples categorized by
chemical/physical property.

| | non-preserved in | preserved in | | |
|---|---|---|---|---|
| Propriety | FFPE, n (%) | FFPE, n (%) | p | FDR |
| pka_strongest_basic_ChemAxon | 0.36 | 2.53 | 1.07E−02 | 5.34E−02 |
| mono_mass_ChemAxon | 359.37 | 292.58 | 1.39E−02 | 5.34E−02 |
| average_mass_ChemAxon | 359.59 | 292.76 | 1.40E−02 | 5.34E−02 |
| refractivity_ChemAxon | 98.51 | 76.04 | 1.45E−02 | 5.34E−02 |
| polarizability_ChemAxon | 39.54 | 30.93 | 1.67E−02 | 5.34E−02 |
| rotatable_bond_count_ChemAxon | 13.64 | 9.90 | 2.33E−02 | 6.21E−02 |
| logs_ALOGPS | −3.38 | −2.74 | 4.78E−02 | 1.09E−01 |
| solubility_ALOGPS | 55.65 | 63.28 | 7.95E−02 | 1.59E−01 |
| physiological_charge_ChemAxon | −0.45 | −0.63 | 1.17E−01 | 2.08E−01 |
| polar_surface_area_ChemAxon | 108.56 | 105.91 | 1.50E−01 | 2.16E−01 |
| formal_charge_ChemAxon | 0.04 | 0.00 | 1.59E−01 | 2.16E−01 |
| pka_strongest_acidic_ChemAxon | 4.03 | 4.56 | 1.62E−01 | 2.16E−01 |
| donor_count_ChemAxon | 2.61 | 2.99 | 2.66E−01 | 3.27E−01 |
| acceptor_count_ChemAxon | 4.89 | 5.01 | 4.55E−01 | 5.20E−01 |
| logp_ALOGPS | 0.62 | 0.92 | 7.83E−01 | 8.35E−01 |
| logp_ChemAxon | 0.25 | 0.23 | 9.02E−01 | 9.02E−01 |

TABLE 57
Metabolites found in FF and missed in FFPE cell samples categorized by pathway.

| | non-preserved | preserved in | | | |
|---|---|---|---|---|---|
| Pathway | in FFPE, n (%) | FFPE, n (%) | FFPE/FF, % | p | FDR |
| Transcription/Translation | 1 (1.4%) | 22 (11%) | 95.7% | 6.97E−03 | 6.13E−01 |
| Purine Metabolism | 1 (1.4%) | 16 (8%) | 94.1% | 4.80E−02 | 1.00E+00 |
| Lactose Synthesis | 4 (5.4%) | 3 (1.5%) | 42.9% | 8.78E−02 | 1.00E+00 |
| Arginine and Proline Metabolism | 0 (0%) | 8 (4%) | 100.0% | 1.13E−01 | 1.00E+00 |
| Alpha Linolenic Acid and | 0 (0%) | 8 (4%) | 100.0% | 1.13E−01 | 1.00E+00 |
| Linoleic Acid Metabolism | | | | | |
| Glutamate Metabolism | 0 (0%) | 9 (4.5%) | 100.0% | 1.19E−01 | 1.00E+00 |
| Ammonia Recycling | 1 (1.4%) | 11 (5.5%) | 91.7% | 1.90E−01 | 1.00E+00 |
| Valine, Leucine and Isoleucine | 0 (0%) | 7 (3.5%) | 100.0% | 1.95E−01 | 1.00E+00 |
| Degradation | | | | | |
| Glutathione Metabolism | 0 (0%) | 6 (3%) | 100.0% | 1.95E−01 | 1.00E+00 |
| Aspartate Metabolism | 0 (0%) | 6 (3%) | 100.0% | 1.95E−01 | 1.00E+00 |
| Glucose-Alanine Cycle | 0 (0%) | 6 (3%) | 100.0% | 1.95E−01 | 1.00E+00 |
| Glycine and Serine Metabolism | 3 (4.1%) | 17 (8.5%) | 85.0% | 2.97E−01 | 1.00E+00 |
| Urea Cycle | 1 (1.4%) | 10 (5%) | 90.9% | 2.98E−01 | 1.00E+00 |
| Galactose Metabolism | 3 (4.1%) | 4 (2%) | 57.1% | 3.92E−01 | 1.00E+00 |
| Citric Acid Cycle | 2 (2.7%) | 10 (5%) | 83.3% | 5.23E−01 | 1.00E+00 |
| Phospholipid Biosynthesis | 2 (2.7%) | 4 (2%) | 66.7% | 6.63E−01 | 1.00E+00 |
| Gluconeogenesis | 1 (1.4%) | 6 (3%) | 85.7% | 6.78E−01 | 1.00E+00 |
| Transfer of Acetyl Groups into | 1 (1.4%) | 7 (3.5%) | 87.5% | 6.87E−01 | 1.00E+00 |
| Mitochondria | | | | | |
| Carnitine Synthesis | 3 (4.1%) | 6 (3%) | 66.7% | 7.06E−01 | 1.00E+00 |
| Amino Sugar Metabolism | 3 (4.1%) | 7 (3.5%) | 70.0% | 7.33E−01 | 1.00E+00 |
| Methionine Metabolism | 2 (2.7%) | 9 (4.5%) | 81.8% | 7.33E−01 | 1.00E+00 |
| Pyrimidine Metabolism | 3 (4.1%) | 12 (6%) | 80.0% | 7.66E−01 | 1.00E+00 |
| Histidine Metabolism | 1 (1.4%) | 5 (2.5%) | 83.3% | 1.00E+00 | 1.00E+00 |
| Beta-Alanine Metabolism | 1 (1.4%) | 5 (2.5%) | 83.3% | 1.00E+00 | 1.00E+00 |
| Glycerolipid Metabolism | 2 (2.7%) | 5 (2.5%) | 71.4% | 1.00E+00 | 1.00E+00 |
| Mitochondrial Beta-Oxidation of | 1 (1.4%) | 5 (2.5%) | 83.3% | 1.00E+00 | 1.00E+00 |
| Long Chain Saturated Fatty Acids | | | | | |

The major depletion was found for membrane lipids, such as glycerophospholipids (60%, P=4.76×10⁻²; FDR=3.33×10⁻¹). Some metabolites with substituents, such as quaternary ammonium salt (42%, P=2.68×10⁻⁶; FDR=6.13×10⁴) and phosphocholine (33%, P=2.35×10⁻⁵; FDR=2.69×10⁻³), were severely affected by the paraffin-embedding procedure.

The relative susceptibility of each class of metabolites to each factor described above (solubility in formalin, the covalent bonding to cellular component, and solubility in ethanol and xylene) is summarized in Table 58.

TABLE 58
Metabolite score values of reliability

| Diluition in formalin solution | Reactivity to formaldehyde | Dilution in ethanol/xylene |
|---|---|---|
| Subclass | Superclass | Class |
| Alpha Amino Acids nd Derivatives | Carbohydrate | Glycerophospholipids |
| Branched Fatty Acids | Peptides | Sphingolipids |
| Dicarboxylic Acids and Derivatives | Substituent | Subclass |
| Beta Hydroxy Acids and Derivatives | n-substituted-alpha-amino acid | Phosphatidylcholine |
| | | Sphingomyelins |
| | | Phosphatidylserine |

Taking this into account, a score to rank the reliability of each metabolite on the basis of sensitivity to each factor and to highlight the most stable metabolites during the procedure of formalin fixation and paraffin-embedding was defined. To each metabolite was assigned a score to rank the reliability of its concentration value in extract from FFPE samples. This score ranges from 0 to 3, and it is defined as the sum of the 3 parts. Each part is equal to 1 if the metabolite belongs at the least to one of the selected classes listed in Table 46, otherwise is counted as 0. The basal set of metabolites, that is unchanged despite tissue processing, is represented by the metabolites ranked with a score equal to 0.
1. Priolo C, Pyne S, Rose J, Regan E R, Zadra G, Photopoulos C, et al. AKT1 and MYC Induce Distinctive Metabolic Fingerprints in Human Prostate Cancer. Cancer research. 2014.
2. Cacciatore S, Hu X, Viertler C, Kap M, Bernhardt G A, Mischinger H J, et al. Effects of intra- and post-operative ischemia on the metabolic profile of clinical liver tissue specimens monitored by NMR. Journal of proteome research. 2013; 12:5723-9.
3. Aimetti M, Cacciatore S, Graziano A, Tenori L. Metabonomic analysis of saliva reveals generalized chronic periodontitis signature. Metabolomics: Official journal of the Metabolomic Society. 2012; 8:465-74.
4. Bertini I, Cacciatore S, Jensen B V, Schou J V, Johansen J S, Kruhoffer M, et al. Metabolomic NMR fingerprinting to identify and predict survival of patients with metastatic colorectal cancer. Cancer research. 2012; 72:356-64.
5. Cacciatore S, Tenori L. Brain cholesterol homeostasis in Wilson disease. Medical hypotheses. 2013; 81:1127-9.
6. Maclntyre D A, Jimenez B, Lewintre E J, Martin C R, Schafer H, Ballesteros C G, et al. Serum metabolome analysis by 1H-NMR reveals differences between chronic lymphocytic leukaemia molecular subgroups. Leukemia. 2010; 24:788-97.
7. Maccaferri S, Klinder A, Cacciatore S, Chitarrari R, Honda H, Luchinat C, et al. In vitro fermentation of potential prebiotic flours from natural sources: impact on the human colonic microbiota and metabolome. Molecular nutrition & food research. 2012; 56:1342-52.
8. Cacciatore S, Loda M. Innovation in Metabolomics to Improve Personalized Healthcare. Ann N Y Acad Sci. 2015; 1346:57-62.
9. Kelly A D, Breitkopf S B, Yuan M, Goldsmith J, Spentzos D, Asara J M. Metabolomic profiling from formalin-fixed, paraffin-embedded tumor tissue using targeted LC/MS/MS: application in sarcoma. PloS one. 2011; 6:e25357.
10. Wojakowska A, Marczak L, Jelonek K, Polanski K, Widlak P, Pietrowska M. An Optimized Method of Metabolite Extraction from Formalin-Fixed Paraffin-Embedded Tissue for G C/M S Analysis. PloS one. 2015; 10:e0136902.
11. Buck A, Ly A, Balluff B, Sun N, Gorzolka K, Feuchtinger A, et al. High-resolution MALDI-FT-ICR MS imaging for the analysis of metabolites from formalin-fixed, paraffin-embedded clinical tissue samples. The Journal of pathology. 2015; 237:123-32.
12. Yuan M, Breitkopf S B, Yang X, Asara J M. A positive/negative ion-switching, targeted mass spectrometry-based metabolomics platform for bodily fluids, cells, and fresh and fixed tissue. Nature protocols. 2012; 7:872-81.
13. Evans A M, DeHaven C D, Barrett T, Mitchell M, Milgram E. Integrated, nontargeted ultrahigh performance liquid chromatography/electrospray ionization tandem mass spectrometry platform for the identification and relative quantification of the small-molecule complement of biological systems. Anal Chem. 2009; 81:6656-67.
14. Storey J D. A direct approach to false discovery rates. J R Stat Soc [Ser B]. 2002; 64:479-98.
15. Wold S, Antti H, Lindgren F, Ohman J. Orthogonal signal correction of near-infrared spectra. Chem Intell Lab Sys. 1998; 44:175-85.
16. Cacciatore S, Luchinat C, Tenori L. Knowledge discovery by accuracy maximization. Proceedings of the National Academy of Sciences of the United States of America. 2014; 111:5117-22.
17. Ihaka R, Gentleman R. R: a language for data analysis and graphics. J Comput Graph Stat. 1996; 5:299-314.
18. Kaushik A K, Vareed S K, Basu S, Putluri V, Putluri N, Panzitt K, et al. Metabolomic profiling identifies biochemical pathways associated with castration-resistant prostate cancer. Journal of proteome research. 2014; 13:1088-100.
19. Putluri N, Shojaie A, Vasu V T, Nalluri S, Vareed S K, Putluri V, et al. Metabolomic profiling reveals a role for androgen in activating amino acid metabolism and methylation in prostate cancer cells. PloS one. 2011; 6:e21417.

### EQUIVALENTS AND SCOPE

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents, and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that embodiments described in this document using an open-ended transitional phrase (e.g., “comprising”) are also contemplated, in alternative embodiments, as “consisting of” and “consisting essentially of” the feature described by the open-ended transitional phrase. For example, if the disclosure describes “a composition comprising A and B,” the disclosure also contemplates the alternative embodiments “a composition consisting of A and B” and “a composition consisting essentially of A and B.”

What is claimed is:

1. A method to evaluate a biological sample, the method comprising:
obtaining a formalin-fixed paraffin-embedded (FFPE) preparation of the biological sample; and
detecting the presence of one or more metabolites in the FFPE preparation, wherein the one or more metabolites are members of a class selected from the classes listed in Table 1.

2. A method to evaluate a biological sample, the method comprising:
obtaining a formalin-fixed paraffin-embedded (FFPE) preparation of the biological sample; and
detecting the presence of one or more metabolites in the FFPE preparation, wherein the one or more metabolites are members of a subclass selected from the subclasses listed in Table 1.

3. A method to evaluate a biological sample, the method comprising:
obtaining a formalin-fixed paraffin-embedded (FFPE) preparation of the biological sample; and
detecting the presence of one or more metabolites in the FFPE preparation, wherein the one or more metabolites comprise a substituent group selected from the substituents listed in Table 1.

4. The method of any one of claims 1-3, wherein the one or more metabolites are lipids.

5. The method of any one of claims 1-A3, wherein the one or more metabolites are unsaturated fatty acids.

6. The method of any one of claims 1-3, wherein the one or more metabolites are hydrophobic metabolites.

7. The method of any one of claims 1-3, wherein the one or more metabolites are selected from taurine, 1-palmitoylglycerophosphoinositol, pyroglutamine, oxidized glutathione, dihomo-linoleate, creatinine, 1-linoleoylglycerophosphoethanolamine, eicosenoate, and 10-nonadecenoate.

8. The method of any one of claims 1-3, wherein the one or more metabolites do not include one or more metabolites that are members of a class listed in Table 2.

9. The method of any one of claims 1-3, wherein the one or more metabolites do not include one or more metabolites that are members of a subclass listed in Table 2.

10. The method of any one of claims 1-3, wherein the one or more metabolites are not peptides.

11. The method of any one of claims 1-3, wherein the one or more metabolites are not steroids.

12. The method of any one of claims 1-3, wherein the presence of 2 or more metabolites are detected in the FFPE preparation.

13. The method of any one of claims 1-3, wherein the presence of 5 or more metabolites are detected in the FFPE preparation.

14. The method of any one of claims 1-3, wherein the presence of 10 or more metabolites are detected in the FFPE preparation.

15. The method of any one of claims 1-3, wherein the presence of 25 or more metabolites are detected in the FFPE preparation.

16. The method of any one of claims 1-3, further comprising measuring an expression level of the one or more metabolites in the FFPE preparation.

17. The method of claim 16, further comprising comparing the expression level of the one or more metabolites measured in the FFPE preparation to an expression level of the one or more metabolites measured in a control sample.

18. The method of claim 17, wherein the one or more metabolites are selected from the metabolites listed in Table 3.

19. The method of claim 17, wherein the FFPE preparation and the control sample are biological samples of the same subject.

20. The method of claim 17, wherein the FFPE preparation and the control sample are biological samples of different subjects.

21. The method of claim 17, wherein the control sample is a biological sample of non-cancerous tissue.

22. The method of claim 21, further comprising identifying the FFPE preparation as comprising cancerous tissue when the one or more metabolites are differentially expressed in the FFPE preparation when compared to the control sample.

23. The method of claim 17, wherein the control sample is a biological sample of cancerous tissue.

24. The method of claim 23, further comprising identifying the FFPE preparation as not comprising cancerous tissue when the one or more metabolites are differentially expressed in the FFPE preparation when compared to the control sample.

25. The method of claim 22 or 24, wherein the one or more differentially expressed metabolites are selected using a criteria of p-value <0.05.

26. The method of claim 22 or 24, wherein the one or more differentially expressed metabolites are selected using a criteria of false discovery rate <0.1.

27. The method of any one of claims 21-24, further comprising determining tumor status of the biological sample based on the measuring.

28. The method of any one of claims 1-27, wherein the biological sample is a tissue sample.

29. The method of claim 28, wherein the tissue sample is a prostate tissue sample.

30. The method of any one of the preceding claims, further comprising extracting the one or more metabolites from the FFPE biological sample.

31. The method of claim 30, wherein the one or more metabolites are extracted using a methanol solution.

32. The method of claim 31, wherein the methanol solution comprises 80% methanol.

33. The method of any one of the preceding claims, further comprising staining the FFPE biological sample for histological analysis.

34. The method of claim 33, wherein the FFPE biological sample is stained using H&E stain.

35. The method of any one of the preceding claims, further comprising measuring the one or more metabolites in two or more portions of the FFPE preparation of the biological sample.

36. The method of any one of the preceding claims, where the FFPE preparation is mounted on a slide.

37. The method of claim 36, wherein the extracting the one or more metabolites from the FFPE biological sample comprises extracting the one or more metabolites when the slide in situated in a cassette.

38. The method of claim 37, wherein the cassette has the design depicted in FIG. 6.

39. A cassette for metabolite extraction comprising:
a housing including a chamber with an opening;
one or more restraints extending at least partially across a width of the cassette within the chamber, wherein the one or more restraints extends along at least a portion of a length of the chamber between the opening and an opposing interior surface of the chamber, and wherein the one or more restraints retain a slide against a first side of the chamber and spaced from a second opposing side of the chamber when the slide is positioned within the chamber.
